(19) United States
(12) Patent Application Publication
(10) Pub. No.: US 2026/0001877 A1
(43) Pub. Date: Jan. 1, 2026

(54) HETEROARYL COMPOUNDS FOR THE TREATMENT OF PAIN

(71) Applicant: Vertex Pharmaceuticals Incorporated, Boston, MA (US)

(72) Inventors: Elizabeth BECK, Abingdon (GB); Ewa Iwona CHUDYK, Berlin (DE); Thomas CLEVELAND, San Marcos, CA (US); Senait G. GHIRMAI, San Diego, CA (US); Sara Sabina HADIDA RUAH, La Jolla, CA (US); Dennis James HURLEY, San Marcos, CA (US); Mark Thomas MILLER, Rancho Santa Fe, CA (US); Timothy Donald NEUBERT, San Diego, CA (US); Prasuna PARASELLI, San Diego, CA (US); Urvi PATEL, San Diego, CA (US); Joanne Louise PINDER, Goring (GB); Johnny UY, San Diego, CA (US); Roman VALIULIN, San Diego, CA (US); Peter WEBB, San Diego, CA (US); Jinglan ZHOU, San Diego, CA (US)

(73) Assignee: Vertex Pharmaceuticals Incorporated, Boston, MA (US)

(21) Appl. No.: 19/252,181

(22) Filed: Jun. 27, 2025

Related U.S. Application Data
(60) Provisional application No. 63/665,695, filed on Jun. 28, 2024.
(60) Provisional application No. 63/748,757, filed on Jan. 23, 2025.

(51) Int. Cl. C07D 471/04; A61K 31/4375; A61K 31/444; C07D 519/00

(52) U.S. Cl. CPC C07D 471/04; A61K 31/4375; A61K 31/444; C07D 519/00

(57) ABSTRACT

Compounds, and pharmaceutically acceptable salts thereof, useful as inhibitors of sodium channels are provided. Also provided are pharmaceutical compositions comprising the compounds or pharmaceutically acceptable salts and methods of using the compounds, pharmaceutically acceptable salts, and pharmaceutical compositions in the treatment of various disorders, including pain.

### CROSS-REFERENCE TO RELATED APPLICATIONS

This application claims the benefit of U.S. Provisional Application No. 63/665,695, filed Jun. 28, 2024, and U.S. Provisional Application No. 63/748,757, filed Jan. 23, 2025, each of which are incorporated by reference herein in their entirety.

### BACKGROUND

Pain is a protective mechanism to avoid tissue damage and to prevent further damage to injured tissue. Nonetheless, there are many conditions where pain persists beyond its usefulness, or where patients would benefit from inhibition of pain. Acute and chronic pain are two common pain states and can be distinguished by the duration of the pain. Acute pain can arise for many reasons (e.g., a hospital procedure) and treatment options are generally limited by poor efficacy and/or adverse events. Like acute pain, chronic pain can arise for many reasons and treatment options are limited by poor efficacy and/or adverse events.

Neuropathic pain is a form of chronic pain caused by an injury to the sensory nerves (Dieleman, J. P., et al., Incidence rates and treatment of neuropathic pain conditions in the general population. Pain, 2008. 137(3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain.

Voltage-gated sodium channels (Na_{V}s) are involved in pain signaling. Na_{V}s are biological mediators of electrical signaling as they mediate the rapid upstroke of the action potential of many excitable cell types, for example, neurons, skeletal myocytes, cardiac myocytes (Hille, Bertil, Ion Channels of Excitable Membranes, Third ed. (Sinauer Associates, Inc., Sunderland, MA, 2001)). The evidence for the role of these channels in normal physiology, the pathological states arising from mutations in sodium channel genes, preclinical work in animal models, and the clinical pharmacology of known sodium channel modulating agents all point to the central role of Na_{V}s in pain sensation (Rush, A. M. and T. R. Cummins, Painful Research: Identification of a Small-Molecule Inhibitor that Selectively Targets Na_{V}1.8 Sodium Channels. Mol. Interv., 2007. 7(4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Because of the role Na_{V}s play in the initiation and propagation of neuronal signals, antagonists that reduce Nav currents can prevent or reduce neural signaling and Nav channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Nav channels. The local anesthetic drugs such as lidocaine block pain by inhibiting Nav channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Na_{V}s form a subfamily of the voltage-gated ion channel super-family and comprises 9 isoforms, designated Na_{V}1.1-Na_{V}1.9. The tissue localizations of the nine isoforms vary. Na_{V}1.4 is the primary sodium channel of skeletal muscle, and Na_{V}1.5 is primary sodium channel of cardiac myocytes. Na_{V}s 1.7, 1.8 and 1.9 are primarily localized to the peripheral nervous system, while Na_{V}s 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Na_{V}1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379(6562): p. 257-62). Since then, Na_{V}1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Na_{V}1.8 is involved in spontaneous firing in damaged neurons, like those that drive neuropathic pain (Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6; Jarvis, M. F., et al., A-803467, a potent and selective Na_{V}1.8 sodium channel blocker, attenuates neuropathic and inflammatory pain in the rat. Proc. Natl. Acad. Sci. USA, 2007. 104(20): p. 8520-5; Joshi, S. K., et al., Involvement of the TTX-resistant sodium channel Na_{V}1.8 in inflammatory and neuropathic, but not post-operative, pain states. Pain, 2006. 123(1-2): pp. 75-82; Lai, J., et al., Inhibition of neuropathic pain by decreased expression of the tetrodotoxin-resistant sodium channel, Na_{V}1.8. Pain, 2002. 95(1-2): p. 143-52; Dong, X. W., et al., Small interfering RNA-mediated selective knockdown of Na_{V}1.8 tetrodotoxin-resistant sodium channel reverses mechanical allodynia in neuropathic rats. Neuroscience, 2007. 146(2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64(6): p. 644-53; Coward, K., et al., Immunolocalization of SNS/PN3 and NaN/SNS2 sodium channels in human pain states. Pain, 2000. 85(1-2): p. 41-50; Yiangou, Y., et al., SNS/PN3 and SNS2/NaN sodium channel-like immunoreactivity in human adult and neonate injured sensory nerves. FEBS Lett., 2000. 467(2-3): p. 249-52; Ruangsri, S., et al., Relationship of axonal voltage-gated sodium channel 1.8 (Na_{V}1.8) mRNA accumulation to sciatic nerve injury-induced painful neuropathy in rats. J. Biol. Chem. 286(46): p. 39836-47). The small DRG neurons where Na_{V}1.8 is expressed include the nociceptors involved in pain signaling, where Na_{V}1.8 mediates large amplitude action potentials (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Na_{V}1.8 is necessary for rapid repetitive action potentials in nociceptors, and for spontaneous activity of damaged neurons. (Choi, J. S. and S. G. Waxman, Physiological interactions between Na_{V}1.7 and Na_{V}1.8 sodium channels: a computer simulation study. J. Neurophysiol. 106(6): p. 3173-84; Renganathan, M., T. R. Cummins, and S. G. Waxman, Contribution of Na(_{V})1.8 sodium channels to action potential electrogenesis in DRG neurons. J. Neurophysiol., 2001. 86(2): p. 629-40; Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6). In depolarized or damaged DRG neurons, Na_{V}1.8 appears to be a driver of hyper-excitablility (Rush, A. M., et al., A single sodium channel mutation produces hyper- or hypoexcitability in different types of neurons. Proc. Natl. Acad. Sci. USA, 2006. 103(21): p. 8245-50). In some animal pain models, Na_{V}1.8 mRNA expression levels have been shown to increase in the DRG (Sun, W., et al., Reduced conduction failure of the main axon of polymodal nociceptive C-fibers contributes to painful diabetic neuropathy in rats. Brain, 135(Pt 2): p. 359-75; Strickland, I. T., et al., Changes in the expression of Na_{V}1.7, Na_{V}1.8 and Na_{V}1.9 in a distinct population of dorsal root ganglia innervating the rat knee joint in a model of chronic inflammatory joint pain. Eur. J Pain, 2008. 12(5): p. 564-72; Qiu, F., et al., Increased expression of tetrodotoxin-resistant sodium channels Na_{V}1.8 and Na_{V}1.9 within dorsal root ganglia in a rat model of bone cancer pain. Neurosci. Lett., 512(2): p. 61-6).

The inventors have discovered that some voltage-gated sodium channel inhibitors have limitations as therapeutic agents due to, for example, a poor therapeutic window (e.g., due to a lack of Nav isoform selectivity, low potency, and/or other reasons). Accordingly, there remains a need to develop selective voltage-gated sodium channel inhibitors, such as selective Na_{V}1.8 inhibitors.

### SUMMARY

In one aspect, the invention relates to a compound described herein (e.g., a compound of Formula (I), (II), (II-A), (II-B3), (II-B3-1), (II-B3-2), (II-C), (II-D), (II-E), (II-F), (III), (III-A-1), (III-A-2), or (III-A-3), or a compound of any one of Tables A, B, and C), or a pharmaceutically acceptable salt thereof.

In another aspect, the invention relates to a pharmaceutical composition comprising the compound, or a pharmaceutically acceptable salt thereof, and one or more pharmaceutically acceptable carriers or vehicles.

In still another aspect, the invention relates to a method of inhibiting a voltage gated sodium channel in a subject by administering the compound, pharmaceutically acceptable salt, or pharmaceutical composition to the subject.

In yet another aspect, the invention relates to a method of treating or lessening the severity in a subject of a variety of diseases, disorders, or conditions, including, but not limited to, chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, and cardiac arrhythmia, by administering the compound, pharmaceutically acceptable salt, or pharmaceutical composition to the subject.

### DETAILED DESCRIPTION

In one aspect, the invention relates to a compound of formula (I):

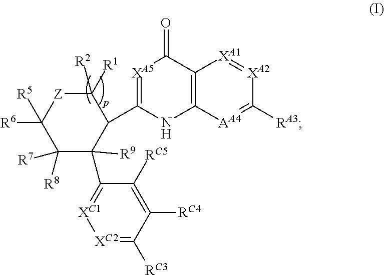

or a pharmaceutically acceptable salt thereof, wherein:
X^{A1} is C—R^{A1}, N, or N⁺—O⁻;
X^{A2} is C—R^{A2}, N, or N⁺—O⁻;
X^{A4} is C—R^{A4}, N, or N⁺—O⁻;
X^{A5} is C—R^{A5} or N;
X^{C1} is C—R^{C1} or N;
X^{C2} is C—R^{C2} or N;
R^{A1}, R^{A2}, R^{A3}, and R^{A4} are each independently H, OH, CN, halo, C₁-C₆ alkyl, C₁-C₆ alkoxy, C₁-C₆ haloalkyl, —NHS(O)₂(C₁-C₆ alkyl), —C(O)OR^{b}, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{A5} is H, OH, halo, CN, C₁-C₆ alkyl, C₁-C₆ haloalkyl, C₁-C₆ alkoxy, C₁-C₆ haloalkoxy, —(CH₂)ₘOₙ(CH₂)ₒOCH₃, —(CH₂)ₘR^{a}, —C(O)(CH₂)ₘR^{a}, —C(O)OR^{b}, —C(O)R^{b}, —C(O)NR^{b}R^{c}, —NR^{b}R^{c}, —CR^{d}R^{e}R^{f}, —CR^{b}R^{c}NR^{g}C(O)CR^{h}RⁱR^{j}, —NR^{b}C(O)CR^{c}R^{g}R^{j}, C₃-C₆ cycloalkyl, 5-6 membered heteroaryl, or 4-10 membered heterocyclyl, wherein said C₃-C₆ cycloalkyl, 5-6 membered heteroaryl, or 4-10 membered heterocyclyl may be unsubstituted or may be substituted with 1-4 substituents independently selected from halo, C₁-C₆ alkyl, C₁-C₆ haloalkyl, C₁-C₆ alkoxy, and C₁-C₆ haloalkoxy;
R¹ and R² are each independently H, halo, OH, C₁-C₆ alkyl, C₁-C₆ haloalkyl, C₃-C₆ cycloalkyl, or C₁-C₆ alkoxy;
R³ and R⁴ are each independently H, halo, OH, C₁-C₆ alkyl, C₁-C₆ haloalkyl, C₃-C₆ cycloalkyl, or C₁-C₆ alkoxy;
p, Z, R⁵, R⁶, R⁷, and R⁸ are defined as follows:
   (i) p is 0 or 1;
      Z is C(—R³)(—R⁴);
      R⁵ and R⁶ are each independently H, halo, OH, C₁-C₆ alkyl, C₁-C₆ haloalkyl, C₃-C₆ cycloalkyl, or C₁-C₆ alkoxy; and
   R⁷ and R⁸ are each independently H, halo, OH, C₁-C₆ alkyl, C₁-C₆ haloalkyl, C₃-C₆ cycloalkyl, or C₁-C₆ alkoxy; or
   (ii) p is 0 or 1;
      Z is C(—R³)(—R⁴);
      R⁷ and R⁸ are each independently H, halo, OH, C₁-C₆ alkyl, C₁-C₆ haloalkyl, C₃-C₆ cycloalkyl, or C₁-C₆ alkoxy; and
      R⁵ and R⁶, together with the carbon atom to which they are attached, form a C₃-C₆ cycloalkyl optionally substituted with 1-6 R^{y}; or
   (iii) p is 0 or 1;
      Z is C(−R³)(−R⁴);
      R⁵ and R⁷, together with the carbon atoms to which they are attached, form a fused C₃-C₆ cycloalkyl optionally substituted with 1-6 R^{y}; and
      R⁶ and R⁸ are each independently H, halo, OH, C₁-C₆ alkyl, C₁-C₆ haloalkyl, C₃-C₆ cycloalkyl, or C₁-C₆ alkoxy; or
   (iv) p is 0;
      Z is C(—R³)(—R⁴) or 0;
      R⁵ and R⁷, together with the carbon atoms to which they are attached, form a fused ring of formula

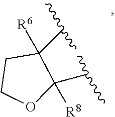

optionally substituted with 1-4 R^{y}; and
R⁶ and R⁸ are each independently H, halo, OH, C₁-C₆ alkyl, C₁-C₆ haloalkyl, C₃-C₆ cycloalkyl, or C₁-C₆ alkoxy;
R⁹ is H or CH₃;
R^{C1} and R^{C2} are each independently H, OH, halo, C₁-C₆ alkyl, C₁-C₆ alkoxy, C₁-C₆ haloalkyl, C₁-C₆ haloalkoxy, —[CH₂]_{q}—C₃-C₆ cycloalkyl, —[CH₂]_{q}—O—C₃-C₆ cycloalkyl, —[CH₂]_{q}—NR^{b}R^{c}, —(CH₂)ₘOR, or —O(CH₂)ₘOR^{b}, wherein said C₁-C₆ alkyl and C₁-C₆ alkoxy of R^{C1} and R^{C2} are each optionally substituted with an independently selected CN or 5-6 membered heteroaryl, or R^{C1} and R^{C2}, together with the carbon atoms to which they are attached, form a ring of formula:

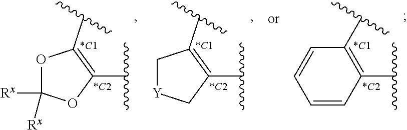

Y is O or CH₂;
R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
each R^{x} is independently H, halo, C₁-C₆ alkyl, or C₁-C₆ haloalkyl;
each R^{y} is independently halo, OH, C₁-C₆ alkyl, or C₁-C₆ haloalkyl;
R^{a} is OH, halo, C₁-C₆ alkoxy, or —NR^{b}R^{c};
R^{b}, R^{c}, R^{g}, R^{h}, and Rⁱ are each independently H or C₁-C₆ alkyl, or R^{b} and R^{c}, together with the nitrogen atom to which they are attached, form a 3-6 membered heterocyclyl;
R^{d}, R^{e}, and R^{f} are each independently H, OH, halo, C₁-C₆ alkyl, or C₁-C₆ alkoxy;
R^{j} is H, C₁-C₆ alkyl, —NR^{b}R^{c}, or —N(CH₃)₃⁺;
m and o are each independently 0, 1, 2, or 3;
q is 0 or 1; and
n is 0 or 1.

In some embodiments, the invention relates to a compound of formula (I), or a pharmaceutically acceptable salt thereof, wherein:
p is 0 or 1;
Z is C(—R³)(—R⁴);
R⁵, R⁶, R⁷, and R⁸ are defined as follows:
   (i) R⁵ and R⁶ are each independently H, halo, OH, C₁-C₆ alkyl, C₁-C₆ haloalkyl, C₃-C₆ cycloalkyl, or C₁-C₆ alkoxy; and
      R⁷ and R⁸ are each independently H, halo, OH, C₁-C₆ alkyl, C₁-C₆ haloalkyl, C₃-C₆ cycloalkyl, or C₁-C₆ alkoxy; or
   (ii) R⁷ and R⁸ are each independently H, halo, OH, C₁-C₆ alkyl, C₁-C₆ haloalkyl, C₃-C₆ cycloalkyl, or C₁-C₆ alkoxy; and
      R⁵ and R⁶, together with the carbon atom to which they are attached, form a C₃-C₆ cycloalkyl optionally substituted with 1-6 R^{y}; or
   (iii) R⁵ and R⁶, together with the carbon atoms to which they are attached, form a fused C₃-C₆ cycloalkyl optionally substituted with 1-6 R^{y}; and
      R⁶ and R⁸ are each independently H, halo, OH, C₁-C₆ alkyl, C₁-C₆ haloalkyl, C₃-C₆ cycloalkyl, or C₁-C₆ alkoxy.

In some embodiments, the invention relates to a compound of formula (I), or a pharmaceutically acceptable salt thereof, wherein:
p is 0 or 1;
Z is C(—R³)(—R⁴);
R⁵, R⁶, R⁷, and R⁸ are defined as follows:
   (i) R⁵ and R⁶ are each independently H, halo, OH, C₁-C₆ alkyl, C₁-C₆ haloalkyl, C₃-C₆ cycloalkyl, or C₁-C₆ alkoxy; and
      R⁷ and R⁸ are each independently H, halo, OH, C₁-C₆ alkyl, C₁-C₆ haloalkyl, C₃-C₆ cycloalkyl, or C₁-C₆ alkoxy; or
   (ii) R⁷ and R⁸ are each independently H, halo, OH, C₁-C₆ alkyl, C₁-C₆ haloalkyl, C₃-C₆ cycloalkyl, or C₁-C₆ alkoxy; and
      R⁵ and R⁶, together with the carbon atom to which they are attached, form a C₃-C₆ cycloalkyl optionally substituted with 1-6 R^{y}; or
   (iii) R⁵ and R⁷, together with the carbon atoms to which they are attached, form a fused C₃-C₆ cycloalkyl optionally substituted with 1-6 R^{y}; and
      R⁶ and R⁸ are each independently H, halo, OH, C₁-C₆ alkyl, C₁-C₆ haloalkyl, C₃-C₆ cycloalkyl, or C₁-C₆ alkoxy; and
R^{C1} and R^{C2} are each independently H, OH, halo, C₁-C₆ alkyl, C₁-C₆ alkoxy, C₁-C₆ haloalkyl, C₁-C₆ haloalkoxy, —[CH₂]_{q}—C₃-C₆ cycloalkyl, —[CH₂]_{q}—O—C₃-C₆ cycloalkyl, —[CH₂]_{q}—NR^{b}R^{c}, —(CH₂)ₘOR^{b}, or —O(CH₂)ₘOCH₃, or R^{C1} and R^{C2}, together with the carbon atoms to which they are attached, form a ring of formula:

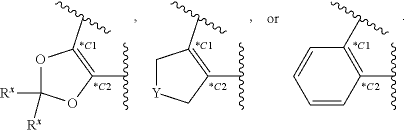

In some embodiments, the invention relates to a compound of formula (I), or a pharmaceutically acceptable salt thereof, wherein R^{C1} and R^{C2} are each independently H, OH, halo, C₁-C₆ alkyl, C₁-C₆ alkoxy, C₁-C₆ haloalkyl, C₁-C₆ haloalkoxy, —[CH₂]_{q}—C₃-C₆ cycloalkyl, —[CH₂]_{q}—O—C₃-C₆ cycloalkyl, —[CH₂]_{q}—NR^{b}R^{c}, —(CH₂)ₘOR^{b}, or —O(CH₂)ₘOCH₃, or R^{C1} and R^{C2}, together with the carbon atoms to which they are attached, form a ring of formula:

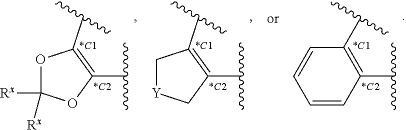

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, the term “compounds of the invention” refers to the compounds of formula (I) and all of the embodiments thereof (e.g., Formula (II), (II-A), (II-B), (II-C), (II-D), (II-E), and (II-F)), as described herein, and to the compounds identified in Table A, Table B, and Table C.

As described herein, the compounds of the invention comprise multiple variable groups (e.g., X^{A1}, R^{A3}, R⁵, R⁶, X^{C1}, R^{C3}, etc.). As one of ordinary skill in the art will recognize, combinations of groups envisioned by this invention are those combinations that result in the formation of stable or chemically feasible compounds. The term “stable,” in this context, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, the term “halo” means F, Cl, Br or I.

As used herein, the term “alkyl” refers to a straight or branched hydrocarbon chain radical group consisting solely of carbon and hydrogen atoms, containing no unsaturation, and having the specified number of carbon atoms, which is attached to the rest of the molecule by a single bond. For example, a “C₁-C₆ alkyl” group is an alkyl group having between one and six carbon atoms.

As used herein, the term “cycloalkyl” refers to a stable, non-aromatic, mono- or bicyclic (fused, bridged, or spiro) saturated hydrocarbon radical consisting solely of carbon and hydrogen atoms, having the specified number of carbon ring atoms, and which is attached to the rest of the molecule by a single bond. For example, a “C₃-C₈ cycloalkyl” group is a cycloalkyl group having between three and eight carbon atoms.

As used herein, the term “alkoxy” refers to a radical of the formula —OR where R is an alkyl group having the specified number of carbon atoms. For example, a “C₁-C₆ alkoxy” group is a radical of the formula —OR where R is an alkyl group having the between one and six carbon atoms.

As used herein, the term “haloalkyl” refers to an alkyl group having the specified number of carbon atoms, wherein one or more of the hydrogen atoms of the alkyl group are replaced by halo groups. For example, a “C₁-C₆ haloalkyl” group is an alkyl group having between one and six carbon atoms, wherein one or more of the hydrogen atoms of the alkyl group are replaced by halo groups.

As used herein, the term “haloalkoxy” refers to an alkoxy group having the specified number of carbon atoms, wherein one or more of the hydrogen atoms of the of the alkyl group are replaced by halo groups.

As used herein, the term “heterocyclyl” refers to a stable, non-aromatic, mono-, bi-, or tricyclic (fused, bridged, or spiro) radical in which one or more ring atoms is a heteroatom (e.g., a heteroatom independently selected from N, O, P, and S), which has the specified number of ring atoms, and which is attached to the rest of the molecule by a single bond. Heterocyclic rings can be saturated, or can contain one or more double or triple bonds. In some embodiments, the “heterocyclyl” group has the indicated number of ring members, in which one or more ring members is a heteroatom independently selected from oxygen, sulfur, nitrogen, and phosphorus, and each ring in the ring system contains 3 to 7 ring members. For example, a 6-membered heterocyclyl includes a total of 6 ring members, at least one of which is a heteroatom (e.g., a heteroatom independently selected from N, O, P, and S).

As used herein, the term “heteroaryl” refers to a stable mono-, bi-, or tricyclic ring radical having the specified number of ring atoms, wherein at least one ring in the system is aromatic, at least one aromatic ring in the system contains one or more heteroatoms (e.g., one or more heteroatoms independently selected from N, O, P, and S). In some embodiments, each ring in the system contains 3 to 7 ring members. For example, a 6-membered heteroaryl includes a total of 6 ring members, at least one of which is a heteroatom selected from N, S, O, and P. The term “heteroaryl” may be used interchangeably with the term “heteroaryl ring” or the term “heteroaromatic”.

As used herein, labels such as “*C1” and “*C2”, such as those shown in the following structures, designate the carbon atoms to which the corresponding R groups (in this case, the R^{C1} and R^{C2} groups, respectively) are attached:

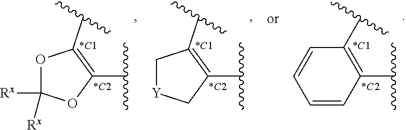

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

As used herein, in any chemical structure or formula, a non-bold, straight bond attached to a stereocenter of a compound, such as in

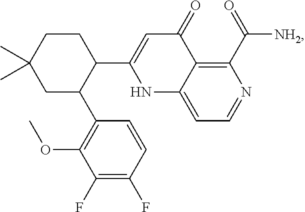 [SMILES: COc1c(C2CC(C)(C)CCC2c2cc(=O)c3c(C(N)=O)nccc3[nH]2)ccc(F)c1F]

denotes that the configuration of the stereocenter is unspecified. The compound may have any configuration, or a mixture of configurations, at the stereocenter.

As used herein, in any chemical structure or formula, a bold or hashed straight bond attached to a stereocenter of a compound, such as in

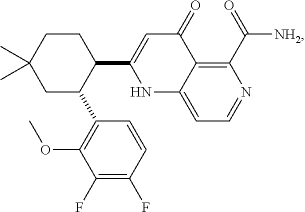

denotes the relative stereochemistry of the stereocenter, relative to other stereocenter(s) to which bold or hashed straight bonds are attached.

As used herein, in any chemical structure or formula, a bold or hashed wedge bond attached to a stereocenter of a compound, such as in

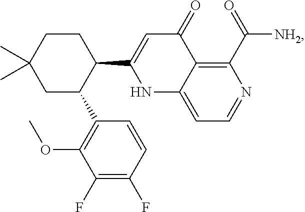 [SMILES: COc1c([C@@H]2CC(C)(C)CC[C@H]2c2cc(=O)c3c(C(N)=O)nccc3[nH]2)ccc(F)c1F]

denotes the absolute stereochemistry of the stereocenter, as well as the relative stereochemistry of the stereocenter, relative to other stereocenter(s) to which bold or hashed wedge bonds are attached.

As used herein, the prefix “rac-,” when used in connection with a chiral compound, refers to a racemic mixture of the compound. In a compound bearing the “rac-” prefix, the (R)- and (S)-designators in the chemical name reflect the relative stereochemistry of the compound.

As used herein, the prefix “dia-,” when used in connection with a chiral compound, refers to a mixture of two or more diastereomers of the compound. In a compound bearing the “dia-” prefix, the (R)- and (S)-designators are assigned arbitrarily and do not necessarily reflect the absolute or relative configuration of the corresponding stereocenters in any component of the mixture. In some instances, all of the diastereomers in a mixture have a single absolute configuration at one or more stereocenters. In these instances, the stereochemical designators associated with the stereocenters having a single absolute configuration are marked with an asterisk (*), e.g., (R*)- and (S*)-. In some instances, all of the diastereomers in a mixture have the same relative stereochemistry at two or more stereocenters. In these instances, the stereochemical designators associated with such stereocenters are marked with a superscript number (ⁿ), e.g., (Rⁿ)- and (Sⁿ)-. The stereochemical designators marked with a superscript number reflect the relative stereochemistry of the corresponding stereocenters with respect to other stereocenters associated with stereochemical designators having the same superscript number, e.g., (R¹)- and (S¹)-, but they do not necessarily reflect the absolute configuration of such stereocenters. The stereochemical designators marked with an asterisk (*) do not necessarily reflect the relative stereochemistry of the corresponding stereocenters with respect to stereocenters associated with stereochemical designators marked with a superscript number (ⁿ) (and vice versa) or with respect to stereocenters associated with unmarked stereochemical designators. Likewise, the stereochemical designators marked with a superscript number (ⁿ) do not necessarily reflect the relative stereochemistry of the corresponding stereocenters with respect to stereocenters associated with stereochemical designators marked with a different superscript number (ⁿ) or with respect to stereocenters associated with unmarked stereochemical designators.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound. In some instances, the absolute configuration of some stereocenters is known, while only the relative configuration of the other stereocenters is known. In these instances, the stereochemical designators associated with the stereocenters of known absolute configuration are marked with an asterisk (*), e.g., (R*)- and (S*)-, while the stereochemical designators associated with stereocenters of unknown absolute configuration are not so marked. The unmarked stereochemical designators associated with the stereocenters of unknown absolute configuration reflect the relative stereochemistry of those stereocenters with respect to other stereocenters of unknown absolute configuration, but do not necessarily reflect the relative stereochemistry with respect to the stereocenters of known absolute configuration. Where a compound contains two or more stereocenters of unknown absolute configuration, and the relative stereochemistry of such stereocenters with respect to each other is unknown, no stereochemical designator is provided for such stereocenters.

As used herein, the term “compound,” when referring to the compounds of the invention, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance isotopic composition of hydrogen.

As used herein, “¹H” refers to protium. Where an atom in a compound of the invention, or a pharmaceutically acceptable salt thereof, is designated as protium, protium is present at the specified position with at least the natural abundance concentration of protium.

As used herein, “D,” “d,” and “²H” refer to deuterium.

In some embodiments, the compounds of the invention, and pharmaceutically acceptable salts thereof, include each constituent atom at approximately the natural abundance isotopic composition of the specified element.

In some embodiments, the compounds of the invention, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (²H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the Examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (˜45% deuterium incorporation). In some embodiments, the isotopic enrichment factor is at least 3500 (˜52.5% deuterium incorporation), at least 4000 (˜60% deuterium incorporation), at least 4500 (˜67.5% deuterium incorporation), at least 5000 (˜75% deuterium incorporation), at least 5500 (˜82.5% deuterium incorporation), at least 6000 (˜90% deuterium incorporation), at least 6333.3 (˜95% deuterium incorporation), at least 6466.7 (˜97% deuterium incorporation), at least 6600 (˜99% deuterium incorporation), or at least 6633.3 (˜99.5% deuterium incorporation).

In some embodiments, the invention relates to a compound of formula (II):

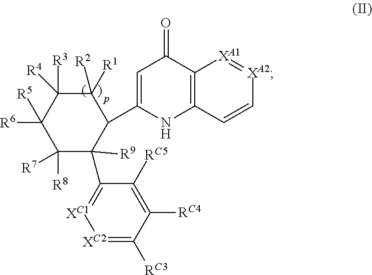

or a pharmaceutically acceptable salt thereof, wherein:
X^{A1} is C—R^{A1}, N, or N⁺—O⁻;
X^{A2} is C—R^{A2}, N, or N⁺—O⁻;
p is 0 or 1;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R¹ and R² are each independently H, OH, C₁-C₆ alkyl, C₁-C₆ haloalkyl, C₃-C₆ cycloalkyl, or C₁-C₆ alkoxy;
R³ and R⁴ are each independently H, halo, OH, C₁-C₆ alkyl, C₁-C₆ haloalkyl, C₃-C₆ cycloalkyl, or C₁-C₆ alkoxy;
R⁵, R⁶, R⁷, and R⁸ are defined as follows:
(i) R⁵ and R⁶ are each independently H, OH, C₁-C₆ alkyl, C₁-C₆ haloalkyl, C₃-C₆ cycloalkyl, or C₁-C₆ alkoxy; and
   R⁷ and R⁸ are each independently H, OH, C₁-C₆ alkyl, C₁-C₆ haloalkyl, C₃-C₆ cycloalkyl, or C₁-C₆ alkoxy; or
(ii) R⁷ and R⁸ are each independently H, OH, C₁-C₆ alkyl, C₁-C₆ haloalkyl, C₃-C₆ cycloalkyl, or C₁-C₆ alkoxy; and
   R⁵ and R⁶, together with the carbon atom to which they are attached, form a C₃-C₆ cycloalkyl optionally substituted with 1-6 R^{y}; or
(iii) R⁶ and R⁸, together with the carbon atoms to which they are attached, form a fused C₃-C₆ cycloalkyl optionally substituted with 1-6 R^{y}; and
   R⁶ and R are each independently H, OH, C₁-C₆ alkyl, C₁-C₆ haloalkyl, C₃-C₆ cycloalkyl, or C₁-C₆ alkoxy;
R⁹ is H or CH₃;
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR^{b}, or —O(CH₂)ₘOCH₃;
each R^{y} is independently halo, OH, C₁-C₆ alkyl, or C₁-C₆ haloalkyl;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (III):

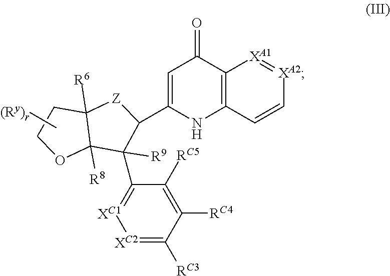

or a pharmaceutically acceptable salt thereof, wherein:
X^{A1} is C—R^{A1}, N, or N⁺—O⁻;
X^{A2} is C—R^{A2}, N, or N⁺—O⁻;
Z is C(—R³)(—R⁴) or O;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR^{b}, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R³ and R⁴ are each independently H, halo, OH, C₁-C₆ alkyl, C₁-C₆ haloalkyl, C₃-C₆ cycloalkyl, or C₁-C₆ alkoxy;
R⁶ and R⁸ are each independently H, OH, C₁-C₆ alkyl, C₁-C₆ haloalkyl, C₃-C₆ cycloalkyl, or C₁-C₆ alkoxy;
R⁹ is H or CH₃;
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR^{b}, or —O(CH₂)ₘOCH₃;
each R^{y} is independently halo, OH, C₁-C₆ alkyl, or C₁-C₆ haloalkyl;
R^{b} and R^{c} are each independently H or C₁-C₆ alkyl, or R^{b} and R^{c}, together with the nitrogen atom to which they are attached, form a 3-6 membered heterocyclyl;
m and o are each independently 0, 1, 2, or 3; and
r is 0, 1, 2, 3, or 4.

In some embodiments, the invention relates to a compound of formula (I), (II), or (III), or a pharmaceutically acceptable salt thereof, wherein X^{A1} is C—R^{A1}. In some embodiments, the invention relates to a compound of formula (I), (II), or (III), or a pharmaceutically acceptable salt thereof, wherein X^{A1} is C—R^{A1}, and R^{A1} is —CN or —C(O)NR^{b}R^{c}. In some embodiments, the invention relates to a compound of formula (I), (II), or (III), or a pharmaceutically acceptable salt thereof, wherein X^{A1} is C—R^{A1}, and R^{A1} is —C(O)NR^{b}R^{c}. In some embodiments, the invention relates to a compound of formula (I), (II), or (III), or a pharmaceutically acceptable salt thereof, wherein X^{A1} is C—R^{A1}, and R^{A1} is —C(O)NH₂. In some embodiments, the invention relates to a compound of formula (I), (II), or (III), or a pharmaceutically acceptable salt thereof, wherein X^{A1} is C—R^{A1}, and R^{A1} is —CN.

In some embodiments, the invention relates to a compound of formula (I), (II), or (III), or a pharmaceutically acceptable salt thereof, wherein X^{A2} is N.

In some embodiments, the invention relates to a compound of formula (I), (II), or (III), or a pharmaceutically acceptable salt thereof, wherein X^{A1} is C—R^{A1}; R^{A1} is —C(O)NH₂; and X^{A2} is N. In some embodiments, the invention relates to a compound of formula (I), (II), or (III), or a pharmaceutically acceptable salt thereof, wherein X^{A1} is C—R^{A1}; R^{A1} is —CN; and X^{A2} is N.

In some embodiments, the invention relates to a compound of formula (I), (II), or (III), or a pharmaceutically acceptable salt thereof, wherein X^{A1} is C—R^{A1}; R^{A1} is —C(O)NH₂; X^{A2} is N; R^{A3} is H; X^{A4} is C—R^{A4}; R^{A4} is H; X^{A5} is C—R^{A5}; and R^{A5} is H. In some embodiments, the invention relates to a compound of formula (I), (II), or (III), or a pharmaceutically acceptable salt thereof, wherein X^{A1} is C—R^{A1}; R^{A1} is —CN; X^{A2} is N; R^{A3} is H; X^{A4} is C—R^{A4}; R^{A4} is H; X^{A5} is C—R^{A5}; and R^{A5} is H.

In some embodiments, the invention relates to a compound of formula (I) or (II), or a pharmaceutically acceptable salt thereof, wherein p=0, and R³ and R⁴ are each H. In some embodiments, the invention relates to a compound of formula (I) or (II), or a pharmaceutically acceptable salt thereof, wherein p=1, and R¹ and R² are each H.

In some embodiments, the invention relates to a compound of formula (I) or (II), or a pharmaceutically acceptable salt thereof, wherein p=1, and R³ and R⁴ are each H. In some embodiments, the invention relates to a compound of formula (I) or (II), or a pharmaceutically acceptable salt thereof, wherein p=1, and R³ and R⁴ are each F. In some embodiments, the invention relates to a compound of formula (I) or (II), or a pharmaceutically acceptable salt thereof, wherein p=1, and R³ and R⁴ are each independently H, C₁-C₃ alkyl, or C₁-C₃ haloalkyl. In some embodiments, the invention relates to a compound of formula (I) or (II), or a pharmaceutically acceptable salt thereof, wherein p=1, and R³ is —CH₃ and R⁴ is —CF₃.

In some embodiments, the invention relates to a compound of formula (I) or (II), or a pharmaceutically acceptable salt thereof, wherein p=0, and R⁵ and R⁶ are each independently H, C₁-C₃ alkyl, or C₁-C₃ haloalkyl. In some embodiments, the invention relates to a compound of formula (I) or (II), or a pharmaceutically acceptable salt thereof, wherein p=0, and R⁵ and R⁶ are each independently H, —CH₃, or —CF₃. In some embodiments, the invention relates to a compound of formula (I) or (II), or a pharmaceutically acceptable salt thereof, wherein p=0, and R⁵ and R⁶ are each H. In some embodiments, the invention relates to a compound of formula (I) or (II), or a pharmaceutically acceptable salt thereof, wherein p=0, and R⁵ and R⁶ are each —CH₃. In some embodiments, the invention relates to a compound of formula (I) or (II), or a pharmaceutically acceptable salt thereof, wherein p=0, and R is —CH₃ and R⁶ is —CF₃.

In some embodiments, the invention relates to a compound of formula (I) or (II), or a pharmaceutically acceptable salt thereof, wherein p=1, and R⁵ and R⁶ are each independently H, C₁-C₄ alkyl, or C₁-C₃ haloalkyl. In some embodiments, the invention relates to a compound of formula (I) or (II), or a pharmaceutically acceptable salt thereof, wherein p=1, and R⁵ and R⁶ are each independently H, —CH₃, -tBu, or —CF₃. In some embodiments, the invention relates to a compound of formula (I) or (II), or a pharmaceutically acceptable salt thereof, wherein p=1, and R⁵ is H and R⁶ is -tBu. In some embodiments, the invention relates to a compound of formula (I) or (II), or a pharmaceutically acceptable salt thereof, wherein p=1, and R⁵ and R⁶ are each H. In some embodiments, the invention relates to a compound of formula (I) or (II), or a pharmaceutically acceptable salt thereof, wherein p=1, and R and R⁶ are each —CH₃. In some embodiments, the invention relates to a compound of formula (I) or (II), or a pharmaceutically acceptable salt thereof, wherein p=1, and R⁵ is —CH₃ and R⁶ is —CF₃.

In some embodiments, the invention relates to a compound of formula (I) or (II), or a pharmaceutically acceptable salt thereof, wherein p=0, and R5 and R⁶, together with the carbon atom to which they are attached, form a C₃-C₆ cycloalkyl optionally substituted with 1-6 R^{y}. In some embodiments, each R^{y} is independently halo. In other embodiments, each R^{y} is F. In some embodiments, the invention relates to a compound of formula (I) or (II), or a pharmaceutically acceptable salt thereof, wherein p=0, and R5 and R⁶, together with the carbon atom to which they are attached, form a C₃-C₆ cycloalkyl optionally substituted with 1 or 2 R^{y}. In some embodiments, the invention relates to a compound of formula (I) or (II), or a pharmaceutically acceptable salt thereof, wherein p=0, and R and R⁶, together with the carbon atom to which they are attached, form a C₃ cycloalkyl optionally substituted with 1 or 2 F. In some embodiments, the invention relates to a compound of formula (I) or (II), or a pharmaceutically acceptable salt thereof, wherein p=0, and R⁵ and R⁶, together with the carbon atom to which they are attached, form a C₄ cycloalkyl optionally substituted with 1 or 2 F. In some embodiments, the invention relates to a compound of formula (I) or (II), or a pharmaceutically acceptable salt thereof, wherein p=0, and R⁵ and R⁶, together with the carbon atom to which they are attached, form a C₆ cycloalkyl optionally substituted with 1 or 2 R^{y}.

In some embodiments, the invention relates to a compound of formula (I) or (II), or a pharmaceutically acceptable salt thereof, wherein p=0, and R⁵ and R⁷, together with the carbon atoms to which they are attached, form a fused C₃-C₆ cycloalkyl optionally substituted with 1-6 R^{y}. In some embodiments, each R^{y} is independently halo. In other embodiments, each R^{y} is F. In some embodiments, the invention relates to a compound of formula (I) or (II), or a pharmaceutically acceptable salt thereof, wherein p=0, R⁵ and R⁷, together with the carbon atoms to which they are attached, form a fused C₃-C₆ cycloalkyl optionally substituted with 1 or 2 R^{y}. In some embodiments, the invention relates to a compound of formula (I) or (II), or a pharmaceutically acceptable salt thereof, wherein p=0, R⁵ and R⁷, together with the carbon atoms to which they are attached, form a fused C₅ cycloalkyl optionally substituted with 1 or 2 R^{y}. In some embodiments, the invention relates to a compound of formula (I) or (II), or a pharmaceutically acceptable salt thereof, wherein p=0, R and R, together with the carbon atoms to which they are attached, form a fused Cₛ cycloalkyl optionally substituted with 1 or 2 F. In some embodiments, the invention relates to a compound of formula (I) or (II), or a pharmaceutically acceptable salt thereof, wherein p=0, R and R, together with the carbon atoms to which they are attached, form a fused C₆ cycloalkyl optionally substituted with 1 or 2 R^{y}. In some embodiments, the invention relates to a compound of formula (I) or (II), or a pharmaceutically acceptable salt thereof, wherein p=0, R⁵ and R⁷, together with the carbon atoms to which they are attached, form a fused C₆ cycloalkyl optionally substituted with 1 or 2 F.

In some embodiments, the invention relates to a compound of formula (I) or (II), or a pharmaceutically acceptable salt thereof, wherein p=0, Z is C(—R³)(—R⁴), and R⁵ and R⁷, together with the carbon atoms to which they are attached, form a fused C₃-C₆ cycloalkyl optionally substituted with 1-6 R^{y}. In some embodiments, each R^{y} is independently halo. In other embodiments, each R^{y} is F. In some embodiments, the invention relates to a compound of formula (I) or (II), or a pharmaceutically acceptable salt thereof, wherein p=0, Z is C(—R³)(—R⁴), and R⁵ and R⁷, together with the carbon atoms to which they are attached, form a fused C₃-C₆ cycloalkyl optionally substituted with 1 or 2 R^{y}. In some embodiments, the invention relates to a compound of formula (I) or (II), or a pharmaceutically acceptable salt thereof, wherein p=0, Z is C(—R³)(—R⁴), and R and R, together with the carbon atoms to which they are attached, form a fused C₅ cycloalkyl optionally substituted with 1 or 2 R^{y}. In some embodiments, the invention relates to a compound of formula (I) or (II), or a pharmaceutically acceptable salt thereof, wherein p=0, Z is C(—R³)(—R⁴), and R⁵ and R⁷, together with the carbon atoms to which they are attached, form a fused C₅ cycloalkyl optionally substituted with 1 or 2 F. In some embodiments, the invention relates to a compound of formula (I) or (II), or a pharmaceutically acceptable salt thereof, wherein p=0, Z is C(—R³)(—R⁴), and R⁵ and R⁷, together with the carbon atoms to which they are attached, form a fused C₆ cycloalkyl optionally substituted with 1 or 2 R^{y}. In some embodiments, the invention relates to a compound of formula (I) or (II), or a pharmaceutically acceptable salt thereof, wherein p=0, Z is C(—R³)(—R⁴), and R^{S} and R, together with the carbon atoms to which they are attached, form a fused C₆ cycloalkyl optionally substituted with 1 or 2 F.

In some embodiments, the invention relates to a compound of formula (I) or (II), or a pharmaceutically acceptable salt thereof, wherein p=1, Z is C(—R³)(—R⁴), and R^{S} and R⁷, together with the carbon atoms to which they are attached, form a fused C₃-C₆ cycloalkyl optionally substituted with 1-6 R^{y}. In some embodiments, each R^{y} is independently halo. In other embodiments, each R^{y} is F. In some embodiments, the invention relates to a compound of formula (I) or (II), or a pharmaceutically acceptable salt thereof, wherein p=1, Z is C(—R³)(—R⁴), and R^{S} and R, together with the carbon atoms to which they are attached, form a fused C₃-C₆ cycloalkyl optionally substituted with 1 or 2 R^{y}. In some embodiments, the invention relates to a compound of formula (I) or (II), or a pharmaceutically acceptable salt thereof, wherein p=1, Z is C(—R³)(—R⁴), and R and R, together with the carbon atoms to which they are attached, form a fused C₅ cycloalkyl optionally substituted with 1 or 2 R^{y}. In some embodiments, the invention relates to a compound of formula (I) or (II), or a pharmaceutically acceptable salt thereof, wherein p=1, Z is C(—R³)(—R⁴), and R⁵ and R⁷, together with the carbon atoms to which they are attached, form a fused C₅ cycloalkyl optionally substituted with 1 or 2 F. In some embodiments, the invention relates to a compound of formula (I) or (II), or a pharmaceutically acceptable salt thereof, wherein p=1, Z is C(—R³)(—R⁴), and R⁵ and R, together with the carbon atoms to which they are attached, form a fused C₆ cycloalkyl optionally substituted with 1 or 2 R^{y}. In some embodiments, the invention relates to a compound of formula (I) or (II), or a pharmaceutically acceptable salt thereof, wherein p=1, Z is C(—R³)(—R⁴), and R⁵ and R, together with the carbon atoms to which they are attached, form a fused C₆ cycloalkyl optionally substituted with 1 or 2 F.

In some embodiments, the invention relates to a compound of formula (I) or (II), or a pharmaceutically acceptable salt thereof, wherein p=0, Z is —CH₂—, and R⁵ and R, together with the carbon atoms to which they are attached, form a fused C₃-C₆ cycloalkyl optionally substituted with 1-6 R^{y}. In some embodiments, each R^{y} is independently halo. In other embodiments, each R^{y} is F. In some embodiments, the invention relates to a compound of formula (I) or (II), or a pharmaceutically acceptable salt thereof, wherein p=0, Z is —CH₂—, and R⁵ and R⁷, together with the carbon atoms to which they are attached, form a fused C₃-C₆ cycloalkyl optionally substituted with 1 or 2 R^{y}. In some embodiments, the invention relates to a compound of formula (I) or (II), or a pharmaceutically acceptable salt thereof, wherein p=0, Z is —CH₂—, and R and R, together with the carbon atoms to which they are attached, form a fused C₅ cycloalkyl optionally substituted with 1 or 2 R^{y}. In some embodiments, the invention relates to a compound of formula (I) or (II), or a pharmaceutically acceptable salt thereof, wherein p=0, Z is —CH₂—, and R⁵ and R⁷, together with the carbon atoms to which they are attached, form a fused C₅ cycloalkyl optionally substituted with 1 or 2 F. In some embodiments, the invention relates to a compound of formula (I) or (II), or a pharmaceutically acceptable salt thereof, wherein p=0, Z is —CH₂—, and R⁵ and R⁷, together with the carbon atoms to which they are attached, form a fused C₆ cycloalkyl optionally substituted with 1 or 2 R^{y}. In some embodiments, the invention relates to a compound of formula (I) or (II), or a pharmaceutically acceptable salt thereof, wherein p=0, Z is —CH₂—, and R⁵ and R⁷, together with the carbon atoms to which they are attached, form a fused C₆ cycloalkyl optionally substituted with 1 or 2 F.

In some embodiments, the invention relates to a compound of formula (I) or (II), or a pharmaceutically acceptable salt thereof, wherein p=1, Z is —CH₂—, and R^{S} and R⁷, together with the carbon atoms to which they are attached, form a fused C₃-C₆ cycloalkyl optionally substituted with 1-6 R^{y}. In some embodiments, each R^{y} is independently halo. In other embodiments, each R^{y} is F. In some embodiments, the invention relates to a compound of formula (I) or (II), or a pharmaceutically acceptable salt thereof, wherein p=1, Z is —CH₂—, and R^{S} and R⁷, together with the carbon atoms to which they are attached, form a fused C₃-C₆ cycloalkyl optionally substituted with 1 or 2 R^{y}. In some embodiments, the invention relates to a compound of formula (I) or (II), or a pharmaceutically acceptable salt thereof, wherein p=1, Z is —CH₂—, and R⁵ and R⁷, together with the carbon atoms to which they are attached, form a fused Cₛ cycloalkyl optionally substituted with 1 or 2 R^{y}. In some embodiments, the invention relates to a compound of formula (I) or (II), or a pharmaceutically acceptable salt thereof, wherein p=1, Z is —CH₂—, and R⁵ and R⁷, together with the carbon atoms to which they are attached, form a fused C₅ cycloalkyl optionally substituted with 1 or 2 F. In some embodiments, the invention relates to a compound of formula (I) or (II), or a pharmaceutically acceptable salt thereof, wherein p=1, Z is —CH₂—, and R⁵ and R⁷, together with the carbon atoms to which they are attached, form a fused C₆ cycloalkyl optionally substituted with 1 or 2 R^{y}. In some embodiments, the invention relates to a compound of formula (I) or (II), or a pharmaceutically acceptable salt thereof, wherein p=1, Z is —CH₂—, and R⁵ and R⁷, together with the carbon atoms to which they are attached, form a fused C₆ cycloalkyl optionally substituted with 1 or 2 F.

In some embodiments, the invention relates to a compound of formula (I) or (II), or a pharmaceutically acceptable salt thereof, wherein R⁷ and R⁸ are each independently H or C₁-C₃ alkyl. In some embodiments, the invention relates to a compound of formula (I) or (II), or a pharmaceutically acceptable salt thereof, wherein R⁷ and R⁸ are each independently H or —CH₃. In some embodiments, the invention relates to a compound of formula (I) or (II), or a pharmaceutically acceptable salt thereof, wherein R⁷ and R are each H.

In some embodiments, the invention relates to a compound of formula (I) or (III), or a pharmaceutically acceptable salt thereof, wherein Z is O.

In some embodiments, the invention relates to a compound of formula (I) or (III), or a pharmaceutically acceptable salt thereof, wherein R⁶ and R⁸ are each H.

In some embodiments, the invention relates to a compound of formula (I) or (III), or a pharmaceutically acceptable salt thereof, wherein each R^{y} is independently halo, and r is 2. In some embodiments, the invention relates to a compound of formula (I) or (III), or a pharmaceutically acceptable salt thereof, wherein each R^{y} is F, and r is 2.

In some embodiments, the invention relates to a compound of formula (I), (II), or (III), or a pharmaceutically acceptable salt thereof, wherein R⁹ is H.

In some embodiments, the invention relates to a compound of formula (I), (II), or (III), or a pharmaceutically acceptable salt thereof, wherein X^{C1} is C—R^{C1}. In some embodiments, the invention relates to a compound of formula (I), (II), or (III), or a pharmaceutically acceptable salt thereof, wherein X^{C2} is C—R^{C2}. In some embodiments, the invention relates to a compound of formula (I), (II), or (III), or a pharmaceutically acceptable salt thereof, wherein X^{C2} is N. In some embodiments, the invention relates to a compound of formula (I), (II), or (III), or a pharmaceutically acceptable salt thereof, wherein X^{C1} is C—R^{C1} and X^{C2} is C—R^{C2}. In some embodiments, the invention relates to a compound of formula (I), (II), or (III), or a pharmaceutically acceptable salt thereof, wherein X^{C1} is C—R^{C1} and X^{C2} is N.

In some embodiments, the invention relates to a compound of formula (I), (II), or (III), or a pharmaceutically acceptable salt thereof, wherein R^{C1} R^{C2}, R^{C3}, R^{C4}, and R^{C5} are each independently H, OH, halo, —C₁-C₃ alkyl, —C₃-C₆ cycloalkyl, or —(CH₂)ₘOR^{b}. In some embodiments, the invention relates to a compound of formula (I), (II), or (III), or a pharmaceutically acceptable salt thereof, wherein R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, or —CH₂OH. In some embodiments, the invention relates to a compound of formula (I), (II), or (III), or a pharmaceutically acceptable salt thereof, wherein R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, or —CH₂OH. In some embodiments, the invention relates to a compound of formula (I), (II), or (III), or a pharmaceutically acceptable salt thereof, wherein R^{C1}, R^{C2}, R^{C3}, R^{C4}, and R^{C5} are each independently H, OH, F, Cl, —CH₃, —CH₂CH₃, -cyclopropyl, —OCH₃, or —CH₂OH. In some embodiments, the invention relates to a compound of formula (I), (II), or (III), or a pharmaceutically acceptable salt thereof, wherein R^{C1}, R^{C2}, R^{C3}, R^{C4}, and R^{C5} are each independently H, OH, F, Cl, —CH₃, —CH₂CH₃, —OCH₃, —CF₃, C₃ cycloalkyl, —O—C₃ cycloalkyl, or —CH₂OH. In some embodiments, the invention relates to a compound of formula (I), (II), or (III), or a pharmaceutically acceptable salt thereof, wherein R^{C1}, R^{C2}, R^{C3}, R^{C4}, and R^{C5} are each independently H, OH, F, Cl, —CH₃, —CH₂CH₃, —OCH₃, C₃ cycloalkyl, or —CH₂OH.

In some embodiments, the invention relates to a compound of formula (I), (II), or (III), or a pharmaceutically acceptable salt thereof, wherein R^{C3} is H, halo, or C₁-C₆ alkyl. In some embodiments, the invention relates to a compound of formula (I), (II), or (III), or a pharmaceutically acceptable salt thereof, wherein R^{C3} is H. In some embodiments, the invention relates to a compound of formula (I), (II), or (III), or a pharmaceutically acceptable salt thereof, wherein R^{C3} is halo. In some embodiments, the invention relates to a compound of formula (I), (II), or (III), or a pharmaceutically acceptable salt thereof, wherein R^{C3} is C₁-C₆ alkyl. In some embodiments, the invention relates to a compound of formula (I), (II), or (III), or a pharmaceutically acceptable salt thereof, wherein R^{C3} is H, F, or —CH₃. In some embodiments, the invention relates to a compound of formula (I), (II), or (III), or a pharmaceutically acceptable salt thereof, wherein R^{C3} is F. In some embodiments, the invention relates to a compound of formula (I), (II), or (III), or a pharmaceutically acceptable salt thereof, wherein R^{C3} is —CH₃.

In some embodiments, the invention relates to a compound of formula (I), (II), or (III), or a pharmaceutically acceptable salt thereof, wherein R^{C4} is halo. In some embodiments, the invention relates to a compound of formula (I), (II), or (III), or a pharmaceutically acceptable salt thereof, wherein R^{C4} is F or Cl. In some embodiments, the invention relates to a compound of formula (I), (II), or (III), or a pharmaceutically acceptable salt thereof, wherein R^{C4} is —CH₃.

In some embodiments, the invention relates to a compound of formula (I), (II), or (III), or a pharmaceutically acceptable salt thereof, wherein R^{C5} is halo. In some embodiments, the invention relates to a compound of formula (I), (II), or (III), or a pharmaceutically acceptable salt thereof, wherein R^{C5} is F or Cl. In some embodiments, the invention relates to a compound of formula (I), (II), or (III), or a pharmaceutically acceptable salt thereof, wherein R^{C5} is —C₁-C₃ alkyl. In some embodiments, the invention relates to a compound of formula (I), (II), or (III), or a pharmaceutically acceptable salt thereof, wherein R^{C5} is —CH₃ or —CH₂CH₃. In some embodiments, the invention relates to a compound of formula (I), (II), or (III), or a pharmaceutically acceptable salt thereof, wherein R^{C5} is —C₃-C₆ cycloalkyl. In some embodiments, the invention relates to a compound of formula (I), (II), or (III), or a pharmaceutically acceptable salt thereof, wherein R^{C5} is cyclopropyl. In some embodiments, the invention relates to a compound of formula (I), (II), or (III), or a pharmaceutically acceptable salt thereof, wherein R^{C5} is —OCH₃ or —CH₂OH. In some embodiments, the invention relates to a compound of formula (I), (II), or (III), or a pharmaceutically acceptable salt thereof, wherein R^{C5} is —OCH₃. In some embodiments, the invention relates to a compound of formula (I), (II), or (III), or a pharmaceutically acceptable salt thereof, wherein R^{C5} is —CH₂OH. In some embodiments, the invention relates to a compound of formula (I), (II), or (III), or a pharmaceutically acceptable salt thereof, wherein R^{C5} is H.

In some embodiments, the invention relates to a compound of formula (I), or a pharmaceutically acceptable salt thereof, wherein p=0:

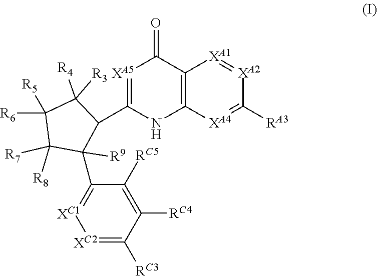

In some embodiments, the invention relates to a compound of formula (I), or a pharmaceutically acceptable salt thereof, wherein p=1:

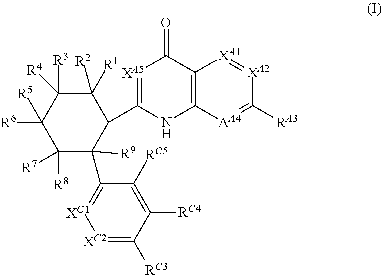

In some embodiments, the invention relates to a compound of formula (II), or a pharmaceutically acceptable salt thereof, wherein p=0:

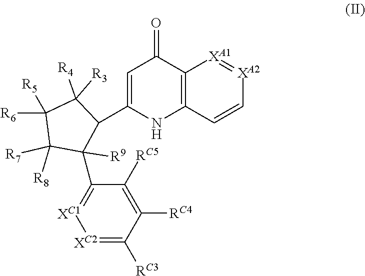

In some embodiments, the invention relates to a compound of formula (II), or a pharmaceutically acceptable salt thereof, wherein p=1:

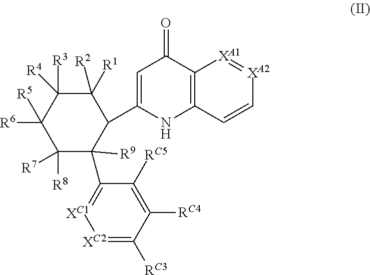

In some embodiments, the invention relates to a compound of formula (II-A):

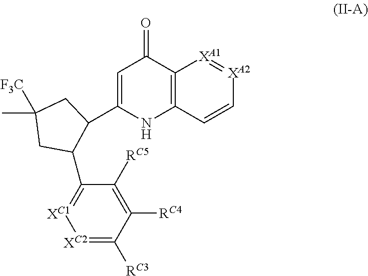

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR^{b}, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (II-B):

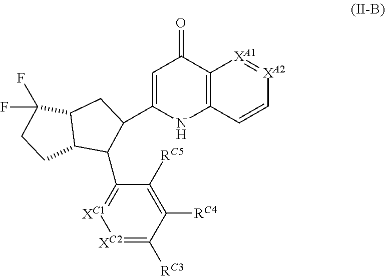

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR^{b}, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (II-B-1):

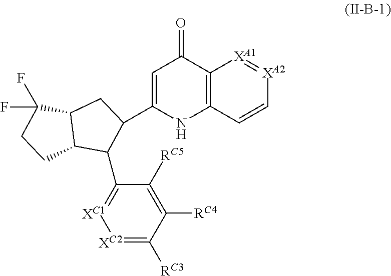

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (II-B-2):

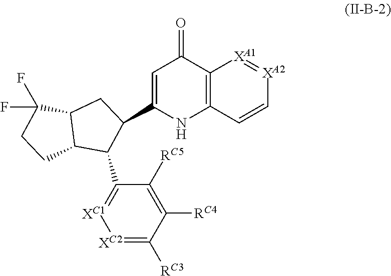

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR^{b}, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (II-C):

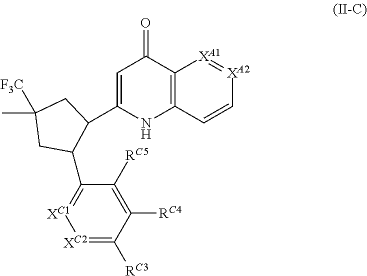

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (II-D):

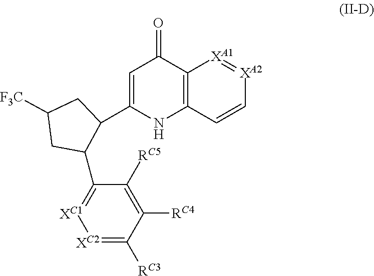

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR^{b}, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (II-E):

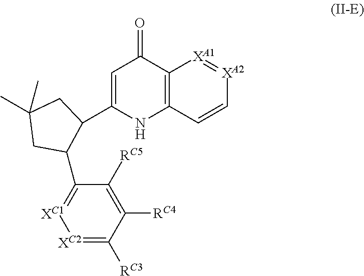

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (II-F):

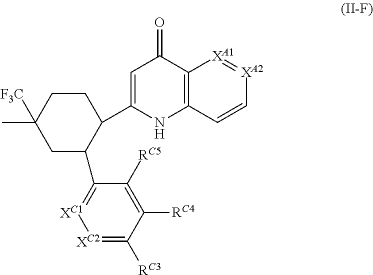

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR^{b}, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (III-A):

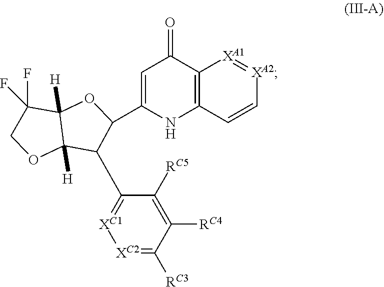

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (III-A-1):

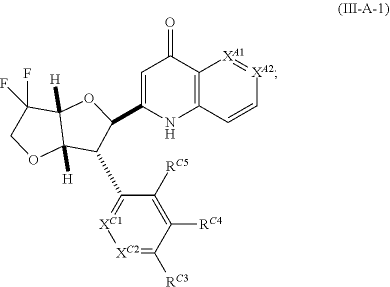

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C2} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (III-A-2):

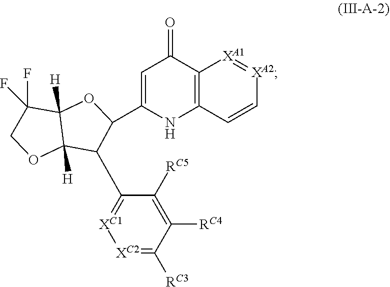

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (III-A-3):

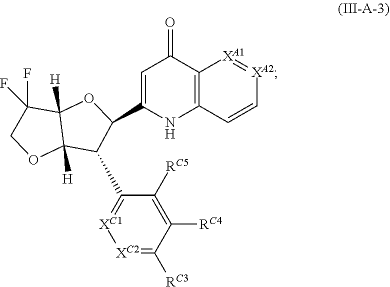

or a pharmaceutically acceptable salt thereof, wherein:
X^{A1} is C—R^{A1}, N, or N⁺—O⁻;
X^{A2} is C—R^{A2}, N, or N⁺—O⁻;
X^{C1} is C—R^{C2} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
R^{b} and R^{c} are each independently H or C₁-C₆ alkyl, or R^{b} and R^{c}, together with the nitrogen atom to which they are attached, form a 3-6 membered heterocyclyl; and
m and o are each independently 0, 1, 2, or 3.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{A1} is C—R^{A1}. In some embodiments, the invention relates to a compound of formula (II-A), (II-B), (II-B3-1), (II-B3-2), (II-C), (II-D), (II-E), (II-F), (III-A), (III-A-1), (III-A-2), or (III-A-3), or a pharmaceutically acceptable salt thereof, wherein X^{A1} is C—R^{A1}, and R^{AI} is —CN or —C(O)NR^{b}R^{c}. In some embodiments, the invention relates to a compound of formula (II-A), (II-B), (II-B-1), (II-B3-2), (II-C), (II-D), (II-E), (II-F), (III-A), (III-A-1), (III-A-2), or (III-A-3), or a pharmaceutically acceptable salt thereof, wherein X^{A1} is C—R^{A1}, and R^{A1} is —C(O)NR^{b}R^{c}. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{A1} is C—R^{A1}, and R^{A1} is —C(O)NH₂. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{A1} is C—R^{A1}, and R^{A1} is —CN.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{A2} is N.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{A1} is C—R^{A1}; and X^{A2} is N. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{A1} is C—R^{A1}, and R^{A1} is —C(O)NR^{b}R^{c}; and X^{A2} is N. In some embodiments, the invention relates to a compound of formula (II-A), (II-B), (II-B3-1), (II-B3-2), (II-C), (II-D), (II-E), (II-F), (III-A), (III-A-1), (III-A-2), or (III-A-3), or a pharmaceutically acceptable salt thereof, wherein X^{A1} is C—R^{A}; R^{A1} is —C(O)NH₂; and X^{A2} is N.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is N. In some embodiments, the invention relates to a compound of formula (II-A), (II-B), (II-B-1), (II-B3-2), (II-C), (II-D), (II-E), (II-F), (III-A), (III-A-1), (III-A-2), or (III-A-3), or a pharmaceutically acceptable salt thereof, wherein X^{C1} is C—R^{C1}. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C2} is N. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C2} is C—R^{C2}. In some embodiments, the invention relates to a compound of formula (II-A), (II-B), (II-B3-1), (II-B3-2), (II-C), (II-D), (II-E), (II-F), (III-A), (III-A-1), (III-A-2), or (III-A-3), or a pharmaceutically acceptable salt thereof, wherein X^{C1} is C—R^{C1} and X^{C2} is C—R^{C2}. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is N and X^{C2} is C—R^{C2}. In some embodiments, the invention relates to a compound of formula (II-A), (II-B), (II-B-1), (II-B3-2), (II-C), (II-D), (II-E), (II-F), (III-A), (III-A-1), (III-A-2), or (III-A-3), or a pharmaceutically acceptable salt thereof, wherein X^{C1} is C—R^{C1} and X^{C2} is N.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} R^{C2}, R^{C3}, R^{C4}, and R^{C5} are each independently H, OH, halo, —C₁-C₃ alkyl, —C₃-C₆ cycloalkyl, or —(CH₂)ₘOR^{b}. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} R^{C2}, R^{C3}, R^{C4}, and R^{C5} are each independently H, OH, F, Cl, —CH₃, —CH₂CH₃, -cyclopropyl, —OCH₃, or —CH₂OH.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} is H, —C₁-C₃ alkyl, or —C₁-C₆ alkoxy. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} is H, —CH₃, —CH₂CH₃, or —OCH₃. In some embodiments, the invention relates to a compound of formula (II-A), (II-B), (II-B-1), (II-B3-2), (II-C), (II-D), (II-E), (II-F), (III-A), (III-A-1), (III-A-2), or (III-A-3), or a pharmaceutically acceptable salt thereof, wherein R^{C1} is H. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} is —C₁-C₃ alkyl. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} is —CH₃, or —CH₂CH₃. In some embodiments, the invention relates to a compound of formula (II-A), (II-B), (II-B-1), (II-B3-2), (II-C), (II-D), (II-E), (II-F), (III-A), (III-A-1), (III-A-2), or (III-A-3), or a pharmaceutically acceptable salt thereof, wherein R^{C1} is —CH₃. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} is —CH₂CH₃. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} is —C₁-C₆ alkoxy. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C1} is —OCH₃.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C2} is halo or C₁-C₆ alkyl. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C2} is halo. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C2} is F or Cl. In some embodiments, the invention relates to a compound of formula (II-A), (II-B), (II-B3-1), (II-B3-2), (II-C), (II-D), (II-E), (II-F), (III-A), (III-A-1), (III-A-2), or (III-A-3), or a pharmaceutically acceptable salt thereof, wherein R^{C2} is F. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C2} is Cl. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C2} is C₁-C₆ alkyl. In some embodiments, the invention relates to a compound of formula (II-A), (II-B3), (II-B-1), (II-B3-2), (II-C), (II-D), (II-E), (II-F), (III-A), (III-A-1), (III-A-2), or (III-A-3), or a pharmaceutically acceptable salt thereof, wherein R^{C2} is —CH₃.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C3} is H, halo, or C₁-C₆ alkyl. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C3} is H. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C3} is halo. In some embodiments, the invention relates to a compound of formula (II-A), (II-B), (II-B3-1), (II-B3-2), (II-C), (II-D), (II-E), (II-F), (III-A), (III-A-1), (III-A-2), or (III-A-3), or a pharmaceutically acceptable salt thereof, wherein R^{C3} is F. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C3} is C₁-C₆ alkyl. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C3} is —CH₃.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C4} is H. In some embodiments, the invention relates to a compound of formula (II-A), (II-B3), (II-B-1), (II-B3-2), (II-C), (II-D), (II-E), (II-F), (III-A), (III-A-1), (III-A-2), or (III-A-3), or a pharmaceutically acceptable salt thereof, wherein R^{C4} is halo. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C4} is F or Cl. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C4} is —CH₃.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C5} is halo. In some embodiments, the invention relates to a compound of formula (II-A), (II-B3), (II-B3-1), (II-B3-2), (II-C), (II-D), (II-E), (II-F), (III-A), (III-A-1), (III-A-2), or (III-A-3), or a pharmaceutically acceptable salt thereof, wherein R^{C5} is F or Cl. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C5} is —C₁-C₃ alkyl. In some embodiments, the invention relates to a compound of formula (II-A), (II-B), (II-B-1), (II-B3-2), (II-C), (II-D), (II-E), (II-F), (III-A), (III-A-1), (III-A-2), or (III-A-3), or a pharmaceutically acceptable salt thereof, wherein R^{C5} is —CH₃ or —CH₂CH₃. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C5} is —C₃-C₆ cycloalkyl. In some embodiments, the invention relates to a compound of formula (II-A), (II-B), (II-B3-1), (II-B3-2), (II-C), (II-D), (II-E), (II-F), (III-A), (III-A-1), (III-A-2), or (III-A-3), or a pharmaceutically acceptable salt thereof, wherein R^{C5} is cyclopropyl. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C5} is —OCH₃ or —CH₂OH. In some embodiments, the invention relates to a compound of formula (II-A), (II-B), (II-B-1), (II-B3-2), (II-C), (II-D), (II-E), (II-F), (III-A), (III-A-1), (III-A-2), or (III-A-3), or a pharmaceutically acceptable salt thereof, wherein R^{C5} is —OCH₃. In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C5} is —CH₂OH. In some embodiments, the invention relates to a compound of (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein R^{C5} is H.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is C—R^{C1}, X^{C2} is C—R^{C2}, R^{C1} is —CH₃, R^{C2} is F, R^{C3} is F, R^{C4} is H, and R^{C5} is H.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is C—R^{C1}, X^{C2} is C—R^{C2}, R^{C1} is —OCH₃, R^{C2} is F, R^{C3} is F, R^{C4} is H, and R^{C5} is H.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is C—R^{C1}, X^{C2} is C—R^{C2}, R^{C1} is —OCH₃, R^{C2} is Cl, R^{C3} is F, R^{C4} is H, and R^{C5} is H.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is C—R^{C1}, X^{C2} is C—R^{C2}, R^{C1} is —CH₃, R^{C2} is Cl, R^{C3} is F, R^{C4} is H, and R^{C5} is H.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is C—R^{C1}, X^{C2} is C—R^{C2}, R^{C1} is —CH₂CH₃, R^{C2} is F, R^{C3} is F, R^{C4} is H, and R^{C5} is H.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is N, X^{C2} is C—R^{C2}, R^{C2} is —CH₃, R^{C3} is F, R^{C4} is H, and R^{C5} is H.

In some embodiments, the invention relates to a compound of formula (II-A), (II-B), (II-B-1), (II-B-2), (II-C), (II-D), (II-E), (II-F), (III-A), (III-A-1), (III-A-2), or (III-A-3), or a pharmaceutically acceptable salt thereof, wherein X^{C1} is C—R^{C1}, X^{C2} is N, R^{C1} is —OCH₃, R^{C3} is —CH₃, R^{C4} is H, and R^{C5} is H.

In some embodiments, the invention relates to a compound selected from Table A, or a pharmaceutically acceptable salt thereof. In other embodiments, the invention relates to a compound selected from Table A, i.e., the compound in non-salt form.

TABLE A
Compound Structures and Names.

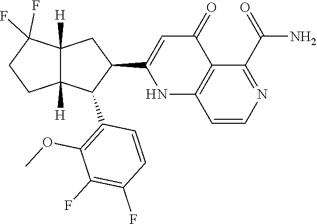 [SMILES: COc1c([C@@H]2[C@@H]3CCC(F)(F)[C@@H]3C[C@H]2c2cc(=O)c3c(C(N)=O)nccc3[nH]2)ccc(F)c1F]

2-((1R,2R,3aR,6aS)-1-(3,4-
difluoro-2-methoxyphenyl)-4,4-
difluorooctahydropentalen-2-
yl)-4-oxo-1,4-dihydro-1,6-
naphthyridine-5-carboxamide

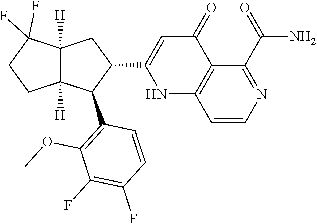 [SMILES: COc1c([C@H]2[C@H]3CCC(F)(F)[C@H]3C[C@@H]2c2cc(=O)c3c(C(N)=O)nccc3[nH]2)ccc(F)c1F]

2-((1S,2S,3aS,6aR)-1-(3,4-
difluoro-2-methoxyphenyl)-4,4-
difluorooctahydropentalen-2-
yl)-4-oxo-1,4-dihydro-1,6-
naphthyridine-5-carboxamide

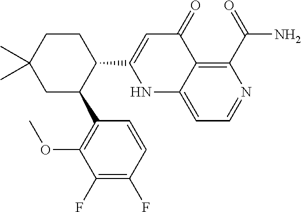 [SMILES: COc1c([C@H]2CC(C)(C)CC[C@@H]2c2cc(=O)c3c(C(N)=O)nccc3[nH]2)ccc(F)c1F]

2-((1S,2S)-2-(3,4-difluoro-2-
methoxyphenyl)-4,4-
dimethylcyclohexyl)-4-oxo-1,4-
dihydro-1,6-naphthyridine-5-
carboxamide

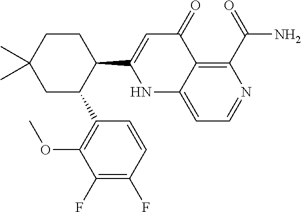 [SMILES: COc1c([C@@H]2CC(C)(C)CC[C@H]2c2cc(=O)c3c(C(N)=O)nccc3[nH]2)ccc(F)c1F]

2-((1R,2R)-2-(3,4-difluoro-2-
methoxyphenyl)-4,4-
dimethylcyclohexyl)-4-oxo-1,4-
dihydro-1,6-naphthyridine-5-
carboxamide

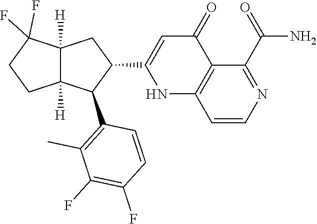 [SMILES: Cc1c([C@H]2[C@H]3CCC(F)(F)[C@H]3C[C@@H]2c2cc(=O)c3c(C(N)=O)nccc3[nH]2)ccc(F)c1F]

2-((1S,2S,3aS,6aR)-1-(3,4-
difluoro-2-methylphenyl)-4,4-
difluorooctahydropentalen-2-
yl)-4-oxo-1,4-dihydro-1,6-
naphthyridine-5-carboxamide

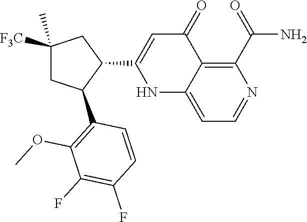 [SMILES: COc1c([C@H]2C[C@@](C)(C(F)(F)F)C[C@@H]2c2cc(=O)c3c(C(N)=O)nccc3[nH]2)ccc(F)c1F]

2-((1S,2S,4R)-2-(3,4-difluoro-2-
methoxyphenyl)-4-methyl-4-
(trifluoromethyl)cyclopentyl)-4-
oxo-1,4-dihydro-1,6-
naphthyridine-5-carboxamide

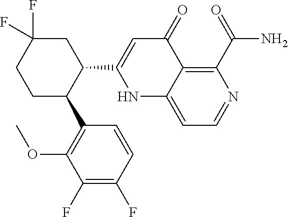 [SMILES: COc1c([C@H]2CCC(F)(F)C[C@@H]2c2cc(=O)c3c(C(N)=O)nccc3[nH]2)ccc(F)c1F]

2-((1S,2S)-2-(3,4-difluoro-2-
methoxyphenyl)-5,5-
difluorocyclohexyl)-4-oxo-1,4-
dihydro-1,6-naphthyridine-5-
carboxamide

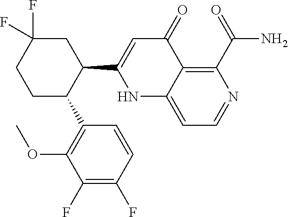 [SMILES: COc1c([C@@H]2CCC(F)(F)C[C@H]2c2cc(=O)c3c(C(N)=O)nccc3[nH]2)ccc(F)c1F]

2-((1R,2R)-2-(3,4-difluoro-2-
methoxyphenyl)-5,5-
difluorocyclohexyl)-4-oxo-1,4-
dihydro-1,6-naphthyridine-5-
carboxamide

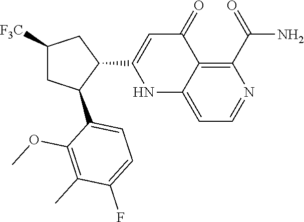 [SMILES: COc1c([C@H]2C[C@@H](C(F)(F)F)C[C@@H]2c2cc(=O)c3c(C(N)=O)nccc3[nH]2)ccc(F)c1C]

2-((1S,2S,4R)-2-(4-fluoro-2-
methoxy-3-methylphenyl)-4-
(trifluoromethyl)cyclopentyl)-4-
oxo-1,4-dihydro-1,6-
naphthyridine-5-carboxamide

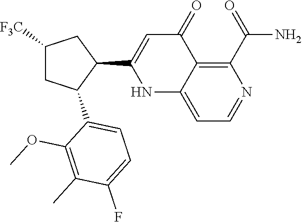 [SMILES: COc1c([C@@H]2C[C@H](C(F)(F)F)C[C@H]2c2cc(=O)c3c(C(N)=O)nccc3[nH]2)ccc(F)c1C]

2-((1R,2R,4S)-2-(4-fluoro-2-
methoxy-3-methylphenyl)-4-
(trifluoromethyl)cyclopentyl)-4-
oxo-1,4-dihydro-1,6-
naphthyridine-5-carboxamide

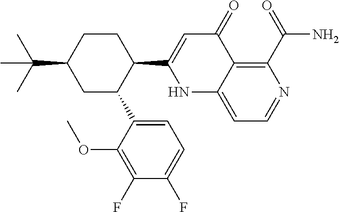 [SMILES: COc1c([C@@H]2C[C@@H](C(C)(C)C)CC[C@H]2c2cc(=O)c3c(C(N)=O)nccc3[nH]2)ccc(F)c1F]

2-((1R,2R,4S)-4-(tert-butyl)-2-
(3,4-difluoro-2-
methoxyphenyl)cyclohexyl)-4-
oxo-1,4-dihydro-1,6-
naphthyridine-5-carboxamide

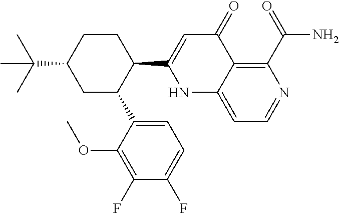 [SMILES: COc1c([C@@H]2C[C@H](C(C)(C)C)CC[C@H]2c2cc(=O)c3c(C(N)=O)nccc3[nH]2)ccc(F)c1F]

2-((1R,2R,4R)-4-(tert-butyl)-2-
(3,4-difluoro-2-
methoxyphenyl)cyclohexyl)-4-
oxo-1,4-dihydro-1,6-
naphthyridine-5-carboxamide

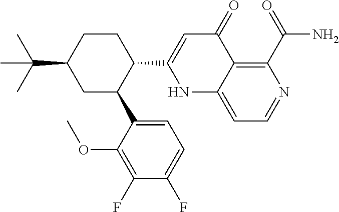 [SMILES: COc1c([C@H]2C[C@@H](C(C)(C)C)CC[C@@H]2c2cc(=O)c3c(C(N)=O)nccc3[nH]2)ccc(F)c1F]

2-((1S,2S,4S)-4-(tert-butyl)-2-
(3,4-difluoro-2-
methoxyphenyl)cyclohexyl)-4-
oxo-1,4-dihydro-1,6-
naphthyridine-5-carboxamide

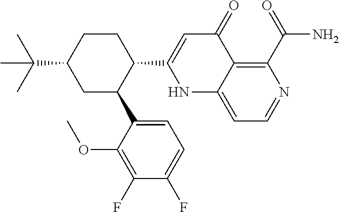 [SMILES: COc1c([C@H]2C[C@H](C(C)(C)C)CC[C@@H]2c2cc(=O)c3c(C(N)=O)nccc3[nH]2)ccc(F)c1F]

2-((1S,2S,4R)-4-(tert-butyl)-2-
(3,4-difluoro-2-
methoxyphenyl)cyclohexyl)-4-
oxo-1,4-dihydro-1,6-
naphthyridine-5-carboxamide

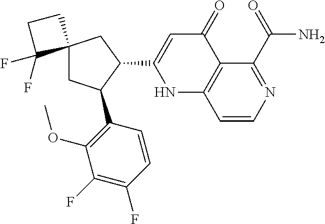 [SMILES: COc1c([C@H]2C[C@]3(CCC3(F)F)C[C@@H]2c2cc(=O)c3c(C(N)=O)nccc3[nH]2)ccc(F)c1F]

2-((4R,6S,7S)-7-(3,4-difluoro-2-
methoxyphenyl)-1,1-
difluorospiro[3.4]octan-6-yl)-4-
oxo-1,4-dihydro-1,6-
naphthyridine-5-carboxamide

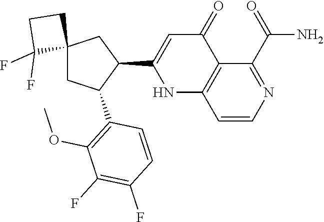 [SMILES: COc1c([C@@H]2C[C@@]3(CCC3(F)F)C[C@H]2c2cc(=O)c3c(C(N)=O)nccc3[nH]2)ccc(F)c1F]

2-((4S,6R,7R)-7-(3,4-difluoro-2-
methoxyphenyl)-1,1-
difluorospiro[3.4]octan-6-yl)-4-
oxo-1,4-dihydro-1,6-
naphthyridine-5-carboxamide

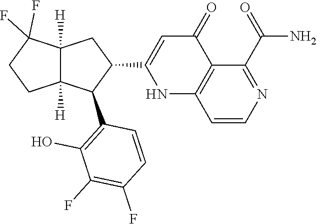 [SMILES: NC(=O)c1nccc2[nH]c([C@H]3C[C@H]4[C@H](CCC4(F)F)[C@@H]3c3ccc(F)c(F)c3O)cc(=O)c12]

2-((1S,2S,3aS,6aR)-1-(3,4-
difluoro-2-hydroxyphenyl)-4,4-
difluorooctahydropentalen-2-
yl)-4-oxo-1,4-dihydro-1,6-
naphthyridine-5-carboxamide

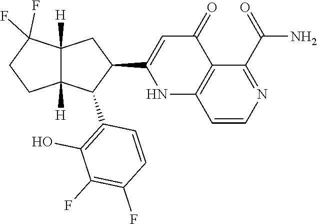 [SMILES: NC(=O)c1nccc2[nH]c([C@@H]3C[C@@H]4[C@@H](CCC4(F)F)[C@H]3c3ccc(F)c(F)c3O)cc(=O)c12]

2-((1R,2R,3aR,6aS)-1-(3,4-
difluoro-2-hydroxyphenyl)-4,4-
difluorooctahydropentalen-2-
yl)-4-oxo-1,4-dihydro-1,6-
naphthyridine-5-carboxamide

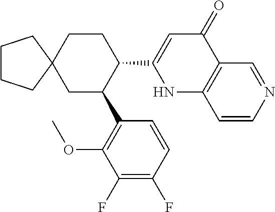 [SMILES: COc1c([C@H]2CC3(CCCC3)CC[C@@H]2c2cc(=O)c3cnccc3[nH]2)ccc(F)c1F]

2-((7S,8S)-7-(3,4-difluoro-2-
methoxyphenyl)spiro[4.5]decan-
8-yl)-1,6-naphthyridin-4(1H)-
one

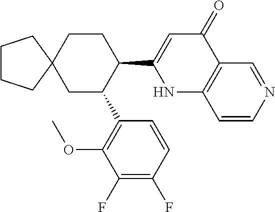 [SMILES: COc1c([C@@H]2CC3(CCCC3)CC[C@H]2c2cc(=O)c3cnccc3[nH]2)ccc(F)c1F]

2-((7R,8R)-7-(3,4-difluroo-2-
methoxyphenyl)spiro[4.5]decan-
8-yl)-1,6-naphthyridin-4(1H)-
one

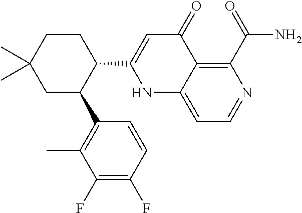 [SMILES: Cc1c([C@H]2CC(C)(C)CC[C@@H]2c2cc(=O)c3c(C(N)=O)nccc3[nH]2)ccc(F)c1F]

2-((1S,2S)-2-(3,4-difluoro-2-
methylphenyl)-4,4-
dimethylcyclohexyl)-4-oxo-1,4-
dihydro-1,6-naphthyridine-5-
carboxamide

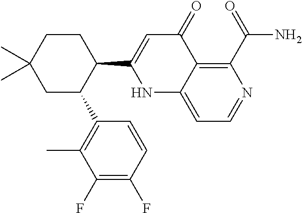 [SMILES: Cc1c([C@@H]2CC(C)(C)CC[C@H]2c2cc(=O)c3c(C(N)=O)nccc3[nH]2)ccc(F)c1F]

2-((1R,2R)-2-(3,4-difluoro-2-
methylphenyl)-4,4-
dimethylcyclohexyl)-4-oxo-1,4-
dihydro-1,6-naphthyridine-5-
carboxamide

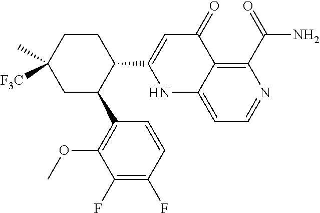 [SMILES: COc1c([C@H]2C[C@@](C)(C(F)(F)F)CC[C@@H]2c2cc(=O)c3c(C(N)=O)nccc3[nH]2)ccc(F)c1F]

2-((1S,2S,4S)-2-(3,4-difluoro-2-
methoxyphenyl)-4-methyl-4-
(trifluoromethyl)cyclohexyl)-4-
oxo-1,4-dihydro-1,6-
naphthyridine-5-carboxamide

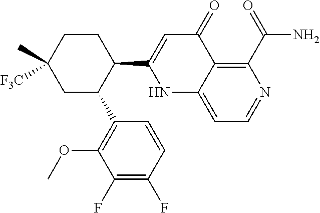 [SMILES: COc1c([C@@H]2C[C@](C)(C(F)(F)F)CC[C@H]2c2cc(=O)c3c(C(N)=O)nccc3[nH]2)ccc(F)c1F]

2-((1R,2R,4R)-2-(3,4-difluoro-2-
methoxyphenyl)-4-methyl-4-
(trifluoromethyl)cyclohexyl)-4-
oxo-1,4-dihydro-1,6-
naphthyridine-5-carboxamide

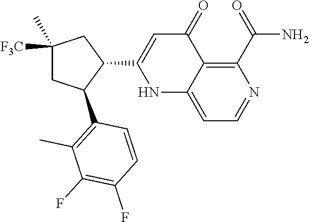 [SMILES: Cc1c([C@H]2C[C@@](C)(C(F)(F)F)C[C@@H]2c2cc(=O)c3c(C(N)=O)nccc3[nH]2)ccc(F)c1F]

2-((1S,2S,4R)-2-(3,4-difluoro-2-
methylphenyl)-4-methyl-4-
(trifluoromethyl)cyclopentyl)-4-
oxo-1,4-dihydro-1,6-
naphthyridine-5-carboxamide

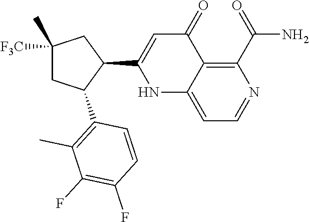 [SMILES: Cc1c([C@@H]2C[C@](C)(C(F)(F)F)C[C@H]2c2cc(=O)c3c(C(N)=O)nccc3[nH]2)ccc(F)c1F]

2-((1R,2R,4S)-2-(3,4-difluoro-2-
methylphenyl)-4-methyl-4-
(trifluoromethyl)cyclopentyl)-4-
oxo-1,4-dihydro-1,6-
naphthyridine-5-carboxamide

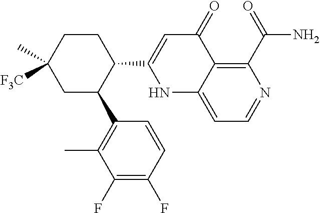 [SMILES: Cc1c([C@H]2C[C@@](C)(C(F)(F)F)CC[C@@H]2c2cc(=O)c3c(C(N)=O)nccc3[nH]2)ccc(F)c1F]

2-((1S,2S,4S)-2-(3,4-difluoro-2-
methylphenyl)-4-methyl-4-
(trifluoromethyl)cyclohexyl)-4-
oxo-1,4-dihydro-1,6-
naphthyridine-5-carboxamide

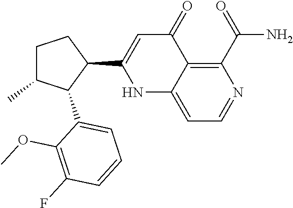 [SMILES: COc1c(F)cccc1[C@@H]1[C@H](C)CC[C@H]1c1cc(=O)c2c(C(N)=O)nccc2[nH]1]

2-((1R,2R,3R)-2-(3-fluoro-2-
methoxyphenyl)-3-
methylcyclopentyl)-4-oxo-1,4-
dihydro-1,6-naphthyridine-5-
carboxamide

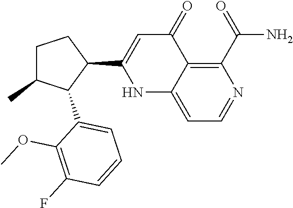 [SMILES: COc1c(F)cccc1[C@@H]1[C@@H](C)CC[C@H]1c1cc(=O)c2c(C(N)=O)nccc2[nH]1]

2-((1R,2R,3S)-2-(3-fluoro-2-
methoxyphenyl)-3-
methylcyclopentyl)-4-oxo-1,4-
dihydro-1,6-naphthyridine-5-
carboxamide

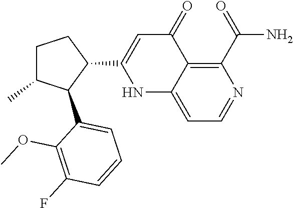 [SMILES: COc1c(F)cccc1[C@H]1[C@H](C)CC[C@@H]1c1cc(=O)c2c(C(N)=O)nccc2[nH]1]

2-((1S,2S,3R)-2-(3-fluoro-2-
methoxypehnyl)-3-
methylcyclopentyl)-4-oxo-1,4-
dihydro-1,6-naphthyridine-5-
carboxamide

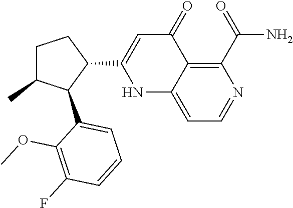 [SMILES: COc1c(F)cccc1[C@@H]1[C@@H](c2cc(=O)c3c(C(N)=O)nccc3[nH]2)CC[C@@H]1C]

2-((1S,2S,3S)-2-(3-fluoro-2-
methoxyphenyl)-3-
methylcyclopentyl)-4-oxo-1,4-
dihydro-1,6-naphthyridine-5-
carboxamide

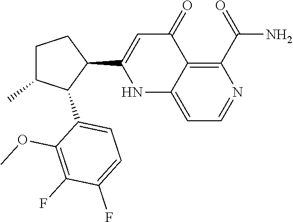 [SMILES: COc1c([C@@H]2[C@H](C)CC[C@H]2c2cc(=O)c3c(C(N)=O)nccc3[nH]2)ccc(F)c1F]

2-((1R,2R,3R)-2-(3,4-difluoro-2-
methoxyphenyl)-3-
methylcyclopentyl)-4-oxo-1,4-
dihydro-1,6-naphthyridine-5-
carboxamide

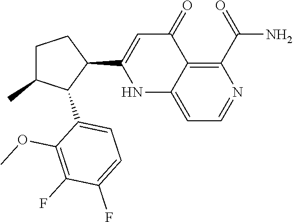 [SMILES: COc1c([C@@H]2[C@@H](C)CC[C@H]2c2cc(=O)c3c(C(N)=O)nccc3[nH]2)ccc(F)c1F]

2-((1R,2R,3S)-2-(3,4-difluoro-2-
methoxyphenyl)-3-
methylcyclopentyl)-4-oxo-1,4-
dihydro-1,6-naphthyridine-5-
carboxamide

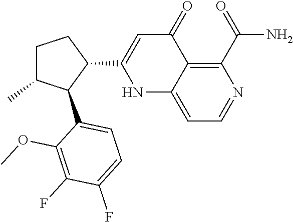 [SMILES: COc1c([C@H]2[C@H](C)CC[C@@H]2c2cc(=O)c3c(C(N)=O)nccc3[nH]2)ccc(F)c1F]

2-((1S,2S,3R)-2-(3,4-difluoro-2-
methoxyphenyl)-3-
methylcyclopentyl)-4-oxo-1,4-
dihydro-1,6-naphthyridine-5-
carboxamide

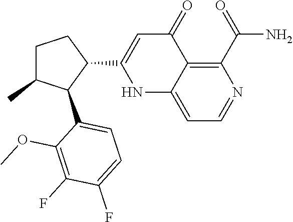 [SMILES: COc1c([C@@H]2[C@@H](c3cc(=O)c4c(C(N)=O)nccc4[nH]3)CC[C@@H]2C)ccc(F)c1F]

2-((1S,2S,3S)-2-(3,4-difluoro-2-
methoxyphenyl)-3-
methylcyclopentyl)-4-oxo-1,4-
dihydro-1,6-naphthyridine-5-
carboxamide

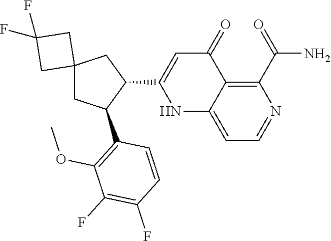 [SMILES: COc1c([C@H]2CC3(C[C@@H]2c2cc(=O)c4c(C(N)=O)nccc4[nH]2)CC(F)(F)C3)ccc(F)c1F]

2-((6S,7S)-7-(3,4-difluoro-2-
methoxyphenyl)-2,2-
difluorospiro[3.4]octan-6-yl)-4-
oxo-1,4-dihydro-1,6-
naphthyridine-5-carboxamide

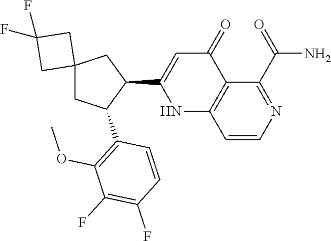 [SMILES: COc1c([C@@H]2CC3(C[C@H]2c2cc(=O)c4c(C(N)=O)nccc4[nH]2)CC(F)(F)C3)ccc(F)c1F]

2-((6R,7R)-7-(3,4-difluoro-2-
methoxyphenyl)-2,2-
difluorospiro[3.4]octan-6-yl)-4-
oxo-1,4-dihdyro-1,6-
naphthyridine-5-carboxamide

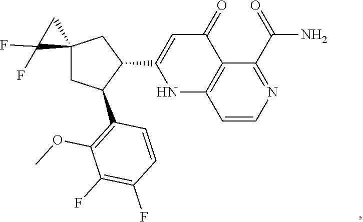 [SMILES: COc1c([C@H]2C[C@@]3(C[C@@H]2c2cc(=O)c4c(C(N)=O)nccc4[nH]2)CC3(F)F)ccc(F)c1F]

2-((3R,5S,6S)-6-(3,4-difluoro-2-
methoxyphenyl)-1,1-
difluorospiro[2.4]heptan-5-yl)-
4-oxo-1,4-dihydro-1,6-
naphthyridine-5-carboxamide

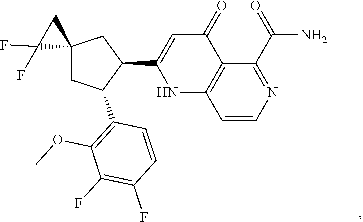 [SMILES: COc1c([C@@H]2C[C@]3(C[C@H]2c2cc(=O)c4c(C(N)=O)nccc4[nH]2)CC3(F)F)ccc(F)c1F]

2-((3S,5R,6R)-6-(3,4-difluoro-2-
methoxyphenyl)-1,1-
difluorospiro[2.4]heptan-5-yl)-
4-oxo-1,4-dihydro-1,6-
naphthyridine-5-carboxamide

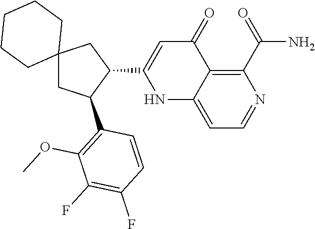 [SMILES: COc1c([C@H]2CC3(CCCCC3)C[C@@H]2c2cc(=O)c3c(C(N)=O)nccc3[nH]2)ccc(F)c1F]

2-((2S,3S)-3-(3,4-difluoro-2-
methoxyphenyl)spiro[4.5]decan-
2-yl)-4-oxo-1,4-dihydro-1,6-
naphthyridine-5-carboxamide

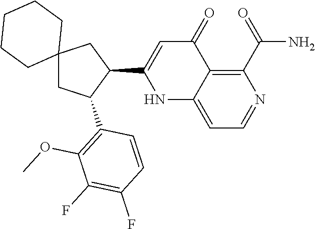 [SMILES: COc1c([C@@H]2CC3(CCCCC3)C[C@H]2c2cc(=O)c3c(C(N)=O)nccc3[nH]2)ccc(F)c1F]

2-((2R,3R)-3-(3,4-difluoro-2-
methoxyphenyl)spiro[4.5]decan-
2-yl)-4-oxo-1,4-dihydro-1,6-
naphthyridine-5-carboxamide

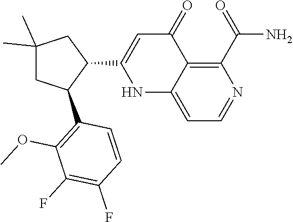 [SMILES: COc1c([C@H]2CC(C)(C)C[C@@H]2c2cc(=O)c3c(C(N)=O)nccc3[nH]2)ccc(F)c1F]

2-((1S,2S)-2-(3,4-difluoro-2-
methoxyphenyl)-4,4-
dimethylcyclopentyl)-4-oxo-1,4-
dihydro-1,6-naphthyridine-5-
carboxamide

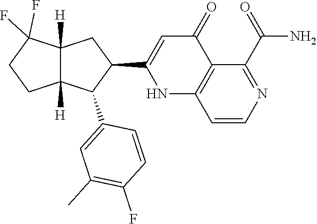 [SMILES: Cc1cc([C@@H]2[C@@H]3CCC(F)(F)[C@@H]3C[C@H]2c2cc(=O)c3c(C(N)=O)nccc3[nH]2)ccc1F]

2-((1R,2R,3aR,6aS)-4,4-
difluoro-1-(4-fluoro-3-
methylphenyl)octahydropentalen-
2-yl)-4-oxo-1,4-dihydro-1,6-
naphthyridine-5-carboxamide

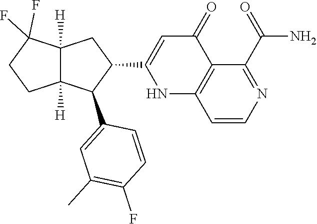 [SMILES: Cc1cc([C@H]2[C@H]3CCC(F)(F)[C@H]3C[C@@H]2c2cc(=O)c3c(C(N)=O)nccc3[nH]2)ccc1F]

2-((1S,2S,3aS,6aR)-4,4-difluoro-
1-(4-fluoro-3-
methylphenyl)octahydropentalen-
2-yl)-4-oxo-1,4-dihydro-1,6-
naphthyridine-5-carboxamide

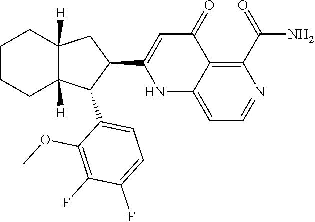 [SMILES: COc1c([C@@H]2[C@@H]3CCCC[C@@H]3C[C@H]2c2cc(=O)c3c(C(N)=O)nccc3[nH]2)ccc(F)c1F]

2-((1R,2R,3aR,7aR)-1-(3,4-
difluoro-2-
methoxyphenyl)octahydro-1H-
inden-2-yl)-4-oxo-1,4-dihydro-
1,6-naphthyridine-5-
carboxamide

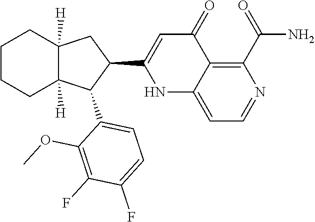 [SMILES: COc1c([C@@H]2[C@H]3CCCC[C@H]3C[C@H]2c2cc(=O)c3c(C(N)=O)nccc3[nH]2)ccc(F)c1F]

2-((1R,2R,3aS,7aS)-1-(3,4-
difluoro-2-
methoxyphenyl)octahydro-1H-
inden-2-yl)-4-oxo-1,4-dihydro-
1,6-naphthyridine-5-
carboxamide

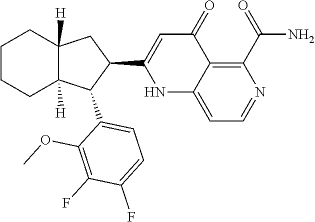 [SMILES: COc1c([C@@H]2[C@H]3CCCC[C@@H]3C[C@H]2c2cc(=O)c3c(C(N)=O)nccc3[nH]2)ccc(F)c1F]

2-((1R,2R,3aR,7aS)-1-(3,4-
difluoro-2-
methoxyphenyl)octahydro-1H-
inden-2-yl)-4-oxo-1,4-dihydro-
1,6-naphthyridine-5-
carboxamide

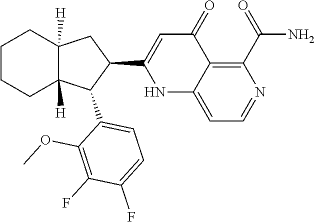 [SMILES: COc1c([C@@H]2[C@@H]3CCCC[C@H]3C[C@H]2c2cc(=O)c3c(C(N)=O)nccc3[nH]2)ccc(F)c1F]

2-((1R,2R,3aS,7aR)-1-(3,4-
difluoro-2-
methoxyphenyl)octahydro-1H-
inden-2-yl)-4-oxo-1,4-dihydro-
1,6-naphthyridine-5-
carboxamide

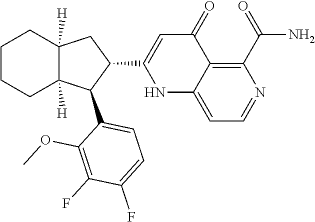 [SMILES: COc1c([C@H]2[C@H]3CCCC[C@H]3C[C@@H]2c2cc(=O)c3c(C(N)=O)nccc3[nH]2)ccc(F)c1F]

2-((1S,2S,3aS,7aS)-1-(3,4-
difluoro-2-
methoxyphenyl)octahydro-1H-
inden-2-yl)-4-oxo-1,4-dihydro-
1,6-naphthyridine-5-
carboxamide

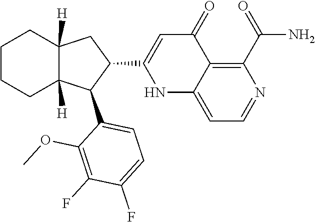 [SMILES: COc1c([C@H]2[C@@H]3CCCC[C@@H]3C[C@@H]2c2cc(=O)c3c(C(N)=O)nccc3[nH]2)ccc(F)c1F]

2-((1S,2S,3aR,7aR)-1-(3,4-
difluoro-2-
methoxyphenyl)octahydro-1H-
inden-2-yl)-4-oxo-1,4-dihydro-
1,6-naphthyridine-5-
carboxamide

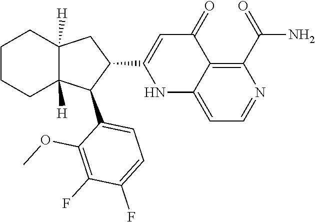 [SMILES: COc1c([C@H]2[C@@H]3CCCC[C@H]3C[C@@H]2c2cc(=O)c3c(C(N)=O)nccc3[nH]2)ccc(F)c1F]

2-((1S,2S,3aS,7aR)-1-(3,4-
difluoro-2-
methoxyphenyl)octahydro-1H-
inden-2-yl)-4-oxo-1,4-dihydro-
1,6-naphthyridine-5-
carboxamide

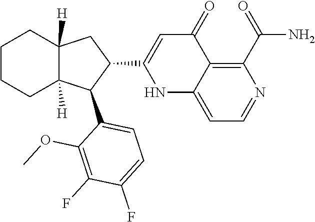 [SMILES: COc1c([C@H]2[C@H]3CCCC[C@@H]3C[C@@H]2c2cc(=O)c3c(C(N)=O)nccc3[nH]2)ccc(F)c1F]

2-((1S,2S,3aR,7aS)-1-(3,4-
difluoro-2-
methoxyphenyl)octahydro-1H-
inden-2-yl)-4-oxo-1,4-dihydro-
1,6-naphthyridine-5-
carboxamide

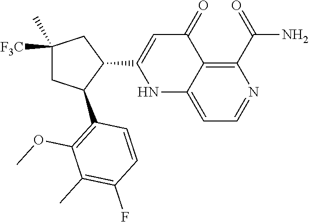 [SMILES: COc1c([C@H]2C[C@@](C)(C(F)(F)F)C[C@@H]2c2cc(=O)c3c(C(N)=O)nccc3[nH]2)ccc(F)c1C]

2-((1S,2S,4R)-2-(4-fluoro-2-
methoxy-3-methylphenyl)-4-
methyl-4-
(trifluoromethyl)cyclopentyl)-4-
oxo-1,4-dihydro-1,6-
naphthyridine-5-carboxamide

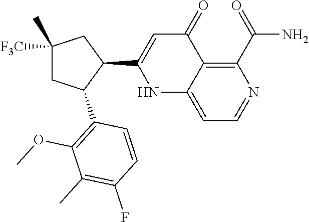 [SMILES: COc1c([C@@H]2C[C@](C)(C(F)(F)F)C[C@H]2c2cc(=O)c3c(C(N)=O)nccc3[nH]2)ccc(F)c1C]

2-((1R,2R,4S)-2-(4-fluoro-2-
methoxy-3-methylphenyl)-4-
methyl-4-
(trifluromethyl)cyclohexyl)-4-
oxo-1,4-dihydro-1,6-
naphthyridine-5-carboxamide

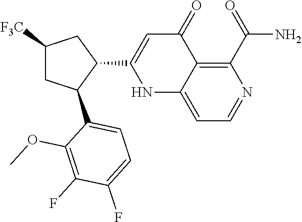 [SMILES: COc1c([C@H]2C[C@@H](C(F)(F)F)C[C@@H]2c2cc(=O)c3c(C(N)=O)nccc3[nH]2)ccc(F)c1F]

2-((1S,2S,4R)-2-(3,4-difluoro-2-
methoxyphenyl)-4-
(trifluoromethyl)cyclopentyl)-4-
oxo-1,4-dihydro-1,6-
naphthyridine-5-carboxamide

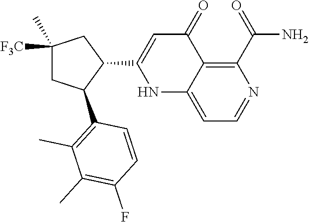 [SMILES: Cc1c(F)ccc([C@H]2C[C@@](C)(C(F)(F)F)C[C@@H]2c2cc(=O)c3c(C(N)=O)nccc3[nH]2)c1C]

2-((1S,2S,4R)-2-(4-fluoro-2,3-
dimethylphenyl)-4-methyl-4-
(trifluoromethyl)cyclopentyl)-4-
oxo-1,4-dihydro-1,6-
naphthyridine-5-carboxamide

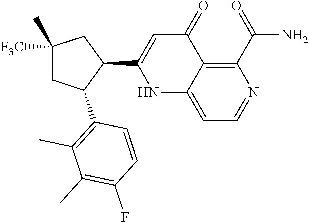 [SMILES: Cc1c(F)ccc([C@@H]2C[C@](C)(C(F)(F)F)C[C@H]2c2cc(=O)c3c(C(N)=O)nccc3[nH]2)c1C]

2-((1R,2R,4S)-2-(4-fluoro-2,3-
dimethylphenyl)-4-methyl-4-
(trifluoromethyl)cyclopentyl)-4-
oxo-1,4-dihydro-1,6-
naphthyridine-5-carboxamide

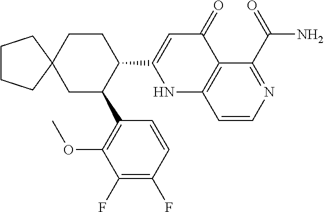 [SMILES: COc1c([C@H]2CC3(CCCC3)CC[C@@H]2c2cc(=O)c3c(C(N)=O)nccc3[nH]2)ccc(F)c1F]

2-((7S,8S)-7-(3,4-difluoro-2-
methoxyphenyl)spiro[4.5]decan-
8-yl)-4-oxo-1,4-dihydro-1,6-
naphthyridine-5-carboxamide

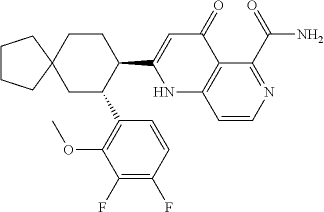 [SMILES: COc1c([C@@H]2CC3(CCCC3)CC[C@H]2c2cc(=O)c3c(C(N)=O)nccc3[nH]2)ccc(F)c1F]

2-((7R,8R)-7-(3,4-difluoro-2-
methoxyphenyl)spiro[4.5]decan-
8-yl)-4-oxo-1,4-dihydro-1,6-
naphthyridine-5-carboxamide

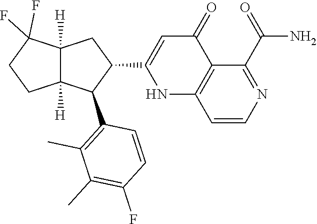 [SMILES: Cc1c(F)ccc([C@H]2[C@H]3CCC(F)(F)[C@H]3C[C@@H]2c2cc(=O)c3c(C(N)=O)nccc3[nH]2)c1C]

2-((1S,2S,3aS,6aR)-4,4-difluoro-
1-(4-fluoro-2,3-
dimethylphenyl)octahydropentalen-
2-yl)-4-oxo-1,4-dihydro-1,6-
naphthyridine-5-carboxamide

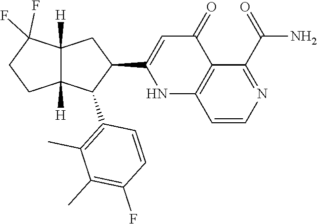 [SMILES: Cc1c(F)ccc([C@@H]2[C@@H]3CCC(F)(F)[C@@H]3C[C@H]2c2cc(=O)c3c(C(N)=O)nccc3[nH]2)c1C]

2-((1R,2R,3aR,6aS)-4,4-
difluoro-1-(4-fluoro-2,3-
dimethylphenyl)octahydropentalen-
2-yl)-4-oxo-1,4-dihydro-1,6-
naphthyridine-5-carboxamide

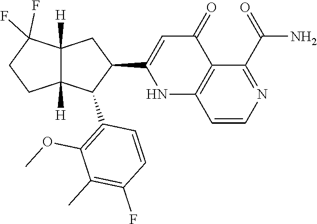 [SMILES: COc1c([C@@H]2[C@@H]3CCC(F)(F)[C@@H]3C[C@H]2c2cc(=O)c3c(C(N)=O)nccc3[nH]2)ccc(F)c1C]

2-((1R,2R,3aR,6aS)-4,4-
difluoro-1-(4-fluoro-2-methoxy-
3-
methylphenyl)octahydropentalen-
2-yl)-4-oxo-1,4-dihydro-1,6-
naphthyridine-5-carboxamide

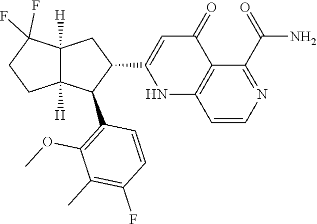 [SMILES: COc1c([C@H]2[C@H]3CCC(F)(F)[C@H]3C[C@@H]2c2cc(=O)c3c(C(N)=O)nccc3[nH]2)ccc(F)c1C]

2-((1S,2S,3aS,6aR)-4,4-difluoro-
1-(4-fluoro-2-methoxy-3-
mehtylphenyl)octahydropentalen-
2-yl)-4-oxo-1,4-dihydro-1,6-
naphthyridine-5-carboxamide

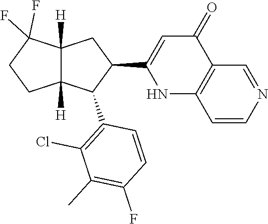 [SMILES: Cc1c(F)ccc([C@@H]2[C@@H]3CCC(F)(F)[C@@H]3C[C@H]2c2cc(=O)c3cnccc3[nH]2)c1Cl]

2-((1R,2R,3aR,6aS)-1-(2-chloro-
4-fluoro-3-methylphenyl)-4,4-
difluorooctahydropentalen-2-
yl)-1,6-naphthyridin-4(1H)-one

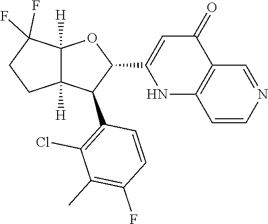 [SMILES: Cc1c(F)ccc([C@H]2[C@H]3CCC(F)(F)[C@H]3O[C@@H]2c2cc(=O)c3cnccc3[nH]2)c1Cl]

2-((1S,2S,3aS,6aR)-1-(2-chloro-
4-fluoro-3-methylphenyl)-4,4-
difluorooctahydropentalen-2-
yl)-1,6-naphthyridin-4(1H)-one

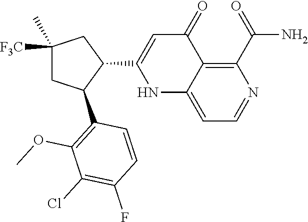 [SMILES: COc1c([C@H]2C[C@@](C)(C(F)(F)F)C[C@@H]2c2cc(=O)c3c(C(N)=O)nccc3[nH]2)ccc(F)c1Cl]

2-((1S,2S,4R)-2-(3-chlroo-4-
fluoro-2-methoxyphenyl)-4-
methyl-4-
(trifluoromethyl)cyclopentyl)-4-
oxo-1,4-dihydro-1,6-
naphthyridine-5-carboxamide

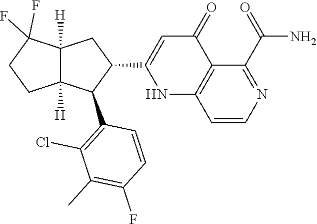 [SMILES: Cc1c(F)ccc([C@H]2[C@H]3CCC(F)(F)[C@H]3C[C@@H]2c2cc(=O)c3c(C(N)=O)nccc3[nH]2)c1Cl]

2-((1S,2S,3aS,6aR)-1-(2-chloro-
4-fluoro-3-methylphenyl)-4,4-
difluorooctahydropentalen-2-
yl)-4-oxo-1,4-dihydro-1,6-
naphthyridine-5-carboxamide

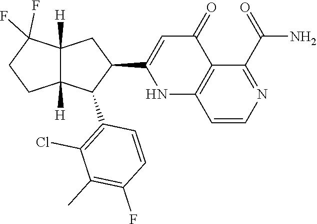 [SMILES: Cc1c(F)ccc([C@@H]2[C@@H]3CCC(F)(F)[C@@H]3C[C@H]2c2cc(=O)c3c(C(N)=O)nccc3[nH]2)c1Cl]

2-((1R,2R,3aR,6aS)-1-(2-chloro-
4-fluoro-3-methylphenyl)-4,4-
difluorooctahydropentalen-2-
yl)-4-oxo-1,4-dihydro-1,6-
naphthyridine-5-carboxamide

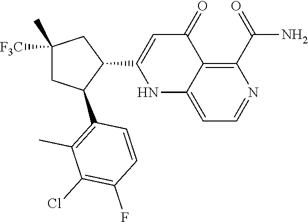 [SMILES: Cc1c([C@H]2C[C@](C)(C(F)(F)F)C[C@@H]2c2cc(=O)c3c(C(N)=O)nccc3[nH]2)ccc(F)c1Cl]

2-((1S,2S,4S)-2-(3-chloro-4-
fluoro-2-methylphenyl)-4-
methyl-4-
(trifluoromethyl)cyclopentyl)-4-
oxo-1,4-dihydro-1,6-
naphthyridine-5-carboxamide

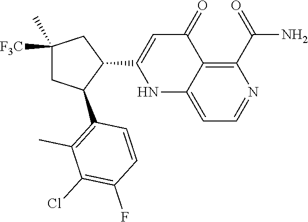 [SMILES: Cc1c([C@H]2C[C@@](C)(C(F)(F)F)C[C@@H]2c2cc(=O)c3c(C(N)=O)nccc3[nH]2)ccc(F)c1Cl]

2-((1S,2S,4R)-2-(3-chloro-4-
fluoro-2-methylphenyl)-4-
methyl-4-
(trifluoromethyl)cyclopentyl)-4-
oxo-1,4-dihydro-1,6-
naphthyridine-5-carboxamide

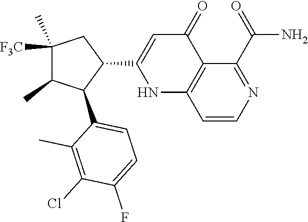 [SMILES: Cc1c([C@@H]2[C@@H](c3cc(=O)c4c(C(N)=O)nccc4[nH]3)C[C@](C)(C(F)(F)F)[C@@H]2C)ccc(F)c1Cl]

2-((1S,2S,3R,4S)-2-(3-chloro-4-
fluoro-2-methylphenyl)-3,4-
dimethyl-4-
(trifluoromethyl)cyclopentyl)-4-
oxo-1,4-dihydro-1,6-
naphthyridine-5-carboxamide

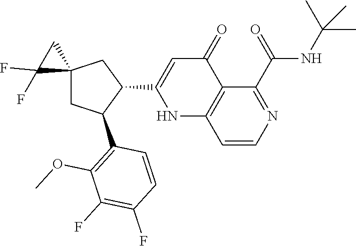 [SMILES: COc1c([C@H]2C[C@@]3(C[C@@H]2c2cc(=O)c4c(C(=O)NC(C)(C)C)nccc4[nH]2)CC3(F)F)ccc(F)c1F]

N-(tert-butyl)-2-((3R,5S,6S)-6-
(3,4-difluoro-2-
methoxyphenyl)-1,1-
difluorospiro[2.4]heptan-5-yl)-
4-oxo-1,4-dihydro-1,6-
naphthyridine-5-carboxamide

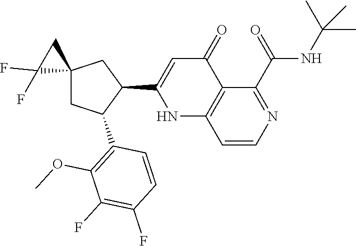 [SMILES: COc1c([C@@H]2C[C@]3(C[C@H]2c2cc(=O)c4c(C(=O)NC(C)(C)C)nccc4[nH]2)CC3(F)F)ccc(F)c1F]

N-(tert-butyl)-2-((3S,5R,6R)-6-
(3,4-difluoro-2-
methoxyphenyl)-1,1-
difluorospiro[2.4]heptan-5-yl)-
4-oxo-1,4-dihydro-1,6-
naphthyridine-5-carboxamide

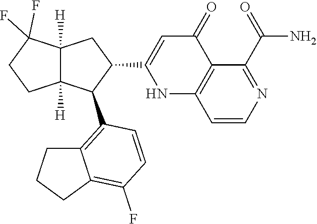 [SMILES: NC(=O)c1nccc2[nH]c([C@H]3C[C@H]4[C@H](CCC4(F)F)[C@@H]3c3ccc(F)c4c3CCC4)cc(=O)c12]

2-((1S,2S,3aS,6aR)-4,4-difluoro-
1-(7-fluoro-2,3-dihydro-1H-
inden-4-yl)octahydropentalen-2-
yl)-4-oxo-1,4-dihydro-1,6-
naphthyridine-5-carboxamide

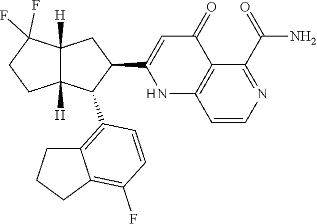 [SMILES: NC(=O)c1nccc2[nH]c([C@@H]3C[C@@H]4[C@@H](CCC4(F)F)[C@H]3c3ccc(F)c4c3CCC4)cc(=O)c12]

2-((1R,2R,3aR,6aS)-4,4-
difluoro-1-(7-fluoro-2,3-
dihydro-1H-inden-4-
yl)octahydropentalen-2-yl)-4-
oxo-1,4-dihydro-1,6-
naphthyridine-5-carboxamide

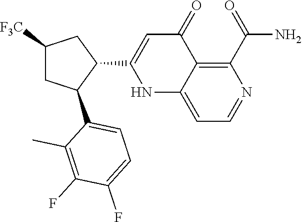 [SMILES: Cc1c([C@H]2C[C@@H](C(F)(F)F)C[C@@H]2c2cc(=O)c3c(C(N)=O)nccc3[nH]2)ccc(F)c1F]

2-((1S,2S,4R)-2-(3,4-difluoro-2-
methylphenyl)-4-
(trifluoromethyl)cyclopentyl)-4-
oxo-1,4-dihydro-1,6-
naphthyridine-5-carboxamide

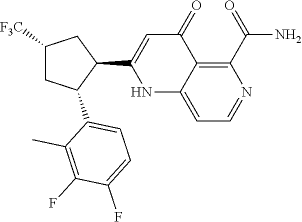 [SMILES: Cc1c([C@@H]2C[C@H](C(F)(F)F)C[C@H]2c2cc(=O)c3c(C(N)=O)nccc3[nH]2)ccc(F)c1F]

2-((1R,2R,4S)-2-(3,4-difluoro-2-
methylphenyl)-4-
(trifluoromethyl)cyclopentyl)-4-
oxo-1,4-dihydro-1,6-
naphthyridine-5-carboxamide

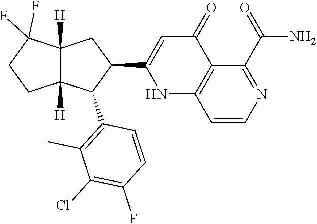 [SMILES: Cc1c([C@@H]2[C@@H]3CCC(F)(F)[C@@H]3C[C@H]2c2cc(=O)c3c(C(N)=O)nccc3[nH]2)ccc(F)c1Cl]

2-((1R,2R,3aR,6aS)-1-(3-chloro-
4-fluoro-2-methylphenyl)-4,4-
difluorooctahydropentalen-2-
yl)-4-oxo-1,4-dihydro-1,6-
naphthyridine-5-carboxamide

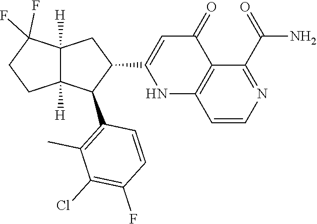 [SMILES: Cc1c([C@H]2[C@H]3CCC(F)(F)[C@H]3C[C@@H]2c2cc(=O)c3c(C(N)=O)nccc3[nH]2)ccc(F)c1Cl]

2-((1S,2S,3aS,6aR)-1-(3-chloro-
4-fluoro-2-methylphenyl)-4,4-
difluorooctahydropentalen-2-
yl)-4-oxo-1,4-dihydro-1,6-
naphthyridine-5-carboxamide

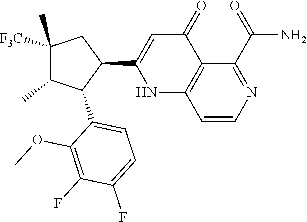 [SMILES: COc1c([C@H]2[C@H](c3cc(=O)c4c(C(N)=O)nccc4[nH]3)C[C@@](C)(C(F)(F)F)[C@H]2C)ccc(F)c1F]

2-((1R,2R,3S,4R)-2-(3,4-
difluoro-2-methoxyphenyl)-3,4-
dimethyl-4-
(trifluoromethyl)cyclopentyl)-4-
oxo-1,4-dihydro-1,6-
naphthyridine-5-carboxamide

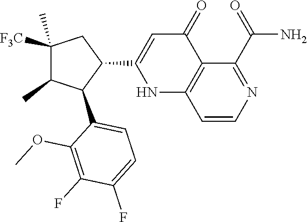 [SMILES: COc1c([C@@H]2[C@@H](c3cc(=O)c4c(C(N)=O)nccc4[nH]3)C[C@](C)(C(F)(F)F)[C@@H]2C)ccc(F)c1F]

2-((1S,2S,3R,4S)-2-(3,4-
difluoro-2-methoxyphenyl)-3,4-
dimethyl-4-
(trifluoromethyl)cyclopentyl)-4-
oxo-1,4-dihydro-1,6-
naphthyridine-5-carboxamide

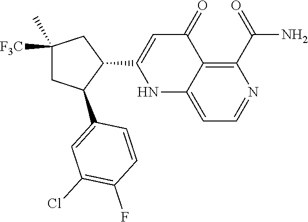 [SMILES: C[C@@]1(C(F)(F)F)C[C@H](c2ccc(F)c(Cl)c2)[C@@H](c2cc(=O)c3c(C(N)=O)nccc3[nH]2)C1]

2-((1S,2S,4R)-2-(3-chloro-4-
fluorophenyl)-4-methyl-4-
(trifluoromethyl)cyclopentyl)-4-
oxo-1,4-dihydro-1,6-
naphthyridine-5-carboxamide

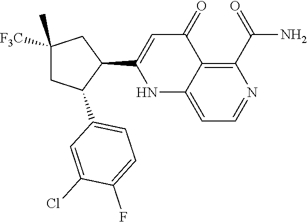 [SMILES: C[C@]1(C(F)(F)F)C[C@@H](c2ccc(F)c(Cl)c2)[C@H](c2cc(=O)c3c(C(N)=O)nccc3[nH]2)C1]

2-((1R,2R,4S)-2-(3-chloro-4-
fluorophenyl)-4-methyl-4-
(trifluoromethyl)cyclopentyl)-4-
oxo-1,4-dihydro-1,6-
naphthyridine-5-carboxamide

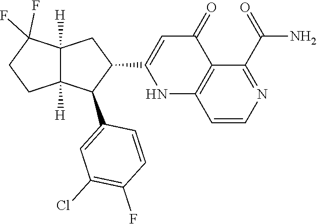 [SMILES: NC(=O)c1nccc2[nH]c([C@H]3C[C@H]4[C@H](CCC4(F)F)[C@@H]3c3ccc(F)c(Cl)c3)cc(=O)c12]

2-((1S,2S,3aS,6aR)-1-(3-chloro-
4-fluorophenyl)-4,4-
difluorooctahydropentalen-2-
yl)-4-oxo-1,4-dihydro-1,6-
naphthyridine-5-carboxamide

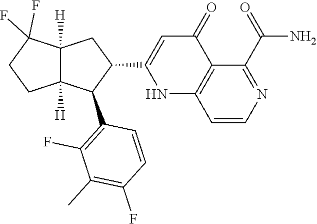 [SMILES: Cc1c(F)ccc([C@H]2[C@H]3CCC(F)(F)[C@H]3C[C@@H]2c2cc(=O)c3c(C(N)=O)nccc3[nH]2)c1F]

2-((1S,2S,3aS,6aR)-1-(2,4-
difluoro-3-methylphenyl)-4,4-
difluorooctahydropentalen-2-
yl)-4-oxo-1,4-dihydro-1,6-
naphthyridine-5-carboxamide

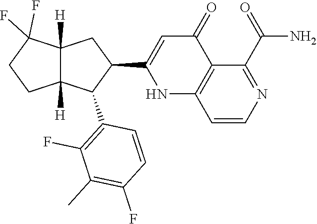 [SMILES: Cc1c(F)ccc([C@@H]2[C@@H]3CCC(F)(F)[C@@H]3C[C@H]2c2cc(=O)c3c(C(N)=O)nccc3[nH]2)c1F]

2-((1R,2R,3aR,6aS)-1-(2,4-
difluoro-3-methylphenyl)-4,4-
difluorooctahydropentalen-2-
yl)-4-oxo-1,4-dihydro-1,6-
naphthyridine-5-carboxamide

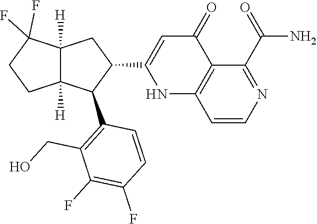 [SMILES: NC(=O)c1nccc2[nH]c([C@H]3C[C@H]4[C@H](CCC4(F)F)[C@@H]3c3ccc(F)c(F)c3CO)cc(=O)c12]

2-((1S,2S,3aS,6aR)-1-(3,4-
difluoro-2-
(hydroxymethyl)phenyl)-4,4-
difluorooctahydropentalen-2-
yl)-4-oxo-1,4-dihydro-1,6-
naphthyridine-5-carboxamide

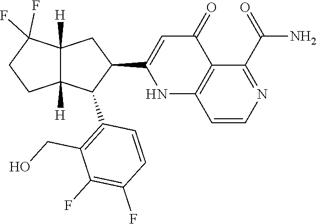 [SMILES: NC(=O)c1nccc2[nH]c([C@@H]3C[C@@H]4[C@@H](CCC4(F)F)[C@H]3c3ccc(F)c(F)c3CO)cc(=O)c12]

2-((1R,2R,3aR,6aS)-1-(3,4-
difluoro-2-
(hydroxymethyl)phenyl)-4,4-
difluorooctahydropentalen-2-
yl)-4-oxo-1,4-dihydro-1,6-
naphthyridine-5-carboxamide

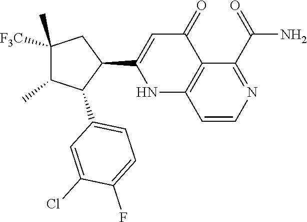 [SMILES: C[C@H]1[C@@H](c2ccc(F)c(Cl)c2)[C@H](c2cc(=O)c3c(C(N)=O)nccc3[nH]2)C[C@@]1(C)C(F)(F)F]

2-((1R,2R,3S,4R)-2-(3-chloro-4-
fluorophenyl)-3,4-dimethyl-4-
(trifluoromethyl)cyclopentyl)-4-
oxo-1,4-dihydro-1,6-
naphthyridine-5-carboxamide

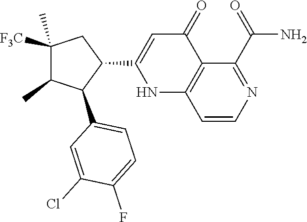 [SMILES: C[C@@H]1[C@H](c2ccc(F)c(Cl)c2)[C@@H](c2cc(=O)c3c(C(N)=O)nccc3[nH]2)C[C@]1(C)C(F)(F)F]

2-((1S,2S,3R,4S)-2-(3-chloro-4-
fluorophenyl)-3,4-dimethyl-4-
(trifluoromethyl)cyclopentyl)-4-
oxo-1,4-dihydro-1,6-
naphthyridine-5-carboxamide

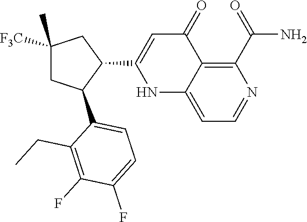 [SMILES: CCc1c([C@H]2C[C@](C)(C(F)(F)F)C[C@@H]2c2cc(=O)c3c(C(N)=O)nccc3[nH]2)ccc(F)c1F]

2-((1S,2S,4S)-2-(2-ethyl-3,4-
difluorophenyl)-4-methyl-4-
(trifluoromethyl)cyclopentyl)-4-
oxo-1,4-dihydro-1,6-
naphthyridine-5-carboxamide

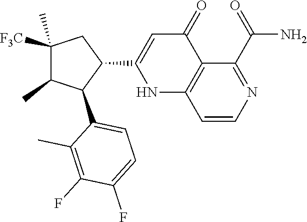 [SMILES: Cc1c([C@@H]2[C@@H](c3cc(=O)c4c(C(N)=O)nccc4[nH]3)C[C@](C)(C(F)(F)F)[C@@H]2C)ccc(F)c1F]

2-((1S,2S,3R,4S)-2-(3,4-
difluoro-2-methylphenyl)-3,4-
dimethyl-4-
(trifluoromethyl)cyclopentyl)-4-
oxo-1,4-dihydro-1,6-
naphthyridine-5-carboxamide

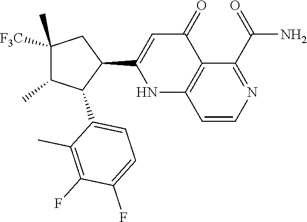 [SMILES: Cc1c([C@H]2[C@H](c3cc(=O)c4c(C(N)=O)nccc4[nH]3)C[C@@](C)(C(F)(F)F)[C@H]2C)ccc(F)c1F]

2-((1R,2R,3S,4R)-2-(3,4-
difluoro-2-methylphenyl)-3,4-
dimethyl-4-
(trifluoromethyl)cyclopentyl)-4-
oxo-1,4-dihydro-1,6-
naphthyridine-5-carboxamide

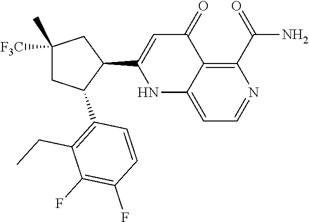 [SMILES: CCc1c([C@@H]2C[C@](C)(C(F)(F)F)C[C@H]2c2cc(=O)c3c(C(N)=O)nccc3[nH]2)ccc(F)c1F]

2-((1R,2R,4S)-2-(2-ethyl-3,4-
difluorophenyl)-4-methyl-4-
(trifluoromethyl)cyclopentyl)-4-
oxo-1,4-dihydro-1,6-
naphthyridine-5-carboxamide

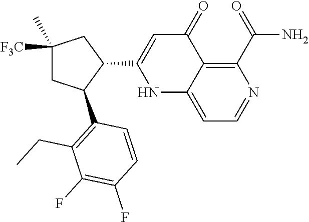 [SMILES: CCc1c([C@H]2C[C@@](C)(C(F)(F)F)C[C@@H]2c2cc(=O)c3c(C(N)=O)nccc3[nH]2)ccc(F)c1F]

2-((1S,2S,4R)-2-(2-ethyl-3,4-
difluorophenyl)-4-methyl-4-
(trifluoromethyl)cyclopentyl)-4-
oxo-1,4-dihydro-1,6-
naphthyridine-5-carboxamide

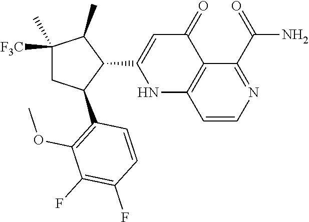 [SMILES: COc1c([C@H]2C[C@@](C)(C(F)(F)F)[C@@H](C)[C@@H]2c2cc(=O)c3c(C(N)=O)nccc3[nH]2)ccc(F)c1F]

2-((1S,2S,3R,5S)-5-(3,4-
difluoro-2-methoxyphenyl)-2,3-
dimethyl-3-
(trifluoromethyl)cyclopentyl)-4-
oxo-1,4-dihydro-1,6-
naphthyridine-5-carboxamide

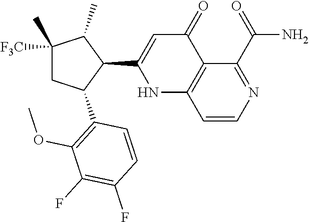 [SMILES: COc1c([C@@H]2C[C@](C)(C(F)(F)F)[C@H](C)[C@H]2c2cc(=O)c3c(C(N)=O)nccc3[nH]2)ccc(F)c1F]

2-((1R,2R,3S,5R)-5-(3,4-
difluoro-2-methoxyphenyl)-2,3-
dimethyl-3-
(trifluoromethyl)cyclopentyl)-4-
oxo-1,4-dihydro-1,6-
naphthyridine-5-carboxamide

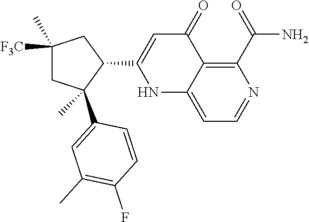 [SMILES: Cc1cc([C@@]2(C)C[C@@](C)(C(F)(F)F)C[C@@H]2c2cc(=O)c3c(C(N)=O)nccc3[nH]2)ccc1F]

2-((1S,2S,4S)-2-(4-fluoro-3-
methylphenyl)-2,4-dimethyl-4-
(trifluoromethyl)cyclopentyl)-4-
oxo-1,4-dihydro-1,6-
naphthyridine-5-carboxamide

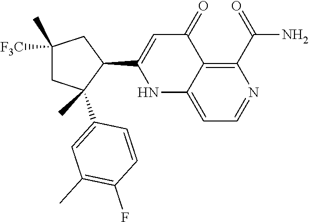 [SMILES: Cc1cc([C@]2(C)C[C@](C)(C(F)(F)F)C[C@H]2c2cc(=O)c3c(C(N)=O)nccc3[nH]2)ccc1F]

2-((1R,2R,4R)-2-(4-fluoro-3-
methylphenyl)-2,4-dimethyl-4-
(trifluoromethyl)cyclopentyl)-4-
oxo-1,4-dihydro-1,6-
naphthyridine-5-carboxamide

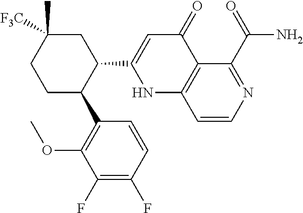 [SMILES: COc1c([C@H]2CC[C@](C)(C(F)(F)F)C[C@@H]2c2cc(=O)c3c(C(N)=O)nccc3[nH]2)ccc(F)c1F]

2-((1S,2S,5S)-2-(3,4-difluoro-2-
methoxyphenyl)-5-methyl-5-
(trifluoromethyl)cyclohexyl)-4-
oxo-1,4-dihydro-1,6-
naphthyridine-5-carboxamide

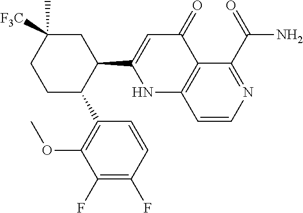 [SMILES: COc1c([C@@H]2CC[C@@](C)(C(F)(F)F)C[C@H]2c2cc(=O)c3c(C(N)=O)nccc3[nH]2)ccc(F)c1F]

2-((1R,2R,5R)-2-(3,4-difluoro-2-
methoxyphenyl)-5-methyl-5-
(trifluoromethyl)cyclohexyl)-4-
oxo-1,4-dihydro-1,6-
naphthyridine-5-carboxamide

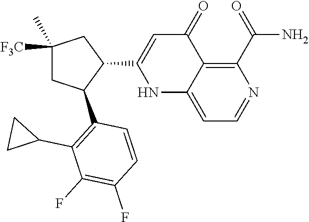 [SMILES: C[C@]1(C(F)(F)F)C[C@H](c2cc(=O)c3c(C(N)=O)nccc3[nH]2)[C@@H](c2ccc(F)c(F)c2C2CC2)C1]

2-((1S,2S,4R)-2-(2-cyclopropyl-
3,4-difluorophenyl)-4-methyl-4-
(trifluoromethyl)cyclopentyl)-4-
oxo-1,4-dihydro-1,6-
naphthyridine-5-carboxamide

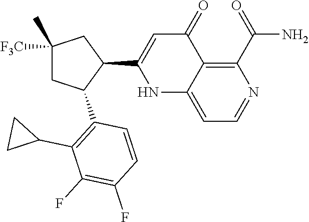 [SMILES: C[C@@]1(C(F)(F)F)C[C@@H](c2cc(=O)c3c(C(N)=O)nccc3[nH]2)[C@H](c2ccc(F)c(F)c2C2CC2)C1]

2-((1R,2R,4S)-2-(2-cyclopropyl-
3,4-difluorophenyl)-4-methyl-4-
(trifluoromethyl)cyclopentyl)-4-
oxo-1,4-dihydro-1,6-
naphthyridine-5-carboxamide

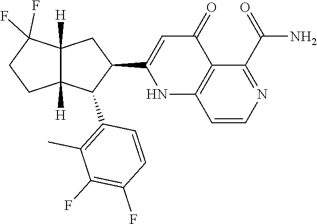 [SMILES: Cc1c([C@@H]2[C@@H]3CCC(F)(F)[C@@H]3C[C@H]2c2cc(=O)c3c(C(N)=O)nccc3[nH]2)ccc(F)c1F]

2-((1R,2R,3aR,6aS)-1-(3,4-
difluoro-2-methylphenyl)-4,4-
difluorooctahydropentalen-2-
yl)-4-oxo-1,4-dihydro-1,6-
naphthyridine-5-carboxamide

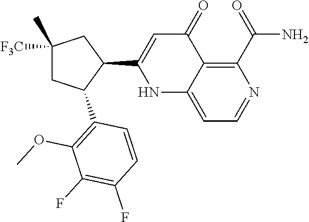 [SMILES: COc1c([C@@H]2C[C@](C)(C(F)(F)F)C[C@H]2c2cc(=O)c3c(C(N)=O)nccc3[nH]2)ccc(F)c1F]

2-((1R,2R,4S)-2-(3,4-difluoro-2-
methoxyphenyl)-4-methyl-4-
(trifluoromethyl)cyclopentyl)-4-
oxo-1,4-dihydro-1,6-
naphthyridine-5-carboxamide

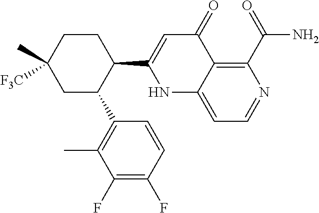 [SMILES: Cc1c([C@@H]2C[C@](C)(C(F)(F)F)CC[C@H]2c2cc(=O)c3c(C(N)=O)nccc3[nH]2)ccc(F)c1F]

2-((1R,2R,4R)-2-(3,4-difluoro-2-
methylphenyl)-4-methyl-4-
(trifluoromethyl)cyclohexyl)-4-
oxo-1,4-dihdyro-1,6-
naphthyridine-5-carboxamide

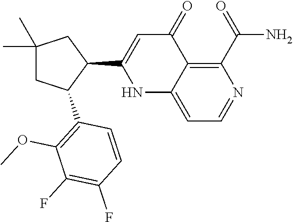 [SMILES: COc1c([C@@H]2CC(C)(C)C[C@H]2c2cc(=O)c3c(C(N)=O)nccc3[nH]2)ccc(F)c1F]

2-((1R,2R)-2-(3,4-difluoro-2-
methoxyphenyl)-4,4-
dimethylcyclopentyl)-4-oxo-1,4-
dihydro-1,6-naphthyridine-5-
carboxamide

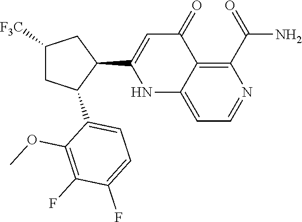 [SMILES: COc1c([C@@H]2C[C@H](C(F)(F)F)C[C@H]2c2cc(=O)c3c(C(N)=O)nccc3[nH]2)ccc(F)c1F]

2-((1R,2R,4S)-2-(3,4-difluoro-2-
methoxyphenyl)-4-
(trifluoromethyl)cyclopentyl)-4-
oxo-1,4-dihydro-1,6-
naphthyridine-5-carboxamide

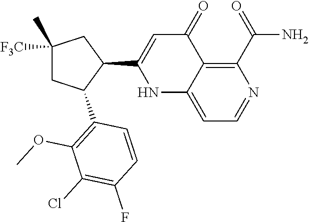 [SMILES: COc1c([C@@H]2C[C@](C)(C(F)(F)F)C[C@H]2c2cc(=O)c3c(C(N)=O)nccc3[nH]2)ccc(F)c1Cl]

2-((1R,2R,4S)-2-(3-chloro-4-
fluoro-2-methoxyphenyl)-4-
methyl-4-
(trifluoromethyl)cyclopentyl)-4-
oxo-1,4-dihydro-1,6-
naphthyridine-5-carboxamide

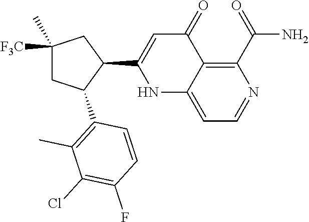 [SMILES: Cc1c([C@@H]2C[C@@](C)(C(F)(F)F)C[C@H]2c2cc(=O)c3c(C(N)=O)nccc3[nH]2)ccc(F)c1Cl]

2-((1R,2R,4R)-2-(3-chloro-4-
fluoro-2-methylphenyl)-4-
methyl-4-
(trifluoromethyl)cyclopentyl)-4-
oxo-1,4-dihydro-1,6-
naphthyridine-5-carboxamide

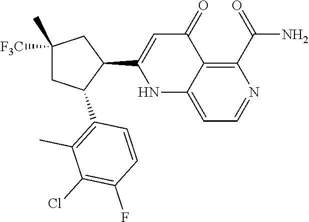 [SMILES: Cc1c([C@@H]2C[C@](C)(C(F)(F)F)C[C@H]2c2cc(=O)c3c(C(N)=O)nccc3[nH]2)ccc(F)c1Cl]

2-((1R,2R,4S)-2-(3-chloro-4-
fluoro-2-methylphenyl)-4-
methyl-4-
(trifluoromethyl)cyclopentyl)-4-
oxo-1,4-dihydro-1,6-
naphthyridine-5-carboxamide

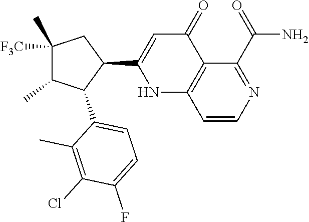 [SMILES: Cc1c([C@H]2[C@H](c3cc(=O)c4c(C(N)=O)nccc4[nH]3)C[C@@](C)(C(F)(F)F)[C@H]2C)ccc(F)c1Cl]

2-((1R,2R,3S,4R)-2-(3-chloro-4-
fluoro-2-methylphenyl)-3,4-
dimethyl-4-
(trifluoromethyl)cyclopentyl)-4-
oxo-1,4-dihydro-1,6-
naphthyridine-5-carboxamide

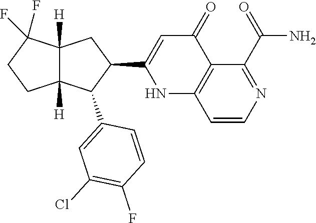 [SMILES: NC(=O)c1nccc2[nH]c([C@@H]3C[C@@H]4[C@@H](CCC4(F)F)[C@H]3c3ccc(F)c(Cl)c3)cc(=O)c12]

2-((1R,2R,3aR,6aS)-1-(3-chloro-
4-fluorophenyl)-4,4-
difluorooctahydropentalen-2-
yl)-4-oxo-1,4-dihydro-1,6-
naphthyridine-5-carboxamide

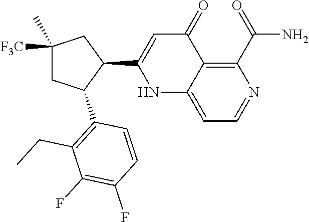 [SMILES: CCc1c([C@@H]2C[C@@](C)(C(F)(F)F)C[C@H]2c2cc(=O)c3c(C(N)=O)nccc3[nH]2)ccc(F)c1F]

2-((1R,2R,4R)-2-(2-ethyl-3,4-
difluorophenyl)-4-methyl-4-
(trifluoromethyl)cyclopentyl)-4-
oxo-1,4-dihydro-1,6-
naphthyridine-5-carboxamide

In some embodiments, the invention relates to a compound selected from Table B, or a pharmaceutically acceptable salt there. In other embodiments, the invention relates to a compound selected from Table B, i.e., the compound in non-salt form.

TABLE B
Compound Structures and Names.

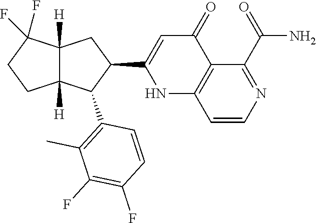 [SMILES: Cc1c([C@@H]2[C@@H]3CCC(F)(F)[C@@H]3C[C@H]2c2cc(=O)c3c(C(N)=O)nccc3[nH]2)ccc(F)c1F]

2-((1R,2R,3aR,6aS)-1-(3,4-
difluoro-2-methylphenyl)-4,4-
difluorooctahydropentalen-2-
yl)-4-oxo-1,4-dihydro-1,6-
naphthyridine-5-carboxamide

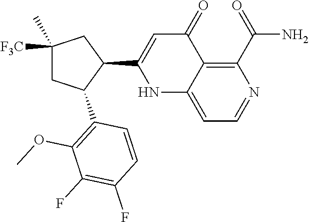 [SMILES: COc1c([C@@H]2C[C@@](C)(C(F)(F)F)C[C@H]2c2cc(=O)c3c(C(N)=O)nccc3[nH]2)ccc(F)c1F]

2-((1R,2R,4S)-2-(3,4-difluoro-2-
methoxyphenyl)-4-methyl-4-
(trifluoromethyl)cyclopentyl)-4-
oxo-1,4-dihydro-1,6-
naphthyridine-5-carboxamide

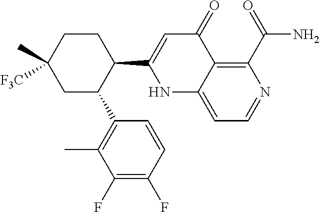 [SMILES: Cc1c([C@@H]2C[C@](C)(C(F)(F)F)CC[C@H]2c2cc(=O)c3c(C(N)=O)nccc3[nH]2)ccc(F)c1F]

2-((1R,2R,4R)-2-(3,4-difluoro-2-
methylphenyl)-4-methyl-4-
(trifluoromethyl)cyclohexyl)-4-
oxo-1,4-dihydro-1,6-
naphthyridine-5-carboxamide

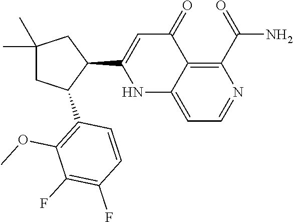 [SMILES: COc1c([C@@H]2CC(C)(C)C[C@H]2c2cc(=O)c3c(C(N)=O)nccc3[nH]2)ccc(F)c1F]

2-((1R,2R)-2-(3,4-difluoro-2-
methoxyphenyl)-4,4-
dimethylcyclopentyl)-4-oxo-
1,4-dihydro-1,6-naphthyridine-
5-carboxamide

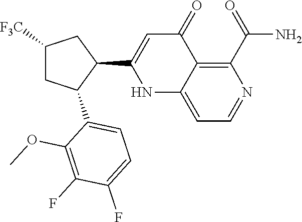 [SMILES: COc1c([C@@H]2C[C@H](C(F)(F)F)C[C@H]2c2cc(=O)c3c(C(N)=O)nccc3[nH]2)ccc(F)c1F]

2-((1R,2R,4S)-2-(3,4-difluoro-2-
methoxyphenyl)-4-
(trifluoromethyl)cyclopentyl)-4-
oxo-1,4-dihydro-1,6-
naphthyridine-5-carboxamide

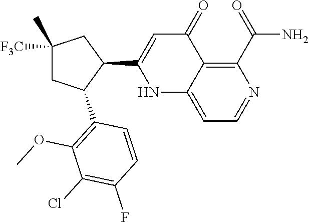 [SMILES: COc1c([C@@H]2C[C@](C)(C(F)(F)F)C[C@H]2c2cc(=O)c3c(C(N)=O)nccc3[nH]2)ccc(F)c1Cl]

2-((1R,2R,4S)-2-(3-chloro-4-
fluoro-2-methoxyphenyl)-4-
methyl-4-
(trifluoromethyl)cyclopentyl)-4-
oxo-1,4-dihydro-1,6-
naphthyridine-5-carboxamide

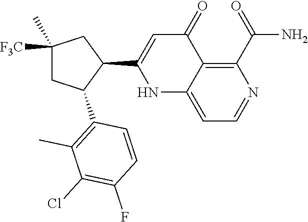 [SMILES: Cc1c([C@@H]2C[C@@](C)(C(F)(F)F)C[C@H]2c2cc(=O)c3c(C(N)=O)nccc3[nH]2)ccc(F)c1Cl]

2-((1R,2R,4R)-2-(3-chloro-4-
fluoro-2-methylphenyl)-4-
methyl-4-
(trifluoromethyl)cyclopentyl)-4-
oxo-1,4-dihydro-1,6-
naphthyridine-5-carboxamide

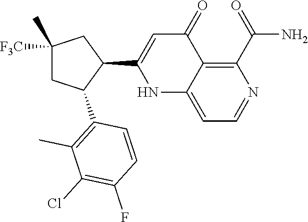 [SMILES: Cc1c([C@@H]2C[C@](C)(C(F)(F)F)C[C@H]2c2cc(=O)c3c(C(N)=O)nccc3[nH]2)ccc(F)c1Cl]

2-((1R,2R,4S)-2-(3-chloro-4-
fluoro-2-methylphenyl)-4-
methyl-4-
(trifluoromethyl)cyclopentyl)-4-
oxo-1,4-dihydro-1,6-
naphthyridine-5-carboxamide

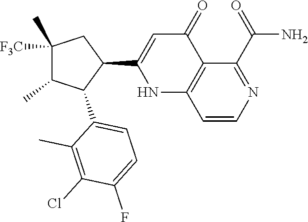 [SMILES: Cc1c([C@H]2[C@H](c3cc(=O)c4c(C(N)=O)nccc4[nH]3)C[C@@](C)(C(F)(F)F)[C@H]2C)ccc(F)c1Cl]

2-((1R,2R,3S,4R)-2-(3-chloro-4-
fluoro-2-methylphenyl)-3,4-
dimethyl-4-
(trifluoromethyl)cyclopentyl)-4-
oxo-1,4-dihydro-1,6-
naphthyridine-5-carboxamide

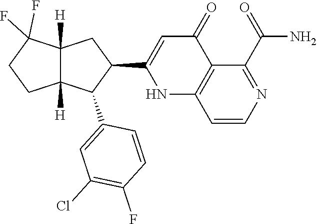 [SMILES: NC(=O)c1nccc2[nH]c([C@@H]3C[C@@H]4[C@@H](CCC4(F)F)[C@H]3c3ccc(F)c(Cl)c3)cc(=O)c12]

2-((1R,2R,3aR,6aS)-1-(3-chloro-4-fluorophenyl)-
4,4-difluorooctahydropentalen-2-yl)-4-oxo-1,4-
dihydro-1,6-naphthyridine-5-carboxamide

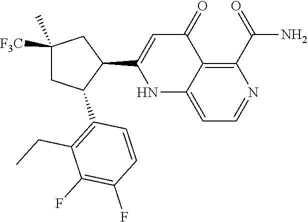 [SMILES: CCc1c([C@@H]2C[C@@](C)(C(F)(F)F)C[C@H]2c2cc(=O)c3c(C(N)=O)nccc3[nH]2)ccc(F)c1F]

2-((1R,2R,4R)-2-(2-ethyl-3,4-difluorophenyl)-4-
methyl-4-(trifluoromethyl)cyclopentyl)-4-oxo-
1,4-dihydro-1,6-naphthyridine-5-carboxamide

In some embodiments, the invention relates to a compound selected from Table C, or a pharmaceutically acceptable salt thereof. In other embodiments, the invention relates to a compound selected from Table C, i.e., the compound in non-salt form.

TABLE C
Compound Structures and Names.

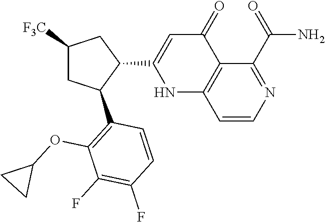 [SMILES: NC(=O)c1nccc2[nH]c([C@H]3C[C@H](C(F)(F)F)C[C@@H]3c3ccc(F)c(F)c3OC3CC3)cc(=O)c12]

2-((1S,2S,4R)-2-(2-
cyclopropoxy-3,4-
difluorophenyl)-4-
(trifluoromethyl)cyclopentyl)-4-
oxo-1,4-dihydro-1,6-
naphthyridine-5-carboxamide

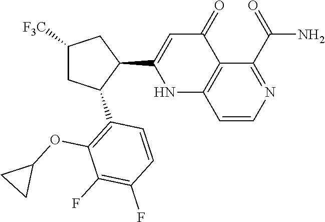 [SMILES: NC(=O)c1nccc2[nH]c([C@@H]3C[C@@H](C(F)(F)F)C[C@H]3c3ccc(F)c(F)c3OC3CC3)cc(=O)c12]

2-((1R,2R,4S)-2-(2-
cyclopropoxy-3,4-
difluorophenyl)-4-
(trifluoromethyl)cyclopentyl)-4-
oxo-1,4-dihydro-1,6-
naphthyridine-5-carboxamide

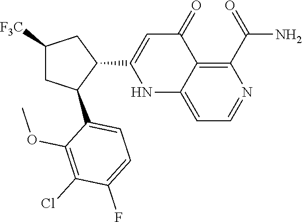 [SMILES: COc1c([C@H]2C[C@@H](C(F)(F)F)C[C@@H]2c2cc(=O)c3c(C(N)=O)nccc3[nH]2)ccc(F)c1Cl]

2-((1S,2S,4R)-2-(3-chloro-4-
fluoro-2-methoxyphenyl)-4-
(trifluoromethyl)cyclopentyl)-4-
oxo-1,4-dihydro-1,6-
naphthyridine-5-carboxamide

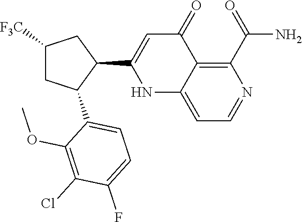 [SMILES: COc1c([C@@H]2C[C@H](C(F)(F)F)C[C@H]2c2cc(=O)c3c(C(N)=O)nccc3[nH]2)ccc(F)c1Cl]

2-((1R,2R,4S)-2-(3-chloro-4-
fluoro-2-methoxyphenyl)-4-
(trifluoromethyl)cyclopentyl)-4-
oxo-1,4-dihydro-1,6-
naphthyridine-5-carboxamide

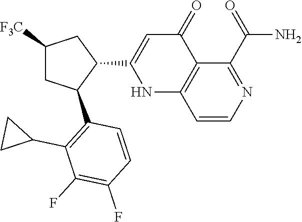 [SMILES: NC(=O)c1nccc2[nH]c([C@H]3C[C@H](C(F)(F)F)C[C@@H]3c3ccc(F)c(F)c3C3CC3)cc(=O)c12]

2-((1S,2S,4R)-2-(2-cyclopropyl-
3,4-difluorophenyl)-4-
(trifluoromethyl)cyclopentyl)-4-
oxo-1,4-dihydro-1,6-
naphthyridine-5-carboxamide

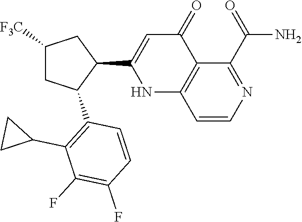 [SMILES: NC(=O)c1nccc2[nH]c([C@@H]3C[C@@H](C(F)(F)F)C[C@H]3c3ccc(F)c(F)c3C3CC3)cc(=O)c12]

2-((1R,2R,4S)-2-(2-cyclopropyl-
3,4-difluorophenyl)-4-
(trifluoromethyl)cyclopentyl)-4-
oxo-1,4-dihydro-1,6-
naphthyridine-5-carboxamide

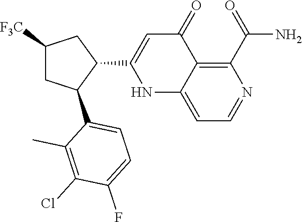 [SMILES: Cc1c([C@H]2C[C@@H](C(F)(F)F)C[C@@H]2c2cc(=O)c3c(C(N)=O)nccc3[nH]2)ccc(F)c1Cl]

2-((1S,2S,4R)-2-(3-chloro-4-
fluoro-2-methylphenyl)-4-
(trifluoromethyl)cyclopentyl)-4-
oxo-1,4-dihydro-1,6-
naphthyridine-5-carboxamide

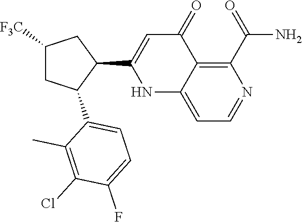 [SMILES: Cc1c([C@@H]2C[C@H](C(F)(F)F)C[C@H]2c2cc(=O)c3c(C(N)=O)nccc3[nH]2)ccc(F)c1Cl]

2-((1R,2R,4S)-2-(3-chloro-4-
fluoro-2-methylphenyl)-4-
(trifluoromethyl)cyclopentyl)-4-
oxo-1,4-dihydro-1,6-
naphthyridine-5-carboxamide

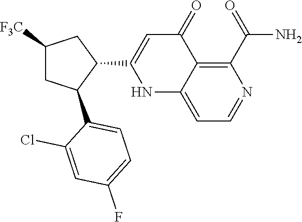 [SMILES: NC(=O)c1nccc2[nH]c([C@H]3C[C@H](C(F)(F)F)C[C@@H]3c3ccc(F)cc3Cl)cc(=O)c12]

2-((1S,2S,4R)-2-(2-chloro-4-
fluorophenyl)-4-
(trifluoromethyl)cyclopentyl)-4-
oxo-1,4-dihydro-1,6-
naphthyridine-5-carboxamide

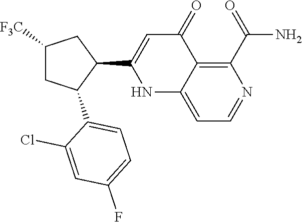 [SMILES: NC(=O)c1nccc2[nH]c([C@@H]3C[C@@H](C(F)(F)F)C[C@H]3c3ccc(F)cc3Cl)cc(=O)c12]

2-((1R,2R,4S)-2-(2-chloro-4-
fluorophenyl)-4-
(trifluoromethyl)cyclopentyl)-4-
oxo-1,4-dihydro-1,6-
naphthyridine-5-carboxamide

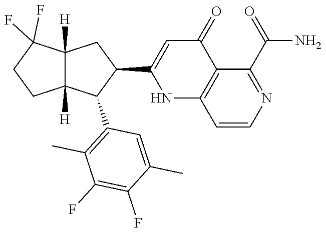 [SMILES: Cc1cc([C@@H]2[C@@H]3CCC(F)(F)[C@@H]3C[C@H]2c2cc(=O)c3c(C(N)=O)nccc3[nH]2)c(C)c(F)c1F]

2-((1R,2R,3aR,6aS)-1-(3,4-
difluoro-2,5-dimethylphenyl)-
4,4-difluorooctahydropentalen-
2-yl)-4-oxo-1,4-dihydro-1,6-
naphthyridine-5-carboxamide

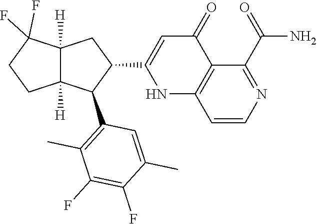 [SMILES: Cc1cc([C@H]2[C@H]3CCC(F)(F)[C@H]3C[C@@H]2c2cc(=O)c3c(C(N)=O)nccc3[nH]2)c(C)c(F)c1F]

2-((1S,2S,3aS,6aR)-1-(3,4-
difluoro-2,5-dimethylphenyl)-
4,4-difluorooctahydropentalen-
2-yl)-4-oxo-1,4-dihydro-1,6-
naphthyridine-5-carboxamide

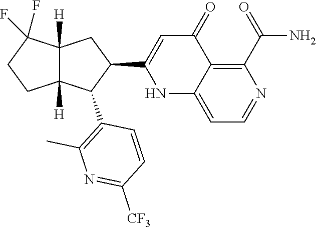 [SMILES: Cc1nc(C(F)(F)F)ccc1[C@@H]1[C@@H]2CCC(F)(F)[C@@H]2C[C@H]1c1cc(=O)c2c(C(N)=O)nccc2[nH]1]

2-((1R,2R,3aR,6aS)-4,4-
difluoro-1-(2-methyl-6-
(trifluoromethyl)pyridin-3-
yl)octahydropentalen-2-yl)-4-
oxo-1,4-dihydro-1,6-
naphthyridine-5-carboxamide

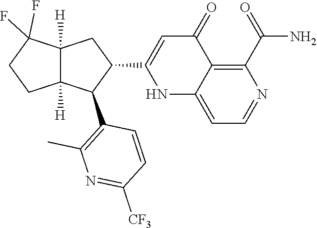 [SMILES: Cc1nc(C(F)(F)F)ccc1[C@H]1[C@H]2CCC(F)(F)[C@H]2C[C@@H]1c1cc(=O)c2c(C(N)=O)nccc2[nH]1]

2-((1S,2S,3aS,6aR)-4,4-difluoro-
1-(2-methyl-6-
(trifluoromethyl)pyridin-3-
yl)octahydropentalen-2-yl)-4-
oxo-1,4-dihydro-1,6-
naphthyridine-5-carboxamide

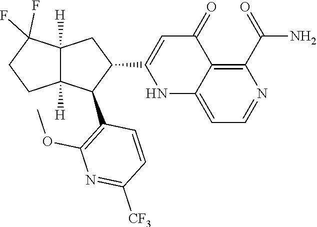 [SMILES: COc1nc(C(F)(F)F)ccc1[C@H]1[C@H]2CCC(F)(F)[C@H]2C[C@@H]1c1cc(=O)c2c(C(N)=O)nccc2[nH]1]

2-((1S,2S,3aS,6aR)-4,4-difluoro-
1-(2-methoxy-6-
(trifluoromethyl)pyridin-3-
yl)octahydropentalen-2-yl)-4-
oxo-1,4-dihydro-1,6-
naphthyridine-5-carboxamide

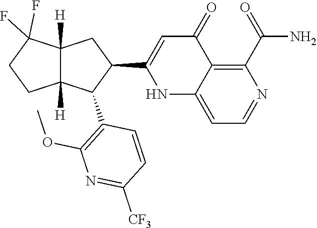 [SMILES: COc1nc(C(F)(F)F)ccc1[C@@H]1[C@@H]2CCC(F)(F)[C@@H]2C[C@H]1c1cc(=O)c2c(C(N)=O)nccc2[nH]1]

2-((1R,2R,3aR,6aS)-4,4-
difluoro-1-(2-methoxy-6-
(trifluoromethyl)pyridin-3-
yl)octahydropentalen-2-yl)-4-
oxo-1,4-dihydro-1,6-
naphthyridine-5-carboxamide

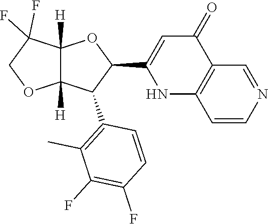 [SMILES: Cc1c([C@@H]2[C@@H]3OCC(F)(F)[C@@H]3O[C@H]2c2cc(=O)c3cnccc3[nH]2)ccc(F)c1F]

2-((2R,3S,3aS,6aR)-3-(3,4-
difluoro-2-methylphenyl)-6,6-
difluorohexahydrofuro[3,2-
b]furan-2-yl)-1,6-naphthyridin-
4(1H)-one

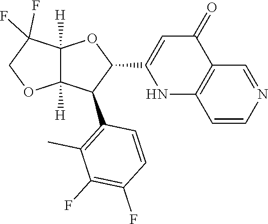 [SMILES: Cc1c([C@H]2[C@H]3OCC(F)(F)[C@H]3O[C@@H]2c2cc(=O)c3cnccc3[nH]2)ccc(F)c1F]

2-((2S,3R,3aR,6aS)-3-(3,3-
difluoro-2-methylphenyl)-6,6-
difluorohexahydrofuro[3,2-
b]furan-2-yl)-1,6-naphthyridin-
4(1H)-one

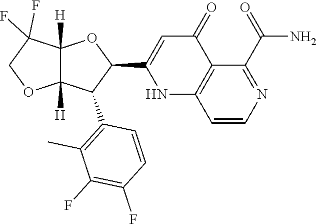 [SMILES: Cc1c([C@@H]2[C@@H]3OCC(F)(F)[C@@H]3O[C@H]2c2cc(=O)c3c(C(N)=O)nccc3[nH]2)ccc(F)c1F]

2-((2R,3S,3aS,6aR)-3-(3,4-difluoro-2-
methylphenyl)-6,6-difluorohexahydrofuro[3,2-
b]furan-2-yl)-4-oxo-1,4-dihydro-1,6-
naphthyridine-5-carboxamide

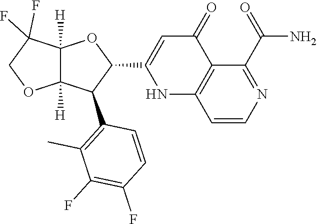 [SMILES: Cc1c([C@H]2[C@H]3OCC(F)(F)[C@H]3O[C@@H]2c2cc(=O)c3c(C(N)=O)nccc3[nH]2)ccc(F)c1F]

2-
((2S,3R,3aR,6aS)-3-(3,4-difluoro-2-
methylphenyl)-6,6-difluorohexahydrofuro[3,2-
b]furan-2-yl)-4-oxo-1,4-dihydro-1,6-
naphthyridine-5-carboxamide

In some embodiments, the invention relates to a compound selected from Table D, or a pharmaceutically acceptable salt thereof. In other embodiments, the invention relates to a compound selected from Table D, i.e., the compound in non-salt form.

TABLE D
Compound Structures and Names.

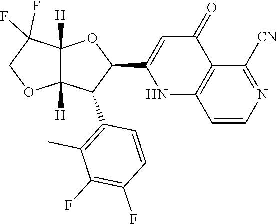 [SMILES: Cc1c([C@@H]2[C@@H]3OCC(F)(F)[C@@H]3O[C@H]2c2cc(=O)c3c(C#N)nccc3[nH]2)ccc(F)c1F]

2-((2R,3S,3aS,6aR)-3-(3,4-
difluoro-2-methylphenyl)-6,6-
difluorohexahydrofuro[3,2-
b]furan-2-yl)-4-oxo-1,4-
dihydro-1,6-naphthyridine-5-
carbonitrile

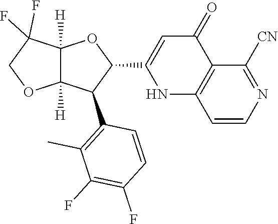 [SMILES: Cc1c([C@H]2[C@H]3OCC(F)(F)[C@H]3O[C@@H]2c2cc(=O)c3c(C#N)nccc3[nH]2)ccc(F)c1F]

2-((2S,3R,3aR,6aS)-3-(3,4-
difluoro-2-methylphenyl)-6,6-
difluorohexahydrofuro[3,2-
b]furan-2-yl)-4-oxo-1,4-
dihydro-1,6-naphthyridine-5-
carbonitrile

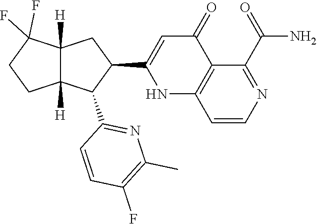 [SMILES: Cc1nc([C@@H]2[C@@H]3CCC(F)(F)[C@@H]3C[C@H]2c2cc(=O)c3c(C(N)=O)nccc3[nH]2)ccc1F]

2-((1R,2R,3aR,6aS)-4,4-
difluoro-1-(5-fluoro-6-
methylpyridin-2-
yl)octahydropentalen-2-yl)-4-
oxo-1,4-dihydro-1,6-
naphthyridine-5-carobxamide

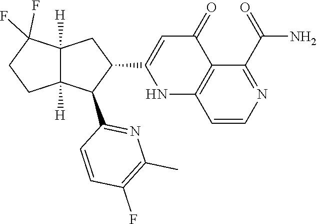 [SMILES: Cc1nc([C@H]2[C@H]3CCC(F)(F)[C@H]3C[C@@H]2c2cc(=O)c3c(C(N)=O)nccc3[nH]2)ccc1F]

2-((1S,2S,3aS,6aR)-4,4-difluoro-
1-(5-fluoro-6-methylpyridin-2-
yl)octahydropentalen-2-yl)-4-
oxo-1,4-dihydro-1,6-
naphthyridine-5-carboxamide

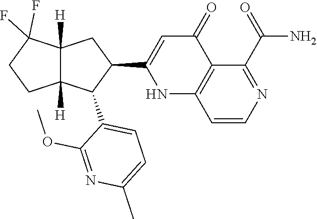 [SMILES: COc1nc(C)ccc1[C@@H]1[C@@H]2CCC(F)(F)[C@@H]2C[C@H]1c1cc(=O)c2c(C(N)=O)nccc2[nH]1]

2-((1R,2R,3aR,6aS)-4,4-
difluoro-1-(2-methoxy-6-
methylpyridin-3-
yl)octahydropentalen-2-yl)-4-
oxo-1,4-dihydro-1,6-
naphthyridine-5-carboxamide

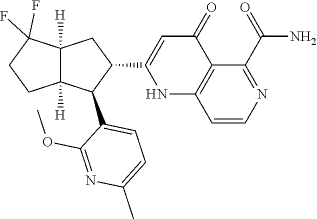 [SMILES: COc1nc(C)ccc1[C@H]1[C@H]2CCC(F)(F)[C@H]2C[C@@H]1c1cc(=O)c2c(C(N)=O)nccc2[nH]1]

2-((1S,2S,3aS,6aR)-4,4-difluoro-
1-(2-methoxy-6-methylpyridin-
3-yl)octahydropentalen-2-yl)-4-
oxo-1,4-dihydro-1,6-
naphthyridine-5-carboxamide

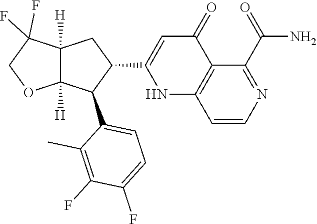 [SMILES: Cc1c([C@H]2[C@H]3OCC(F)(F)[C@H]3C[C@@H]2c2cc(=O)c3c(C(N)=O)nccc3[nH]2)ccc(F)c1F]

2-((3aS,5S,6S,6aR)-6-(3,4-difluoro-2-
methylphenyl)-3,3-difluorohexahydro-2H-
cyclopenta[b]furan-5-yl)-4-oxo-1,4-dihydro-1,6-
naphthyridine-5-carobxamide

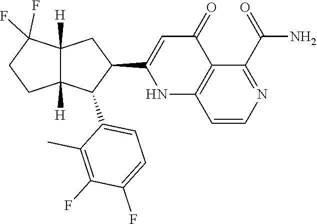 [SMILES: Cc1c([C@@H]2[C@@H]3CCC(F)(F)[C@@H]3C[C@H]2c2cc(=O)c3c(C(N)=O)nccc3[nH]2)ccc(F)c1F]

2-((3aR,5R,6R,6aS)-6-(3,4-difluoro-2-
methylphenyl)-3,3-difluorohexahydro-2H-
cyclopenta[b]furan-5-yl)-4-oxo-1,4-dihydro-
1,6-naphthyridine-5-carboxamide

### Salts, Compositions, Uses, Formulation, Administration and Additional Agents

### Pharmaceutically Acceptable Salts and Compositions

As discussed herein, the invention provides compounds, and pharmaceutically acceptable salts thereof, that are inhibitors of voltage-gated sodium channels, and thus the present compounds, and pharmaceutically acceptable salts thereof, are useful for the treatment of diseases, disorders, and conditions including, but not limited to chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia. Accordingly, in another aspect of the invention, pharmaceutical compositions are provided, wherein these compositions comprise a compound as described herein, or a pharmaceutically acceptable salt thereof, and optionally comprise a pharmaceutically acceptable carrier, adjuvant or vehicle. In certain embodiments, these compositions optionally further comprise one or more additional therapeutic agents. In some embodiments, the additional therapeutic agent is a sodium channel inhibitor.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof. The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J Pharmaceutical Sciences, 1977, 66, 1-19, incorporated herein by reference. Pharmaceutically acceptable salts of the compound of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the invention features a pharmaceutical composition comprising a compound of the invention, or a pharmaceutically acceptable salt thereof, and a pharmaceutically acceptable carrier.

In another aspect, the invention features a pharmaceutical composition comprising a therapeutically effective amount of a compound, or a pharmaceutically acceptable salt thereof, and one or more pharmaceutically acceptable carriers or vehicles.

### Uses of Compounds and Pharmaceutically Acceptable Salts and Compositions

In another aspect, the invention features a method of inhibiting a voltage-gated sodium channel in a subject comprising administering to the subject a compound of the invention or a pharmaceutically acceptable salt thereof or a pharmaceutical composition thereof. In another aspect, the voltage-gated sodium channel is Na_{V}1.8.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of gut pain, wherein gut pain comprises inflammatory bowel disease pain, Crohn's disease pain, irritable bowel syndrome, endometriosis, polycystic ovarian disease, salpingitis, cervicitis or interstitial cystitis pain wherein said method comprises administering an effective amount of a compound of the invention, a pharmaceutically acceptable salt thereof or a pharmaceutical composition thereof.

In yet another aspect, the invention features a method of treating or lessening the severity in a subject of neuropathic pain comprising administering an effective amount of a compound of the invention, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the invention features a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia wherein said method comprises administering an effective amount of a compound of the invention, a pharmaceutically acceptable salt thereof or a pharmaceutical composition thereof.

In yet another aspect, the invention features a method of treating or lessening the severity in a subject of musculoskeletal pain comprising administering an effective amount of a compound of the invention, a pharmaceutically acceptable salt thereof or a pharmaceutical composition thereof. In some aspects, the musculoskeletal pain comprises osteoarthritis pain.

In yet another aspect, the invention features a method of treating or lessening the severity in a subject of musculoskeletal pain, wherein musculoskeletal pain comprises osteoarthritis pain, back pain, cold pain, burn pain or dental pain wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain, ankylosing spondylitis or vulvodynia wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises fibromyalgia pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises reflex sympathetic dystrophy pain, wherein said method comprises administering an effective amount of a compound of the invention, a pharmaceutically acceptable salt thereof or a pharmaceutical composition thereof.

In yet another aspect, the invention features a method of treating or lessening the severity in a subject of pathological cough wherein said method comprises administering an effective amount of a compound of the invention, a pharmaceutically acceptable salt thereof or a pharmaceutical composition thereof.

In yet another aspect, the invention features a method of treating or lessening the severity in a subject of acute pain comprising administering an effective amount of a compound of the invention, a pharmaceutically acceptable salt thereof or a pharmaceutical composition thereof. In some aspects, the acute pain comprises acute post-operative pain.

In yet another aspect, the invention features a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain) comprising administering an effective amount of a compound of the invention, a pharmaceutically acceptable salt thereof or a pharmaceutical composition thereof.

In yet another aspect, the invention features a method of treating or lessening the severity in a subject of bunionectomy pain comprising administering an effective amount of a compound of the invention, a pharmaceutically acceptable salt thereof or a pharmaceutical composition thereof.

In yet another aspect, the invention features a method of treating or lessening the severity in a subject of shoulder arthroplasty pain or shoulder arthroscopy pain comprising administering an effective amount of a compound of the invention, a pharmaceutically acceptable salt thereof or a pharmaceutical composition thereof.

In yet another aspect, the invention features a method of treating or lessening the severity in a subject of herniorrhaphy pain comprising administering an effective amount of a compound of the invention, a pharmaceutically acceptable salt thereof or a pharmaceutical composition thereof.

In yet another aspect, the invention features a method of treating or lessening the severity in a subject of abdominoplasty pain comprising administering an effective amount of a compound of the invention, a pharmaceutically acceptable salt thereof or a pharmaceutical composition thereof.

In yet another aspect, the invention features a method of treating or lessening the severity in a subject of visceral pain comprising administering an effective amount of a compound of the invention, a pharmaceutically acceptable salt thereof or a pharmaceutical composition thereof. In some aspects, the visceral pain comprises visceral pain from abdominoplasty.

In yet another aspect, the invention features a method of treating or lessening the severity in a subject of a neurodegenerative disease comprising administering an effective amount of a compound of the invention, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the invention features a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/hemiation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility, comprising administering an effective amount of a compound of the invention, a pharmaceutically acceptable salt thereof or a pharmaceutical composition thereof.

In another aspect, the invention features a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain, comprising administering an effective amount of a compound of the invention, a pharmaceutically acceptable salt thereof or a pharmaceutical composition thereof.

In another aspect, the invention features a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain, comprising administering an effective amount of a compound of the invention, a pharmaceutically acceptable salt thereof or a pharmaceutical composition thereof.

### Compounds, Pharmaceutically Acceptable Salts, and Compositions for Use

In another aspect, the invention features a compound of the invention, or a pharmaceutically acceptable salt or pharmaceutical composition thereof, for use as a medicament.

In another aspect, the invention features a compound of the invention, or a pharmaceutically acceptable salt or pharmaceutical composition thereof, for use in a method of inhibiting a voltage-gated sodium channel in a subject. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In another aspect, the invention features a compound of the invention, or a pharmaceutically acceptable salt or pharmaceutical composition thereof, for use in a method of treating or lessening the severity in a subject of gut pain, wherein gut pain comprises inflammatory bowel disease pain, Crohn's disease pain, irritable bowel syndrome, endometriosis, polycystic ovarian disease, salpingitis, cervicitis or interstitial cystitis pain.

In another aspect, the invention features a compound of the invention, or a pharmaceutically acceptable salt or pharmaceutical composition thereof, for use in a method of treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the invention features a compound of the invention, or a pharmaceutically acceptable salt or pharmaceutical composition thereof, for use in a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia.

In another aspect, the invention features a compound of the invention, or a pharmaceutically acceptable salt or pharmaceutical composition thereof, for use in a method of treating or lessening the severity in a subject of musculoskeletal pain. In some aspects, the musculoskeletal pain comprises osteoarthritis pain.

In another aspect, the invention features a compound of the invention, or a pharmaceutically acceptable salt or pharmaceutical composition thereof, for use in a method of treating or lessening the severity in a subject of musculoskeletal pain, wherein musculoskeletal pain comprises osteoarthritis pain, back pain, cold pain, burn pain or dental pain.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain, ankylosing spondylitis or vulvodynia.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises fibromyalgia pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises reflex sympathetic dystrophy pain.

In another aspect, the invention features a compound of the invention, or a pharmaceutically acceptable salt or pharmaceutical composition thereof, for use in a method of treating or lessening the severity in a subject of pathological cough.

In another aspect, the invention features a compound of the invention, or a pharmaceutically acceptable salt or pharmaceutical composition thereof, for use in a method of treating or lessening the severity in a subject of acute pain. In some aspects, the acute pain comprises acute post-operative pain.

In another aspect, the invention features a compound of the invention, or a pharmaceutically acceptable salt or pharmaceutical composition thereof, for use in a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In another aspect, the invention features a compound of the invention, or a pharmaceutically acceptable salt or pharmaceutical composition thereof, for use in a method of treating or lessening the severity in a subject of bunionectomy pain.

In another aspect, the invention features a compound of the invention, or a pharmaceutically acceptable salt or pharmaceutical composition thereof, for use in a method of treating or lessening the severity in a subject of shoulder arthroplasty pain or shoulder arthroscopy pain.

In another aspect, the invention features a compound of the invention, or a pharmaceutically acceptable salt or pharmaceutical composition thereof, for use in a method of treating or lessening the severity in a subject of herniorrhaphy pain.

In another aspect, the invention features a compound of the invention, or a pharmaceutically acceptable salt or pharmaceutical composition thereof, for use in a method of treating or lessening the severity in a subject of abdominoplasty pain.

In another aspect, the invention features a compound of the invention, or a pharmaceutically acceptable salt or pharmaceutical composition thereof, for use in a method of treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for use in a method of treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In another aspect, the invention features a compound of the invention, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a compound of the invention, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention features a compound of the invention, or a pharmaceutically acceptable salt or pharmaceutical composition thereof, for use in a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention features a compound of the invention, or a pharmaceutically acceptable salt or pharmaceutical composition thereof, for use in a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Manufacture of Medicaments

In another aspect, the invention provides the use of a compound of the invention, or a pharmaceutically acceptable salt or pharmaceutical composition thereof, for the manufacture of a medicament.

In another aspect, the invention provides the use of a compound of the invention, a pharmaceutically acceptable salt thereof, or a pharmaceutical composition thereof for the manufacture of a medicament for use in inhibiting a voltage-gated sodium channel. In another aspect, the voltage-gated sodium channel is Na_{V}1.8.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In yet another aspect, the invention provides the use of the compound, pharmaceutically acceptable salt, or pharmaceutical composition described herein for the manufacture of a medicament for use in treating or lessening the severity in a subject of gut pain, wherein gut pain comprises inflammatory bowel disease pain, Crohn's disease pain, irritable bowel syndrome, endometriosis, polycystic ovarian disease, salpingitis, cervicitis or interstitial cystitis pain.

In yet another aspect, the invention provides a compound of the invention, a pharmaceutically acceptable salt thereof, or a pharmaceutical composition thereof for the manufacture of a medicament for use in treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the invention provides the use of a compound of the invention, a pharmaceutically acceptable salt thereof, or a pharmaceutical composition thereof for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic neuropathy.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain. In some aspects, the musculoskeletal pain comprises osteoarthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain, wherein musculoskeletal pain comprises osteoarthritis pain, back pain, cold pain, burn pain or dental pain.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain, ankylosing spondylitis or vulvodynia.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises fibromyalgia pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises reflex sympathetic dystrophy pain.

In yet another aspect, the invention provides the use of a compound of the invention, a pharmaceutically acceptable salt thereof, or a pharmaceutical composition thereof for the manufacture of a medicament for use in treating or lessening the severity in a subject of pathological cough.

In yet another aspect, the invention provides the use of a compound of the invention, a pharmaceutically acceptable salt thereof, or a pharmaceutical composition thereof for the manufacture of a medicament for use in treating or lessening the severity in a subject of acute pain. In some aspects, the acute pain comprises acute post-operative pain.

In yet another aspect, the invention provides the use of a compound of the invention, a pharmaceutically acceptable salt thereof, or a pharmaceutical composition thereof for the manufacture of a medicament for use in treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In yet another aspect, the invention provides the use of a compound of the invention, a pharmaceutically acceptable salt thereof, or a pharmaceutical composition thereof for the manufacture of a medicament for use in treating or lessening the severity in a subject of herniorrhaphy pain.

In yet another aspect, the invention provides the use of a compound of the invention, a pharmaceutically acceptable salt thereof, or a pharmaceutical composition thereof for the manufacture of a medicament for use in treating or lessening the severity in a subject of bunionectomy pain.

In yet another aspect, the invention provides the use of a compound of the invention, a pharmaceutically acceptable salt thereof, or a pharmaceutical composition thereof for the manufacture of a medicament for use in treating or lessening the severity in a subject of shoulder arthroplasty pain or shoulder arthroscopy pain.

In yet another aspect, the invention provides the use of a compound of the invention, a pharmaceutically acceptable salt thereof, or a pharmaceutical composition thereof for the manufacture of a medicament for use in treating or lessening the severity in a subject of abdominoplasty pain.

In yet another aspect, the invention provides the use of a compound of the invention, a pharmaceutically acceptable salt thereof, or a pharmaceutical composition thereof for the manufacture of a medicament for use in treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for the manufacture of a medicament for use in treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In yet another aspect, the invention provides the use of a compound of the invention, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the invention provides the use of a compound of the invention, a pharmaceutically acceptable salt thereof, or a pharmaceutical composition thereof for the manufacture of a medicament for use in treating or lessening the severity of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound of the invention, a pharmaceutically acceptable salt thereof, or a pharmaceutical composition thereof for the manufacture of a medicament for use in treating or lessening the severity of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention provides the use of a compound of the invention, a pharmaceutically acceptable salt thereof, or a pharmaceutical composition thereof for the manufacture of a medicament for use in treating or lessening the severity of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Administration of Compounds, Pharmaceutically Acceptable Salts, and Compositions

In certain embodiments of the invention, an “effective amount” of a compound of the invention, a pharmaceutically acceptable salt thereof, or a pharmaceutical composition thereof is that amount effective for treating or lessening the severity of one or more of the conditions recited above.

The compounds, salts, and compositions, according to the method of the invention, may be administered using any amount and any route of administration effective for treating or lessening the severity of one or more of the pain or non-pain diseases recited herein. The exact amount required will vary from subject to subject, depending on the species, age, and general condition of the subject, the severity of the condition, the particular agent, its mode of administration, and the like. The compounds, salts, and compositions of the invention are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds, salts, and compositions of the invention will be decided by the attending physician within the scope of sound medical judgment. The specific effective dose level for any particular subject or organism will depend upon a variety of factors including the disorder being treated and the severity of the disorder; the activity of the specific compound or salt employed; the specific composition employed; the age, body weight, general health, sex and diet of the subject; the time of administration, route of administration, and rate of excretion of the specific compound or salt employed; the duration of the treatment; drugs used in combination or coincidental with the specific compound or salt employed, and like factors well known in the medical arts. The term “subject” or “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the condition being treated. In certain embodiments, the compound, salts, and compositions of the invention may be administered orally or parenterally at dosage levels of about 0.001 mg/kg to about 1000 mg/kg, one or more times a day, effective to obtain the desired therapeutic effect.

Liquid dosage forms for oral administration include, but are not limited to, pharmaceutically acceptable emulsions, microemulsions, solutions, suspensions, syrups and elixirs. In addition to the active compound or salt, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of the compounds of the invention, it is often desirable to slow the absorption of the compounds from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound or salt of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound or salt is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound or salt can also be in microencapsulated form with one or more excipients as noted above. The solid dosage forms of tablets, dragees, capsules, pills, and granules can be prepared with coatings and shells such as enteric coatings, release-controlling coatings and other coatings well known in the pharmaceutical formulating art. In such solid dosage forms, the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound or salt of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of voltage-gated sodium channels. In one embodiment, the compounds are inhibitors of Na_{V}1.8 and thus, without wishing to be bound by any particular theory, the compounds, salts, and compositions are particularly useful for treating or lessening the severity of a disease, condition, or disorder where activation or hyperactivity of Na_{V}1.8 is implicated in the disease, condition, or disorder. When activation or hyperactivity of Na_{V}1.8 is implicated in a particular disease, condition, or disorder, the disease, condition, or disorder may also be referred to as a “Na_{V}1.8-mediated disease, condition or disorder.” Accordingly, in another aspect, the invention provides a method for treating or lessening the severity of a disease, condition, or disorder where activation or hyperactivity of Na_{V}1.8 is implicated in the disease state.

The activity of a compound utilized in this invention as an inhibitor of Na_{V}1.8 may be assayed according to methods described generally in International Publication No. WO 2014/120808 A9 and U.S. Publication No. 2014/0213616 A1, both of which are incorporated by reference in their entirety, methods described herein, and other methods known and available to one of ordinary skill in the art.

### Additional Therapeutic Agents

It will also be appreciated that the compounds, salts, and pharmaceutically acceptable compositions of the invention can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin; naphthylalkanones such as Nabumetone; oxicams such as Piroxicam; para-aminophenol derivatives, such as Acetaminophen; propionic acids such as Fenoprofen, Flurbiprofen, Ibuprofen, Ketoprofen, Naproxen, Naproxen sodium, Oxaprozin; salicylates such as Aspirin, Choline magnesium trisalicylate, Diflunisal; fenamates such as meclofenamic acid, Mefenamic acid; and pyrazoles such as Phenylbutazone); or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp &Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-l-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat®) or valproate;
(12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT_{1B/1D} agonist such as eletriptan, sumatriptan, naratriptan, zolmitriptan or rizatriptan;
(22) a 5-HT_{2A} receptor antagonist such as R(+)-alpha-(2,3-dimethoxy-phenyl)-1-[2-(4-fluorophenylethyl)]-4-piperidinemethanol (MDL-100907);
(23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ER®), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (1[alpha],3[alpha],5[alpha])(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluR1) antagonist;
(29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-l-(5-thiazolyl)-butyl]thio]-S-chloro-S-pyridinecarbonitrile; 2-[[(1R,3S)-3-amino-4-hydroxy-l-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-l-(5-thiazolyl) butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504);
(37) a sodium channel blocker, such as lidocaine, lidocaine plus tetracaine cream (ZRS-201) or eslicarbazepine acetate;
(38) a Na_{V}1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607); WO2012/106499 (US2012196869); WO2012/112743 (US2012245136); WO2012/125613 (US2012264749), WO2012/116440 (US2014187533), WO2011026240 (US2012220605), U.S. Pat. Nos. 8,883,840, 8,466,188, WO2013/109521 (US2015005304), CN111217776, WO2020/117626, WO2021/252822, WO2021/252818, WO2021/252820, WO2014/201173, WO2012/125973, WO2013/086229, WO2013/134518, WO2014/201206, or WO2016/141035 the entire contents of each application hereby incorporated by reference;
(38a) a Na_{V}1.7 blocker such as (2-benzylspiro[3,4-dihydropyrrolo[1,2-a]pyrazine-1,4′-piperidine]-1′-yl)-(4-isopropoxy-3-methyl-phenyl)methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-yl]ethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl)methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl)methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone.
(39) a Na_{V}1.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/144375, WO2020/261114, WO2020/140959, WO2020/151728, WO2021/032074, WO2021/047622 (CN112479996), WO2021/257490, WO2021/257420, WO2021/257418, WO2022/263498, WO2022/235558, WO2022/235859, WO2023/138599, WO2024/041613, WO2024/046253, WO2024/046409, CN112390745, CN111808019, CN112225695, CN112457294, CN112300051, CN112300069, CN112441969, and CN114591293, the entire contents of each application hereby incorporated by reference;
(39a) a Na_{V}1.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl)quinoline-3-carboxamide, 2-(4-fluoro-2-methoxyphenoxy)-N-(3-sulfamoylphenyl)quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy)quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxy]-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, or 4-[[2-fluoro-6-[3-fluoro-2-methoxy-4-(trifluoromethoxy)phenoxy]-3-(trifluoromethyl)benzoyl]amino]pyridine-2-carboxamide;
(40) a combined Na_{V}1.7 and Na_{V}1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®); and the pharmaceutically acceptable salts and solvates thereof;
(43) a nicotinic receptor antagonist, such as varenicline;
(44) an N-type calcium channel antagonist, such as Z-160;
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ-10234094 (Carisbamate), BMS-954561 or ARC-4558.

In another embodiment, the additional appropriate therapeutic agents are selected from N-(6-amino-5-(2,3,5-trichlorophenyl)pyridin-2-yl)acetamide; N-(6-amino-5-(2-chloro-5-methoxyphenyl)pyridin-2-yl)-1-methyl-1H-pyrazole-5-carboxamide; or 3-((4-(4-(trifluoromethoxy)phenyl)-1H-imidazol-2-yl)methyl)oxetan-3-amine.

In another embodiment, the additional therapeutic agent is selected from a GlyT2/5HT2 inhibitor, such as Operanserin (VVZ149), a TRPV modulator such as CA008, CMX-020, NE06860, FTABS, CNTX4975, MCP101, MDR16523, or MDR652, a EGRI inhibitor such as Brivoglide (AYX1), an NGF inhibitor such as Tanezumab, Fasinumab, ASP6294, MED17352, a Mu opioid agonist such as Cebranopadol, NKTR181 (oxycodegol), a CB-1 agonist such as NEO1940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Na_{V}1.7 blocker such as ST-2427, ST-2578 and those disclosed in WO2010/129864, WO2015/157559, WO2017/059385, WO2018/183781, WO2018/183782, WO2020/072835, and/or WO2022/036297 the entire contents of each application hereby incorporated by reference.

In another embodiment, the additional therapeutic agent is selected from ASP18071, CC-8464, ANP-230, ANP-231, NOC-100, NTX-1175, ASN008, NW3509, AM-6120, AM-8145, AM-0422, BL-017881, NTM-006, Opiranserin (Unafra™), brivoligide, SR419, NRD.E1, LX9211, LY3016859, ISC-17536, NFX-88, LAT-8881, AP-235, NYX 2925, CNTX-6016, S-600918, S-637880, RQ-00434739, KLS-2031, MEDI 7352, and XT-150.

In another embodiment, the additional therapeutic agent is selected from Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623/ODM-111, ETX-801, OLP-1002, ANP-230/DSP-2230, iN1011-N17, DSP-3905 and ACD440.

In another embodiment, the additional therapeutic agent is selected from HRS4800, ODM-111/JMKX000623, LX9211, LY3556050, LY3857210, CFTX01554/CFTX-1554, MED17352, MEDI0618, BAY3178275, BAY2395840, GSK3858279, STC-004, HALNEURON, OLP-1002, ATX01, ANP230, CC-8464, iN1011-N17, ST-2427, MSD199, FZ008, VYNAV-01, BL-017881, Profervia (Cilnidipine), LS-04, vixotrigine, FX301/PCRX-301, PF-04531083, PF-01247324, and DSP-3905.

In another embodiment, the additional therapeutic agent is a sodium channel inhibitor (also known as a sodium channel blocker), such as the Na_{V}1.7 and Na_{V}1.8 blockers identified above.

The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

The compounds and salts of this invention or pharmaceutically acceptable compositions thereof may also be incorporated into compositions for coating an implantable medical device, such as prostheses, artificial valves, vascular grafts, stents and catheters. Accordingly, the invention, in another aspect, includes a composition for coating an implantable device comprising a compound or salt of the invention as described generally above, and in classes and subclasses herein, and a carrier suitable for coating said implantable device. In still another aspect, the invention includes an implantable device coated with a composition comprising a compound or salt of the invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

Another aspect of the invention relates to inhibiting Na_{V}1.8 activity in a biological sample or a subject, which method comprises administering to the subject, or contacting said biological sample with a compound of the invention, a pharmaceutically acceptable salt thereof, or a pharmaceutical composition thereof. The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

Inhibition of Na_{V}1.8 activity in a biological sample is useful for a variety of purposes that are known to one of skill in the art. Examples of such purposes include, but are not limited to, the study of sodium channels in biological and pathological phenomena; and the comparative evaluation of new sodium channel inhibitors.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T. G. M. Wuts et al., Greene 's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P, ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)- and/or carbon-14 (¹⁴C)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### ENUMERATED EMBODIMENTS

Additional embodiments, features, and advantages of the disclosure will be apparent from the following detailed description and through practice of the disclosure. The compounds and methods of the present disclosure can be described as embodiments in any of the following enumerated clauses. It will be understood that any of the embodiments described herein can be used in connection with any other embodiments described herein to the extent that the embodiments do not contradict one another.

1. A compound of formula (I):

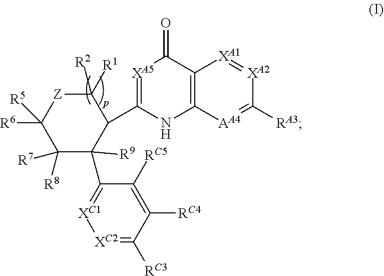

or a pharmaceutically acceptable salt thereof, wherein:
X^{A1} is C—R^{A1}, N, or N⁺—O⁻;
X^{A2} is C—R^{A2}, N, or N⁺—O⁻;
X^{A4} is C—R^{A4}, N, or N⁺—O⁻;
X^{A5} is C—R^{A} or N;
X^{C1} is C—R^{C1} or N;
X^{C2} is C—R^{C2} or N;
R^{A1}, R^{A2}, R^{A3}, and R^{A4} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{A1} is H, OH, halo, CN, C₁-C₆ alkyl, C₁-C₆ haloalkyl, C₁-C₆ alkoxy, C₁-C₆ haloalkoxy, —(CH₂)ₘOₙ(CH₂)ₒOCH₃, —(CH₂)ₘR^{a}, —C(O)(CH₂)ₘR^{a}, —C(O)OR, —C(O)R, —C(O)NR^{b}R^{c}, —NR^{b}R^{c}, —CR^{d}R^{e}R^{f}, —CR^{b}R^{c}NR^{g}C(O)CR^{h}RⁱR^{j}, —NR^{b}C(O)CR^{c}R^{g}R^{j}, C₃-C₆ cycloalkyl, 5-6 membered heteroaryl, or 4-10 membered heterocyclyl, wherein said C₃-C₆ cycloalkyl, 5-6 membered heteroaryl, or 4-10 membered heterocyclyl may be unsubstituted or may be substituted with 1-4 substituents independently selected from halo, C₁-C₆ alkyl, C₁-C₆ haloalkyl, C₁-C₆ alkoxy, and C₁-C₆ haloalkoxy;
R¹ and R² are each independently H, halo, OH, C₁-C₆ alkyl, C₁-C₆ haloalkyl, C₃-C₆ cycloalkyl, or C₁-C₆ alkoxy;
R³ and R⁴ are each independently H, halo, OH, C₁-C₆ alkyl, C₁-C₆ haloalkyl, C₃-C₆ cycloalkyl, or C₁-C₆ alkoxy;
p, Z, R⁵, R⁶, R⁷, and R⁸ are defined as follows:
   (i) p is 0 or 1;
      Z is C(—R³)(—R⁴);
      R⁵ and R⁶ are each independently H, halo, OH, C₁-C₆ alkyl, C₁-C₆ haloalkyl, C₃-C₆ cycloalkyl, or C₁-C₆ alkoxy; and
      R⁷ and R⁸ are each independently H, halo, OH, C₁-C₆ alkyl, C₁-C₆ haloalkyl, C₃-C₆ cycloalkyl, or C₁-C₆ alkoxy; or
   (ii) p is 0 or 1;
      Z is C(—R³)(—R⁴);
      R⁷ and R⁸ are each independently H, halo, OH, C₁-C₆ alkyl, C₁-C₆ haloalkyl, C₃-C₆ cycloalkyl, or C₁-C₆ alkoxy; and
      R⁵ and R⁶, together with the carbon atom to which they are attached, form a C₃-C₆ cycloalkyl optionally substituted with 1-6 R^{y}; or
   (iii) p is 0 or 1;
      Z is C(—R³)(—R⁴);
      R⁵ and R⁷, together with the carbon atoms to which they are attached, form a fused C₃-C₆ cycloalkyl optionally substituted with 1-6 R^{y}; and
      R⁶ and R⁸ are each independently H, halo, OH, C₁-C₆ alkyl, C₁-C₆ haloalkyl, C₃-C₆ cycloalkyl, or C₁-C₆ alkoxy; or
   (iv) p is 0;
      Z is C(—R³)(—R⁴) or O;
      R⁵ and R⁷, together with the carbon atoms to which they are attached, form a fused ring of formula

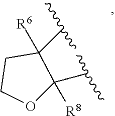

optionally substituted with 1-4 R^{y}; and
R⁶ and R⁸ are each independently H, halo, OH, C₁-C₆ alkyl, C₁-C₆ haloalkyl, C₃-C₆ cycloalkyl, or C₁-C₆ alkoxy;
R⁹ is H or CH₃;
R^{C1} and R^{C2} are each independently H, OH, halo, C₁-C₆ alkyl, C₁-C₆ alkoxy, C₁-C₆ haloalkyl, C₁-C₆ haloalkoxy, —[CH₂]_{q}—C₃-C₆ cycloalkyl, —[CH₂]_{q}—O—C₃-C₆ cycloalkyl, —[CH₂]_{q}—NR^{b}R^{c}, —(CH₂)ₘOR, or —O(CH₂)ₘOCH₃, or R^{C1} and R^{C2}, together with the carbon atoms to which they are attached, form a ring of formula:

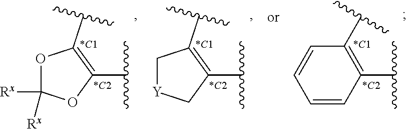

Y is O or CH₂;
R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
each R^{x} is independently H, halo, C₁-C₆ alkyl, or C₁-C₆ haloalkyl;
each R^{y} is independently halo, OH, C₁-C₆ alkyl, or C₁-C₆ haloalkyl;
R^{a} is OH, halo, C₁-C₆ alkoxy, or —NR^{b}R^{c};
R^{b}, R^{c}, R^{g}, R^{h}, and Rⁱ are each independently H or C₁-C₆ alkyl, or R^{b} and R^{c}, together with the nitrogen atom to which they are attached, form a 3-6 membered heterocyclyl; R^{d}, R^{e}, and R^{f} are each independently H, OH, halo, C₁-C₆ alkyl, or C₁-C₆ alkoxy;
R^{j} is H, C₁-C₆ alkyl, —NR^{b}R^{c}, or —N(CH₃)₃⁺;
m and o are each independently 0, 1, 2, or 3;
q is 0 or 1; and
n is 0 or 1.
   2. The compound of clause 1, or a pharmaceutically acceptable salt thereof, wherein:
p is 0 or 1;
Z is C(—R³)(—R⁴);
R⁵, R⁶, R⁷, and R⁸ are defined as follows:
   (i) R⁵ and R⁶ are each independently H, halo, OH, C₁-C₆ alkyl, C₁-C₆ haloalkyl, C₃-C₆ cycloalkyl, or C₁-C₆ alkoxy; and
      R⁷ and R⁸ are each independently H, halo, OH, C₁-C₆ alkyl, C₁-C₆ haloalkyl, C₃-C₆ cycloalkyl, or C₁-C₆ alkoxy; or
   (ii) R⁷ and R⁸ are each independently H, halo, OH, C₁-C₆ alkyl, C₁-C₆ haloalkyl, C₃-C₆ cycloalkyl, or C₁-C₆ alkoxy; and
      R⁵ and R⁶, together with the carbon atom to which they are attached, form a C₃-C₆ cycloalkyl optionally substituted with 1-6 R^{y}; or
   (iii) R⁵ and R⁷, together with the carbon atoms to which they are attached, form a fused C₃-C₆ cycloalkyl optionally substituted with 1-6 R^{y}; and
      R⁶ and R⁸ are each independently H, halo, OH, C₁-C₆ alkyl, C₁-C₆ haloalkyl, C₃-C₆ cycloalkyl, or C₁-C₆ alkoxy.
         3. The compound of clause 1, or a pharmaceutically acceptable salt thereof, wherein the compound is a compound of formula (II):

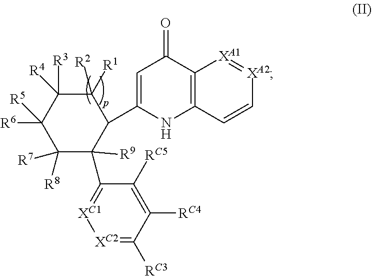

wherein:
X^{A1} is C—R^{A1}, N, or N⁺—O⁻;
X^{A2} is C—R^{A2}, N, or N⁺—O⁻;
p is 0 or 1;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R¹ and R² are each independently H, OH, C₁-C₆ alkyl, C₁-C₆ haloalkyl, C₃-C₆ cycloalkyl, or C₁-C₆ alkoxy;
R³ and R⁴ are each independently H, halo, OH, C₁-C₆ alkyl, C₁-C₆ haloalkyl, C₃-C₆ cycloalkyl, or C₁-C₆ alkoxy;
R⁵, R⁶, R⁷, and R⁸ are defined as follows:
   (i) R⁵ and R⁶ are each independently H, OH, C₁-C₆ alkyl, C₁-C₆ haloalkyl, C₃-C₆ cycloalkyl, or C₁-C₆ alkoxy; and
      R⁷ and R⁸ are each independently H, OH, C₁-C₆ alkyl, C₁-C₆ haloalkyl, C₃-C₆ cycloalkyl, or C₁-C₆ alkoxy; or
   (ii) R⁷ and R⁸ are each independently H, OH, C₁-C₆ alkyl, C₁-C₆ haloalkyl, C₃-C₆ cycloalkyl, or C₁-C₆ alkoxy; and
      R⁵ and R⁶, together with the carbon atom to which they are attached, form a C₃-C₆ cycloalkyl optionally substituted with 1-6 R^{y}; or
   (iii) R⁵ and R⁷, together with the carbon atoms to which they are attached, form a fused C₃-C₆ cycloalkyl optionally substituted with 1-6 R^{y}; and
      R⁶ and R⁸ are each independently H, OH, C₁-C₆ alkyl, C₁-C₆ haloalkyl, C₃-C₆ cycloalkyl, or C₁-C₆ alkoxy;
R⁹ is H or CH₃;
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
each R^{y} is independently halo, OH, C₁-C₆ alkyl, or C₁-C₆ haloalkyl;
R^{b} and R^{c} are each independently H or C₁-C₆ alkyl, or R and R^{o}, together with the nitrogen atom to which they are attached, form a 3-6 membered heterocyclyl; and
m and o are each independently 0, 1, 2, or 3.
   4. The compound of clause 1, or a pharmaceutically acceptable salt thereof, wherein the compound is a compound of formula (III):

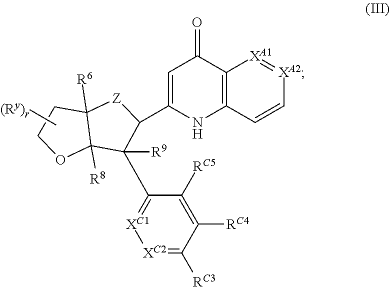

wherein:
X^{A1} is C—R^{A1}, N, or N⁺—O⁻;
X^{A2} is C—R^{A2}, N, or N⁺—O⁻;
Z is C(—R³)(—R⁴) or O;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R³ and R⁴ are each independently H, halo, OH, C₁-C₆ alkyl, C₁-C₆ haloalkyl, C₃-C₆ cycloalkyl, or C₁-C₆ alkoxy;
R⁶ and R⁸ are each independently H, OH, C₁-C₆ alkyl, C₁-C₆ haloalkyl, C₃-C₆ cycloalkyl, or C₁-C₆ alkoxy;
R⁹ is H or CH₃;
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
each R^{y} is independently halo, OH, C₁-C₆ alkyl, or C₁-C₆ haloalkyl;
R^{b} and R^{c} are each independently H or C₁-C₆ alkyl, or R and R^{o}, together with the nitrogen atom to which they are attached, form a 3-6 membered heterocyclyl;
m and o are each independently 0, 1, 2, or 3; and
r is 0, 1, 2, 3, or 4.
   5. The compound of any one of clauses 1 to 4, or a pharmaceutically acceptable salt thereof, wherein X^{A1} is C—R^{A1}.
   6. The compound of any one of clauses 1 to 5, or a pharmaceutically acceptable salt thereof, wherein R^{A1} is —C(O)NR^{b}R^{c}, and R^{b} and R^{c} are each independently H or C₁-C₆ alkyl.
   7. The compound of any one of clauses 1 to 6, or a pharmaceutically acceptable salt thereof, wherein X^{A2} is N.
   8. The compound of any one of clauses 1 to 7, or a pharmaceutically acceptable salt thereof, wherein:
X^{A1} is C—R^{A1};
R^{A1} is —C(O)NR^{b}R^{c};
R^{b} and R^{c} are each H; and
X^{A2} is N.
   9. The compound of any one of clauses 1 to 3 and 5 to 8, or a pharmaceutically acceptable salt thereof, wherein p is 0.
   10. The compound of any one of clauses 1 to 3 and 5 to 8, or a pharmaceutically acceptable salt thereof, wherein p is 1.
   11. The compound of any one of clauses 1 to 3, 5 to 8 and 10, or a pharmaceutically acceptable salt thereof, wherein R¹ and R² are each H.
   12. The compound of any one of clauses 1 to 3 and 5 to 11, or a pharmaceutically acceptable salt thereof, wherein R³ and R⁴ are each independently H or C₁-C₂ haloalkyl.
   13. The compound of any one of clauses 1 to 3 and 5 to 12, or a pharmaceutically acceptable salt thereof, wherein R³ and R⁴ are each independently H or CF₃.
   14. The compound of any one of clauses 1 to 3 and 5 to 13, or a pharmaceutically acceptable salt thereof, wherein R³ and R⁴ are each H.
   15. The compound of any one of clauses 1 to 3 and 5 to 14, or a pharmaceutically acceptable salt thereof, wherein R⁵ and R⁶ are each independently H, OH, C₁-C₆ alkyl, C₁-C₆ haloalkyl, C₃-C₆ cycloalkyl, or C₁-C₆ alkoxy or, together with the carbon atom to which they are attached, form a C₃-C₆ cycloalkyl optionally substituted with 1-6 R^{y}, where each R^{y} is independently halo or C₁ haloalkyl.
   16. The compound of any one of clauses 1 to 3 and 5 to 15, or a pharmaceutically acceptable salt thereof, wherein R⁵ and R⁶ are each independently H, C₁-C₄ alkyl, or C₁ haloalkyl.
   17. The compound of any one of clauses 1 to 3 and 5 to 16, or a pharmaceutically acceptable salt thereof, wherein R⁵ and R⁶ are each independently H, —CH₃, —C(CH₃)₃ or —CF₃.
   18. The compound of any one of clauses 1 to 3 and 5 to 15, or a pharmaceutically acceptable salt thereof, wherein R⁵ and R⁶, together with the carbon atom to which they are attached, form a C₃-C₆ cycloalkyl optionally substituted with 1-3 R^{y}, where each R^{y} is independently halo or C₁ haloalkyl.
   19. The compound of any one of clauses 1 to 3, 5 to 15, and 18, or a pharmaceutically acceptable salt thereof, wherein R⁵ and R⁶, together with the carbon atom to which they are attached, form a C₃-C₆ cycloalkyl optionally substituted with 2 R^{y}, where each R^{y} is independently F.
   20. The compound of any one of clauses 1 to 3 and 5 to 19, or a pharmaceutically acceptable salt thereof, wherein R⁷ and R⁸ are each independently H, OH, C₁-C₃ alkyl, C₁-C₃ haloalkyl, or C₁-C₃ alkoxy.
   21. The compound of any one of clauses 1 to 3 and 5 to 20, or a pharmaceutically acceptable salt thereof, wherein R⁷ and R⁸ are each independently H, OH, —CH₃, —CF₃, or —O—CH₃.
   22. The compound of any one of clauses 1 to 3 and 5 to 14, or a pharmaceutically acceptable salt thereof, wherein:
R⁵ and R⁷, together with the carbon atoms to which they are attached, form a fused C₃-C₆ cycloalkyl optionally substituted with 1-3 R^{y};
R⁶ and R⁸ are each H; and
each R^{y} is independently halo.
   23. The compound of any one of clauses 1 to 3, 5 to 14, and 22, or a pharmaceutically acceptable salt thereof, wherein:
R⁵ and R, together with the carbon atoms to which they are attached, form a fused Cₛ or C₆ cycloalkyl optionally substituted with 2 F; and
R⁶ and R⁸ are each H.
   24. The compound of any one of clauses 1 and 4 to 8, or a pharmaceutically acceptable salt thereof, wherein Z is O.
   25. The compound of any one of clauses 1, 4 to 8, and 24, or a pharmaceutically acceptable salt thereof, wherein R⁶ and R⁸ are each H.
   26. The compound of any one of clauses 1, 4 to 8, 24, and 25, or a pharmaceutically acceptable salt thereof, wherein each R^{y} is independently halo, and r is 2.
   27. The compound of any one of clauses 1, 4 to 8, and 24 to 26, or a pharmaceutically acceptable salt thereof, wherein each R^{y} is F, and r is 2.
   28. The compound of any one of clauses 1 to 27, or a pharmaceutically acceptable salt thereof, wherein R⁹ is H.
   29. The compound of any one of clauses 1 to 28, or a pharmaceutically acceptable salt thereof, wherein:
X^{C1} is C—R^{C1}; and X^{C2} is C—R^{C2}.
   30. The compound of any one of clauses 1 to 28, or a pharmaceutically acceptable salt thereof, wherein:
X^{C1} is C—R^{C1}; and
X^{C2} is N.
   31. The compound of any one of clauses 1 to 30, or a pharmaceutically acceptable salt thereof, wherein R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, or —CH₂OH.
   32. The compound of any one of clauses 1 to 31, or a pharmaceutically acceptable salt thereof, wherein R^{C1}, R^{C2}, R^{C3}, R^{C4}, and RCs are each independently H, OH, halo, C₁-C₆ alkyl, C₁-C₆ alkoxy, C₁-C₆ haloalkyl, C₁-C₆ haloalkoxy, C₃-C₆ cycloalkyl, or —CH₂OH.
   33. The compound of any one of clauses 1 to 32, or a pharmaceutically acceptable salt thereof, wherein R^{C1}, R^{C2}, R^{C3}, R^{C4}, and RCs are each independently H, OH, F, Cl, —CH₃, —CH₂CH₃, —OCH₃, —CF₃, C₃ cycloalkyl, —O—C₃ cycloalkyl, or —CH₂OH.
   34. The compound of any one of clauses 1 to 33, or a pharmaceutically acceptable salt thereof, wherein R^{C1}, R^{C2}, R^{C3}, R^{C4}, and RCs are each independently H, OH, F, Cl, —CH₃, —CH₂CH₃, —OCH₃, C₃ cycloalkyl, or —CH₂OH.
   35. The compound of any one of clauses 1 to 34, or a pharmaceutically acceptable salt thereof, wherein R^{C1} is —CH₃, R^{C2} is F, R^{C3} is F, R^{C4} is H, and RCs is H.
   36. The compound of any one of clauses 1 to 34, or a pharmaceutically acceptable salt thereof, wherein R^{C1} is —OCH₃, R^{C2} is F, R^{C3} is F, R^{C4} is H, and RCs is H.
   37. The compound of any one of clauses 1 to 34, or a pharmaceutically acceptable salt thereof, wherein R^{C1} is —OCH₃, R^{C2} is Cl, R^{C3} is F, R^{C4} is H, and RCs is H.
   38. The compound of any one of clauses 1 to 34, or a pharmaceutically acceptable salt thereof, wherein R^{C1} is —CH₃, R^{C2} is Cl, R^{C3} is F, R^{C4} is H, and RCs is H.
   39. The compound of any one of clauses 1 to 34, or a pharmaceutically acceptable salt thereof, wherein R^{C1} is —CH₂CH₃, R^{C2} is F, R^{C3} is F, R^{C4} is H, and RCs is H.
   40. A compound selected from Table A, or a pharmaceutically acceptable salt thereof.
   41. A compound selected from Table B, or a pharmaceutically acceptable salt thereof.
   42. A compound selected from Table C, or a pharmaceutically acceptable salt thereof.
   43. The compound of any one of clauses 1 to 42 in non-salt form.
   44. A pharmaceutical composition comprising a therapeutically effective amount of the compound of any one of clauses 1 to 42, or a pharmaceutically acceptable salt thereof, or a therapeutically effective amount of the compound of clause 43, and one or more pharmaceutically acceptable carriers or vehicles.
   45. A pharmaceutical composition comprising the compound of any one of clauses 1 to 42, or a pharmaceutically acceptable salt thereof, or the compound of clause 43, and one or more pharmaceutically acceptable carriers or vehicles.
   46. A method of inhibiting a voltage-gated sodium channel in a subject comprising administering to the subject the compound of any one of clauses 1 to 42, or a pharmaceutically acceptable salt thereof, the compound of clause 43, or the pharmaceutical composition of clause 44 or 45.
   47. The method of clause 46, wherein the voltage-gated sodium channel is Na_{V}1.8.
   48. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of any one of clauses 1 to 42, or a pharmaceutically acceptable salt thereof, the compound of clause 43, or the pharmaceutical composition of clause 44 or 45.
   49. The method of clause 48, where the method comprises treating or lessening the severity in the subject of neuropathic pain.
   50. The method of clause 49, wherein the neuropathic pain comprises post-herpetic neuralgia.
   51. The method of clause 49, wherein the neuropathic pain comprises small-fiber neuropathy.
   52. The method of clause 49, wherein the neuropathic pain comprises idiopathic small-fiber neuropathy.
   53. The method of clause 49, wherein the neuropathic pain comprises diabetic neuropathy.
   54. The method of clause 53, wherein the diabetic neuropathy comprises diabetic peripheral neuropathy.
   55. The method of clause 48, wherein the method comprises treating or lessening the severity in the subject of chronic pain.
   56. The method of clause 55, wherein the chronic pain is lumbosacral radiculopathy.
   57. The method of clause 48, wherein the method comprises treating or lessening the severity in the subject of musculoskeletal pain.
   58. The method of clause 57, wherein the musculoskeletal pain comprises osteoarthritis pain.
   59. The method of clause 48, wherein the method comprises treating or lessening the severity in the subject of acute pain.
   60. The method of clause 59, wherein the acute pain comprises acute post-operative pain.
   61. The method of clause 48, wherein the method comprises treating or lessening the severity in the subject of postsurgical pain.
   62. The method of clause 61, wherein the postsurgical pain comprises bunionectomy pain.
   63. The method of clause 61, wherein the postsurgical pain comprises abdominoplasty pain.
   64. The method of clause 61, wherein the postsurgical pain comprises herniorrhaphy pain.
   65. The method of clause 48, wherein the method comprises treating or lessening the severity in the subject of visceral pain.
   66. The method of any one of clauses 46 to 65, wherein said subject is treated with one or more additional therapeutic agents administered concurrently with, prior to, or subsequent to treatment with the compound, pharmaceutically acceptable salt, or pharmaceutical composition.
   67. Use of the compound of any one of clauses 1 to 42, or a pharmaceutically acceptable salt thereof, the compound of clause 43, or the pharmaceutical composition of clause 44 or 45, as a medicament.

### EXAMPLES

General methods. ¹H NMR spectra were obtained as solutions in an appropriate deuterated solvent such as dimethyl sulfoxide-d₆ (DMSO-d₆).

Compound purity, retention time, and electrospray mass spectrometry (ESI-MS) data were determined by LC/MS analysis. LC/MS methods employed in the following examples are described in Table E.

TABLE E
LC/MS Methods.

| LC/MS | |
|---|---|
| Method | Method Description |
| A | Kinetex ® EVO C₁₈ 2.6 μm 2.1 × 50 mm, 2 to 98% MeCN in H₂O (10 mM Ammonium |
| | Formate, pH 9), 45° C., Flow rate 1.0 mL/min over 1.5 min |
| B | Kinetex ® EVO C₁₈ 2.6 μm 4.6 × 50 mm, 5 to 95% MeCN in H₂O (0.1% Formic Acid), |
| | 45° C., Flow rate 2.0 mL/min over 6 min |
| C | Kinetex ® Polar C₁₈ 2.6 μm 3.0 × 50 mm, 5 to 95% MeCN in H₂O (0.1% Formic Acid), |
| | Flow rate 1.2 mL/min over 3 min |
| D | Kinetex ® Polar C₁₈ 2.6 μm 3.0 × 50 mm, 5 to 95% MeCN in H₂O (0.1% Formic Acid), |
| | Flow rate 1.2 mL/min over 6 min |
| E | Acquity BEH C₁₈ 1.7 μm 2.1 × 50 mm, 1 to 99% MeCN in water (0.05% TFA), 60° C., |
| | Flow rate 1.2 mL/min over 4.5 min |
| F | Acquity BEH C₁₈ 1.7 μm 2.1 × 50 mm, 5 to 95% MeCN in water (0.1% Formic Acid), |
| | Flow rate 0.5 mL/min over 6 min |
| G | Waters ™ BEH C₁₈ 1.7 μm 2.1 × 50 mm, 1 to 99% MeCN in H₂O (0.05% Trifluoroacetic |
| | acid), 60° C., Flow rate 1.2 mL/min over 2.9 min |
| H | Waters ™ BEH C₁₈ 1.7 μm 2.1 × 30 mm, 1 to 99% MeCN in H₂O (0.05% Trifluoroacetic |
| | acid), 60° C., Flow rate 1.5 mL/min over 1.0 min |
| I | Onyx Monolithic C₁₈ 4.6 × 50 μm, 1 to 99% MeCN in water (0.05% Formic Acid), 25° C., |
| | Flow rate = 12 mL/min over 2.9 min |
| J | Waters ™ BEH C₁₈ 2.5 μm 2.1 × 50 mm, 50 to 95% MeCN in H₂O (0.1% NH₃), 40° C., |
| | Flow rate 0.8 mL/min over 4.6 min |
| K | Waters ™ BEH C₁₈ 2.5 μm 2.1 × 50 mm, 50 to 95% MeCN in H₂O (0.1% NH₃), 40° C., |
| | Flow rate 0.8 mL/min over 1.4 min |
| L | Waters ™ BEH C₁₈ 1.7 μm 2.1 × 100 mm, 1 to 99% MeCN in H₂O (10 mM Ammonium |
| | formate), 60° C., Flow rate 0.8 mL/min over 13.5 min |
| M | Phenomenex Gemini NX-C₁₈ 3 μm 4.6 × 50 mm, 20 to 95% MeCN in H₂O (0.1% Formic |
| | Acid), Flow rate 0.5 mL/min over 12 min |

### Abbreviations

Unless otherwise noted, or where the context dictates otherwise, the following abbreviations shall be understood to have the following meanings:

| Abbreviation | Meaning |
|---|---|
| NMR | Nuclear magnetic resonance |
| ESI-MS | Electrospray mass spectrometry |
| LC/MS | Liquid chromatography-mass spectrometry |
| HPLC | High performance liquid chromatography |
| SFC | Supercritical fluid chromatography |
| ESI | Electrospray ionization |
| cm | Centimeters |
| g | Grams |
| mg | Milligrams |
| L | Liter(s) |
| mL | Milliliter(s) |
| μL | Microliter(s) |
| mmol | Millimole(s) |
| h | Hour(s) |
| min | Minute(s) |
| mm | Millimeter(s) |
| μm | Micrometer(s) |
| MHz | Megahertz |
| Hz | Hertz |
| N | Normal (concentration) |
| M | Molar (concentration) |
| mM | Millimolar (concentration) |
| ppm | Parts per million |
| % w/v | Weight-volume concentration |
| % w/w | Weight-weight concentration |
| p-ABSA | 4-Acetamidobenzenesulfonyl azide |
| AcCl | Acetyl chloride |
| AcOH | Acetic acid |
| Bn | Benzyl |
| B₂pin₂ | Bis(pinacolato)diboron |
| ^{t}Bu | tert-Butyl |
| m-CPBA | 3-Chloroperbenzoic acid |
| DAST | (Diethylamino)sulfur trifluoride |
| DBU | 1,8-Diazabicyclo[5.4.0]undec-7-ene |
| DCE | Dichloroethane |
| DCM | Dichloromethane |
| DEA | Diethylamine |
| Dess Martin periodinane | 1,1,1-Tris(acetyloxy)-1,1-dihydro-1,2-benziodoxol-3-(1H)-one |
| DIAD | Diisopropyl azodicarboxylate |
| DIBAL | Diisobutylaluminium hydride |
| DIPEA | N,N-Diisopropylethylamine |
| DMAP | 4-(Dimethylamino)pyridine |
| DMF | Dimethylformamide |
| DMSO | Dimethyl sulfoxide |
| dppf | 1,1′-Bis(diphenylphosphino)ferrocene |
| EDC | N-Ethyl-N′-(3-dimethylaminopropyl)carbodiimide |
| EtOH | Ethanol |
| EtOAc | Ethyl acetate |
| HATU | 1-[Bis(dimethylamino)methylene]-1H-1,2,3-triazolo[4,5-b]pyridinium |
| | 3-oxid hexafluorophosphate |
| (Ir[dF(CF₃)ppy]₂(dtbpy))PF₆ | (4,4′-Di-tert-butyl-2,2′-bipyridine)bis[(2-pyridinyl)phenyl]iridium(III) |
| | hexafluorophosphate |
| LED | Light Emitting Diode |
| MeOH | Methanol |
| MeCN | Acetonitrile |
| 2-MeTHF | 2-Methyltetrahydrofuran |
| NiCl₂ glyme | Nickel(II) chloride ethylene glycol dimethyl ether complex |
| NMO | 4-Methylmorpholine N-oxide |
| NMP | N-Methylpyrrolidone |
| OTf | Trifluoromethanesulfonate |
| PCC | Pyridinium chlorochromate |
| PhMe | Toluene |
| Rh2(S-PTAD)₄ | Tetrakis[(S)-(+)-(1-adamantyl)-(N-phthalimido)acetato]dirhodium(II) |
| T3P ® | Propylphosphonic anhydride |
| TBAI | Tetrabutylammonium iodide |
| TBSCl | tert-Butyldimethylsilyl chloride |
| Tf | Trifluoromethanesulfonyl |
| TFA | Trifluoroacetic acid |
| Tf₂O | Trifluoroacetic anhydride |
| THF | Tetrahydrofuran |
| TMS | Trimethylsilyl |
| THF | Tetrahydrofuran |
| RT | Ambient temperature |
| rt | Retention time |

### Example 1

### rel-2-((1R,2R,3aR,6aS)-1-(3,4-Difluoro-2-methoxyphenyl)-4,4-difluorooctahydropentalen-2-yl)-4-oxo-1,4-dihydro-1,6-naphthyridine-5-carboxamide (1) and rel-2-((1S,2S,3aS,6aR)-1-(3,4-difluoro-2-methoxyphenyl)-4,4-difluorooctahydropentalen-2-yl)-4-oxo-1,4-dihydro-1,6-naphthyridine-5-carboxamide (2)

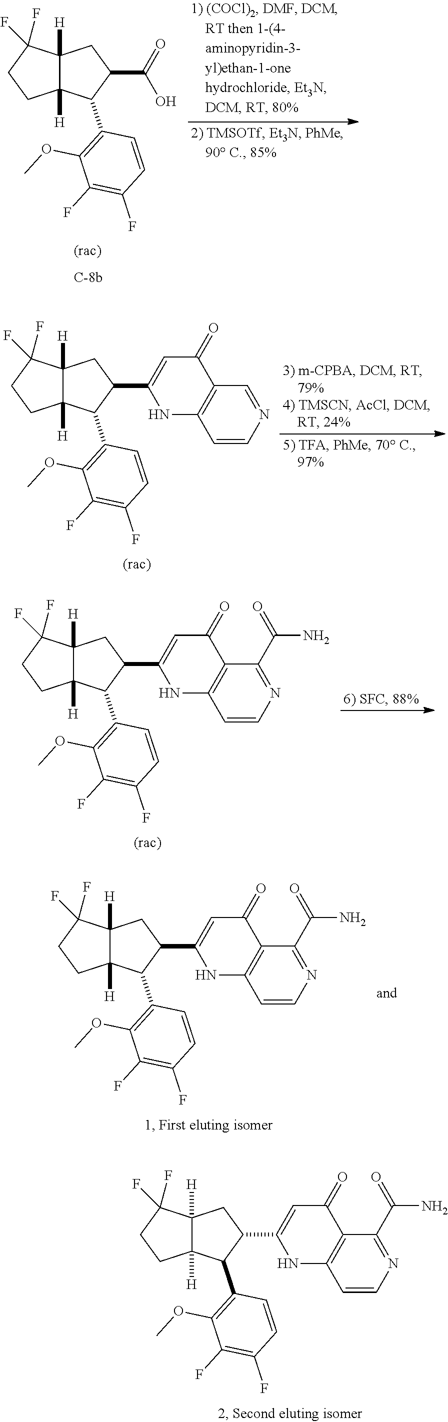 [SMILES: COc1c([C@@H]2[C@@H]3CCC(F)(F)[C@@H]3C[C@H]2C(=O)O)ccc(F)c1F ; COc1c([C@@H]2[C@@H]3CCC(F)(F)[C@@H]3C[C@H]2c2cc(=O)c3cnccc3[nH]2)ccc(F)c1F ; COc1c([C@@H]2[C@@H]3CCC(F)(F)[C@@H]3C[C@H]2c2cc(=O)c3c(C(N)=O)nccc3[nH]2)ccc(F)c1F ; COc1c([C@@H]2[C@@H]3CCC(F)(F)[C@@H]3C[C@H]2c2cc(=O)c3c(C(N)=O)nccc3[nH]2)ccc(F)c1F ; COc1c([C@H]2[C@H]3CCC(F)(F)[C@H]3C[C@@H]2c2cc(=O)c3c(C(N)=O)nccc3[nH]2)ccc(F)c1F]

### Step 1

Oxalyl chloride (436.5 mg, 0.3 mL, 3.439 mmol) was added to a solution of rac-(1R,2R,3aR,6aS)-1-(3,4-difluoro-2-methoxyphenyl)-4,4-difluorooctahydropentalene-2-carboxylic acid (C-8b, 1 g, 3.006 mmol) and DMF (12 μL, 0.155 mmol) in DCM (8 mL) at ambient temperature and the reaction mixture was stirred at ambient temperature for 2 h. 1-(4-Aminopyridin-3-yl)ethan-1-one hydrochloride (575 mg, 3.331 mmol) and Et₃N (1.089 g, 1.5 mL, 10.762 mmol) were successively added and the reaction mixture was stirred for 65 h at ambient temperature. The mixture was quenched by addition of water (100 mL) and extracted with DCM (2×50 mL). The combined organic layers were dried (Na₂SO₄), filtered and concentrated in vacuo. Purification by flash chromatography (SiO₂, 10 to 100% EtOAc in heptane) gave rac-(1R,2R,3aR,6aS)—N-(3-acetylpyridin-4-yl)-1-(3,4-difluoro-2-methoxyphenyl)-4,4-difluorooctahydropentalene-2-carboxamide (1.08 g, 80%) as an off-white solid. ¹H NMR (400 MHz, Chloroform-d) δ 12.03 (s, 1H), 9.09 (s, 1H), 8.64-8.48 (m, 2H), 6.94-6.85 (m, 1H), 6.84-6.72 (m, 1H), 4.00 (d, J=2.4 Hz, 3H), 3.82 (dd, J=12.2, 8.3 Hz, 1H), 3.22 (quin, J=8.8 Hz, 1H), 3.10 (td, J=12.1, 6.6 Hz, 1H), 3.00-2.83 (m, 1H), 2.73 (s, 3H), 2.50 (dd, J=13.3, 6.7 Hz, 1H), 2.12-1.83 (m, 3H), 1.40-1.29 (m, 1H), 1.22-1.08 (m, 1H) ppm. ESI-MS m/z calc. 450.157, found 451.2 (M+1)⁺.

### Step 2

Et₃N (450.12 mg, 0.62 mL, 4.448 mmol) and TMSOTf (2.44 g, 2 mL, 10.978 mmol) were successively added to a solution of rac-(1R,2R,3aR,6aS)—N-(3-acetylpyridin-4-yl)-1-(3,4-difluoro-2-methoxyphenyl)-4,4-difluorooctahydropentalene-2-carboxamide (1 g, 2.218 mmol) in toluene (10 mL) and the reaction mixture was stirred at 90° C. for 4 h. The reaction was cooled to ambient temperature, diluted with 2-MeTHF (30 mL) and washed with a saturated aqueous NaHCO₃ solution (20 mL). The aqueous layer was extracted with 2-MeTHF (3×30 mL). The combined organic layers were dried (Na₂SO₄), filtered and concentrated in vacuo. The residue was dissolved in 2-MeTHF (30 mL), washed with a saturated aqueous NH₄Cl solution (20 mL), water (20 mL) and brine (20 mL), dried (Na₂SO₄), filtered and concentrated in vacuo. Purification by trituration from 2-MeTHF (2 mL) and MTBE (10 mL) gave rac-2-((1R,2R,3aR,6aS)-1-(3,4-difluoro-2-methoxyphenyl)-4,4-difluorooctahydropentalen-2-yl)-1,6-naphthyridin-4(1H)-one (820 mg, 85%) as a tan solid. ¹H NMR (400 MHz, Methanol-d₄) δ 9.24 (s, 1H), 8.56 (d, J=6.0 Hz, 1H), 7.52 (d, J=6.0 Hz, 1H), 6.99-6.94 (m, 1H), 6.90-6.81 (m, 1H), 6.28 (s, 1H), 4.04 (d, J=2.2 Hz, 3H), 3.93 (dd, J=12.8, 8.1 Hz, 1H), 3.47 (td, J=12.4, 6.8 Hz, 1H), 3.39-3.32 (m, 1H, overlapped with solvent), 3.08-2.95 (m, 1H), 2.62 (dd, J=13.4, 6.7 Hz, 1H), 2.10-1.96 (m, 3H), 1.44-1.21 (m, 2H) ppm; exchangeable H not observed. ESI-MS m/z calc. 432.146, found 433.2 (M+1)⁺.

### Step 3

m-CPBA (206 mg, 0.919 mmol) was added to a solution of rac-2-((1R,2R,3aR,6aS)-1-(3,4-difluoro-2-methoxyphenyl)-4,4-difluorooctahydropentalen-2-yl)-1,6-naphthyridin-4(1H)-one (215 mg, 0.455 mmol) in DCM (9 mL) and the reaction mixture was stirred at ambient temperature for 6 h. The reaction was diluted with EtOAc (30 mL) and extracted with a saturated aqueous NaHCO₃ solution (3×30 mL). The combined organic layers were washed with brine (25 mL), dried (Na₂SO₄), filtered and concentrated in vacuo to give rac-2-((1R,2R,3aR,6aS)-1-(3,4-difluoro-2-methoxyphenyl)-4,4-difluorooctahydropentalen-2-yl)-4-oxo-1,4-dihydro-1,6-naphthyridine 6-oxide (170 mg, 79%) as a yellow solid, which was used without further purification in the next step. ¹H NMR (400 MHz, Methanol-d₄) δ 8.86 (d, J=1.8 Hz, 1H), 8.35 (dd, J=7.3, 1.9 Hz, 1H), 7.68 (d, J=7.2 Hz, 1H), 7.00-6.91 (m, 1H), 6.90-6.81 (m, 1H), 6.29 (s, 1H), 4.03 (d, J=2.2 Hz, 3H), 3.93 (dd, J=12.7, 8.1 Hz, 1H), 3.47 (td, J=12.3, 7.0 Hz, 1H), 3.40-3.33 (m, 1H), 3.08-2.93 (m, 1H), 2.66-2.55 (m, 1H), 2.11-1.93 (m, 3H), 1.46-1.34 (m, 1H), 1.32-1.21 (m, 1H) ppm; exchangeable H not observed. ESI-MS m/z calc. 448.141, found 449.2 (M+1)⁺.

### Step 4

Under a nitrogen atmosphere, TMSCN (146.71 mg, 0.185 mL, 1.479 mmol) and AcCl (110.4 mg, 0.1 mL, 1.406 mmol) were successively added to a solution of rac-2-((1R,2R,3aR,6aS)-1-(3,4-difluoro-2-methoxyphenyl)-4,4-difluorooctahydropentalen-2-yl)-4-oxo-1,4-dihydro-1,6-naphthyridine 6-oxide (165 mg, 0.347 mmol) in DCM (2 mL) and the reaction mixture was stirred at ambient temperature for 19 h. The reaction was carefully quenched by pouring the mixture into a stirred saturated aqueous NaHCO₃ solution (10 mL). The aqueous phase was separated and extracted with DCM (15 mL). The combined organic layers were washed with a saturated aqueous NaHCO₃ solution (10 mL) and water (10 mL), dried (Na₂SO₄), filtered and concentrated in vacuo. Purification by reverse phase chromatography (5 to 80% MeCN in water with 0.1% formic acid) gave rac-2-((1R,2R,3aR,6aS)-1-(3,4-difluoro-2-methoxyphenyl)-4,4-difluorooctahydropentalen-2-yl)-4-oxo-1,4-dihydro-1,6-naphthyridine-5-carbonitrile (38 mg, 24%) as a white solid. ¹H NMR (400 MHz, Methanol-d₄) δ 8.65 (d, J=5.9 Hz, 1H), 7.70 (d, J=5.9 Hz, 1H), 6.99-6.92 (m, 1H), 6.91-6.81 (m, 1H), 6.29 (s, 1H), 4.04 (d, J=2.2 Hz, 3H), 3.93 (dd, J=12.9, 8.0 Hz, 1H), 3.51-3.41 (m, 1H), 3.40-3.27 (m, 1H, overlapped with solvent), 3.09-2.93 (m, 1H), 2.61 (dd, J=13.4, 6.5 Hz, 1H), 2.11-1.93 (m, 3H), 1.46-1.33 (m, 1H), 1.32-1.21 (m, 1H) ppm; exchangeable H not observed. ESI-MS m/z calc. 457.141, found 458.2 (M+1)⁺.

### Step 5

In a sealed vial, a solution of rac-2-((1R,2R,3aR,6aS)-1-(3,4-difluoro-2-methoxyphenyl)-4,4-difluorooctahydropentalen-2-yl)-4-oxo-1,4-dihydro-1,6-naphthyridine-5-carbonitrile (100 mg, 0.167 mmol) and TFA (2.22 g, 1.5 mL, 19.470 mmol) in toluene (2.5 mL) was stirred at 70° C. for 48 h. The reaction mixture was concentrated in vacuo. Purification by reverse phase chromatography (C₁₈ column, 5% to 100% MeCN in water with 0.1% formic acid) gave rac-2-((1R,2R,3aR,6aS)-1-(3,4-difluoro-2-methoxyphenyl)-4,4-difluorooctahydropentalen-2-yl)-4-oxo-1,4-dihydro-1,6-naphthyridine-5-carboxamide (80 mg, 97%) as a tan solid. ¹H NMR (400 MHz, DMSO-d₆) δ 11.68 (s, 1H), 8.43 (d, J=5.9 Hz, 1H), 7.43 (d, J=5.9 Hz, 1H), 7.39 (br s, 1H), 7.20 (br s, 1H), 7.16-7.00 (m, 2H), 6.12 (br s, 1H), 3.94 (d, J=1.7 Hz, 3H), 3.87 (dd, J=13.0, 7.8 Hz, 1H), 3.47-3.36 (m, 1H), 3.25-3.15 (m, 1H), 3.04-2.90 (m, 1H), 2.38 (dd, J=13.1, 6.7 Hz, 1H), 2.11-1.95 (m, 3H), 1.38-1.27 (m, 1H), 1.18-1.06 (m, 1H) ppm. ESI-MS m/z calc. 475.152, found 476.2 (M+1)⁺.

### Step 6

The enantiomers of rac-2-((1R,2R,3aR,6aS)-1-(3,4-difluoro-2-methoxyphenyl)-4,4-difluorooctahydropentalen-2-yl)-4-oxo-1,4-dihydro-1,6-naphthyridine-5-carboxamide (56 mg, 0.117 mmol) were separated by chiral SFC using a Lux® i-Cellulose-5 column, 5 μm particle size, 25 cm×21.2 mm from Phenomenex (Mobile phase: 30% MeOH, 70% CO₂; Flow rate 75 mL/min) to give:

First Eluting Isomer: rel-2-((1R,2R,3aR,6aS)-1-(3,4-difluoro-2-methoxyphenyl)-4,4-difluorooctahydropentalen-2-yl)-4-oxo-1,4-dihydro-1,6-naphthyridine-5-carboxamide (Compound 1, 24 mg, 43%). ¹H NMR (400 MHz, Methanol-d₄) δ 8.48 (d, J=6.0 Hz, 1H), 7.53 (d, J=6.1 Hz, 1H), 7.00-6.92 (m, 1H), 6.90-6.80 (m, 1H), 6.24 (br s, 1H), 4.02 (d, J=2.1 Hz, 3H), 3.92 (dd, J=12.8, 8.1 Hz, 1H), 3.51-3.39 (m, 1H), 3.36-3.27 (m, 1H, overlapped with solvent), 3.09-2.94 (m, 1H), 2.60 (dd, J=13.0, 6.8 Hz, 1H), 2.10-1.94 (m, 3H), 1.44-1.21 (m, 2H) ppm, exchangeable H not observed. ESI-MS m/z calc. 475.152, found 476.2 (M+1)⁺.

Second Eluting Isomer: rel-2-((1S,2S,3aS,6aR)-1-(3,4-difluoro-2-methoxyphenyl)-4,4-difluorooctahydropentalen-2-yl)-4-oxo-1,4-dihydro-1,6-naphthyridine-5-carboxamide (Compound 2, 25 mg, 45%). ¹H NMR (400 MHz, Methanol-d₄) δ 8.48 (d, J=5.9 Hz, 1H), 7.53 (d, J=5.7 Hz, 1H), 7.02-6.92 (m, 1H), 6.90-6.79 (m, 1H), 6.24 (br s, 1H), 4.02 (d, J=2.0 Hz, 3H), 3.92 (dd, J=13.0, 7.9 Hz, 1H), 3.50-3.40 (m, 1H), 3.38-3.27 (m, 1H, overlapped with solvent), 3.09-2.93 (m, 1H), 2.60 (dd, J=13.0, 6.5 Hz, 1H), 2.13-1.92 (m, 3H), 1.44-1.23 (m, 2H) ppm, exchangeable H not observed. ESI-MS m/z calc. 475.152, found 476.2 (M+1)⁺.

### Example 2

### rel-2-((1S,2S)-2-(3,4-Difluoro-2-methoxyphenyl)-4,4-dimethylcyclohexyl)-4-oxo-1,4-dihydro-1,6-naphthyridine-5-carboxamide (3) and rel-2-((1R,2R)-2-(3,4-difluoro-2-methoxyphenyl)-4,4-dimethylcyclohexyl)-4-oxo-1,4-dihydro-1,6-naphthyridine-5-carboxamide (4)

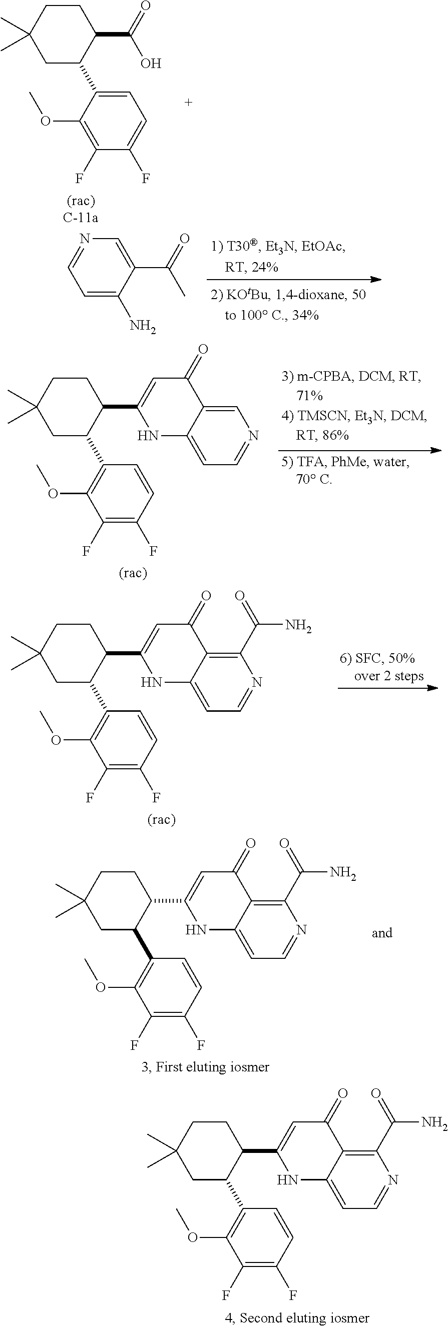

### Step 1

T3P® (3.4 mL, 50% w/v solution in EtOAc, 5.343 mmol) was added to a solution of rac-(1R,2R)-2-(3,4-difluoro-2-methoxyphenyl)-4,4-dimethylcyclohexane-1-carboxylic acid (C-11a, 618 mg, 1.693 mmol), 1-(4-aminopyridin-3-yl)ethan-1-one hydrochloride (470 mg, 2.723 mmol) and Et₃N (1.089 g, 1.5 mL, 10.762 mmol) in EtOAc (7 mL) and reaction mixture was stirred at ambient temperature overnight. The reaction was quenched by addition of a saturated aqueous NaHCO₃ solution (20 mL) and extracted with EtOAc (3×30 mL). The combined organic layers were dried (Na₂SO₄), filtered and concentrated in vacuo. Purification by reverse phase chromatography (C₁₈ column, 5 to 95% MeCN in water with 0.1% formic acid) gave rac-(1R,2R)—N-(3-acetylpyridin-4-yl)-2-(3,4-difluoro-2-methoxyphenyl)-4,4-dimethylcyclohexane-1-carboxamide (193 mg, 24%) as a yellow glassy solid. ¹H NMR (400 MHz, Chloroform-d) δ 11.71 (br s, 1H), 9.06 (s, 1H), 8.57-8.47 (m, 2H), 6.97-6.87 (m, 1H), 6.84-6.71 (m, 1H), 3.97 (d, J=1.7 Hz, 3H), 3.55 (t, J=11.1 Hz, 1H), 2.71 (s, 3H), 2.68-2.56 (m, 1H), 2.02-1.92 (m, 2H), 1.66-1.50 (m, 3H), 1.46-1.34 (m, 1H), 1.16 (s, 3H), 1.00 (s, 3H) ppm. ESI-MS m z calc. 416.191, found 417.2 (M+1)⁺.

### Step 2

In a sealed tube, KO^{t}Bu (100 mg, 0.891 mmol) was added to a solution of rac-(1R,2R)—N-(3-acetylpyridin-4-yl)-2-(3,4-difluoro-2-methoxyphenyl)-4,4-dimethylcyclohexane-1-carboxamide (193 mg, 0.402 mmol) in 1,4-dioxane (4 mL). The tube was sealed and the solution was placed under a nitrogen atmosphere. The reaction was heated at 50° C. for 1 h, at 90° C. for 2 h and at 100° C. for 20 h. The mixture was cooled to ambient temperature, diluted with a saturated aqueous NH₄Cl solution (10 mL) and extracted with DCM (3×15 mL). The combined organic layers were dried (Na₂SO₄), filtered and concentrated in vacuo. Purification by reverse phase chromatography (C₁₈ column, 5 to 95% of MeCN in water with 0.1% formic acid) gave rac-2-((1R,2R)-2-(3,4-difluoro-2-methoxyphenyl)-4,4-dimethylcyclohexyl)-1,6-naphthyridin-4(1H)-one (55 mg, 34%) as a beige solid. ¹H NMR (400 MHz, Chloroform-d) δ 9.41 (s, 1H), 8.59 (d, J=5.6 Hz, 1H), 8.16 (br s, 1H), 7.14 (d, J=5.4 Hz, 1H), 6.98-6.74 (m, 2H), 6.20 (s, 1H), 4.02 (br s, 3H), 3.67-3.43 (m, 1H), 2.83-2.66 (m, 1H), 2.04-1.80 (m, 2H), 1.71-1.39 (m, 4H), 1.14 (s, 3H), 1.05 (s, 3H) ppm. ESI-MS m/z calc. 398.181, found 399.2 (M+1)⁺.

### Step 3

m-CPBA (57 mg, 0.254 mmol) was added to a solution of rac-2-((1R,2R)-2-(3,4-difluoro-2-methoxyphenyl)-4,4-dimethylcyclohexyl)-1,6-naphthyridin-4(1H)-one (50 mg, 0.126 mmol) in DCM (2 mL) and reaction mixture was stirred at ambient temperature for 4 h. The reaction was diluted with EtOAc (20 mL) and washed with a saturated aqueous NaHCO₃ solution (3×30 mL). The organic layer was dried (Na₂SO₄), filtered and concentrated in vacuo to give rac-2-((1R,2R)-2-(3,4-difluoro-2-methoxyphenyl)-4,4-dimethylcyclohexyl)-4-oxo-1,4-dihydro-1,6-naphthyridine 6-oxide (40 mg, 71%) as an orange solid, which was used without further purification in the next step. ESI-MS m/z calc. 414.176, found 415.2 (M+1)⁺.

### Step 4

In a sealed tube, TMSCN (95.160 mg, 0.12 mL, 0.959 mmol) and Et₃N (130.68 mg, 0.18 mL, 1.291 mmol) were successively added to a solution of rac-2-((1R,2R)-2-(3,4-difluoro-2-methoxyphenyl)-4,4-dimethylcyclohexyl)-4-oxo-1,4-dihydro-1,6-naphthyridine 6-oxide (40 mg, 0.090 mmol) in DCM (2 mL) under a nitrogen atmosphere. The tube was sealed and the reaction mixture was stirred at ambient temperature overnight. The mixture was diluted with a saturated NaHCO₃ solution (20 mL) and extracted with EtOAc (3×15 mL). The combined organic layers were dried (Na₂SO₄), filtered and concentrated in vacuo to give rac-2-((1R,2R)-2-(3,4-difluoro-2-methoxyphenyl)-4,4-dimethylcyclohexyl)-4-oxo-1,4-dihydro-1,6-naphthyridine-5-carbonitrile (35 mg, 86%) as a beige solid, which was used without further purification in the next step. ¹H NMR (400 MHz, Methanol-d₄) δ 8.62 (d, J=5.9 Hz, 1H), 7.66 (d, J=5.9 Hz, 1H), 7.12-6.97 (m, 1H), 6.94-6.77 (m, 1H), 6.21 (s, 1H), 3.90 (s, 3H), 3.78-3.58 (m, 1H), 3.00-2.78 (m, 1H), 2.07-1.90 (m, 2H), 1.71-1.45 (m, 4H), 1.22 (s, 3H), 1.04 (s, 3H) ppm; exchangeable H not observed. ESI-MS m/z calc. 423.176, found 424.2 (M+1)⁺.

### Step 5

In a sealed tube, TFA (740 mg, 0.5 mL, 6.490 mmol) and a drop of water were successively added to a solution of rac-2-((1R,2R)-2-(3,4-difluoro-2-methoxyphenyl)-4,4-dimethylcyclohexyl)-4-oxo-1,4-dihydro-1,6-naphthyridine-5-carbonitrile (35 mg, 0.077 mmol) in toluene (2 mL). The tube was sealed and the reaction mixture was stirred at 70° C. overnight. The mixture was cooled to ambient temperature and concentrated in vacuo. The residue was diluted with EtOAc (30 mL), washed with a saturated aqueous NaHCO₃ solution (2×10 mL), dried (Na₂SO₄) and concentrated in vacuo. Purification by reverse phase chromatography (C₁₈ column, 5 to 95% of MeCN in water with 0.1% formic acid) gave rac-2-((1R,2R)-2-(3,4-difluoro-2-methoxyphenyl)-4,4-dimethylcyclohexyl)-4-oxo-1,4-dihydro-1,6-naphthyridine-5-carboxamide.

### Step 6

The enantiomers of rac-2-((1R,2R)-2-(3,4-difluoro-2-methoxyphenyl)-4,4-dimethylcyclohexyl)-4-oxo-1,4-dihydro-1,6-naphthyridine-5-carboxamide (56 mg, 0.117 mmol) were separated by chiral SFC using a Lux® Cellulose-4 column, 5 μm particle size, 15 cm×21.2 mm from Phenomenex (Mobile phase: 30% MeOH, 70% CO₂; Flow rate 75 mL/min) to give:

First Eluting Isomer: rel-2-((1S,2S)-2-(3,4-difluoro-2-methoxyphenyl)-4,4-dimethylcyclohexyl)-4-oxo-1,4-dihydro-1,6-naphthyridine-5-carboxamide (Compound 3, 7.8 mg, 22% over 2 steps) as a white solid. ¹H NMR (400 MHz, Chloroform-d) δ 15.11 (s, 1H), 8.90 (br s, 1H), 8.53 (d, J=5.6 Hz, 1H), 7.95 (d, J=5.6 Hz, 1H), 6.98-6.85 (m, 2H), 6.75-6.57 (m, 1H), 6.03 (br s, 1H), 3.94 (s, 3H), 3.88-3.72 (m, 1H), 3.20-3.06 (m, 1H), 1.99-1.88 (m, 2H), 1.69-1.45 (m, 4H, overlapped with water), 1.21 (s, 3H), 1.02 (s, 3H) ppm. ESI-MS m/z calc. 441.186, found 442.2 (M+1)⁺.

Second Eluting Isomer: rel-2-((1R,2R)-2-(3,4-difluoro-2-methoxyphenyl)-4,4-dimethylcyclohexyl)-4-oxo-1,4-dihydro-1,6-naphthyridine-5-carboxamide (Compound 4, 10 mg, 28% over 2 steps) as a white solid. ¹H NMR (400 MHz, Chloroform-d) δ 15.10 (s, 1H), 8.89 (br s, 1H), 8.51 (d, J=5.9 Hz, 1H), 7.93 (d, J=5.6 Hz, 1H), 6.96-6.85 (m, 2H), 6.74-6.56 (m, 1H), 6.02 (br s, 1H), 3.92 (s, 3H), 3.85-3.72 (m, 1H), 3.18-3.03 (m, 1H), 1.97-1.87 (m, 2H), 1.66-1.45 (m, 4H, overlapped with water), 1.20 (s, 3H), 1.01 (s, 3H) ppm. ESI-MS m/z calc. 441.186, found 442.2 (M+1)⁺.

### Example 3

### rel-2-((1R,2R,3aR,6aS)-1-(3,4-Difluoro-2-methylphenyl)-4,4-difluorooctahydropentalen-2-yl)-4-oxo-1,4-dihydro-1,6-naphthyridine-5-carboxamide (5) and rel-2-((1S,2S,3aS,6aR)-1-(3,4-difluoro-2-methylphenyl)-4,4-difluorooctahydropentalen-2-yl)-4-oxo-1,4-dihydro-1,6-naphthyridine-5-carboxamide (6)

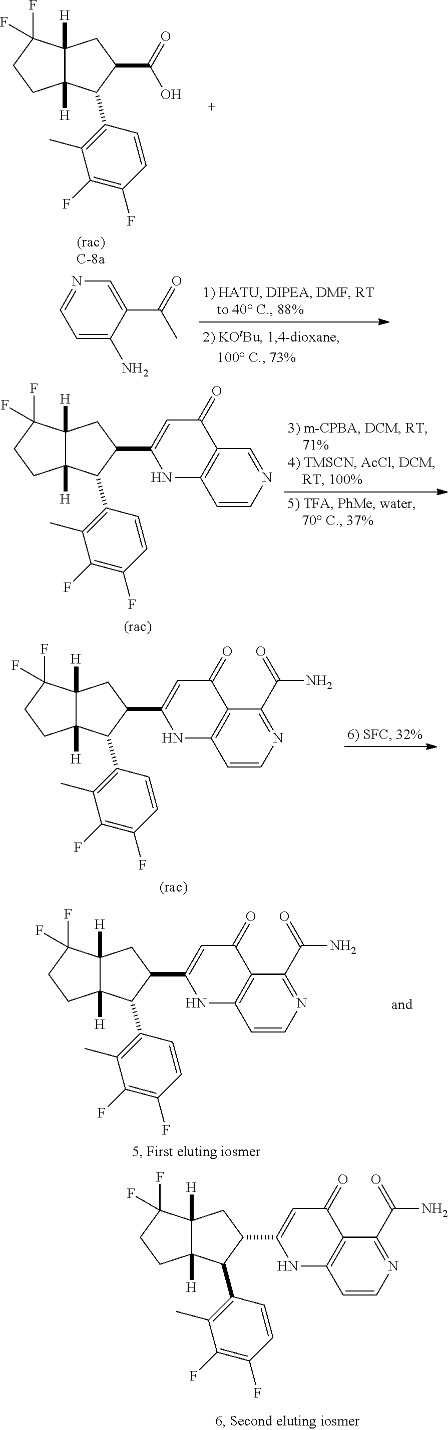

### Step 1

DIPEA (3.33 g, 4.5 mL, 25.765 mmol) was added to a stirred solution of rac-(1R,2R,3aR,6aS)-1-(3,4-difluoro-2-methylphenyl)-4,4-difluorooctahydropentalene-2-carboxylic acid (C-8a, 536 mg, 1.620 mmol) and 1-(4-aminopyridin-3-yl)ethan-1-one hydrochloride (342 mg, 1.981 mmol) in DMF (13 mL) and the reaction mixture was stirred at ambient temperature for 30 min. HATU (936 mg, 2.461 mmol) was added and the reaction was stirred at 40° C. for 2 h. The mixture was cooled to ambient temperature and diluted with EtOAc (50 mL) and water (50 mL). The aqueous phase was separated and extracted with EtOAc (3×50 mL). The combined organic phases were concentrated in vacuo and the residue was dissolved in MTBE (60 mL). The solution was washed with water (4×30 mL), dried (Na₂SO₄), filtered and concentrated in vacuo to give rac-(1R,2R,3aR,6aS)—N-(3-acetylpyridin-4-yl)-1-(3,4-difluoro-2-methylphenyl)-4,4-difluorooctahydropentalene-2-carboxamide (656 mg, 88%) as a yellow solid, which was used without further purification in the next step. ¹H NMR (400 MHz, Chloroform-d) δ 12.06 (s, 1H), 9.08 (s, 1H), 8.62-8.50 (m, 2H), 7.00-6.86 (m, 2H), 3.75-3.65 (m, 1H), 3.23-3.07 (m, 2H), 3.01-2.86 (m, 1H), 2.76-2.68 (m, 3H), 2.55-2.46 (m, 1H), 2.31 (d, J=2.5 Hz, 3H), 2.13-1.85 (m, 3H), 1.34-1.15 (m, 2H) ppm. ESI-MS m/z calc. 434.162, found 435.2 (M+1)⁺.

### Step 2

In a sealed tube, KO^{t}Bu (365 mg, 3.253 mmol) was added to a solution of rac-(1R,2R,3aR,6aS)—N-(3-acetylpyridin-4-yl)-1-(3,4-difluoro-2-methylphenyl)-4,4-difluorooctahydropentalene-2-carboxamide (656 mg, 1.421 mmol) in 1,4-dioxane (15 mL). The solution was placed under a nitrogen atmosphere and the tube was sealed. The reaction mixture was heated at 100° C. for 22 h. The reaction was cooled to ambient temperature, diluted with a saturated aqueous NH₄Cl solution (50 mL) and extracted with EtOAc (3×50 mL). The combined organic layers were dried (Na₂SO₄), filtered and concentrated in vacuo to give rac-2-((1R,2R,3aR,6aS)-1-(3,4-difluoro-2-methylphenyl)-4,4-difluorooctahydropentalen-2-yl)-1,6-naphthyridin-4(1H)-one (463 mg, 73%) as a beige solid, which was used without further purification in the next step. ¹H NMR (400 MHz, Methanol-d₄) δ 9.23 (s, 1H), 8.56 (d, J=5.9 Hz, 1H), 7.51 (d, J=5.9 Hz, 1H), 7.03-6.90 (m, 2H), 6.26 (s, 1H), 3.88-3.79 (m, 1H), 3.60-3.49 (m, 1H), 3.09-2.95 (m, 1H), 2.68-2.58 (m, 1H), 2.38 (d, J=2.5 Hz, 3H), 2.11-1.93 (m, 3H), 1.37-1.21 (m, 3H) ppm; exchangeable H not observed. ESI-MS m/z calc. 416.151, found 417.2 (M+1)⁺.

### Step 3

m-CPBA (472 mg, 2.106 mmol) was added to a solution of rac-2-((1R,2R,3aR,6aS)-1-(3,4-difluoro-2-methylphenyl)-4,4-difluorooctahydropentalen-2-yl)-1,6-naphthyridin-4(1H)-one (463 mg, 1.043 mmol) in DCM (20 mL) and the reaction mixture was stirred at ambient temperature for 2.5 h. The mixture was concentrated in vacuo and the residue was solubilised in EtOAc (30 mL). The mixture was washed with a saturated aqueous NaHCO₃ solution (2×30 mL) and brine (30 mL), dried (Na₂SO₄), filtered and concentrated in vacuo to give rac-2-((1R,2R,3aR,6aS)-1-(3,4-difluoro-2-methylphenyl)-4,4-difluorooctahydropentalen-2-yl)-4-oxo-1,4-dihydro-1,6-naphthyridine 6-oxide (452 mg, 71%) as a yellow solid, which was used in the next step without further purification. ¹H NMR (400 MHz, Methanol-d₄) δ 8.84 (s, 1H), 8.35 (d, J=6.4 Hz, 1H), 7.68 (d, J=6.9 Hz, 1H), 7.02-6.90 (m, 2H), 6.26 (s, 1H), 3.88-3.77 (m, 1H), 3.60-3.48 (m, 1H), 3.06-2.93 (m, 1H), 2.67-2.55 (m, 1H), 2.37 (d, J=2.1 Hz, 3H), 2.10-1.90 (m, 3H), 1.36-1.20 (m, 3H) ppm; exchangeable H not observed. ESI-MS m/z calc. 432.146, found 433.2 (M+1)⁺.

### Step 4

Under a nitrogen atmosphere, TMSCN (317.2 mg, 0.4 mL, 3.197 mmol) and AcCl (220.8 mg, 0.2 mL, 2.813 mmol) were successively added to a solution of rac-2-((1R,2R,3aR,6aS)-1-(3,4-difluoro-2-methylphenyl)-4,4-difluorooctahydropentalen-2-yl)-4-oxo-1,4-dihydro-1,6-naphthyridine 6-oxide (452 mg, 0.739 mmol) in DCM (10 mL) and the reaction mixture was stirred at ambient temperature for 2 h. The reaction was carefully quenched, by pouring the mixture into a stirred saturated aqueous NaHCO₃ solution (20 mL), and diluted with DCM (20 mL). The organic layer was separated, washed with a saturated NaHCO₃ aqueous solution (2×20 mL) and water (20 mL), dried (Na₂SO₄), filtered and concentrated in vacuo to give rac-2-((1R,2R,3aR,6aS)-1-(3,4-difluoro-2-methylphenyl)-4,4-difluorooctahydropentalen-2-yl)-4-oxo-1,4-dihydro-1,6-naphthyridine-5-carbonitrile (450 mg, 100%) as a tan solid, which was used without further purification in the next step. ¹H NMR (400 MHz, DMSO-d₆) δ 12.11 (br s, 1H), 8.66 (d, J=5.6 Hz, 1H), 7.72 (d, J=5.6 Hz, 1H), 7.20-7.03 (m, 2H), 6.28 (s, 1H), 3.88 (dd, J=12.6, 7.7 Hz, 1H), 3.50 (td, J=12.3, 7.0 Hz, 1H), 3.23 (quin, J=8.4 Hz, 1H), 3.04-2.89 (m, 1H), 2.41 (dd, J=13.2, 6.6 Hz, 1H), 2.32 (d, J=2.2 Hz, 3H), 2.13-1.92 (m, 3H), 1.32-1.20 (m, 1H), 1.16-1.02 (m, 1H) ppm. ESI-MS m/z calc. 441.146, found 442.2 (M+1)⁺.

### Step 5

A solution of rac-2-((1R,2R,3aR,6aS)-1-(3,4-difluoro-2-methylphenyl)-4,4-difluorooctahydropentalen-2-yl)-4-oxo-1,4-dihydro-1,6-naphthyridine-5-carbonitrile (455 mg, 0.808 mmol) and TFA (8.88 g, 6 mL, 77.879 mmol) in a mixture of toluene (10 mL) and water (0.2 mL) was stirred at 70° C. for 24 h and at ambient temperature for 3 days. The reaction mixture was concentrated in vacuo. The residue was solubilized in EtOAc (50 mL), washed with a saturated NaHCO₃ aqueous solution (3×50 mL) and brine (50 mL). The organic layer was dried (Na₂SO₄), filtered and concentrated in vacuo. Purification by reverse phase chromatography (C₁₈ column, 2.5 to 60% MeCN in water with 0.1% formic acid) gave rac-2-((1R,2R,3aR,6aS)-1-(3,4-difluoro-2-methylphenyl)-4,4-difluorooctahydropentalen-2-yl)-4-oxo-1,4-dihydro-1,6-naphthyridine-5-carboxamide (136 mg, 37%). ¹H NMR (400 MHz, Methanol-d₄) δ 8.48 (br s, 1H), 7.59-7.48 (m, 1H), 7.02-6.91 (m, 2H), 6.23 (s, 1H), 3.86-3.75 (m, 1H), 3.57-3.45 (m, 1H), 3.10-2.93 (m, 1H), 2.67-2.55 (m, 1H), 2.36 (d, J=2.2 Hz, 3H), 2.10-1.93 (m, 3H), 1.42-1.20 (m, 3H) ppm; exchangeable H not observed. ESI-MS m/z calc. 459.157, found 460.2 (M+1)⁺.

### Step 6

The enantiomers of rac-2-((1R,2R,3aR,6aS)-1-(3,4-difluoro-2-methylphenyl)-4,4-difluorooctahydropentalen-2-yl)-4-oxo-1,4-dihydro-1,6-naphthyridine-5-carboxamide were separated by chiral SFC using a Lux® i-Cellulose-5 column, 5 μm particle size, 25 cm×30 mm from Phenomenex (Mobile phase: 30% MeOH (supplemented with 0.1% DEA), 70% CO₂; Flow rate 100 mL/min) to give:

First Eluting Isomer: rel-2-((1R,2R,3aR,6aS)-1-(3,4-difluoro-2-methylphenyl)-4,4-difluorooctahydropentalen-2-yl)-4-oxo-1,4-dihydro-1,6-naphthyridine-5-carboxamide (Compound 5, 60 mg, 16%) as a white solid. ¹H NMR (400 MHz, DMSO-d₆) δ 11.69 (s, 1H), 8.43 (d, J=5.7 Hz, 1H), 7.48-7.31 (m, 2H), 7.27-7.03 (m, 3H), 6.12 (s, 1H), 3.85 (b dd, J=12.4, 7.9 Hz, 1H), 3.46 (td, J=12.1, 6.8 Hz, 1H), 3.27-3.18 (m, 1H), 3.05-2.89 (m, 1H), 2.40 (br dd, J=13.1, 6.5 Hz, 1H), 2.32 (d, J=1.7 Hz, 3H), 2.09-1.94 (m, 3H), 1.31-1.22 (m, 1H), 1.15-1.04 (m, 1H) ppm. ESI-MS m/z calc. 459.157, found 460.2 (M+1)⁺.

Second Eluting Isomer: rel-2-((1S,2S,3aS,6aR)-1-(3,4-difluoro-2-methylphenyl)-4,4-difluorooctahydropentalen-2-yl)-4-oxo-1,4-dihydro-1,6-naphthyridine-5-carboxamide (Compound 6, 62 mg, 16%) as a white solid. ¹H NMR (400 MHz, Methanol-d₄) δ 8.47 (d, J=5.9 Hz, 1H), 7.53 (d, J=5.9 Hz, 1H), 7.07-6.87 (m, 2H), 6.23 (s, 1H), 3.82 (dd, J=12.7, 8.1 Hz, 1H), 3.58-3.44 (m, 1H), 3.10-2.90 (m, 1H), 2.61 (dd, J=13.3, 6.7 Hz, 1H), 2.37 (d, J=2.4 Hz, 3H), 2.13-1.91 (m, 3H), 1.35-1.23 (m, 3H) ppm; exchangeable H not observed. ESI-MS m/z calc. 459.157, found 460.2 (M+1)⁺.

### Example 4

### rel-2-((1R,2R,4S)-2-(3,4-Difluoro-2-methoxyphenyl)-4-methyl-4-(trifluoromethyl)cyclopentyl)-4-oxo-1,4-dihydro-1,6-naphthyridine-5-carboxamide (7)

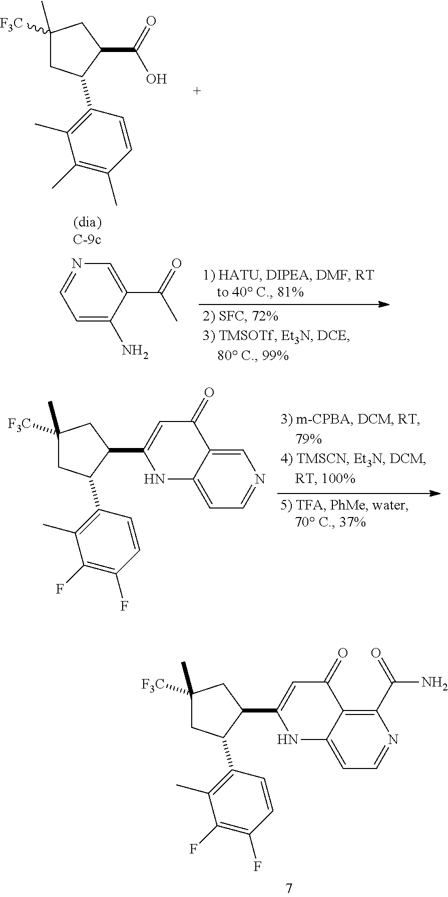

### Step 1

DIPEA (3.6260 g, 4.9 mL, 28.056 mmol) was added to a stirred solution of dia-(1R¹,2R¹,4S²)-2-(3,4-difluoro-2-methoxyphenyl)-4-methyl-4-(trifluoromethyl)cyclopentane-1-carboxylic acid (C-9c, 1.23 g, 2.764 mmol) and 1-(4-aminopyridin-3-yl)ethan-1-one hydrochloride (575 mg, 3.331 mmol) in DMF (10 mL) and the reaction mixture was stirred at ambient temperature for 30 min. HATU (1.58 g, 4.155 mmol) was added and the reaction was stirred at 40° C. for 1 h. The mixture was cooled to ambient temperature. Purification by reverse phase chromatography (C₁₈ column, 5 to 100% MeCN in water with 0.1% formic acid) gave dia-(1R¹,2R¹,4S²)—N-(3-acetylpyridin-4-yl)-2-(3,4-difluoro-2-methoxyphenyl)-4-methyl-4-(trifluoromethyl)cyclopentane-1-carboxamide (1.21 g, 81%) as a light yellow solid. ¹H NMR (400 MHz, Methanol-d₄) δ 9.06 (s, 1H), 8.57 (d, J=6.2 Hz, 1H), 8.54-8.49 (m, 1H), 7.18-7.10 (m, 1H), 7.01-6.90 (m, 1H), 3.93 (d, J=2.2 Hz, 1.5H), 3.91 (d, J=2.2 Hz, 1.5H), 3.80-3.63 (m, 1H), 3.39-3.33 (m, 0.5H), 3.27-3.21 (m, 0.5H), 2.64 (s, 1.5H), 2.63 (s, 1.5H), 2.56-2.47 (m, 1.5H), 2.38-2.30 (m, 0.5H), 2.11-1.92 (m, 1.5H), 1.84-1.75 (m, 0.5H), 1.43 (s, 3H) ppm; exchangeable H not observed. ESI-MS m/z calc. 456.147, found 457.2 (M+1)⁺.

### Step 2

The isomers of dia-(1R¹,2R¹,4S²)—N-(3-acetylpyridin-4-yl)-2-(3,4-difluoro-2-methoxyphenyl)-4-methyl-4-(trifluoromethyl)cyclopentane-1-carboxamide (1.21 g, 2.246 mmol) were separated by chiral SFC using a Lux® Cellulose-2 column, 5 μm particle size, 25 cm×30 mm from Phenomenex (Mobile phase: 3% EtOH, 97% CO₂; Flow rate 100 mL/min) to give:

First Eluting Isomer: rel-(1S,2S,4R)—N-(3-acetylpyridin-4-yl)-2-(3,4-difluoro-2-methoxyphenyl)-4-methyl-4-(trifluoromethyl)cyclopentane-1-carboxamide (160.1 mg, 15%) as an off-white solid. ¹H NMR (400 MHz, Chloroform-d) δ 11.57 (br s, 1H), 9.06 (br s, 1H), 8.59 (br s, 2H), 7.00 (t, J=6.4 Hz, 1H), 6.92-6.78 (m, 1H), 3.97-3.92 (m, 3H), 3.78 (td, J=11.7, 7.6 Hz, 1H), 3.22-3.07 (m, 1H), 2.64 (s, 3H), 2.53 (br dd, J=14.1, 8.4 Hz, 1H), 2.28 (br t, J=12.9 Hz, 1H), 2.09-1.92 (m, 2H), 1.43 (s, 3H) ppm. ESI-MS m/z calc. 456.147, found 457.2 (M+1)⁺.

Second Eluting Isomer: rel-(1R,2R,4S)—N-(3-acetylpyridin-4-yl)-2-(3,4-difluoro-2-methoxyphenyl)-4-methyl-4-(trifluoromethyl)cyclopentane-1-carboxamide (263.5 mg, 23%) as an off-white solid. ¹H NMR (400 MHz, Chloroform-d) δ 11.58 (br s, 1H), 9.05 (br s, 1H), 8.58 (br s, 2H), 7.00 (ddd, J=8.5, 5.9, 2.2 Hz, 1H), 6.89-6.78 (m, 1H), 3.95-3.87 (m, 3H), 3.78 (td, J=11.9, 7.3 Hz, 1H), 3.22-3.07 (m, 1H), 2.67-2.62 (m, 3H), 2.53 (dd, J=14.1, 8.4 Hz, 1H), 2.36-2.19 (m, 1H), 2.07-1.93 (m, 2H), 1.46-1.40 (m, 3H) ppm. ESI-MS m/z calc. 456.147, found 457.1 (M+1)⁺.

Third Eluting Isomer: rel-(1S,2S,4S)—N-(3-acetylpyridin-4-yl)-2-(3,4-difluoro-2-methoxyphenyl)-4-methyl-4-(trifluoromethyl)cyclopentane-1-carboxamide (179.9 mg, 17%) as a colorless sticky solid. ¹H NMR (400 MHz, Chloroform-d) δ 11.64 (br s, 1H), 9.06 (br s, 1H), 8.59 (br s, 2H), 6.93 (ddd, J=8.5, 6.0, 2.1 Hz, 1H), 6.81 (td, J=9.1, 7.3 Hz, 1H), 3.99 (d, J=2.7 Hz, 3H), 3.70 (td, J=11.5, 8.9 Hz, 1H), 3.22 (td, J=11.7, 6.9 Hz, 1H), 2.66 (s, 3H), 2.65 (br s, 1H), 2.62-2.50 (m, 2H), 2.01 (dd, J=13.3, 6.9 Hz, 1H), 1.41 (s, 3H) ppm. ESI-MS m/z calc. 456.147, found 457.2 (M+1)⁺.

Fourth Eluting Isomer: rel-(1R,2R,4R)—N-(3-acetylpyridin-4-yl)-2-(3,4-difluoro-2-methoxyphenyl)-4-methyl-4-(trifluoromethyl)cyclopentane-1-carboxamide (184.5 mg, 17%) as a colorless oil. ¹H NMR (400 MHz, Chloroform-d) δ 11.64 (br s, 1H), 9.06 (br s, 1H), 8.60 (br s, 2H), 6.96-6.89 (m, 1H), 6.86-6.76 (m, 1H), 3.99 (d, J=2.6 Hz, 3H), 3.77-3.65 (m, 1H), 3.22 (td, J=11.7, 7.0 Hz, 1H), 2.66 (s, 3H), 2.62-2.50 (m, 2H), 2.01 (dd, J=13.3, 6.9 Hz, 1H), 1.70 (m, 1H), 1.41 (s, 3H) ppm. ESI-MS m/z calc. 456.147, found 457.2 (M+1)⁺.

### Step 3

Et₃N (68.97 mg, 95 μL, 0.682 mmol) and TMSOTf (305 mg, 0.25 mL, 1.372 mmol) were successively added to a stirred solution of rel-(1R,2R,4S)—N-(3-acetylpyridin-4-yl)-2-(3,4-difluoro-2-methoxyphenyl)-4-methyl-4-(trifluoromethyl)cyclopentane-1-carboxamide (102 mg, 0.223 mmol) in DCE (2 mL) and the reaction mixture was stirred at 80° C. for 5 h. The mixture was cooled to ambient temperature. Additional amounts of Et₃N (29.04 mg, 40 μL, 0.287 mmol) and TMSOTf (122 mg, 0.1 mL, 0.549 mmol) were added and the reaction was stirred at 80° C. for 18 h. The mixture was cooled to ambient temperature, diluted with a 0.5 M aqueous NaOH solution (20 mL) and extracted with a 9:1 mixture of DCM:EtOH (3×20 mL) and 2-MeTHF (3×20 mL). The combined organic layers were dried (Na₂SO₄), filtered and concentrated in vacuo. Purification by reverse phase chromatography (C₁₈ column, 5 to 95% MeCN in water with 0.1% formic acid) gave rel-2-((1R,2R,4S)-2-(3,4-difluoro-2-methoxyphenyl)-4-methyl-4-(trifluoromethyl)cyclopentyl)-1,6-naphthyridin-4(1H)-one (102 mg, 99%) as a white solid. ¹H NMR (400 MHz, Methanol-d₄) δ 9.24 (s, 1H), 8.55 (d, J=6.0 Hz, 1H), 7.48 (d, J=6.1 Hz, 1H), 7.15-7.04 (m, 1H), 7.01-6.86 (m, 1H), 6.31 (s, 1H), 4.01-3.93 (m, 1H), 3.90 (d, J=2.3 Hz, 3H), 3.49 (td, J=12.0, 7.7 Hz, 1H), 2.77 (dd, J=14.0, 7.8 Hz, 1H), 2.31-2.21 (m, 1H), 2.17-2.07 (m, 1H), 1.91 (t, J=13.0 Hz, 1H), 1.49 (s, 3H) ppm; exchangeable H not observed. ESI-MS m/z calc. 438.137, found 439.1 (M+1)⁺.

### Step 4

m-CPBA (104 mg, 0.464 mmol) was added to a solution of rel-2-((1R,2R,4S)-2-(3,4-difluoro-2-methoxyphenyl)-4-methyl-4-(trifluoromethyl)cyclopentyl)-1,6-naphthyridin-4(1H)-one (101 mg, 0.218 mmol) in DCM (4 mL) and the reaction mixture was stirred at ambient temperature for 16 h. The reaction was concentrated in vacuo, diluted with EtOAc (50 mL) and washed with a saturated aqueous NaHCO₃ solution (3×50 mL) and brine (50 mL), dried (Na₂SO₄), filtered and concentrated in vacuo to give rel-2-((1R,2R,4S)-2-(3,4-difluoro-2-methoxyphenyl)-4-methyl-4-(trifluoromethyl)cyclopentyl)-4-oxo-1,4-dihydro-1,6-naphthyridine 6-oxide (90 mg, 79%) as an orange solid, which was used without further purification in the next step. ¹H NMR (400 MHz, Methanol-d₄) δ 8.86 (d, J=2.0 Hz, 1H), 8.34 (dd, J=7.3, 1.9 Hz, 1H), 7.64 (d, J=7.2 Hz, 1H), 7.15-7.04 (m, 1H), 6.98-6.86 (m, 1H), 6.32 (s, 1H), 4.01-3.94 (m, 1H), 3.92 (d, J=2.2 Hz, 3H), 3.55-3.44 (m, 1H), 2.76 (dd, J=14.1, 7.6 Hz, 1H), 2.31-2.20 (m, 1H), 2.16-2.07 (m, 1H), 1.90 (br t, J=12.8 Hz, 1H), 1.48 (s, 3H) ppm; exchangeable H not observed. ESI-MS m/z calc. 454.132, found 455.1 (M+1)⁺.

### Step 5

In a sealed tube, TMSCN (39.65 mg, 50 μL, 0.400 mmol) and Et₃N (54.450 mg, 75 μL, 0.538 mmol) were added to a stirred solution of rel-2-((1R,2R,4S)-2-(3,4-difluoro-2-methoxyphenyl)-4-methyl-4-(trifluoromethyl)cyclopentyl)-4-oxo-1,4-dihydro-1,6-naphthyridine 6-oxide (90 mg, 0.172 mmol) in DCM (1 mL) under a nitrogen atmosphere. The tube was sealed and the reaction mixture was stirred at ambient temperature for 18 h. Additional TMSCN (19.825 mg, 25 μL, 0.200 mmol) and Et₃N (27.588 mg, 38 μL, 0.273 mmol) were added and the reaction was stirred at ambient temperature for 2 h. The mixture was diluted with a saturated NaHCO₃ solution (20 mL) and extracted with EtOAc (3×20 mL). The combined organic layers were dried (Na₂SO₄), filtered and concentrated in vacuo to give rel-2-((1R,2R,4S)-2-(3,4-difluoro-2-methoxyphenyl)-4-methyl-4-(trifluoromethyl)cyclopentyl)-4-oxo-1,4-dihydro-1,6-naphthyridine-5-carbonitrile (88 mg, 100%) as a brown solid, which was used without further purification in the next step. ¹H NMR (400 MHz, Methanol-d₄) δ 8.62 (d, J=5.9 Hz, 1H), 7.66 (d, J=5.9 Hz, 1H), 7.13-7.07 (m, 1H), 6.97-6.88 (m, 1H), 6.35 (s, 1H), 4.01-3.94 (m, 1H), 3.92 (d, J=2.2 Hz, 3H), 3.54-3.43 (m, 1H), 2.80-2.70 (m, 1H), 2.29-2.20 (m, 1H), 2.11 (dd, J=13.5, 7.4 Hz, 1H), 1.90 (br t, J=12.8 Hz, 1H), 1.48 (s, 3H) ppm; exchangeable H not observed. ESI-MS m/z calc. 463.132, found 464.1 (M+1)⁺.

### Step 6

A solution of rel-2-((1R,2R,4S)-2-(3,4-difluoro-2-methoxyphenyl)-4-methyl-4-(trifluoromethyl)cyclopentyl)-4-oxo-1,4-dihydro-1,6-naphthyridine-5-carbonitrile (88 mg, 0.171 mmol) and TFA (1.924 g, 1.3 mL, 16.874 mmol) in a mixture of toluene (2 mL) and water (40 L) was stirred at 70° C. for 4 h. Additional TFA (296 mg, 0.2 mL, 2.596 mmol) was added and the mixture was stirred at 70° C. for a further 15 h. The reaction was cooled to ambient temperature and concentrated in vacuo. The residue was solubilised in EtOAc (10 mL), washed with a saturated aqueous NaHCO₃ solution (3×10 mL) and brine (10 mL). The organic layer was dried (Na₂SO₄), filtered and concentrated in vacuo. Purification by reverse phase chromatography (C₁₈ column, 2.5% to 70% MeCN in water with 0.1% formic acid) followed by flash chromatography (SiO₂, 0 to 100% EtOAc in heptane) gave rel-2-((1R,2R,4S)-2-(3,4-difluoro-2-methoxyphenyl)-4-methyl-4-(trifluoromethyl)cyclopentyl)-4-oxo-1,4-dihydro-1,6-naphthyridine-5-carboxamide (7, 31 mg, 37%) as a white solid. ¹H NMR (400 MHz, DMSO-d₆) δ 11.62 (s, 1H), 8.43 (d, J=5.9 Hz, 1H), 7.42-7.36 (m, 2H), 7.23-7.06 (m, 3H), 6.14 (s, 1H), 3.94 (td, J=11.8, 7.6 Hz, 1H), 3.86 (d, J=1.5 Hz, 3H), 3.43 (td, J=12.0, 7.7 Hz, 1H), 2.64 (dd, J=13.8, 7.7 Hz, 1H), 2.12-1.98 (m, 2H), 1.85 (br t, J=12.9 Hz, 1H), 1.43 (s, 3H) ppm. ESI-MS m/z calc. 481.142, found 482.2 (M+1)⁺.

### Example 5

### rel-2-((1S,2S)-2-(3,4-Difluoro-2-methoxyphenyl)-5,5-difluorocyclohexyl)-4-oxo-1,4-dihydro-1,6-naphthyridine-5-carboxamide (8) and rel-2-((1R,2R)-2-(3,4-difluoro-2-methoxyphenyl)-5,5-difluorocyclohexyl)-4-oxo-1,4-dihydro-1,6-naphthyridine-5-carboxamide (9)

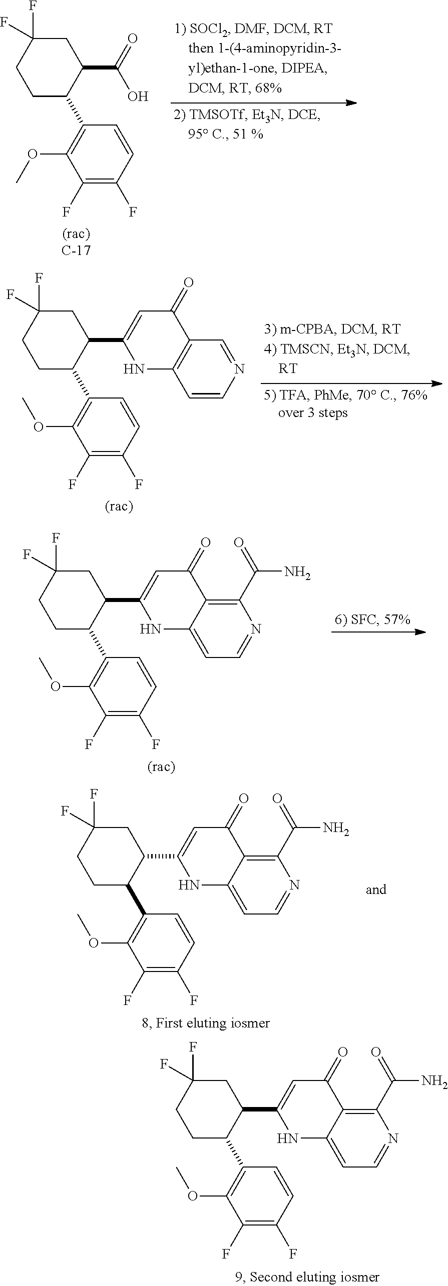

### Step 1

SOCl₂ (500 μL, 6.855 mmol) was added to a stirred solution of rac-(1R,2R)-2-(3,4-difluoro-2-methoxyphenyl)-5,5-difluorocyclohexane-1-carboxylic acid (C-17, 217 mg, 0.709 mmol) and DMF (10 μL, 0.129 mmol) in DCM (4 mL) and the reaction mixture was stirred at ambient temperature for 20 min. The mixture was concentrated in vacuo, coevaporated with toluene (2×3 mL) and the residue was redissolved in DCM (4 mL). 1-(4-Aminopyridin-3-yl)ethan-1-one (97 mg, 0.712 mmol) and DIPEA (500 μL, 2.870 mmol) were successively added and the mixture was stirred at ambient temperature for 20 min. The reaction was concentrated in vacuo. Purification by flash chromatography (12 g SiO₂, 0 to 70% EtOAc in hexanes) gave rac-(1R,2R)—N-(3-acetylpyridin-4-yl)-2-(3,4-difluoro-2-methoxyphenyl)-5,5-difluorocyclohexane-1-carboxamide (205 mg, 68%). ¹H NMR (400 MHz, Chloroform-d) δ 11.60 (s, 1H), 9.03 (s, 1H), 8.53 (d, J=5.9 Hz, 1H), 8.46 (d, J=5.8 Hz, 1H), 6.95-6.89 (m, 1H), 6.77 (q, J=8.6 Hz, 1H), 3.97 (d, J=2.4 Hz, 3H), 3.37-3.23 (m, 1H), 3.19-2.93 (m, 1H), 2.67 (s, 3H), 2.44-2.35 (m, 1H), 2.35-2.14 (m, 2H), 2.03-1.85 (m, 3H) ppm. ESI-MS m/z calc. 486.158, found 487.4 (M+1)⁺.

### Step 2

Et₃N (200 μL, 1.435 mmol) and TMSOTf (550 μL, 3.044 mmol) were successively added to a solution of rac-(1R,2R)—N-(3-acetylpyridin-4-yl)-2-(3,4-difluoro-2-methoxyphenyl)-5,5-difluorocyclohexane-1-carboxamide (205 mg, 0.483 mmol) in DCE (4 mL) and the reaction mixture was heated at 95° C. for 2.5 h. The mixture was cooled to ambient temperature and partitioned between DCM and a saturated aqueous NaHCO₃ solution. The organic layer was separated, dried (MgSO₄), filtered and concentrated in vacuo. Purification by flash chromatography (SiO₂, 0 to 10% MeOH in DCM) gave rac-2-((1R,2R)-2-(3,4-difluoro-2-methoxyphenyl)-5,5-difluorocyclohexyl)-1,6-naphthyridin-4(1H)-one (100 mg, 51%). ¹H NMR (400 MHz, DMSO-d₆) δ 11.64 (s, 1H), 9.03 (s, 1H), 8.53 (d, J=5.8 Hz, 1H), 7.36 (d, J=5.9 Hz, 1H), 7.14 (s, 1H), 7.08-6.96 (m, 1H), 6.12 (d, J=1.4 Hz, 1H), 3.81 (s, 3H), 3.65-3.52 (m, 1H), 3.30-3.21 (m, 1H), 2.48-2.36 (m, 2H), 2.27-2.01 (m, 2H), 1.94-1.87 (m, 1H), 1.72 (br s, 1H) ppm. ESI-MS m/z calc. 406.130, found 407.4 (M+1)⁺.

### Step 3 and 4

m-CPBA (141 mg, 0.629 mmol) was added to a suspension of rac-2-((1R,2R)-2-(3,4-difluoro-2-methoxyphenyl)-5,5-difluorocyclohexyl)-1,6-naphthyridin-4(1H)-one (99 mg, 0.244 mmol) in DCM (5 mL) and the mixture was stirred at ambient temperature for 1 h. Et₃N (1.0 mL, 7.175 mmol) and TMSCN (500 μL, 3.750 mmol) were successively added and the resulting solution was stirred at ambient temperature for 1 h. Additional TMSCN (500 μL, 3.750 mmol) was added and the mixture was stirred for a further 2 h. The mixture was concentrated in vacuo. Purification by flash chromatography (12 g SiO₂, 0 to 6% MeOH in DCM) gave rac-2-((1R,2R)-2-(3,4-difluoro-2-methoxyphenyl)-5,5-difluorocyclohexyl)-4-oxo-1,4-dihydro-1,6-naphthyridine-5-carbonitrile, which was used without further purification in the next step. ESI-MS m/z calc. 431.126, found 432.3 (M+1)⁺.

### Step 5

TFA (3 mL, 38.94 mmol) was added to the residue of rac-2-((1R,2R)-2-(3,4-difluoro-2-methoxyphenyl)-5,5-difluorocyclohexyl)-4-oxo-1,4-dihydro-1,6-naphthyridine-5-carbonitrile in toluene (3 mL) and the reaction mixture was stirred at 70° C. for 16 h. The mixture was concentrated in vacuo and partitioned between EtOAc and a saturated aqueous NaHCO₃ solution. The organic phase was separated, dried (Na₂SO₄), filtered and concentrated in vacuo. Purification by reverse phase HPLC (C₁₈ column, 10 to 99% MeCN in water) gave rac-2-((1R,2R)-2-(3,4-difluoro-2-methoxyphenyl)-5,5-difluorocyclohexyl)-4-oxo-1,4-dihydro-1,6-naphthyridine-5-carboxamide (83 mg, 76% over 3 steps). ESI-MS m/z calc. 449.136, found 450.5 (M+1)⁺.

### Step 6

The enantiomers of rac-2-((1R,2R)-2-(3,4-difluoro-2-methoxyphenyl)-5,5-difluorocyclohexyl)-4-oxo-1,4-dihydro-1,6-naphthyridine-5-carboxamide (55 mg, 0.122 mmol) were separated by chiral SFC using a Chiralpak® IC column, 5 μm particle size, 25 cm×22.1 mm from Daicel Corporation (Mobile phase: 30% MeOH (supplemented with 20 mM ammonia), 70% CO₂; Flow rate 60 mL/min) to give:

First Eluting Isomer: rel-2-((1S,2S)-2-(3,4-difluoro-2-methoxyphenyl)-5,5-difluorocyclohexyl)-4-oxo-1,4-dihydro-1,6-naphthyridine-5-carboxamide (8, 25.0 mg, 45%). ¹H NMR (400 MHz, DMSO-d₆) δ 11.60 (s, 1H), 8.41 (d, J=5.8 Hz, 1H), 7.40 (s, 1H), 7.35 (d, J=5.8 Hz, 1H), 7.25-7.10 (m, 2H), 7.09-7.01 (m, 1H), 6.08 (s, 1H), 3.83 (s, 3H), 3.60 (br s, 1H), 2.45-2.24 (m, 2H), 2.28-1.99 (m, 3H), 1.94-1.86 (m, 1H), 1.69 (br s, 1H) ppm. ESI-MS m/z calc. 449.136, found 450.3 (M+1)⁺.

Second Eluting Isomer: rel-2-((1R,2R)-2-(3,4-difluoro-2-methoxyphenyl)-5,5-difluorocyclohexyl)-4-oxo-1,4-dihydro-1,6-naphthyridine-5-carboxamide (9, 6.5 mg, 12%). ¹H NMR (400 MHz, DMSO-d₆) δ 11.61 (s, 1H), 8.41 (d, J=5.8 Hz, 1H), 7.40 (s, 1H), 7.35 (d, J=5.8 Hz, 1H), 7.27-7.10 (m, 2H), 7.10-6.97 (m, 1H), 6.08 (s, 1H), 3.83 (s, 3H), 3.69-3.53 (m, 1H), 3.30-3.22 (m, 1H), 2.45-2.33 (m, 2H), 2.26-2.04 (m, 2H), 1.90 (d, J=12.8 Hz, 1H), 1.69 (br s, 1H) ppm. ESI-MS m z calc. 449.136, found 450.4 (M+1)⁺.

### Example 6

### rel-2-((1S,2S,4R)-2-(4-Fluoro-2-methoxy-3-methylphenyl)-4-(trifluoromethyl)cyclopentyl)-4-oxo-1,4-dihydro-1,6-naphthyridine-5-carboxamide (10) and rel-2-((1R,2R,4S)-2-(4-fluoro-2-methoxy-3-methylphenyl)-4-(trifluoromethyl)cyclopentyl)-4-oxo-1,4-dihydro-1,6-naphthyridine-5-carboxamide (11)

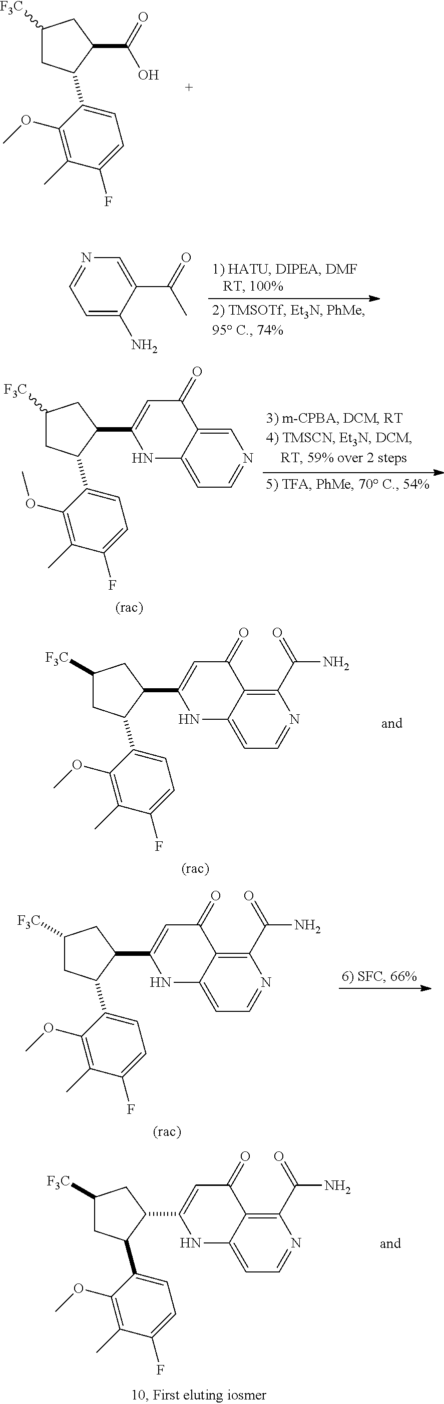

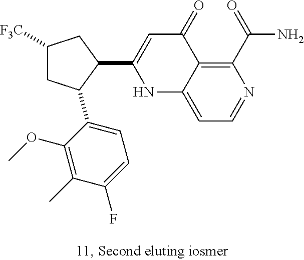 [SMILES: COc1c([C@@H]2C[C@H](C(F)(F)F)C[C@H]2c2cc(=O)c3c(C(N)=O)nccc3[nH]2)ccc(F)c1C]

### Step 1

HATU (776.4 mg, 2.042 mmol) was added to a stirred solution of dia-(1R¹,2R¹,4S²)-2-(4-fluoro-2-methoxy-3-methylphenyl)-4-(trifluoromethyl)cyclopentane-1-carboxylic acid (C-3a, 590 mg, 1.842 mmol), 1-(4-aminopyridin-3-yl)ethan-1-one (278.2 mg, 2.043 mmol) and DIPEA (1 mL, 5.741 mmol) in DCM (3 mL) and the reaction mixture was stirred at ambient temperature for 20 h. The mixture was diluted with EtOAc, washed with water (2×40 mL) and brine (40 mL), dried (MgSO₄), filtered, and concentrated in vacuo. Purification by flash chromatography (SiO₂, 0% to 100% EtOAc in DCM) gave dia-(1R¹,2R¹,4S²)—N-(3-acetylpyridin-4-yl)-2-(4-fluoro-2-methoxy-3-methylphenyl)-4-(trifluoromethyl)cyclopentane-1-carboxamide (1.1 g, 100%) as a yellow foam. ESI-MS m/z calc. 438.157, found 439.4 (M+1)⁺.

### Step 2

Et₃N (770 μL, 5.524 mmol) and TMSOTf (2 mL, 11.07 mmol) were successively added to a stirred solution of dia-(1R¹,2R¹,4S²)—N-(3-acetylpyridin-4-yl)-2-(4-fluoro-2-methoxy-3-methylphenyl)-4-(trifluoromethyl)cyclopentane-1-carboxamide (808 mg, 1.751 mmol) in toluene (9 mL) and the reaction mixture was heated at 95° C. for 4 h under nitrogen. The reaction was cooled to ambient temperature, washed with 2 N aqueous K₂CO₃ and the aqueous phase was extracted with DCM (x 2). The combined organic phases were dried (MgSO₄), filtered and concentrated in vacuo. Purification by flash chromatography (SiO₂, 0 to 50% of MeOH in DCM) gave dia-2-((1R¹,2R¹,4S²)-2-(4-fluoro-2-methoxy-3-methylphenyl)-4-(trifluoromethyl)cyclopentyl)-1,6-naphthyridin-4(1H)-one (576 mg, 74%) as a tan solid. ESI-MS m/z calc. 420.146, found 421.5 (M+1)⁺.

### Step 3

m-CPBA (446.6 mg, 1.993 mmol) was added to a suspension of dia-2-((1R¹,2R¹,4S²)-2-(4-fluoro-2-methoxy-3-methylphenyl)-4-(trifluoromethyl)cyclopentyl)-1,6-naphthyridin-4(1H)-one (576 mg, 1.302 mmol) in DCM (9 mL) and the reaction mixture was stirred at ambient temperature for 4 h to give a solution of dia-2-((1R¹,2R¹,4S²)-2-(4-fluoro-2-methoxy-3-methylphenyl)-4-(trifluoromethyl)cyclopentyl)-4-oxo-1,4-dihydro-1,6-naphthyridine 6-oxide, which was used without further purification in the next step. ESI-MS m/z calc. 436.141, found 437.4 (M+1)⁺.

### Step 4

Et₃N (4 mL, 28.7 mmol) and TMSCN (1.6 mL, 12.79 mmol) were successively added to the DCM solution of dia-2-((1R¹,2R¹,4S²)-2-(4-fluoro-2-methoxy-3-methylphenyl)-4-(trifluoromethyl)cyclopentyl)-4-oxo-1,4-dihydro-1,6-naphthyridine 6-oxide and the reaction mixture was stirred at ambient temperature for 1 h. The reaction was partitioned between a saturated aqueous NaHCO₃ solution (75 mL) and EtOAc (30 mL). The aqueous layer was separated and extracted with EtOAc (2×30 mL). The combined organic layers were washed with water (30 mL) and brine (30 mL), dried (MgSO₄), filtered and concentrated in vacuo. Purification by flash chromatography (SiO₂, 0 to 10% of MeOH in DCM) gave dia-2-((1R¹,2R¹,4S²)-2-(4-fluoro-2-methoxy-3-methylphenyl)-4-(trifluoromethyl)cyclopentyl)-4-oxo-1,4-dihydro-1,6-naphthyridine-5-carbonitrile (344 mg, 59% over 2 steps) as an off-white solid. ESI-MS m/z calc. 445.141, found 446.4 (M+1)⁺.

### Step 5

TFA (3 mL, 38.94 mmol) was added to a suspension of dia-2-((1R¹,2R¹,4S²)-2-(4-fluoro-2-methoxy-3-methylphenyl)-4-(trifluoromethyl)cyclopentyl)-4-oxo-1,4-dihydro-1,6-naphthyridine-5-carbonitrile (344 mg, 0.772 mmol) in toluene (5 mL) and the reaction mixture was stirred at 70° C. for 3 h. The reaction was cooled to ambient temperature and concentrated in vacuo. Purification by reverse phase HPLC (C₁₈ column, 0 to 40% MeCN with 5 mM HCl) gave:

First Eluting Isomers: rac-2-((1R,2R,4R)-2-(4-fluoro-2-methoxy-3-methylphenyl)-4-(trifluoromethyl)cyclopentyl)-4-oxo-1,4-dihydro-1,6-naphthyridine-5-carboxamide (130 mg, 36%) as a white solid. ¹H NMR (400 MHz, DMSO-d₆) δ 11.60 (s, 1H), 8.43 (d, J=5.8 Hz, 1H), 7.44 (d, J=5.8 Hz, 1H), 7.38 (br s, 1H), 7.36-7.30 (m, 1H), 7.20 (br s, 1H), 6.96 (t, J=8.8 Hz, 1H), 5.95 (s, 1H), 3.85-3.72 (m, 1H), 3.60 (s, 3H), 3.43-3.32 (m, 2H), 2.56-2.51 (m, 1H), 2.38-2.26 (m, 1H), 2.08 (d, J=1.6 Hz, 3H), 2.11-2.01 (m, 1H), 1.98-1.88 (m, 1H) ppm. ESI-MS m/z calc. 463.152, found 464.5 (M+1)⁺.

Second Eluting Isomers: rac-2-((1R,2R,4S)-2-(4-fluoro-2-methoxy-3-methylphenyl)-4-(trifluoromethyl)cyclopentyl)-4-oxo-1,4-dihydro-1,6-naphthyridine-5-carboxamide (64 mg, 18%) as a white solid. ¹H NMR (400 MHz, DMSO-d₆) δ 11.63 (s, 1H), 8.43 (d, J=5.8 Hz, 1H), 7.42-7.36 (m, 2H), 7.28 (dd, J=8.6, 6.7 Hz, 1H), 7.19 (br s, 1H), 6.94 (t, J=8.8 Hz, 1H), 6.05 (d, J=1.5 Hz, 1H), 3.88-3.75 (m, 1H), 3.62 (s, 3H), 3.46-3.33 (m, 1H), 3.32-3.25 (m, 1H), 2.48-2.39 (m, 2H), 2.31-2.18 (m, 1H), 2.08 (d, J=1.8 Hz, 3H), 1.76-1.63 (m, 1H) ppm. ESI-MS m/z calc. 463.152, found 464.4 (M+1)⁺.

### Step 6

The enantiomers of rac-2-((1R,2R,4S)-2-(4-fluoro-2-methoxy-3-methylphenyl)-4-(trifluoromethyl)cyclopentyl)-4-oxo-1,4-dihydro-1,6-naphthyridine-5-carboxamide (60 mg, 0.130 mmol) were separated by chiral SFC using a Lux® Cellulose-4 column, 5 μm particle size, 25 cm×21.2 mm from Phenomenex (Mobile phase: 42% MeOH (supplemented with 20 mM ammonia), 58% CO₂; Flow rate 65 mL/min) to give:

First Eluting Isomer: rel-2-((1S,2S,4R)-2-(4-fluoro-2-methoxy-3-methylphenyl)-4-(trifluoromethyl)cyclopentyl)-4-oxo-1,4-dihydro-1,6-naphthyridine-5-carboxamide (10, 20 mg, 33%) as an off-white solid. ¹H NMR (400 MHz, DMSO-d₆) δ 11.62 (br s, 1H), 8.42 (d, J=5.7 Hz, 1H), 7.42-7.34 (m, 2H), 7.28 (dd, J=8.6, 6.7 Hz, 1H), 7.19 (s, 1H), 6.94 (t, J=8.8 Hz, 1H), 6.05 (s, 1H), 3.88-3.76 (m, 1H), 3.61 (s, 3H), 3.43-3.23 (m, 2H), 2.48-2.37 (m, 2H), 2.31-2.18 (m, 1H), 2.08 (d, J=1.6 Hz, 3H), 1.75-1.62 (m, 1H) ppm. ESI-MS m/z calc. 463.152, found 464.5 (M+1)⁺.

Second Eluting Isomer: rel-2-((1R,2R,4S)-2-(4-fluoro-2-methoxy-3-methylphenyl)-4-(trifluoromethyl)cyclopentyl)-4-oxo-1,4-dihydro-1,6-naphthyridine-5-carboxamide (11, 20 mg, 33%) as an off-white solid. ¹H NMR (400 MHz, DMSO-d₆) δ 11.63 (br s, 1H), 8.42 (d, J=5.7 Hz, 1H), 7.42-7.36 (m, 2H), 7.32-7.24 (m, 1H), 7.19 (br s, 1H), 6.94 (t, J=8.8 Hz, 1H), 6.05 (s, 1H), 3.88-3.76 (m, 1H), 3.61 (s, 3H), 3.45-3.23 (m, 2H), 2.49-2.39 (m, 2H), 2.31-2.18 (m, 1H), 2.08 (d, J=1.6 Hz, 3H), 1.75-1.62 (m, 1H) ppm. ESI-MS m/z calc. 463.152, found 464.6 (M+1)⁺.

### Example 7

dia-2-((1R¹,2R¹,4R²)-4-(tert-Butyl)-2-(3,4-difluoro-2-methoxyphenyl)cyclohexyl)-4-oxo-1,4-dihydro-1,6-naphthyridine-5-carboxamide (12)

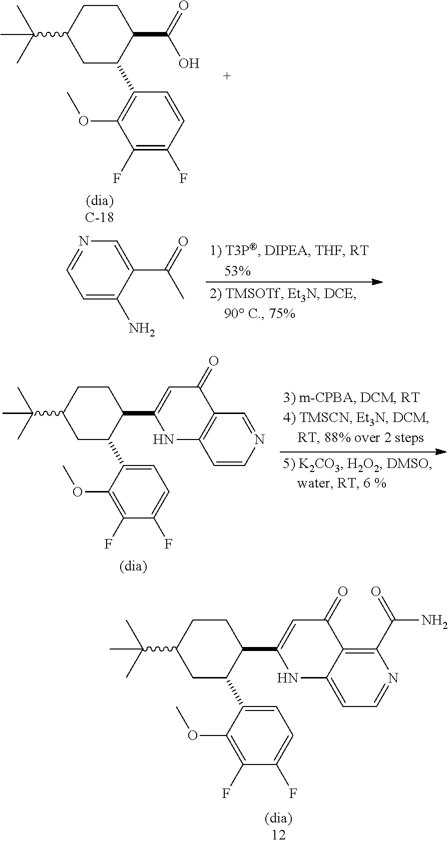

### Step 1

T3P® (2.339 mL, 50 wt % solution in EtOAc, 3.676 mmol) was added to a stirred solution of dia-(1R¹,2R¹,4R²)-4-(tert-butyl)-2-(3,4-difluoro-2-methoxyphenyl)cyclohexane-1-carboxylic acid (C-18, 600 mg, 1.838 mmol), 1-(4-aminopyridin-3-yl)ethan-1-one (500.5 mg, 3.676 mmol) and DIPEA (475.1 mg, 640.3 μL, 3.676 mmol) in THF (10 mL) and the reaction mixture was stirred at ambient temperature for 20 h. The reaction was concentrated in vacuo. Purification by high pressure reverse phase chromatography (SunFire C₁₈ column, 1 to 100% MeCN in water with 5 mM HCl) gave dia-(1R¹,2R¹,4R²)—N-(3-acetylpyridin-4-yl)-4-(tert-butyl)-2-(3,4-difluoro-2-methoxyphenyl)cyclohexane-1-carboxamide (432 mg, 53%) as a light yellow oil. ESI-MS m/z calc. 444.222, found 445.7 (M+1)⁺.

### Step 2

Under a nitrogen atmosphere, Et₃N (1 mL, 7.175 mmol) and TMSOTf (2.5 mL, 13.84 mmol) were successively added to a solution of dia-(1R¹,2R¹,4R²)—N-(3-acetylpyridin-4-yl)-4-(tert-butyl)-2-(3,4-difluoro-2-methoxyphenyl)cyclohexane-1-carboxamide (900 mg, 2.025 mmol) in DCE (10.80 mL) and the reaction mixture was heated at 90° C. for 8 h. The reaction was cooled to ambient temperature and washed with a 2 N aqueous K₂CO₃ solution (10 mL). The aqueous phase was extracted with DCM (2×20 mL). The combined organic phases were dried (Na₂SO₄), filtered and concentrated in vacuo. Purification by high pressure reverse phase chromatography (SunFire C₁₈ column, 1 to 100% MeCN in water with 5 mM HCl) gave dia-2-((1R¹,2R¹,4R²)-4-(tert-butyl)-2-(3,4-difluoro-2-methoxyphenyl)cyclohexyl)-1,6-naphthyridin-4(1H)-one (800 mg, 75%) as a white solid. ESI-MS m/z calc. 426.212, found 427.5 (M+1)⁺.

### Step 3 and 4

m-CPBA (31.53 mg, 0.141 mmol) was added to a suspension of dia-2-((1R¹,2R¹,4R²)-4-(tert-butyl)-2-(3,4-difluoro-2-methoxyphenyl)cyclohexyl)-1,6-naphthyridin-4(1H)-one (30 mg, 0.070 mmol) in DCM (1.263 mL) and the reaction mixture was stirred at ambient temperature for 4 h. Et₃N (163.3 mg, 224.9 μL, 1.614 mmol) and TMSCN (93.02 mg, 125 μL, 0.938 mmol) were successively added and the reaction was stirred at ambient temperature for 2.5 h. The reaction was concentrated in vacuo. Purification by high pressure reverse phase chromatography (Luna C₁₈ column, 1 to 100% MeCN in water with 5 mM HCl) gave dia-2-((1R¹,2R¹,4R²)-4-(tert-butyl)-2-(3,4-difluoro-2-methoxyphenyl)cyclohexyl)-4-oxo-1,4-dihydro-1,6-naphthyridine-5-carbonitrile (27.8 mg, 88% over 2 steps) as a white solid. ESI-MS m/z calc. 451.207, found 452.5 (M+1)⁺.

### Step 5

A mixture of dia-2-((1R¹,2R¹,4R²)-4-(tert-butyl)-2-(3,4-difluoro-2-methoxyphenyl)cyclohexyl)-4-oxo-1,4-dihydro-1,6-naphthyridine-5-carbonitrile (30 mg, 0.066 mmol), K₂CO₃ (30 mg, 0.217 mmol) and H₂O₂(100 μL, 35 wt % solution in water, 1.029 mmol) in DMSO (750 μL) was stirred at ambient temperature for 24 h. Purification by reverse phase chromatography (Luna Cis column, 1 to 100% MeCN in water) gave dia-2-((1R¹,2R¹,4R²)-4-(tert-butyl)-2-(3,4-difluoro-2-methoxyphenyl)cyclohexyl)-4-oxo-1,4-dihydro-1,6-naphthyridine-5-carboxamide (12, 2 mg, 6%) as a colourless solid. ¹H NMR (400 MHz, Methanol-d₄) δ 8.74 (d, J=6.0 Hz, 1H), 7.88-7.83 (m, 1H), 7.07 (dd, J=7.6, 5.0 Hz, 1H), 6.87 (q, J=8.8 Hz, 1H), 6.74 (d, J=12.9 Hz, 1H), 3.97-3.89 (m, 3H), 2.23 (d, J=12.9 Hz, 1H), 2.15-1.93 (m, 4H), 1.90-1.70 (m, 2H), 1.51-1.34 (m, 2H), 1.04 (s, 4H), 0.95 (s, 5H) ppm; exchangeable H not observed. ESI-MS m/z calc. 469.218, found 470.5 (M+1)⁺.

### Example 8

### rel-2-((4R,6S,7S)-7-(3,4-Difluoro-2-methoxyphenyl)-1,1-difluorospiro[3.4]octan-6-yl)-4-oxo-1,4-dihydro-1,6-naphthyridine-5-carboxamide (13) and rel-2-((4S,6R,7R)-7-(3,4-difluoro-2-methoxyphenyl)-1,1-difluorospiro[3.4]octan-6-yl)-4-oxo-1,4-dihydro-1,6-naphthyridine-5-carboxamide (14)

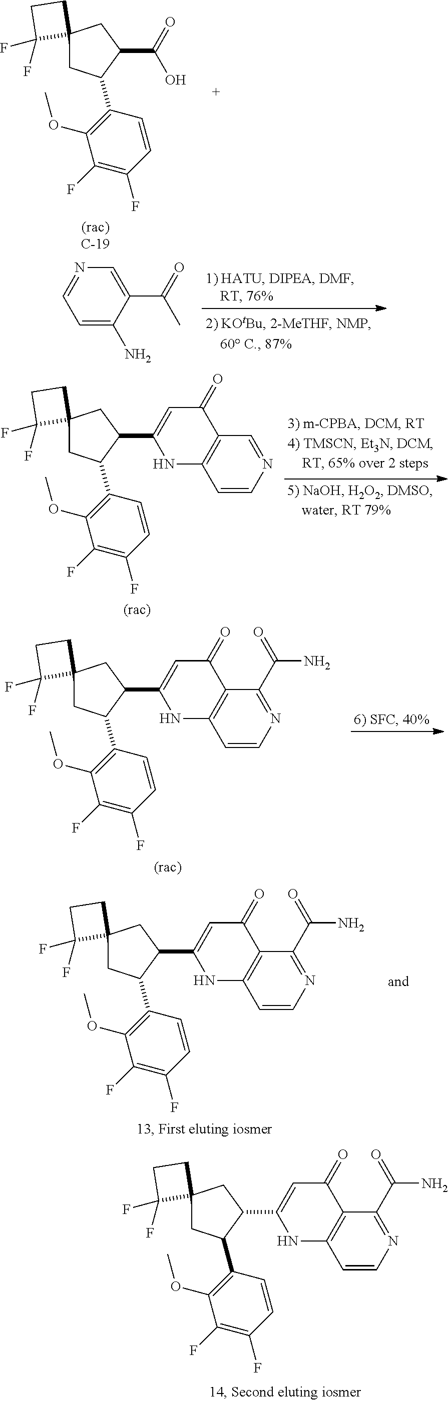

### Step 1

HATU (230 mg, 0.605 mmol) was added to a solution of rac-(4S,6R,7R)-7-(3,4-difluoro-2-methoxyphenyl)-1,1-difluorospiro[3.4]octane-6-carboxylic acid (C-19, 183 mg, 0.452 mmol), 1-(4-aminopyridin-3-yl)ethan-1-one (76 mg, 0.558 mmol) and DIPEA (178.32 mg, 0.24 mL, 1.380 mmol) in DMF (4 mL) and the reaction mixture was stirred at ambient temperature for 2 h. Purification by reverse phase chromatography (5 to 80% MeCN in water with 0.1% formic acid) gave rac-(4S,6R,7R)—N-(3-acetylpyridin-4-yl)-7-(3,4-difluoro-2-methoxyphenyl)-1,1-difluorospiro[3.4]octane-6-carboxamide (167 mg, 76%) as a yellow solid. ¹H NMR (400 MHz, Chloroform-d) δ 11.98 (s, 1H), 9.26 (s, 1H), 9.15 (d, J=6.8 Hz, 1H), 8.58 (d, J=6.8 Hz, 1H), 7.13-7.07 (m, 1H), 6.97-6.89 (m, 1H), 3.90 (d, J=2.3 Hz, 3H), 3.85-3.76 (m, 1H), 3.39 (td, J=11.0, 7.0 Hz, 1H), 2.77-2.62 (m, 4H), 2.56-2.38 (m, 2H), 2.32-2.16 (m, 1H), 1.24-1.12 (m, 2H), 0.99-0.89 (m, 2H) ppm. ESI-MS m/z calc. 450.157, found 451.2 (M+1)⁺.

### Step 2

KO^{t}Bu (78 mg, 0.695 mmol) was added to a solution of rac-(4S,6R,7R)—N-(3-acetylpyridin-4-yl)-7-(3,4-difluoro-2-methoxyphenyl)-1,1-difluorospiro[3.4]octane-6-carboxamide (135 mg, 0.278 mmol) in a mixture of 2-MeTHF (3 mL) and NMP (0.15 mL) and the reaction mixture was stirred at 60° C. for 16 h. The reaction was cooled to ambient temperature and partitioned between a saturated aqueous NH₄Cl solution (10 mL) and EtOAc (20 mL). The aqueous layer was separated and extracted with EtOAc (2×20 mL). The combined organic layers were washed with water (10 mL) and brine (10 mL), dried (Na₂SO₄), filtered and concentrated in vacuo. Purification by reverse phase chromatography (C₁₈ column, 5 to 50% MeCN in water with 0.1% formic acid) gave rac-2-((4S,6R,7R)-7-(3,4-difluoro-2-methoxyphenyl)-1,1-difluorospiro[3.4]octan-6-yl)-1,6-naphthyridin-4(1H)-one (108.5 mg, 87%) as a tan solid. ¹H NMR (400 MHz, DMSO-d₆) δ 11.65 (s, 1H), 9.08 (br s, 1H), 8.55 (br s, 1H), 7.44 (d, J=5.6 Hz, 1H), 7.36-7.29 (m, 1H), 7.19-7.08 (m, 1H), 6.08 (s, 1H), 4.04 (td, J=12.0, 6.4 Hz, 1H), 3.78 (d, J=1.3 Hz, 3H), 3.45-3.35 (m, 1H), 2.73-2.57 (m, 1H), 2.45-2.18 (m, 2H), 2.14-1.98 (m, 1H, overlapped with MeCN), 1.26-1.03 (m, 4H) ppm. ESI-MS m/z calc. 432.146, found 433.2 (M+1)⁺.

### Step 3 and 4

m-CPBA (116 mg, 0.518 mmol) was added to a solution of rac-2-((4S,6R,7R)-7-(3,4-difluoro-2-methoxyphenyl)-1,1-difluorospiro[3.4]octan-6-yl)-1,6-naphthyridin-4(1H)-one (100 mg, 0.223 mmol) in DCM (6 mL) and the reaction mixture was stirred at ambient temperature for 3.5 h. Et₃N (333.96 mg, 0.46 mL, 3.300 mmol) and TMSCN (222.04 mg, 0.28 mL, 2.238 mmol) were successively added and the reaction mixture was stirred at ambient temperature for 16 h. The reaction was quenched by addition of a saturated aqueous NaHCO₃ solution (20 mL) and diluted with DCM (20 mL). The aqueous layer was separated and extracted with DCM (30 mL). The combined organic phases were dried (Na₂SO₄), filtered and concentrated in vacuo. Purification by reverse phase chromatography (C₁₈ column, 5 to 70% MeCN in water with 0.1% formic acid) gave rac-2-((4S,6R,7R)-7-(3,4-difluoro-2-methoxyphenyl)-1,1-difluorospiro[3.4]octan-6-yl)-4-oxo-1,4-dihydro-1,6-naphthyridine-5-carbonitrile (70 mg, 65% over 2 steps) as a brown solid. ¹H NMR (400 MHz, Methanol-d₄) δ 8.65 (d, J=4.2 Hz, 1H), 7.67 (br s, 1H), 7.23-7.14 (m, 1H), 7.03-6.92 (m, 1H), 6.35 (s, 1H), 4.00-3.90 (m, 1H), 3.80 (d, J=2.0 Hz, 3H), 3.67-3.57 (m, 1H), 2.81-2.54 (m, 2H), 2.47-2.29 (m, 1H), 2.25-2.08 (m, 1H), 1.20-1.09 (m, 2H), 1.05-0.97 (m, 2H) ppm; exchangeable H not observed. ESI-MS m/z calc. 457.141, found 458.2 (M+1)⁺.

### Step 5

NaOH (20 mg, 0.5 mmol) and a solution of H₂O₂(44 μL, 35 wt % solution in water, 0.453 mmol) were successively added to a solution of rac-2-((4S,6R,7R)-7-(3,4-difluoro-2-methoxyphenyl)-1,1-difluorospiro[3.4]octan-6-yl)-4-oxo-1,4-dihydro-1,6-naphthyridine-5-carbonitrile (40 mg, 0.082 mmol) in a mixture of DMSO (1.5 mL) and water (0.5 mL) and the reaction mixture was stirred at ambient temperature for 2 h. Additional amounts of NaOH (20 mg, 0.5 mmol) and H₂O₂(40 μL, 35 wt % solution in water, 0.412 mmol) were added and the mixture was further stirred at ambient temperature for 1.5 h. Purification by reverse phase chromatography (C₁₈ column, 5 to 60% MeCN in water with 0.1% formic acid) gave rac-2-((4S,6R,7R)-7-(3,4-difluoro-2-methoxyphenyl)-1,1-difluorospiro[3.4]octan-6-yl)-4-oxo-1,4-dihydro-1,6-naphthyridine-5-carboxamide (31 mg, 79%).

### Step 6

The enantiomers of rac-2-((4S,6R,7R)-7-(3,4-difluoro-2-methoxyphenyl)-1,1-difluorospiro[3.4]octan-6-yl)-4-oxo-1,4-dihydro-1,6-naphthyridine-5-carboxamide were separated by chiral SFC using a Lux® Cellulose-4 column, 5 μm particle size, 250 cm×30 mm from Phenomenex (Mobile phase: 50% MeOH (supplemented with 0.1% mM ammonia), 50% CO₂; Flow rate 80 mL/min) to give:

First Eluting Isomer: rel-2-((4R,6S,7S)-7-(3,4-difluoro-2-methoxyphenyl)-1,1-difluorospiro[3.4]octan-6-yl)-4-oxo-1,4-dihydro-1,6-naphthyridine-5-carboxamide (13, 5.9 mg, 19%) as a white solid, after subsequent purification by reverse phase chromatography (C₁₈ column, 5 to 60% MeCN in water with 0.1% formic acid) followed by SFC separation using a Lux® Cellulose 3 column, 5 m particle size, 250 cm×30 mm from Phenomenex (Mobile phase: 20% MeOH, 80% CO₂, flow rate 100 mL/min). ¹H NMR (400 MHz, DMSO-d₆) δ 11.63 (br s, 1H), 8.44 (d, J=5.7 Hz, 1H), 7.44-7.36 (m, 2H), 7.25-7.13 (m, 3H), 6.15 (s, 1H), 3.94-3.86 (m, 1H), 3.74 (d, J=1.1 Hz, 3H), 3.57 (td, J=12.0, 5.7 Hz, 1H), 2.65-2.55 (m, 1H, overlapped with solvent), 2.54-2.44 (m, 1H, overlapped with solvent), 2.31-2.14 (m, 1H), 2.07-1.91 (m, 1H), 1.23-1.03 (m, 4H) ppm. ESI-MS m/z calc. 475.152, found 476.2 (M+1)⁺.

Second Eluting Isomer: rel-2-((4S,6R,7R)-7-(3,4-difluoro-2-methoxyphenyl)-1,1-difluorospiro[3.4]octan-6-yl)-4-oxo-1,4-dihydro-1,6-naphthyridine-5-carboxamide (14, 8.5 mg, 27%) as a white solid, after subsequent purification by SFC separation using a Lux® Cellulose 3 column, 5 m particle size, 250 cm×30 mm from Phenomenex (Mobile phase: 20% MeOH, 80% CO₂, flow rate 100 mL/min). ¹H NMR (400 MHz, DMSO-d₆) δ 11.63 (br s, 1H), 8.44 (d, J=5.9 Hz, 1H), 7.46-7.34 (m, 2H), 7.27-7.12 (m, 3H), 6.15 (s, 1H), 3.94-3.85 (m, 1H), 3.74 (d, J=1.5 Hz, 3H), 3.57 (td, J=12.0, 5.8 Hz, 1H), 2.66-2.54 (m, 1H, overlapped with solvent), 2.54-2.44 (m, 1H, overlapped with solvent), 2.32-2.12 (m, 1H), 2.08-1.90 (m, 1H), 1.23-1.03 (m, 4H) ppm. ESI-MS m/z calc. 475.152, found 476.2 (M+1)⁺.

### Example 9

### rel-2-((1R,2R,3aR,6aS)-1-(3,4-Difluoro-2-hydroxyphenyl)-4,4-difluorooctahydropentalen-2-yl)-4-oxo-1,4-dihydro-1,6-naphthyridine-5-carboxamide (15)

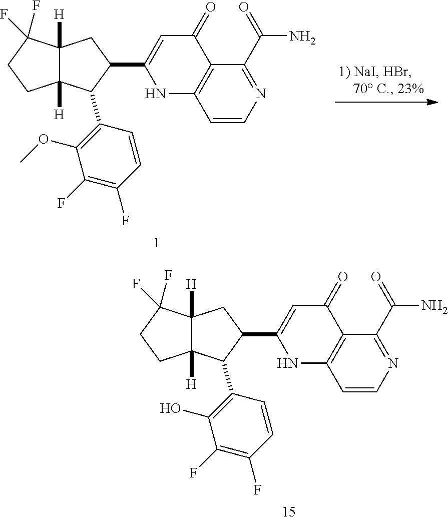 [SMILES: COc1c([C@@H]2[C@@H]3CCC(F)(F)[C@@H]3C[C@H]2c2cc(=O)c3c(C(N)=O)nccc3[nH]2)ccc(F)c1F ; NC(=O)c1nccc2[nH]c([C@@H]3C[C@@H]4[C@@H](CCC4(F)F)[C@H]3c3ccc(F)c(F)c3O)cc(=O)c12]

### Step 1

A vial containing a mixture of rel-2-((1R,2R,3aR,6aS)-1-(3,4-difluoro-2-methoxyphenyl)-4,4-difluorooctahydropentalen-2-yl)-4-oxo-1,4-dihydro-1,6-naphthyridine-5-carboxamide (1, 45 mg, 0.095 mmol), NaI (22 mg, 0.147 mmol) and HBr (450 μL, 30% w/v solution in AcOH, 1.668 mmol) was heated at 70° C. for 4 h. The reaction was quenched by addition of water and extracted with DCM (3 x). The organic layer was separated, washed with a saturated aqueous Na₂SO₃ solution and a saturated aqueous NaHCO₃ solution, dried (MgSO₄), filtered and concentrated in vacuo. Purification by flash chromatography (SiO₂, 0 to 80% EtOAc in DCM) followed by reverse phase chromatography (Cis column, 0 to 40% MeCN in water with HCl) gave rel-2-((1R,2R,3aR,6aS)-1-(3,4-difluoro-2-hydroxyphenyl)-4,4-difluorooctahydropentalen-2-yl)-4-oxo-1,4-dihydro-1,6-naphthyridine-5-carboxamide (15, 10.2 mg, 23%). ¹H NMR (400 MHz, Methanol-d₄) δ 8.84 (d, J=6.0 Hz, 1H), 8.06 (d, J=6.0 Hz, 1H), 6.96 (s, 1H), 6.93-6.86 (m, 1H), 6.59 (td, J=9.4, 7.5 Hz, 1H), 4.14-4.03 (m, 1H), 3.84-3.67 (m, 1H), 3.48 (p, J=8.7 Hz, 1H), 3.16-2.95 (m, 1H), 2.71-2.59 (m, 1H), 2.15-2.05 (m, 2H), 1.47-1.25 (m, 3H) ppm; exchangeable H not observed. ESI-MS m/z calc. 461.136, found 462.5 (M+1)⁺.

### Example 10

### rel-2-((7S,8S)-7-(3,4-Difluoro-2-methoxyphenyl)spiro[4.5]decan-8-yl)-1,6-naphthyridin-4(1H)-one (16) and rel-2-((7R,8R)-7-(3,4-difluoro-2-methoxyphenyl)spiro[4.5]decan-8-yl)-1,6-naphthyridin-4(1H)-one (17)

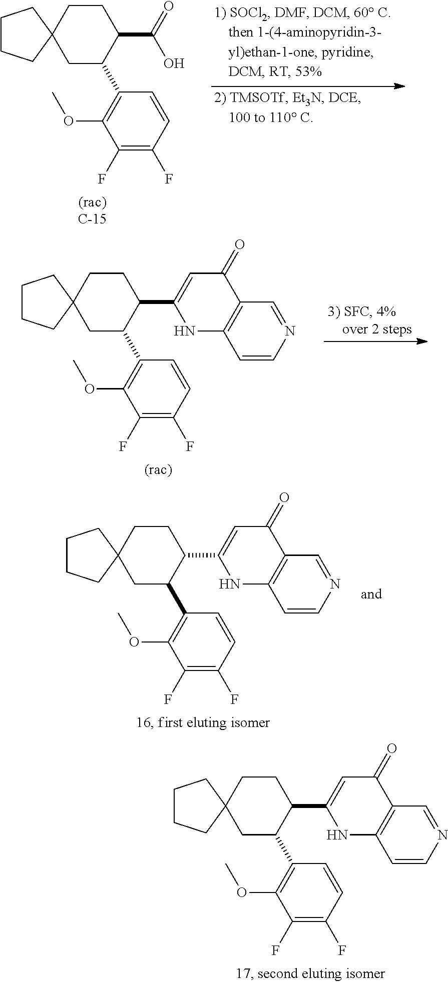 [SMILES: COc1c([C@@H]2CC3(CCCC3)CC[C@H]2C(=O)O)ccc(F)c1F ; COc1c([C@@H]2CC3(CCCC3)CC[C@H]2c2cc(=O)c3cnccc3[nH]2)ccc(F)c1F ; COc1c([C@H]2CC3(CCCC3)CC[C@@H]2c2cc(=O)c3cnccc3[nH]2)ccc(F)c1F ; COc1c([C@@H]2CC3(CCCC3)CC[C@H]2c2cc(=O)c3cnccc3[nH]2)ccc(F)c1F]

### Step 1

A stirred suspension of rac-(7R,8R)-7-(3,4-difluoro-2-methoxyphenyl)spiro[4.5]decane-8-carboxylic acid (C-15, 597 mg, 1.841 mmol) and DMF (15 μL, 0.194 mmol) in a mixture of SOCl₂ (2 mL, 27.42 mmol) and DCM (7 mL) was heated at 60° C. for 1 h. The reaction was cooled to ambient temperature, diluted with toluene (3 mL) and concentrated in vacuo. Pyridine (450 μL, 5.564 mmol) was added to a solution of the residue and 1-(4-amino-3-pyridyl)ethanone (280 mg, 2.057 mmol) in DCM (7 mL) and the reaction mixture was stirred at ambient temperature for 16 h. The mixture was filtered to remove insoluble solids and concentrated in vacuo. Purification by flash chromatography (SiO₂, 0 to 30% EtOAc in hexanes) gave rac-(7R,8R)—N-(3-acetylpyridin-4-yl)-7-(3,4-difluoro-2-methoxyphenyl)spiro[4.5]decane-8-carboxamide (433.3 mg, 53%). ¹H NMR (400 MHz, Chloroform-d) δ 11.68 (s, 1H), 9.03 (s, 1H), 8.52-8.46 (m, 2H), 6.95-6.87 (m, 1H), 6.82-6.69 (m, 1H), 3.94 (d, J=1.8 Hz, 3H), 3.53-3.37 (m, 1H), 2.69 (s, 3H), 2.66-2.58 (m, 1H), 2.06-1.94 (m, 1H), 1.93-1.80 (m, 1H), 1.72-1.56 (m, 8H), 1.50-1.37 (m, 4H) ppm. ESI-MS m/z calc. 442.207, found 443.2 (M+1)⁺.

### Step 2

TMSOTf (700 μL, 3.874 mmol) was added to a stirred solution of rac-(7R,8R)—N-(3-acetylpyridin-4-yl)-7-(3,4-difluoro-2-methoxyphenyl)spiro[4.5]decane-8-carboxamide (433 mg, 0.979 mmol) and Et₃N (275 μL, 1.973 mmol) in DCE (4.5 mL) and the reaction mixture was heated at 100° C. for 16 h and at 110° C. for 24 h. The reaction was cooled to ambient temperature and partitioned between EtOAc and a saturated aqueous NaHCO₃ solution. The organic layer was separated, dried (Na₂SO₄), filtered and concentrated in vacuo. Purification by reverse phase chromatography (C₁₈ column, 20 to 75% MeCN in water with 5 mM HCl) gave rac-2-((7R,8R)-7-(3,4-difluoro-2-methoxyphenyl)spiro[4.5]decan-8-yl)-1,6-naphthyridin-4(1H)-one as a white solid. ESI-MS m/z calc. 424.196, found 425.2 (M+1)⁺.

### Step 3

The enantiomers of rac-2-((7R,8R)-7-(3,4-difluoro-2-methoxyphenyl)spiro[4.5]decan-8-yl)-1,6-naphthyridin-4(1H)-one were separated by chiral SFC using a Chiralpak® IC column, 5 m particle size, 25 cm×21.2 mm from Daicel Corporation (Mobile phase: 28% MeOH (supplemented with 20 mM ammonia), 72% CO₂; Flow rate 70 mL/min) to give:

First Eluting Isomer: rel-2-((7S,8S)-7-(3,4-difluoro-2-methoxyphenyl)spiro[4.5]decan-8-yl)-1,6-naphthyridin-4(1H)-one (16, 7 mg, 2% over 2 steps). ¹H NMR (400 MHz, Chloroform-d) δ 9.39 (s, 1H), 8.89 (s, 1H), 8.55 (d, J=5.8 Hz, 1H), 7.12 (s, 1H), 6.94-6.64 (m, 2H), 6.14 (s, 1H), 4.00 (s, 3H), 3.43 (s, 1H), 2.74 (s, 1H), 2.06-1.95 (m, 1H), 1.83-1.71 (m, 2H), 1.67-1.59 (m, 9H), 1.51-1.41 (m, 2H) ppm. ESI-MS m/z calc. 424.196, found 425.2 (M+1)⁺.

Second Eluting Isomer: rel-2-((7R,8R)-7-(3,4-difluoro-2-methoxyphenyl)spiro[4.5]decan-8-yl)-1,6-naphthyridin-4(1H)-one (17, 7.6 mg, 2% over 2 steps). ¹H NMR (400 MHz, Chloroform-d) δ 9.40 (s, 1H), 8.71 (s, 1H), 8.56 (d, J=5.8 Hz, 1H), 7.08 (d, J=5.9 Hz, 1H), 6.81 (s, 2H), 6.13 (s, 1H), 4.01 (s, 3H), 3.41 (s, 1H), 2.72 (s, 1H), 2.05-1.94 (m, 1H), 1.79-1.69 (m, 2H), 1.67-1.56 (m, 9H), 1.49-1.42 (m, 2H) ppm. ESI-MS m/z calc. 424.196, found 425.2 (M+1)⁺.

### Example 11

### rac-2-((2R,3S,3aS,6aR)-3-(3,4-Difluoro-2-methylphenyl)-6,6-difluorohexahydrofuro[3,2-b]furan-2-yl)-1,6-naphthyridin-4(1H)-one (18)

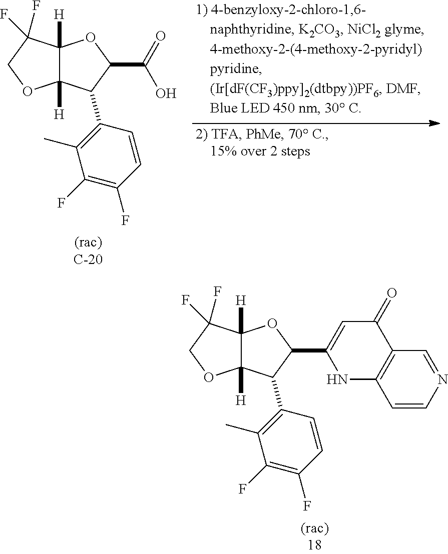 [SMILES: Cc1c([C@@H]2[C@@H]3OCC(F)(F)[C@@H]3O[C@H]2C(=O)O)ccc(F)c1F ; Cc1c([C@@H]2[C@@H]3OCC(F)(F)[C@@H]3O[C@H]2c2cc(=O)c3cnccc3[nH]2)ccc(F)c1F]

### Step 1

Under a nitrogen atmosphere, DMF (1.8 mL) was added to a stirred mixture of (2R,3R,3aS,6aR)-3-(3,4-difluoro-2-methylphenyl)-6,6-difluorohexahydrofuro [3,2-b]furan-2-carboxylic acid (C-20, 50 mg, 0.156 mmol), 4-benzyloxy-2-chloro-1,6-naphthyridine (Int-5, 43 mg, 0.159 mmol), K₂CO₃ (64 mg, 0.463 mmol), 4-methoxy-2-(4-methoxy-2-pyridyl)pyridine (4 mg, 0.019 mmol), NiCl₂ glyme (4 mg, 0.018 mmol), and (Ir[dF(CF₃)ppy]₂(dtbpy))PF₆ (9 mg, 0.008 mmol), and the reaction was degassed for 15 min by passing nitrogen gas through the mixture. The solution was stirred at 30° C. for 19 h under irradiation of a blue LED (450 nm) using a Hepatochem photoreactor equipped with a cooling fan. Purification by preparative reverse phase HPLC (C₁₈ column, 1 to 99% ACN in water with 5 mM HCl) gave rac-4-(benzyloxy)-2-((2R,3S,3aS,6aR)-3-(3,4-difluoro-2-methylphenyl)-6,6-difluorohexahydrofuro[3,2-b]furan-2-yl)-1,6-naphthyridine (39 mg) which was used without further purification in the next step.

### Step 2

TFA (400 μL, 5.192 mmol) was added to a solution of rac-4-(benzyloxy)-2-((2R,3S,3aS,6aR)-3-(3,4-difluoro-2-methylphenyl)-6,6-difluorohexahydrofuro[3,2-b]furan-2-yl)-1,6-naphthyridine (39 mg) in toluene (500 μL). The reaction mixture was heated at 70° C. for 1 h and concentrated in vacuo. Purification by preparative reverse phase HPLC (C₁₈ column, 1 to 99% ACN in water with 5 mM HCl) gave rac-2-((2R,3S,3aS,6aR)-3-(3,4-difluoro-2-methylphenyl)-6,6-difluorohexahydrofuro[3,2-b]furan-2-yl)-1,6-naphthyridin-4(1H)-one hydrochloride (18, 11 mg, 15%) as a white solid. ¹H NMR (400 MHz, Methanol-d₄) δ 9.35 (s, 1H), 8.69 (dd, J=6.9, 1.1 Hz, 1H), 8.13 (d, J=6.9 Hz, 1H), 7.54-7.33 (m, 1H), 7.14 (q, J=9.0 Hz, 1H), 6.21 (s, 1H), 5.53 (d, J=11.0 Hz, 1H), 5.14-5.00 (m, 2H), 4.37-4.16 (m, 1H), 4.05 (dt, J=13.0, 10.4 Hz, 1H), 3.83 (dd, J=11.0, 4.2 Hz, 1H), 2.17 (s, 3H) ppm; exchangeable H not observed. ESI-MS m/z calc. 420.110, found 421.0 (M+1)⁺.

### Example 12

### rel-2-((1R,2R,4S)-2-(3,4-Difluoro-2-methylphenyl)-4-(trifluoromethyl)cyclopentyl)-4-oxo-1,4-dihydro-1,6-naphthyridine-5-carboxamide (19)

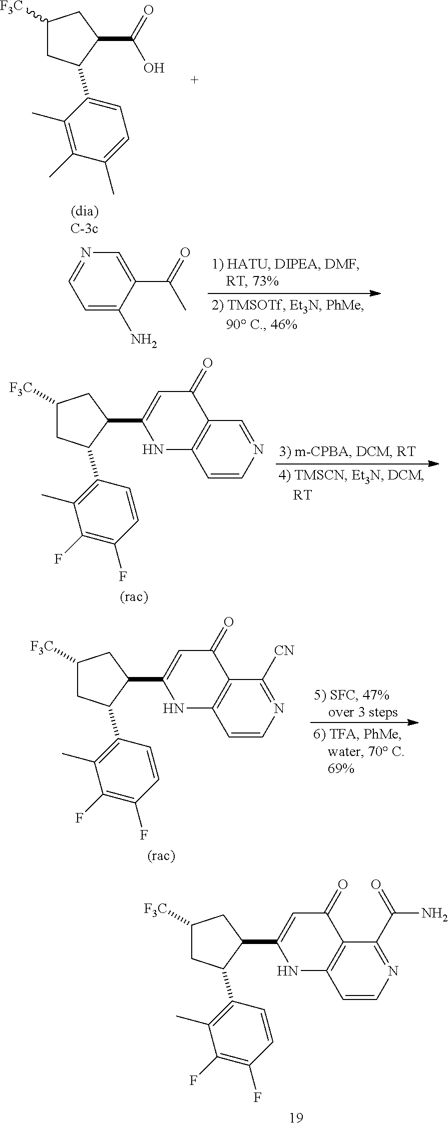

### Step 1

DIPEA (1.187 g, 1.6 mL, 9.186 mmol) was added to a stirred solution of dia-(1R¹,2R¹,4S²)-2-(3,4-difluoro-2-methylphenyl)-4-(trifluoromethyl)cyclopentane-1-carboxylic acid (C-3c, 950 mg, 2.974 mmol), 1-(4-aminopyridin-3-yl)ethan-1-one (515 mg, 3.783 mmol) and HATU (1.5 g, 3.945 mmol) in DMF (20 mL) and the reaction mixture was stirred at ambient temperature for 20 h. The reaction was diluted with EtOAc (200 mL), washed with a 1:1 mixture of water and a saturated aqueous brine solution (2×50 mL), and brine (50 mL), dried (Na₂SO₄), filtered and concentrated in vacuo. Purification by flash chromatography (SiO₂, 10 to 100% EtOAc in heptane) gave:

First Eluting Isomers: rac-(1R,2R,4S)—N-(3-acetylpyridin-4-yl)-2-(3,4-difluoro-2-methylphenyl)-4-(trifluoromethyl)cyclopentane-1-carboxamide (353 mg, 27%) as a light yellow oil. ¹H NMR (400 MHz, DMSO-d₆) δ 11.24 (s, 1H), 9.07 (s, 1H), 8.59 (d, J=5.6 Hz, 1H), 8.25 (d, J=5.6 Hz, 1H), 7.38-7.19 (m, 2H), 3.60 (td, J=11.5, 6.8 Hz, 1H), 3.29-3.11 (m, 2H), 2.62-2.56 (m, 3H), 2.42-2.33 (m, 1H), 2.28 (t, J=8.6 Hz, 2H), 2.18 (d, J=1.7 Hz, 3H), 1.71 (td, J=12.3, 9.9 Hz, 1H) ppm. ESI-MS m/z calc. 426.137, found 427.2 (M+1)⁺.

Second Eluting Isomers: rac-(1R,2R,4R)—N-(3-acetylpyridin-4-yl)-2-(3,4-difluoro-2-methylphenyl)-4-(trifluoromethyl)cyclopentane-1-carboxamide (638 mg, 46%) as a white solid. ¹H NMR (400 MHz, DMSO-d₆) δ 11.34 (s, 1H), 9.10 (s, 1H), 8.59 (d, J=5.9 Hz, 1H), 8.29 (d, J=5.6 Hz, 1H), 7.40-7.21 (m, 2H), 3.62-3.50 (m, 1H), 3.31-3.21 (m, 2H), 2.61 (s, 3H), 2.47-2.40 (m, 1H), 2.31-2.20 (m, 1H), 2.16 (d, J=1.2 Hz, 3H), 2.06-1.92 (m, 2H, overlapped with solvent) ppm. ESI-MS m z calc. 426.137, found 427.1 (M+1)⁺.

### Step 2

Et₃N (290.4 mg, 0.4 mL, 2.870 mmol) and TMSOTf (1.228 g, 1 mL, 5.525 mmol) were successively added to a solution of rac-(1R,2R,4S)—N-(3-acetylpyridin-4-yl)-2-(3,4-difluoro-2-methylphenyl)-4-(trifluoromethyl)cyclopentane-1-carboxamide (350 mg, 0.788 mmol) in toluene (8 mL) and the reaction mixture was stirred at 90° C. for 19 h. The reaction was cooled to ambient temperature and diluted with 2-MeTHF (150 mL). The mixture was washed with a saturated aqueous NaHCO₃ solution (50 mL) and brine (20 mL), dried (Na₂SO₄), filtered and concentrated in vacuo. Purification by reverse phase chromatography (C₁₈ column, 5 to 95% MeCN in water with 0.1% formic acid) gave, after trituration from MTBE and heptane (3:1, 20 mL), rac-2-((1R,2R,4S)-2-(3,4-difluoro-2-methylphenyl)-4-(trifluoromethyl)cyclopentyl)-1,6-naphthyridin-4(1H)-one (111 mg, 33%) as a beige solid. ¹H NMR (400 MHz, DMSO-d₆) δ 11.72 (s, 1H), 9.07 (s, 1H), 8.55 (br d, J=5.9 Hz, 1H), 7.40 (d, J=5.9 Hz, 1H), 7.27-7.16 (m, 2H), 6.08 (s, 1H), 3.76 (td, J=11.7, 6.7 Hz, 1H), 3.51-3.36 (m, 1H), 3.31-3.19 (m, 1H), 2.58-2.39 (m, 2H, overlapped with solvent), 2.31-2.14 (m, 4H), 1.60 (td, J=12.2, 9.5 Hz, 1H) ppm. ESI-MS m/z calc. 408.126, found 409.2 (M+1)⁺. The filtrates were concentrated in vacuo to give a second crop of rac-2-((1R,2R,4S)-2-(3,4-difluoro-2-methylphenyl)-4-(trifluoromethyl)cyclopentyl)-1,6-naphthyridin-4(1H)-one (55 mg, 13%, 75% chemical purity) as a yellow oil. ESI-MS m/z calc. 408.126, found 409.2 (M+1)⁺.

### Steps 3 and 4

m-CPBA (110 mg, 0.491 mmol) was added to a stirred solution of rac-2-((1R,2R,4S)-2-(3,4-difluoro-2-methylphenyl)-4-(trifluoromethyl)cyclopentyl)-1,6-naphthyridin-4(1H)-one (106 mg, 0.245 mmol) in DCM (5 mL) and the reaction mixture was stirred at ambient temperature for 21 h. TMSCN (198.25 mg, 0.25 mL, 1.998 mmol) and Et₃N (254.1 mg, 0.35 mL, 2.511 mmol) were successively added and the reaction mixture was stirred at ambient temperature for 25 h. Additional amounts of TMSCN (198.25 mg, 0.25 mL, 1.998 mmol) and Et₃N (254.1 mg, 0.35 mL, 2.511 mmol) were added and the reaction mixture was stirred at ambient temperature for 4.5 h. The reaction was concentrated in vacuo and the residue was diluted with EtOAc (80 mL). The organic layer was separated, washed with a saturated aqueous NaHCO₃ solution (3×10 mL) and brine (10 mL), dried (Na₂SO₄), filtered and concentrated in vacuo. Purification by reverse phase chromatography (C₁₈ column, 5 to 95% MeCN in water with 0.1% formic acid) gave rac-2-((1R,2R,4S)-2-(3,4-difluoro-2-methylphenyl)-4-(trifluoromethyl)cyclopentyl)-4-oxo-1,4-dihydro-1,6-naphthyridine-5-carbonitrile.

### Step 5

The enantiomers of rac-2-((1R,2R,4S)-2-(3,4-difluoro-2-methylphenyl)-4-(trifluoromethyl)cyclopentyl)-4-oxo-1,4-dihydro-1,6-naphthyridine-5-carbonitrile were separated by chiral SFC using a Lux® Cellulose-2 column, 5 μm particle size, 25 cm×30 mm from Phenomenex (Mobile phase: 35% ethanol, 65% CO₂; Flow rate 100 mL/min) to give:

First Eluting Isomer: rel-2-((1S,2S,4R)-2-(3,4-difluoro-2-methylphenyl)-4-(trifluoromethyl)cyclopentyl)-4-oxo-1,4-dihydro-1,6-naphthyridine-5-carbonitrile (27 mg, 24% over 3 steps) as an off-white solid. ¹H NMR (400 MHz, Methanol-d₄) δ 8.63 (d, J=5.9 Hz, 1H), 7.63 (d, J=5.9 Hz, 1H), 7.17 (ddd, J=8.7, 4.8, 1.6 Hz, 1H), 7.12-7.01 (m, 1H), 6.30 (s, 1H), 3.76 (td, J=11.8, 7.0 Hz, 1H), 3.45 (td, J=11.2, 8.7 Hz, 1H), 3.28-3.17 (m, 1H), 2.65-2.50 (m, 2H), 2.37-2.22 (m, 4H), 1.81 (td, J=12.5, 9.2 Hz, 1H) ppm; exchangeable H not observed. ESI-MS m/z calc. 433.121, found 434.1 (M+1)⁺.

Second Eluting Isomer: rel-2-((1R,2R,4S)-2-(3,4-difluoro-2-methylphenyl)-4-(trifluoromethyl)cyclopentyl)-4-oxo-1,4-dihydro-1,6-naphthyridine-5-carbonitrile (24 mg, 23% over 3 steps) as an off-white solid. ¹H NMR (400 MHz, Methanol-d₄) δ 8.63 (d, J=5.9 Hz, 1H), 7.63 (d, J=5.9 Hz, 1H), 7.22-7.13 (m, 1H), 7.12-7.03 (m, 1H), 6.30 (s, 1H), 3.76 (td, J=11.8, 7.0 Hz, 1H), 3.45 (td, J=11.1, 8.7 Hz, 1H), 3.28-3.17 (m, 1H), 2.64-2.50 (m, 2H), 2.37-2.20 (m, 4H), 1.81 (td, J=12.5, 9.2 Hz, 1H) ppm; exchangeable H not observed. ESI-MS m/z calc. 433.121, found 434.1 (M+1)⁺.

### Step 6

A solution of rel-2-((1R,2R,4S)-2-(3,4-difluoro-2-methylphenyl)-4-(trifluoromethyl)cyclopentyl)-4-oxo-1,4-dihydro-1,6-naphthyridine-5-carbonitrile (21 mg, 0.048 mmol) and TFA (1.036 g, 0.7 mL, 9.086 mmol) in a mixture of toluene (1 mL) and water (0.03 mL, 1.665 mmol) was stirred at 70° C. for 17 h. The reaction was concentrated in vacuo and the residue was diluted with EtOAc (70 mL). The organic layer was separated and washed with a saturated aqueous NaHCO₃ solution (10 mL) and brine (10 mL), dried (Na₂SO₄), filtered and concentrated in vacuo. Purification by reverse phase chromatography (C₁₈ column, 5 to 95% MeCN in water with 0.1% formic acid) gave rel-2-((1R,2R,4S)-2-(3,4-difluoro-2-methylphenyl)-4-(trifluoromethyl)cyclopentyl)-4-oxo-1,4-dihydro-1,6-naphthyridine-5-carboxamide (19, 15 mg, 69%) as a white solid. ¹H NMR (400 MHz, Methanol-d₄) δ 8.48 (br d, J=5.9 Hz, 1H), 7.47 (br d, J=4.9 Hz, 1H), 7.23-7.12 (m, 1H), 7.12-7.01 (m, 1H), 6.23 (br s, 1H), 3.74 (td, J=11.8, 7.0 Hz, 1H), 3.49-3.36 (m, 1H), 3.29-3.16 (m, 1H), 2.62-2.49 (m, 2H), 2.38-2.24 (m, 1H), 2.21 (d, J=2.2 Hz, 3H), 1.82 (td, J=12.5, 9.3 Hz, 1H) ppm; exchangeable H not observed. ESI-MS m/z calc. 451.132, found 452.1 (M+1)⁺.

The following compounds were made using methods analogous to those disclosed in the specification.

TABLE F
Compounds Prepared Using Analogous Methods.

| | | | SFC* | | |
|---|---|---|---|---|---|
| Cmpd | | Starting | Conditions | | |
| No. | Compound Structure/Compound Name | Material | if applicable | LC/MS | NMR (shifts in ppm) |
| 20 | 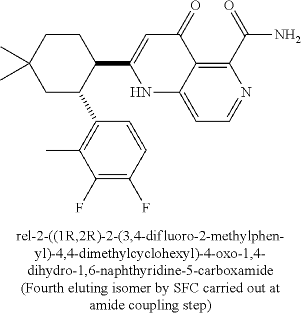 | C-11b | Lux ® i- Cellulose-5 column, 5 μm particle size, 25 cm × 21.2 mm from Phenomene × (Mobile phase: 7% EtOH, 93% CO₂; Flow rate 75 mL/min) | ESI-MS m/z calc. 425.191, found 426.2 (M + 1)+. | ¹H NMR (400 MHz, Chloroform-d) δ 15.13 (br s, 1H), 8.90 (br s, 1H), 8.54 (d, J = 4.6 Hz, 1H), 7.96 (d, J = 4.2 Hz, 1H), 7.03-6.92 (m, 1H), 6.88-6.71 (m, 2H), 6.04 (br s, 1H), 3.67 (t, J = 11.6 Hz, 1H), 3.18-3.04 (m, 1H), 2.28 (br s, 3H), 2.02-1.89 (m, 2H), 1.72- 1.48 (m, 3H, overlapped with water), 1.43-1.32 (m, 1H), 1.22 (s, 3H), 1.03 (s, 3H) ppm. |
| 21 | 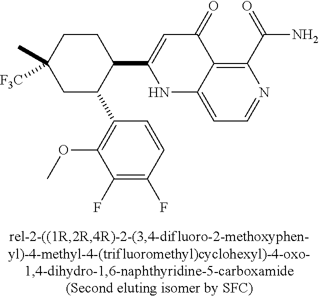 | C-13a | Lux ® i- Cellulose-5 column, 5 μm particle size, 25 cm × 21.2 mm from Phenomene × (Mobile phase: 30% MeOH, 70% CO₂; Flow rate 75 mL/min) | ESI-MS m/z calc. 495.158, found 496.2 (M + 1)+. | ¹H NMR (400 MHz, DMSO-d₆) δ 11.43 (br s, 1H), 8.42 (d, J = 5.9 Hz, 1H), 7.42-7.36 (m, 2H), 7.30-7.16 (m, 2H), 7.11- 7.03 (m, 1H), 5.99 (s, 1H), 3.84 (br s, 3H), 3.74-3.61 (m, 1H), 3.08- 2.98 (m, 1H), 2.07-1.92 (m, 2H), 1.89-1.70 (m, 3H), 1.66-1.60 (m, 1H), 1.45 (s, 3H) ppm. |
| 22 | 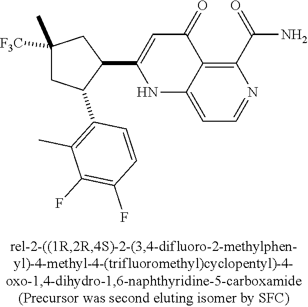 | C-9a | Lux ® Cellulose-2 column, 5 μm particle size, 25 cm × 30 mm from Phenomene × (Mobile phase: 20% MeOH (supplemented with 0.1% DEA), 80% CO₂; Flow rate 100 mL/min) | ESI-MS m/z calc. 465.148, found 466.2 (M + 1)+. | ¹H NMR (400 MHz, DMSO-d₆) δ 11.64 (br s, 1H), 8.43 (d, J = 5.9 Hz, 1H), 7.41-7.36 (m, 2H), 7.24-7.13 (m, 3H), 6.19 (s, 1H), 3.90 (td, J = 12.0, 6.9 Hz, 1H), 3.46 (td, J = 12.0, 7.6 Hz, 1H), 2.65 (dd, J = 13.6, 7.3 Hz, 1H), 2.28 (d, J = 2.1 Hz, 3H), 2.16 (dd, J = 13.1, 7.0 Hz, 1H), 1.91-1.73 (m, 2H), 1.46 (s, 3H) ppm. |
| 23 | 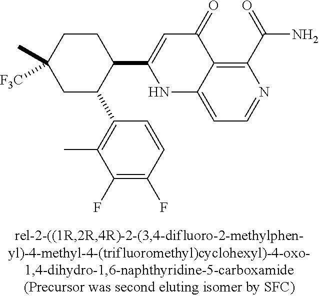 | C-13b | Lux ® Cellulose-2 column, 5 μm particle size, 25 cm × 30 mm from Phenomene × (Mobile phase: 30% MeOH, 70% CO₂; Flow rate 100 mL/min) | ESI-MS m/z calc. 479.163, found 480.2 (M + 1)+. | ¹H NMR (400 MHz, Methanol-d₄) δ 8.46 (d, J = 6.1 Hz, 1H), 7.45 (br d, J = 5.9 Hz, 1H), 7.16- 7.08 (m, 1H), 6.99 (q, J = 8.8 Hz, 1H), 6.21 (s, 1H), 3.52-3.43 (m, 1H), 3.03 (td, J = 11.7, 4.0 Hz, 1H), 2.25 (d, J = 2.2 Hz, 3H), 2.17-1.80 (m, 4H), 1.80-1.73 (m, 2H), 1.50 (s, 3H) ppm; exchangeable H not observed. |
| 24 | 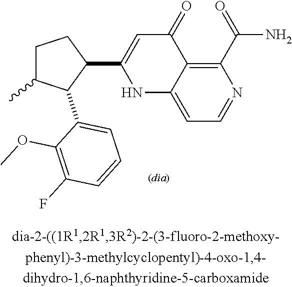 | C-7 | | ESI-MS m/z calc. 395.165, found 396.5 (M + 1)+. | ¹H NMR (400 MHz, DMSO-d₆) δ 11.62- 11.46 (m, 1H), 8.42 (d, J = 5.8 Hz, 1H), 7.44 (s, 1H), 7.37 (br s, 1H), 7.22-6.99 (m, 4H), 5.98 (s, 1H), 3.91-3.82 (m, 0.5H), 3.81-3.61 (m, 3H), 3.57-3.45 (m, 0.5H), 3.36-3.21 (m, 1H), 2.62-2.53 (m, 1H), 2.35-2.04 (m, 2H), 1.97- 1.83 (m, 1H), 1.71-1.42 (m, 1H), 0.95-0.54 (m, 3H) ppm. |
| 25 | 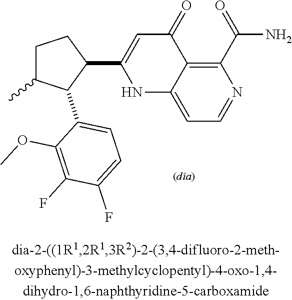 | C-7 | | ESI-MS m/z calc. 413.155, found 414.5 (M + 1)+. | ¹H NMR (400 MHz, DMSO-d₆) δ 11.67- 11.46 (m, 1H), 8.49- 8.37 (m, 1H), 7.47-7.41 (m, 1H), 7.38 (br s, 1H), 7.26-7.01 (m, 3H), 6.13- 5.87 (m, 1H), 3.90-3.66 (m, 3.5H), 3.56-3.44 (m, 0.5H), 3.30-3.19 (m, 1H), 2.59-2.52 (m, 0.5H), 2.30-2.21 (m, 1.5H), 2.19-2.04 (m, 1H), 1.97- 1.81 (m, 1H), 1.68-1.42 (m, 1H), 0.97-0.50 (m, 3H) ppm. |
| 26 | 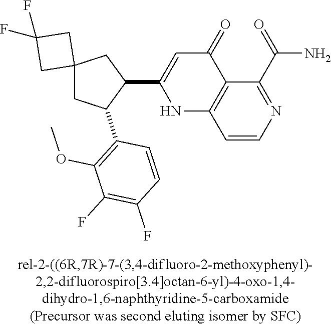 | C-4 | Lux ® Cellulose-2 column, 5 μm particle size, 25 cm × 30 mm from Phenomene × (Mobile phase: 30% MeOH, 70% CO₂; Flow rate 100 mL/min) | ESI-MS m/z calc. 475.152, found 476.2 (M + 1)+. | ¹H NMR (400 MHz, DMSO-d₆) δ 11.52 (s, 1H), 8.43 (d, J = 5.9 Hz, 1H), 7.43-7.36 (m, 2H), 7.23-7.06 (m, 3H), 6.00 (s, 1H), 3.84-3.75 (m, 4H), 3.30-3.24 (m, 1H, overlapped with water), 2.80-2.65 (m, 4H), 2.47- 2.40 (m, 2H, overlapped with solvent), 2.23-2.14 (m, 1H), 2.01-1.93 (m, 1H) ppm. |
| 27 | 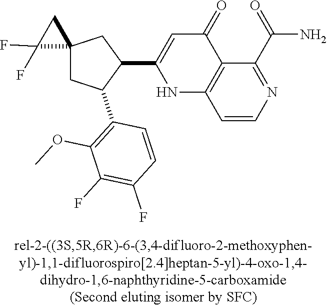 | C-5b | Lux ® Cellulose-2 column, 5 μm particle size, 25 cm × 30 mm from Phenomene × (Mobile phase: 35% 1:1 EtOH:MeCN, 65% CO₂; Flow rate 100 mL/min) | ESI-MS m/z calc. 461.136, found 462.2 (M + 1)+. | ¹H NMR (400 MHz, DMSO-d₆) δ 11.64 (s, 1H), 8.43 (d, J = 5.9 Hz, 1H), 7.43 (d, J = 5.6 Hz, 1H), 7.39 (br s, 1H), 7.29-7.23 (m, 1H), 7.20 (br s, 1H), 7.16-7.07 (m, 1H), 6.04 (s, 1H), 4.12- 4.00 (m, 1H), 3.80 (d, J = 1.5 Hz, 3H), 3.45 (td, J = 11.6, 7.5 Hz, 1H), 2.40 (br dd, J = 13.0, 7.3 Hz, 1H), 2.26- 2.12 (m, 2H), 2.11-2.01 (m, 1H), 1.69-1.59 (m, 1H), 1.59-1.48 (m, 1H) ppm. |
| 28 | 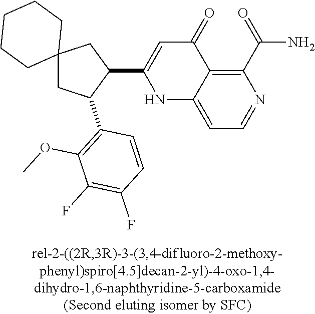 | C-2 | Lux ® Cellulose-4 column, 5 μm particle size, 25 cm × 21.2 mm from Phenomene × (Mobile phase: 42% MeOH (supplemented with 20 mM ammonia), 58% CO₂; Flow rate 70 mL/min) | ESI-MS m/z calc. 467.202, found 468.3 (M + 1)+. | ¹H NMR (400 MHz, DMSO-d₆) δ 11.52 (s, 1H), 8.42 (d, J = 5.8 Hz, 1H), 7.52-7.30 (m, 2H), 7.28-7.14 (m, 2H), 7.14- 7.02 (m, 1H), 5.99 (s, 1H), 3.81 (s, 3H), 3.78- 3.67 (m, 1H), 3.43-3.35 (m, 1H), 2.17 (dd, J = 12.6, 7.1 Hz, 1H), 2.07 (dd, J = 13.2, 8.2 Hz, 1H), 1.70 (t, J = 12.2 Hz, 1H), 1.60-1.36 (m, 11H) ppm. |
| 29 | 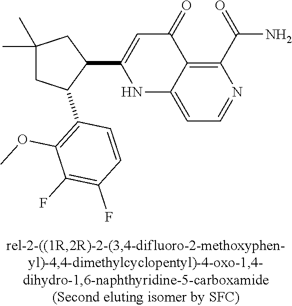 | C-1 | Lux ® Cellulose-4 column, 5 μm particle size, 25 cm × 30 mm from Phenomene × (Mobile phase: 35% 1:1 EtOH:MeCN, 65% CO₂; Flow rate 100 mL/min) | ESI-MS m/z calc. 427.171, found 428.2 (M + 1)+. | ¹H NMR (400 MHz, DMSO-d₆) δ 11.52 (br s, 1H), 8.42 (d, J = 5.6 Hz, 1H), 7.43 (d, J = 5.6 Hz, 1H), 7.38 (br s, 1H), 7.27-7.15 (m, 2H), 7.15- 7.05 (m, 1H), 6.01 (s, 1H), 3.90-3.82 (m, 1H), 3.80 (s, 3H), 3.44 (td, J = 11.6, 7.5 Hz, 1H), 2.12-1.98 (m, 2H), 1.82 (t, J = 12.1 Hz, 1H), 1.54 (t, J = 11.7 Hz, 1H), 1.21 (s, 3H), 1.20 (s, 3H) ppm. |
| 30 | 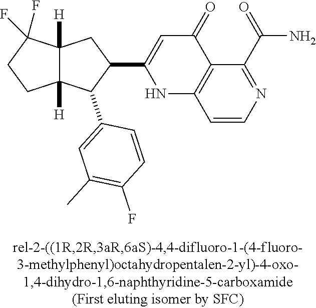 | C-8c | Lux ® i- Cellulose-5 column, 5 μm particle size, 25 cm × 30 mm from Phenomene × (Mobile phase: 30% MeOH (supplemented with 0.1% DEA), 70% CO₂; Flow rate 100 mL/min) | ESI-MS m/z calc. 441.166, found 442.2 (M + 1)+. | ¹H NMR (400 MHz, DMSO-d₆) δ 11.68 (br s, 1H), 8.43 (d, J = 5.9 Hz, 1H), 7.43 (d, J = 5.9 Hz, 1H), 7.38 (br s, 1H), 7.25-7.14 (m, 2H), 7.13- 7.07 (m, 1H), 7.06-6.97 (m, 1H), 6.08 (s, 1H), 3.70 (dd, J = 12.5, 7.8 Hz, 1H), 3.44-3.34 (m, 1H), 3.10 (quin, J = 8.4 Hz, 1H), 3.01-2.84 (m, 1H), 2.40 (dd, J = 13.0, 6.8 Hz, 1H), 2.17 (d, J = 1.0 Hz, 3H), 2.11-1.92 (m, 3H), 1.36-1.14 (m, 2H) ppm. |
| 31 | 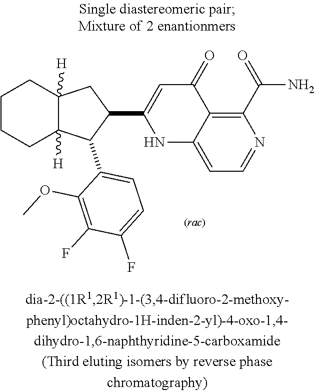 | C-6 | | ESI-MS m/z calc. 453.186, found 454.2 (M + 1)+. | |
| 32 | 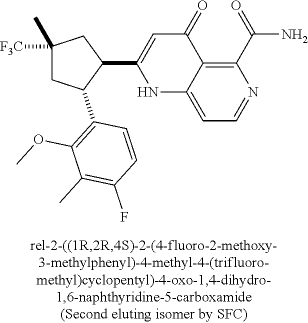 | C-9b | Enantiocel ® C4 column, 5 μm particle size, 25 cm × 30 mm from ColumnTek (Mobile phase: 25% EtOH (supplemented with 0.1% DEA), 75% CO₂; Flow rate 85 mL/min) | ESI-MS m/z calc. 477.168, found 478.1 (M + 1)+. | ¹H NMR (400 MHz, DMSO-d₆) δ 11.61 (br s, 1H), 8.41 (d, J = 5.8 Hz, 1H), 7.45-7.31 (m, 2H), 7.29-7.09 (m, 2H), 6.93 (t, J = 8.8 Hz, 1H), 6.15 (s, 1H), 3.99-3.87 (m, 1H), 3.67 (s, 3H), 3.47- 3.35 (m, 1H), 2.73-2.59 (m, 1H), 2.14-2.05 (m, 4H), 1.98-1.84 (m, 2H), 1.44 (s, 3H) ppm. |
| 33 | 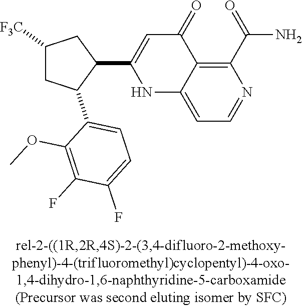 | C-3b | Lux ® Cellulose-2 column, 5 μm particle size, 25 cm × 30 mm from Phenomene × (Mobile phase: 40% iPrOH (supplemented with 0.1% DEA), 60% CO₂; Flow rate 150 mL/min) | ESI-MS m/z calc. 467.127, found 468.2 (M + 1)+. | ¹H NMR (400 MHz, DMSO-d₆) δ 11.67 (br s, 1H), 8.43 (d, J = 5.6 Hz, 1H), 7.40 (br d, J = 5.6 Hz, 2H), 7.26-7.17 (m, 2H), 7.16-7.07 (m, 1H), 6.03 (br s, 1H), 3.89- 3.74 (m, 4H), 3.48-3.20 (m, 2H, overlapped with water), 2.46-2.35 (m, 2H), 2.29-2.17 (m, 1H), 1.83-1.72 (m, 1H) ppm. |
| 34 | 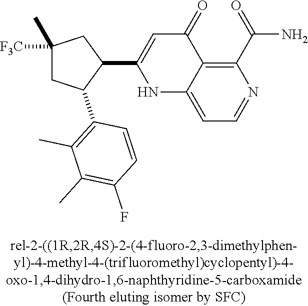 | C-9d | Enantiocel ® C4 column, 5 μm particle size, 25 cm × 30 mm from ColumnTek (Mobile phase: 25% MeOH (supplemented with 0.1% DEA), 75% CO₂; Flow rate 85 mL/min) | ESI-MS m/z calc. 461.173, found 462.1 (M + 1)+. | ¹H NMR (400 MHz, DMSO-d₆) δ 11.76 (br s, 1H), 8.39 (d, J = 5.7 Hz, 1H), 7.51-7.25 (m, 2H), 7.23-7.08 (m, 2H), 6.92 (t, J = 9.0 Hz, 1H), 6.15 (s, 1H), 4.02-3.89 (m, 1H), 3.52-3.39 (m, 1H), 2.70-2.59 (m, 1H), 2.26 (s, 3H), 2.13-2.08 (m, 1H), 2.09 (d, J = 1.6 Hz, 3H), 1.86 (t, J = 12.7 Hz, 1H), 1.74 (t, J = 12.7 Hz, 1H), 1.45 (s, 3H) ppm. |
| 35 | 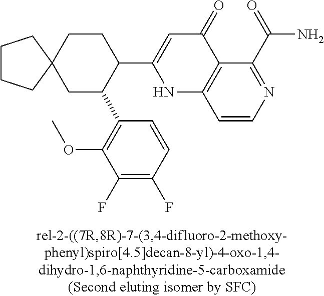 [SMILES: COc1c([C@@H]2CC3(CCCC3)CCC2c2cc(=O)c3c(C(N)=O)nccc3[nH]2)ccc(F)c1F] | C-15 | Chiralpak ® IC column, 5 μm particle size, 25 cm × 21.2 mm from Daicel Corporation (Mobile phase: 30% MeOH (supplemented with 20 mM ammonia), 70% CO₂; Flow rate 70 mL/min) | ESI-MS m/z calc. 467.202, found 468.2 (M + 1)+. | ¹H NMR (400 MHz, Chloroform-d) δ 15.09 (s, 1H), 8.88 (s, 1H), 8.50 (d, J = 5.7 Hz, 1H), 7.92 (d, J = 5.6 Hz, 1H), 6.88 (s, 2H), 6.75-6.48 (m, 1H), 6.06 (s, 1H), 3.92 (s, 3H), 3.80-3.58 (m, 1H), 3.14 (s, 1H), 2.01-1.93 (m, 1H), 1.77- 1.58 (m, 12H), 1.45-1.41 (m, 1H) ppm. |
| 36 | 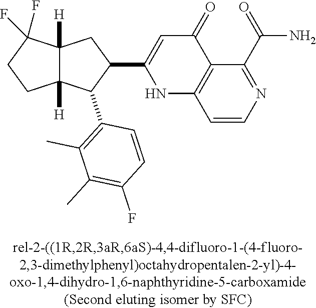 | C-8d | Lux ® Cellulose-4 column, 5 μm particle size, 25 cm × 21.2 mm from Phenomene × (Mobile phase: 52% MeOH (supplemented with 20 mM ammonia), 48% CO₂; Flow rate 78 mL/min) | ESI-MS m/z calc. 455.182, found 456.2 (M + 1)+. | ¹H NMR (400 MHz, Chloroform-d) δ 15.19 (s, 1H), 8.88 (s, 1H), 8.52 (d, J = 5.7 Hz, 1H), 7.97 (d, J = 5.6 Hz, 1H), 7.12-7.04 (m, 1H), 6.97 (s, 1H), 6.70 (t, J = 9.0 Hz, 1H), 6.02 (s, 1H), 4.05-3.94 (m, 1H), 3.77- 3.62 (m, 1H), 3.23 (p, J = 8.2 Hz, 1H), 3.04- 2.84 (m, 1H), 2.53 (dd, J = 13.5, 6.6 Hz, 1H), 2.29 (s, 3H), 2.12 (d, J = 2.0 Hz, 3H), 2.10-1.87 (m, 3H), 1.33-1.27 (m, 2H) ppm. |
| 37 | 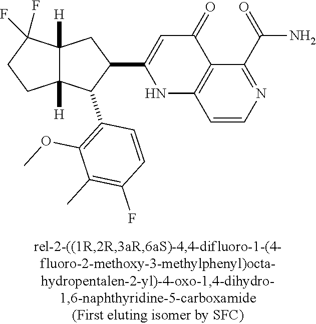 | C-8e | Chiralpak ® IC column, 5 μm particle size, 25 cm × 21.2 mm from Daicel Corporation (Mobile phase: 32% MeOH (supplemented with 20 mM ammonia), 68% CO₂; Flow rate 75 mL/min) | ESI-MS m/z calc. 471.177, found 472.1 (M + 1)+. | ¹H NMR (400 MHz, Chloroform-d) δ 15.21 (s, 1H), 8.88 (s, 1H), 8.51 (d, J = 5.6 Hz, 1H), 7.94 (d, J = 5.6 Hz, 1H), 7.20-6.86 (m, 2H), 6.64 (t, J = 8.7 Hz, 1H), 6.10 (s, 1H), 4.08 (dd, J = 12.5, 8.0 Hz, 1H), 3.76 (s, 3H), 3.69-3.51 (m, 1H), 3.41-3.21 (m, 1H), 3.12-2.82 (m, 1H), 2.52 (dd, J = 13.5, 6.7 Hz, 1H), 2.24-1.85 (m, 6H), 1.45-1.15 (m, 2H) ppm. |
| 38 | 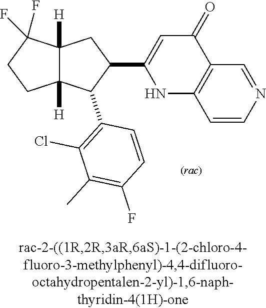 | C-8f | | ESI-MS m/z calc. 432.122, found 433.1 (M + 1)+. | ¹H NMR (400 MHz, Methanol-d₄) δ 9.31 (s, 1H), 8.66 (d, J = 1.1 Hz, 1H), 8.03 (d, J = 6.9 Hz, 1H), 7.46 (dd, J = 8.8, 5.8 Hz, 1H), 7.08 (t, J = 8.8 Hz, 1H), 6.36 (s, 1H), 3.89 (dd, J = 12.1, 9.7 Hz, 1H), 3.62-3.45 (m, 1H), 3.17-2.98 (m, 1H), 2.98-2.84 (m, 1H), 2.68-2.45 (m, 2H), 2.21 (s, 3H), 2.20-2.06 (m, 2H), 1.98-1.71 (m, 2H) ppm; exchangeable H not observed. |
| 39 | 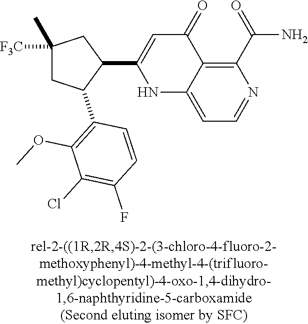 | C-9e | Enantiocel ® C4 column, 5 μm particle size, 25 cm × 30 mm from ColumnTek (Mobile phase: 20% MeOH (supplemented with 0.1% DEA), 75% CO₂; Flow rate 85 mL/min) | ESI-MS m/z calc. 497.113, found 498.5 (M + 1)+. | ¹H NMR (400 MHz, DMSO-d₆) δ 11.61 (br s, 1H), 8.40 (d, J = 5.6 Hz, 1H), 7.46-7.39 (m, 2H), 7.37 (d, J = 5.8 Hz, 1H), 7.24-7.12 (m, 2H), 6.15 (s, 1H), 4.03-3.91 (m, 1H), 3.78 (s, 3H), 3.49- 3.36 (m, 1H), 2.69-2.62 (m, 1H), 2.16-2.06 (m, 1H), 2.01-1.86 (m, 2H), 1.45 (s, 3H) ppm. |
| 40 | 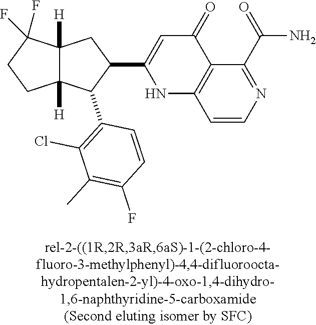 | C-8f | Chiralpak ® IC column, 5 μm particle size, 25 cm × 21.2 mm from Daicel Corporation (Mobile phase: 32% MeOH (supplemented with 20 mM ammonia), 70% CO₂; Flow rate 70 mL/min) | ESI-MS m/z calc. 475.127, found 476.0 (M + 1)+. | ¹H NMR (400 MHz, DMSO-d₆) δ 11.54 (s, 1H), 8.44 (d, J = 5.7 Hz, 1H), 7.51 (dd, J = 8.8, 6.1 Hz, 1H), 7.46-7.33 (m, 2H), 7.20 (t, J = 8.7 Hz, 2H), 5.99 (s, 1H), 3.78 (t, J = 10.8 Hz, 1H), 3.04-2.86 (m, 1H), 2.86- 2.56 (m, 3H), 2.38 (dd, J = 12.8, 7.0 Hz, 1H), 2.24-2.16 (m, 3H), 2.16- 1.92 (m, 2H), 1.84-1.54 (m, 2H) ppm. |
| 41 | 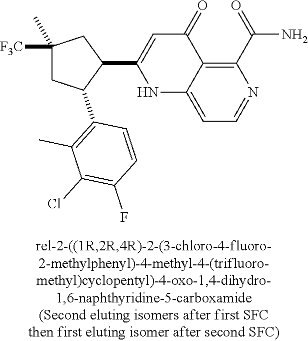 | C-9f | 2 × Enantiocel ® C4 column, 5 μm particle size, 25 cm × 21.2 mm from ColumnTek (Mobile phase: 20% MeOH (supplemented with 0.1% DEA), 80% CO₂; Flow rate 85 mL/min) | ESI-MS m/z calc. 481.118, found 482.5 (M + 1)+. | ¹H NMR (400 MHz, DMSO-d₆) δ 11.61 (s, 1H), 8.44 (d, J = 5.6 Hz, 1H), 7.52-7.29 (m, 3H), 7.27-7.16 (m, 2H), 6.01 (s, 1H), 3.83-3.63 (m, 2H), 2.61 (dd, J = 14.0, 7.7 Hz, 1H), 2.37 (s, 3H), 2.29-2.13 (m, 2H), 1.48-1.43 (m, 1H), 1.41 (s, 3H) ppm. |
| 42 | 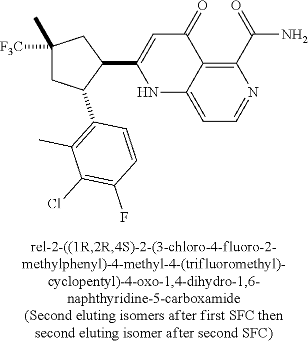 | C-9f | 2 × Enantiocel ® C4 column, 5 μm particle size, 25 cm × 21.2 mm from ColumnTek (Mobile phase: 20% MeOH (supplemented with 0.1% DEA), 80% CO₂; Flow rate 85 mL/min) | ESI-MS m/z calc. 481.118, found 482.5 (M + 1)+. | ¹H NMR (400 MHz, DMSO-d₆) δ 11.67 (s, 1H), 8.44 (d, J = 5.7 Hz, 1H), 7.41-7.30 (m, 3H), 7.25-7.16 (m, 2H), 6.20 (s, 1H), 4.04-3.91 (m, 1H), 3.55-3.41 (m, 1H), 2.65 (dd, J = 13.7, 7.4 Hz, 1H), 2.43 (s, 3H), 2.16 (dd, J = 13.2, 6.9 Hz, 1H), 1.93-1.71 (m, 2H), 1.46 (s, 3H) ppm. |
| 43 | 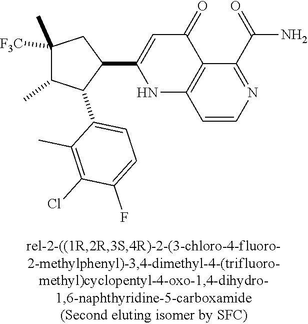 | C-10a | Lux ® Cellulose-4 column, 5 μm particle size, 25 cm × 21.2 mm from Phenomene × (Mobile phase: 32% MeOH (supplemented with 20 mM ammonia), 68% CO₂; Flow rate 70 mL/min) | ESI-MS m/z calc. 495.134, found 496.6 (M + 1)+. | |
| 44 | 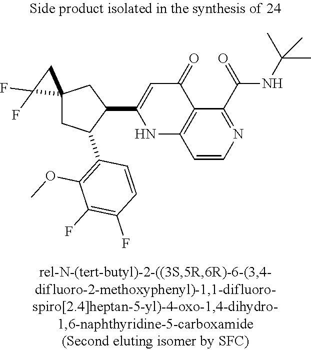 | C-5b | Lux ® Cellulose-4 column, 5 μm particle size, 25 cm × 30 mm from Phenomene × (Mobile phase: 35% MeOH, 65% CO₂; Flow rate 100 mL/min) | ESI-MS m/z calc. 517.199, found 518.2 (M + 1)+. | ¹H NMR (400 MHz, DMSO-d₆) δ 11.58 (s, 1H), 8.41 (br s, 1H), 7.45 (br s, 2H), 7.25 (br t, J = 6.7 Hz, 1H), 7.18- 7.05 (m, 1H), 6.01 (br s, 1H), 4.11-3.97 (m, 1H), 3.82 (d, J = 1.2 Hz, 3H), 3.46 (td, J = 11.4, 7.7 Hz, 1H), 2.40 (br dd, J = 12.8, 7.5 Hz, 1H), 2.25- 2.11 (m, 2H), 2.10-2.00 (m, 1H), 1.71-1.59 (m, 1H), 1.59-1.46 (m, 1H), 1.33 (s, 9H) ppm. |
| 45 | 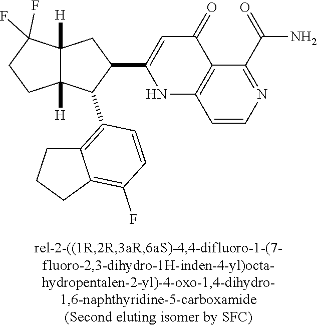 | C-8g | Lux ® Cellulose-4 column, 5 μm particle size, 25 cm × 21.2 mm from Phenomene × (Mobile phase: 50% MeOH (supplemented with 20 mM ammonia), 50% CO₂; Flow rate 70 mL/min) | ESI-MS m/z calc. 467.182, found 468.5 (M + 1)+. | ¹H NMR (400 MHz, DMSO-d₆) δ 11.70 (s, 1H), 8.43 (d, J = 5.8 Hz, 1H), 7.42 (d, J = 5.9 Hz, 1H), 7.36 (s, 1H), 7.18 (s, 1H), 7.07 (dd, J = 8.6, 4.8 Hz, 1H), 6.84 (t, J = 8.6 Hz, 1H), 6.09 (s, 1H), 3.72 (dd, J = 12.6, 7.8 Hz, 1H), 3.50-3.37 (m, 1H), 3.32-3.28 (m, 2H), 3.20 (p, J = 8.2 Hz, 1H), 3.08-2.96 (m, 1H), 2.92 (t, J = 7.8 Hz, 1H), 2.86 (t, J = 7.5 Hz, 2H), 2.39 (dd, J = 13.3, 6.7 Hz, 1H), 2.16-1.93 (m, 4H), 1.34-1.23 (m, 1H), 1.23-1.09 (m, 1H) ppm. |
| 46 | 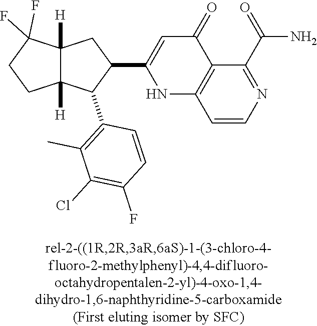 | C-8h | Chiralpak ® IC column, 5 μm particle size, 25 cm × 21.2 mm from Daicel Corporation (Mobile phase: 34% MeOH (supplemented with 20 mM ammonia), 66% CO₂; Flow rate 70 mL/min) | ESI-MS m/z calc. 475.127, found 476.4 (M + 1)+. | ¹H NMR (400 MHz, Methanol-d₄) δ 8.48 (d, J = 5.9 Hz, 1H), 7.52 (d, J = 6.0 Hz, 1H), 7.16 (dd, J = 8.7, 5.2 Hz, 1H), 6.95 (t, J = 8.7 Hz, 1H), 6.21 (s, 1H), 3.86 (dd, J = 12.5, 7.9 Hz, 1H), 3.58-3.48 (m, 1H), 3.39- 3.27 (m, 1H, overlapped with water), 3.09-2.93 (m, 1H), 2.64-2.57 (m, 1H), 2.51 (s, 3H), 2.14- 1.90 (m, 3H), 1.38-1.20 (m, 2H) ppm; exchange- able H not observed. |
| 47 | 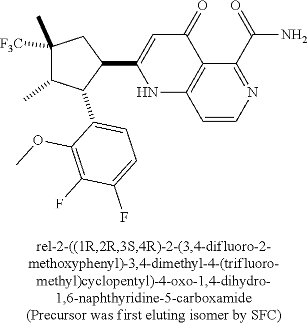 | C-10b | Lux ® i- Cellulose-5 column, 5 μm particle size, 25 cm × 30 mm from Phenomene × (Mobile phase: 30% MeOH, 70% CO₂; Flow rate 100 mL/min) | ESI-MS m/z calc. 495.158, found 496.2 (M + 1)+. | ¹H NMR (400 MHz, Methanol-d₄) δ 8.51 (d, J = 5.9 Hz, 1H), 7.53 (br d, J = 5.6 Hz, 1H), 7.01-6.82 (m, 2H), 6.24 (br s, 1H), 4.21 (br dd, J = 13.2, 7.3 Hz, 1H), 4.04 (d, J = 2.4 Hz, 3H), 3.86-3.72 (m, 1H), 2.86 (dd, J = 14.1, 8.9 Hz, 1H), 2.61-2.49 (m, 1H), 1.82 (dd, J = 13.9, 11.2 Hz, 1H), 1.57 (s, 3H), 0.85-0.77 (m, 3H) ppm; exchangeable H not observed. |
| 48 | 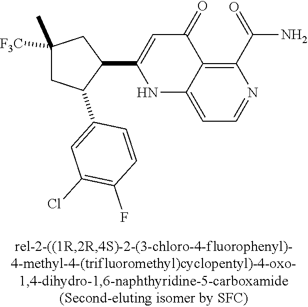 | C-9g | Chiralpak ® IC column, 5 μm particle size, 25 cm × 21.2 mm from Daicel Corporation (Mobile phase: 25% iPrOH (supplemented with 0.1% DEA), 75% CO2; Flow rate 50 mL/min) | ESI-MS m/z calc. 467.102, found 468.5 (M + 1)+. | ¹H NMR (400 MHz, DMSO-d₆) δ 10.95 (br s, 1H), 8.39 (d, J = 5.7 Hz, 1H), 7.54 (d, J = 6.9 Hz, 1H), 7.39 (br s, 1H), 7.35 (d, J = 5.8 Hz, 1H), 7.33-7.26 (m, 2H), 7.19 (br s, 1H), 6.20 (s, 1H), 3.83-3.70 (m, 1H), 3.35- 3.23 (m, 1H), 2.71-2.62 (m, 1H), 2.14-1.98 (m, 2H), 1.84-1.73 (m, 1H), 1.41 (s, 3H) ppm. |
| 49 | 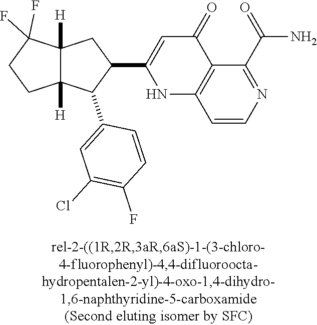 | C-8i | Chiralpak ® IC column, 5 μm particle size, 25 cm × 21.2 mm from Daicel Corporation (Mobile phase: 32% MeOH (supplemented with 20 mM ammonia), 68% CO₂; Flow rate 65 mL/min) | ESI-MS m/z calc. 461.112, found 462.3 (M + 1)+. | ¹H NMR (400 MHz, Methanol-d₄) δ 8.79 (d, J = 6.0 Hz, 1H), 7.96 (d, J = 6.0 Hz, 1H), 7.41 (dd, J = 7.0, 2.2 Hz, 1H), 7.27-7.20 (m, 1H), 7.14 (t, J = 8.8 Hz, 1H), 6.88 (s, 1H), 3.88 (dd, J = 12.7, 8.0 Hz, 1H), 3.64 (td, J = 12.2, 6.7 Hz, 1H), 3.32-3.20 (m, 1H), 3.13-2.96 (m, 1H), 2.67 (dd, J = 13.5, 6.8 Hz, 1H), 2.16-2.07 (m, 2H), 2.07-1.97 (m, 1H), 1.35 (s, 2H) ppm; exchange- able H not observed. |
| 50 | 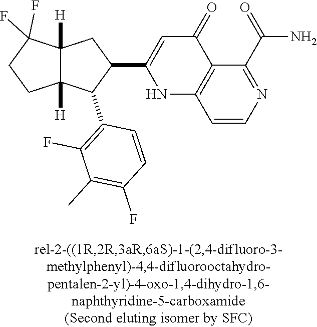 | C-8j | Enantiocel ® C9 column, 5 μm particle size, 25 cm × 21.2 mm from ColumnTek (Mobile phase: 25% MeOH (supplemented with 0.1% DEA), 75% CO₂; Flow rate 64 mL/min) | ESI-MS m/z calc. 459.157, found 460.6 (M + 1)+. | ¹H NMR (400 MHz, DMSO-d₆) δ 11.68 (s, 1H), 8.43 (d, J = 5.7 Hz, 1H), 7.43 (d, J = 5.7 Hz, 1H), 7.38 (br s, 1H), 7.29-7.17 (m, 2H), 6.96 (t, J = 8.7 Hz, 1H), 6.13 (s, 1H), 3.87 (dd, J = 12.5, 8.0 Hz, 1H), 3.52- 3.39 (m, 1H), 3.26-3.13 (m, 1H), 3.06-2.90 (m, 1H), 2.39 (dd, J = 13.0, 6.4 Hz, 1H), 2.12 (s, 3H), 2.10-1.94 (m, 3H), 1.41-1.27 (m, 1H), 1.21- 1.07 (m, 1H) ppm. |
| 51 | 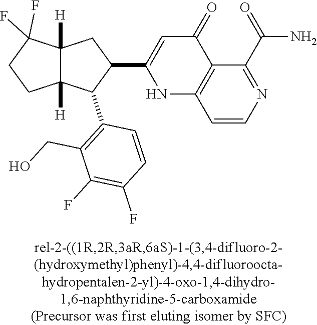 | C-8k | Lux ® i- Amylose-1 column, 5 μm particle size, 25 cm × 30 mm from Phenomene × (Mobile phase: 20% EOH, 80% CO₂; Flow rate 100 mL/min) | ESI-MS m/z calc. 475.152, found 476.2 (M + 1)+. | ¹H NMR (400 MHz, DMSO-d₆) δ 11.66 (br s, 1H), 8.42 (d, J = 5.9 Hz, 1H), 7.41 (d, J = 5.9 Hz, 1H), 7.36 (br s, 1H), 7.31-7.22 (m, 1H), 7.21- 7.10 (m, 2H), 6.12 (s, 1H), 5.34 (br s, 1H), 4.62 (br s, 2H), 4.04 (br dd, J = 12.6, 7.7 Hz, 1H), 3.47 (td, J = 12.2, 6.8 Hz, 1H), 3.30-3.21 (m, 1H), 3.04-2.87 (m, 1H), 2.41 (br dd, J = 12.8, 7.0 Hz, 1H), 2.15- 1.89 (m, 3H), 1.34-1.12 (m, 2H) ppm. |
| 52 | 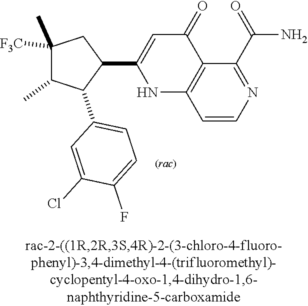 | Mixture of C-12c and C-10c | | ESI-MS m/z calc. 481.118, found 482.5 (M + 1)+. | ¹H NMR (400 MHz, DMSO-d₆) δ 11.66 (s, 1H), 8.45 (d, J = 5.8 Hz, 1H), 7.54 (dd, J = 6.6, 1.8 Hz, 1H), 7.44-7.17 (m, 5H), 6.13 (s, 1H), 4.11 (dd, J = 13.1, 7.0 Hz, 1H), 3.74 (q, J = 10.6 Hz, 1H), 2.74 (dd, J = 14.0, 9.1 Hz, 1H), 2.37 (t, J = 7.3 Hz, 1H), 1.68 (t, J = 12.0 Hz, 1H), 1.51 (s, 3H), 0.69 (d, J = 7.3 Hz, 3H) ppm. |
| 53 | 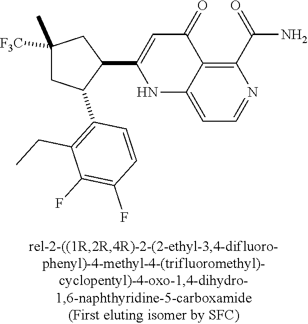 | C-9h | (S,S)- Whelk-O ® 1 column, 5μm particle size, 25 cm × 21.1 mm from Regis Tech- nologies, Inc. (Mobile phase: 18% MeOH (supplemented with 20 mM ammonia), 82% CO₂; Flow rate 72 mL/min) | ESI-MS m/z calc. 479.163, found 480.6 (M + 1)+. | ¹H NMR (400 MHz, Methanol-d₄) δ 8.47 (d, J = 5.9 Hz, 1H), 7.53- 7.48 (m, 1H), 7.29-7.21 (m, 1H), 7.08 (q, J = 8.9 Hz, 1H), 6.21 (s, 1H), 3.75-3.63 (m, 1H), 3.63- 3.51 (m, 1H), 2.80-2.59 (m, 3H), 2.44 (t, J = 12.6 Hz, 1H), 2.25 (dd, J = 13.0, 6.4 Hz, 1H), 1.70- 1.59 (m, 1H), 1.48 (s, 3H), 1.07 (t, J = 7.5 Hz, 3H) ppm; exchangeable H not observed. |
| 54 | 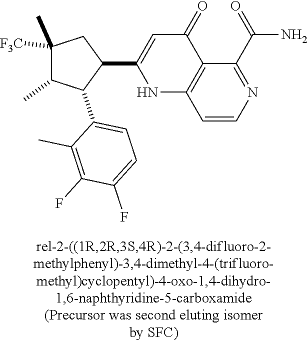 | C-10d | Lux ® Cellulose-2 column, 5 μm particle size, 25 cm × 30 mm from Phenomene × (Mobile phase: 20% MeOH, 80% CO₂; Flow rate 150 mL/min) | ESI-MS m/z calc. 479.163, found 480.2 (M + 1)+. | ¹H NMR (400 MHz, DMSO-d₆) δ 11.55 (br s, 1H), 8.43 (d, J = 5.6 Hz, 1H), 7.40 (br s, 1H), 7.34 (d, J = 5.9 Hz, 1H), 7.31-7.17 (m, 3H), 6.20 (s, 1H), 3.59-3.48 (m, 1H), 3.36-3.24 (m, 1H overlapped with water), 2.70-2.62 (m, 1H), 2.29- 2.18 (m, 4H), 1.92-1.81 (m, 1H), 1.35 (s, 3H), 0.85 (d, J = 6.8 Hz, 3H) ppm. |
| 55 | 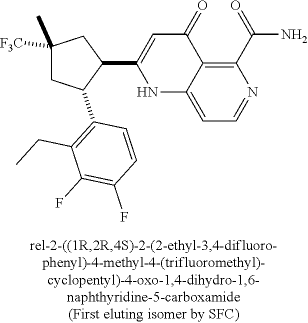 | C-9i | (S,S)- Whelk-O ® 1 column, 5 μm particle size, 25 cm × 21.1 mm from RegisTech- nologies, Inc. (Mobile phase: 18% MeOH (supplemented with 20 mM ammonia), 82% CO₂; Flow rate 72 mL/min) | ESI-MS m/z calc. 479.163, found 480.6 (M + 1)+. | ¹H NMR (400 MHz, Methanol-d₄) δ 8.46 (d, J = 5.9 Hz, 1H), 7.46 (d, J = 5.1 Hz, 1H), 7.20 (dd, J = 8.8, 4.6 Hz, 1H), 7.08 (app q, J = 8.9 Hz, 1H), 6.29 (s, 1H), 3.85- 3.73 (m, 1H), 3.56-3.44 (m, 1H), 2.80-2.67 (m, 3H), 2.21-2.05 (m, 2H), 2.04-1.93 (m, 1H), 1.50 (s, 3H), 1.09 (t, J = 7.5 Hz, 3H) ppm; exchange- able H not observed. |
| 56 | 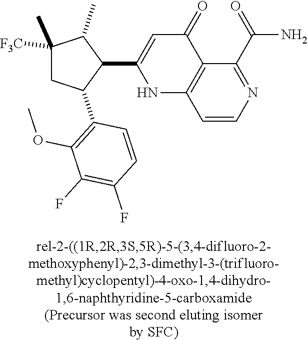 | C-12a | Lux ® Cellulose-2 column, 5 μm particle size, 25 cm × 30 mm from Phenomene × (Mobile phase: 20% MeOH, 80% CO₂; Flow rate 150 mL/min) | ESI-MS m/z calc. 495.158, found 496.1 (M + 1)+. | ¹H NMR (400 MHz, DMSO-d₆) δ 11.58 (br s, 1H), 8.44 (d, J = 5.9 Hz, 1H), 7.48-7.34 (m, 2H), 7.21 (br s, 1H), 7.17- 7.08 (m, 2H), 6.25 (s, 1H), 4.01-3.88 (m, 1H), 3.81 (d, J = 1.2 Hz, 3H), 3.11 (br t, J = 12.1 Hz, 1H), 2.42-2.34 (m, 1H overlapped with solvent), 2.26-2.15 (m, 1H), 2.02 (dd, J = 13.8, 11.1 Hz, 1H), 1.44 (s, 3H), 1.02 (br d, J = 6.6 Hz, 3H) ppm. |
| 57 | 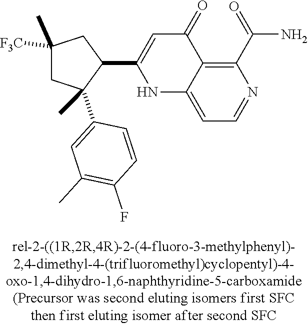 | C-14 | Lux ® i- Amylose-3 column, 5 μm particle size, 25 cm × 30 mm from Phenomene × (Mobile phase: 10% MeOH, 90% CO₂; Flow rate 100 mL/min); rt = 12.87 min then Lux ® Cellulose-4 column, 5 μm particle size, 25 cm × 21.2 mm from Phenomene × | ESI-MS m/z calc. 461.173, found 462.2 (M + 1)+. | ¹H NMR (400 MHz, DMSO-d₆) δ 10.98 (s, 1H), 8.42 (d, J = 5.9 Hz, 1H), 7.45 (br s, 1H), 7.34 (d, J = 5.9 Hz, 1H), 7.24 (br s, 1H), 7.17 (dd, J = 7.5, 2.1 Hz, 1H), 7.14- 7.07 (m, 1H), 7.07-7.00 (m, 1H), 5.98 (d, J = 1.5 Hz, 1H), 3.39 (dd, J = 12.7, 7.3 Hz, 1H), 2.60- 2.52 (m, 2H), 2.48-2.40 (m, 1H), 2.17 (d, J = 1.5 Hz, 3H), 1.94 (d, J = 14.2 Hz, 1H), 1.47 (s, 3H), 1.43 (s, 3H) ppm. |
| | | | (Mobile phase: | | |
| | | | 20% MeOH, | | |
| | | | 80% CO₂; | | |
| | | | Flow rate | | |
| | | | 75 mL/min) | | |
| 58 | 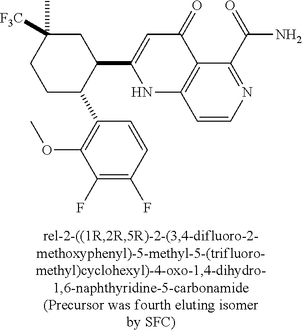 | C-16 | Lux ® Cellulose-3 column, 5 μm particle size, 25 cm × 30 mm from Phenomene × (Mobile phase: 20% MeOH (supplemented with 0.1% DEA), 80% CO₂; Flow rate 100 mL/min) | ESI-MS m/z calc. 495.158, found 496.2 (M + 1)+. | ¹H NMR (400 MHz, DMSO-d₆) δ 11.49 (s, 1H), 8.41 (d, J = 5.7 Hz, 1H), 7.44-7.02 (m, 5H), 6.00 (s, 1H), 3.81 (s, 3H), 3.51-3.38 (m, 1H), 3.29-3.20 (m, 1H, over- lapped with water), 2.19-2.06 (m, 1H), 1.87- 1.67 (m, 5H), 1.38 (s, 3H) ppm. |
| 59 | 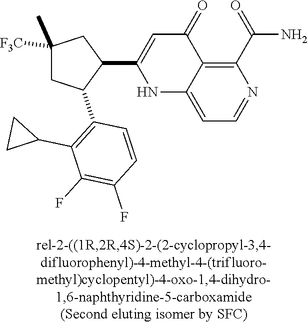 | C-9j | (R,R)- Whelk-O ® 1 column, 5 μm particle size, 25 cm × 21.1 mm from Regis Tech- nologies, Inc. (Mobile phase: 30% 1:1 MeCN:MeOH (supplemented with 0.1% DEA), 70% CO₂; Flow rate 45mL/min) | ESI-MS m/z calc. 491.163, found 492.6 (M + 1)+. | ¹H NMR (400 MHz, Methanol-d₄) δ 8.45 (d, J = 5.9 Hz, 1H), 7.46 (d, J = 6.0 Hz, 1H), 7.16 (dd, J = 9.0, 5.1 Hz, 1H), 7.13-7.04 (m, 1H), 6.30 (s, 1H), 4.30 (td, J = 12.1, 7.1 Hz, 1H), 3.46 (td, J = 11.9, 7.8 Hz, 1H), 2.78 (dd, J = 14.0, 7.7 Hz, 1H), 2.21 (dd, J = 13.4, 7.1 Hz, 1H), 2.15-2.04 (m, 1H), 1.99- 1.89 (m, 1H), 1.71-1.60 (m, 1H), 1.50 (s, 3H), 1.19-1.02 (m, 2H), 0.73- 0.58 (m, 2H) ppm; exchangeable H not observed. |
| 60 | 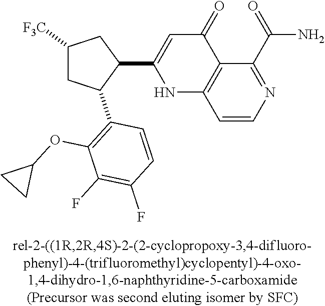 | C-3d | Lux ® Cellulose-2 column, 5 μm particle size, 25 cm × 30 mm from Phenomene × (Mobile phase: 35% MeOH, 65% CO₂; Flow rate 100 mL/min) | ESI-MS m/z calc. 493.142, found 494.1 (M + 1)⁺. | ¹H NMR (400 MHz, DMSO-d₆) δ 11.58 (br s, 1H), 8.44 (d, J = 5.7 Hz, 1H), 7.44-7.36 (m, 2H), 7.24-7.09 (m, 3H), 5.97 (d, J = 1.3 Hz, 1H), 4.19- 4.14 (m, 1H), 3.68 (td, J = 12.1, 6.8 Hz, 1H), 3.38-3.28 (m, 2H, over- lapped with water), 2.43-2.35 (m, 2H), 2.27- 2.19 (m, 1H), 1.77-1.68 (m, 1H), 0.83-0.75 (m, 1H), 0.63-0.50 (m, 3H) ppm. |
| 61 | 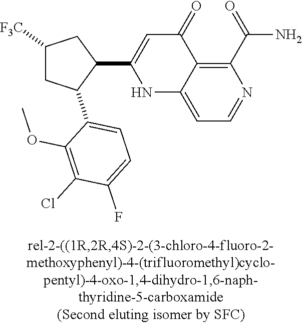 | C-3f | Lux ® Cellulose-4 column, 5 μm particle size, 25 cm × 21.2 mm from Phenomene × (Mobile phase: 32% MeOH (supplemented with 20 mM ammonia), 68% CO₂; Flow rate 68 mL/min) | ESI-MS m/z calc. 483.097, found 484.4 (M + 1)⁺. | ¹H NMR (400 MHz, Methanol-d₄) δ 8.47 (d, J = 5.9 Hz, 1H), 7.48 (d, J = 5.9 Hz, 1H), 7.37 (dd, J = 8.9, 6.0 Hz, 1H), 7.03 (t, J = 8.6 Hz, 1H), 6.25 (s, 1H), 3.95-3.83 (m, 1H), 3.77 (s, 3H), 3.45-3.35 (m, 1H), 3.29- 3.16 (m, 1H), 2.63-2.46 (m, 2H), 2.37-2.24 (m, 1H), 2.01-1.88 (m, 1H) ppm; exchangeable H not observed. |
| 62 | 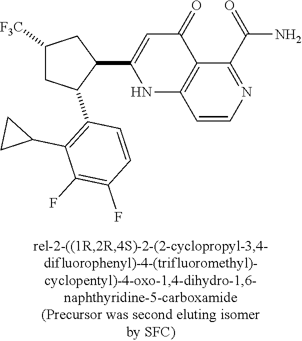 | C-3e | Lux ® Cellulose-2 column, 5 μm particle size, 25 cm × 30 mm from Phenomene × (Mobile phase: 35% MeOH, 65% CO₂; Flow rate 100 mL/min) | ESI-MS m/z calc. 477.148, found 478.2 (M + 1)⁺. | ¹H NMR (400 MHz, DMSO-d₆) δ 11.63 (s, 1H), 8.44 (d, J = 5.9 Hz, 1H), 7.44-7.34 (m, 2H), 7.31-7.23 (m, 2H), 7.20 (br s, 1H), 6.02 (s, 1H), 4.20-4.08 (m, 1H), 3.47- 3.36 (m, 1H), 3.35-3.26 (m, 1H, overlapped with water), 2.61-2.52 (m, 1H), 2.46-2.38 (m, 1H), 2.37-2.25 (m, 1H), 1.72- 1.53 (m, 2H), 1.11-0.96 (m, 2H), 0.78-0.65 (m, 1H), 0.45-0.30 (m, 1H) ppm. |
| 63 | 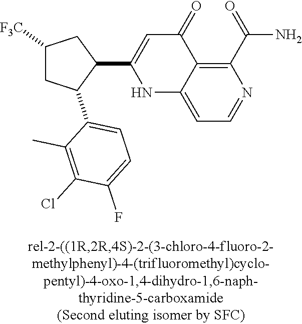 | C-3g | Chiralpak ® IC column, 5 μm particle size, 25 cm × 21.2 mm from Daicel Corporation (Mobile phase: 26% MeOH (supplemented with 20 mM ammonia), 74% CO₂; Flow rate 62 mL/min) | ESI-MS m/z calc. 467.102, found 468.7 (M + 1)⁺. | ¹H NMR (400 MHz, Methanol-d₄) δ 8.47 (d, J = 5.9 Hz, 1H), 7.47 (d, J = 5.9 Hz, 1H), 7.35 (dd, J = 8.8, 5.2 Hz, 1H), 7.06 (t, J = 8.7 Hz, 1H), 6.23 (s, 1H), 3.88-3.76 (m, 1H), 3.50-3.38 (m, 1H), 3.28-3.16 (m, 1H), 2.62-2.49 (m, 2H), 2.37 (s, 3H), 2.35-2.24 (m, 1H), 1.87-1.74 (m, 1H) ppm; exchangeable H not observed. |
| 64 | 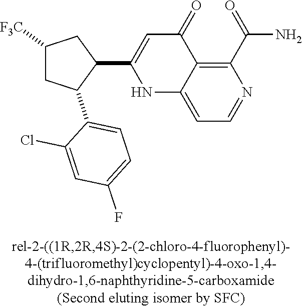 | C-3h | Lux ® Cellulose-4 column, 5 μm particle size, 15 cm × 30 mm from Phenomene × (Mobile phase: 34% MeOH (supplemented with 20 mM ammonia), 66% CO₂; Flow rate 95 mL/min) | ESI-MS m/z calc. 453.087, found 453.9 (M + 1)⁺. | ¹H NMR (400 MHz, Methanol-d₄) δ 8.48 (d, J = 5.9 Hz, 1H), 7.57- 7.45 (m, 2H), 7.17-7.04 (m, 2H), 6.19 (s, 1H), 4.10-3.97 (m, 1H), 3.50- 3.38 (m, 1H), 3.29-3.18 (m, 1H), 2.63-2.51 (m, 2H), 2.39-2.26 (m, 1H), 1.98-1.85 (m, 1H) ppm; exchangeable H not observed. |
| 65 | 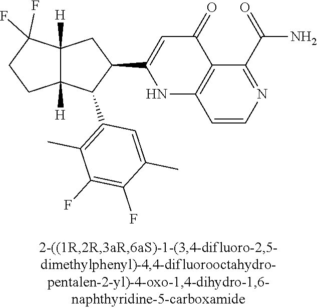 [SMILES: Cc1cc([C@@H]2[C@@H]3CCC(F)(F)[C@@H]3C[C@H]2c2cc(=O)c3c(C(N)=O)nccc3[nH]2)c(C)c(F)c1F] | C-8l | | ESI-MS m/z calc. 473.173, found 474.2 (M + 1)⁺. | ¹H NMR (400 MHz, DMSO-d₆) δ 11.69 (br s, 1H), 8.43 (d, J = 5.9 Hz, 1H), 7.50-7.32 (m, 2H), 7.20 (br s, 1H), 7.02 (br d, J = 6.6 Hz, 1H), 6.17- 6.10 (m, 1H), 3.86-3.75 (m, 1H), 3.50-3.40 (m, 1H), 3.25-3.13 (m, 1H), 3.03-2.87 (m, 1H), 2.45- 2.36 (m, 1H), 2.30-2.22 (m, 3H), 2.20-2.14 (m, 3H), 2.12-1.90 (m, 3H), 1.33-1.07 (m, 2H) ppm. |
| 66 | 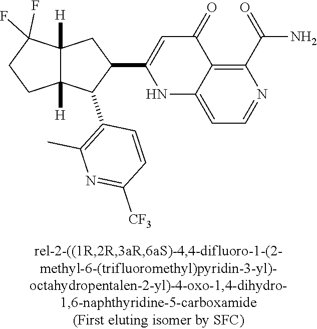 | C-8m | Chiralpak ® IC column, 5 μm particle size, 25 cm × 21.2 mm from Daicel Corporation (Mobile phase: 30% MeOH (supplemented with 20 mM ammonia), 70% CO₂; Flow rate 70 mL/min) | ESI-MS m/z calc. 492.158, found 493.5 (M + 1)⁺. | ¹H NMR (400 MHz, Methanol-d₄) δ 8.50 (d, J = 5.9 Hz, 1H), 7.85 (d, J = 8.0 Hz, 1H), 7.56 (d, J = 5.9 Hz, 1H), 7.52 (d, J = 8.0 Hz, 1H), 6.27 (s, 1H), 3.96 (dd, J = 12.5, 7.9 Hz, 1H), 3.67-3.56 (m, 1H), 3.51-3.41 (m, 1H), 3.17-3.02 (m, 1H), 2.74 (s, 3H), 2.68 (dd, J = 13.4, 6.6 Hz, 1H), 2.17-1.97 (m, 3H), 1.43-1.18 (m, 2H) ppm; exchangeable H not observed. |
| 67 | 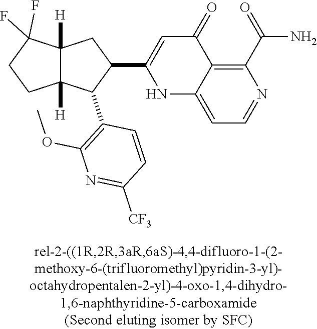 | C-8n | Chiralpak ® IC column, 5 μm particle size, 25 cm × 21.2 mm from Daicel Corporation (Mobile phase: 24% MeOH (supplemented with 20 mM ammonia), 74% CO₂; Flow rate 70 mL/min) | ESI-MS m/z calc. 508.153, found 509.6 (M + 1)⁺. | ¹H NMR (400 MHz, Methanol-d₄) δ 8.50 (d, J = 5.9 Hz, 1H), 7.90 (d, J = 7.5 Hz, 1H), 7.55 (d, J = 6.0 Hz, 1H), 7.31 (d, J = 7.6 Hz, 1H), 6.21 (s, 1H), 3.91 (s, 3H), 3.57- 3.51 (m, 2H), 3.13-2.97 (m, 2H), 2.62-2.42 (m, 2H), 2.22-2.03 (m, 2H), 1.98-1.84 (m, 1H), 1.76- 1.68 (m, 1H) ppm; exchangeable H not observed. |
| 68 | 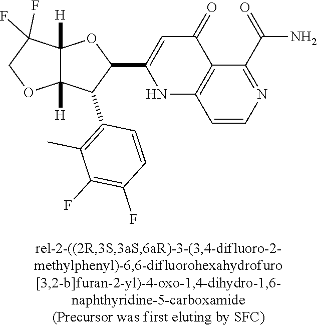 | C-20 | Lux ® i- Amylose- 3 column, 5 μm particle size, 25 cm × 30 mm from Phenomene × (Mobile phase: 20% MeOH, 80% CO₂; Flow rate 80 mL/min) | ESI-MS m/z calc. 463.116, found 464.2 (M + 1)⁺. | ¹H NMR (400 MHz, DMSO-d₆) δ 11.67 (br s, 1H), 8.48 (d, J = 5.9 Hz, 1H), 7.55 (br d, J = 5.6 Hz, 1H), 7.49-7.34 (m, 2H), 7.33-7.15 (m, 2H), 6.06 (br s, 1H), 5.48 (d, J = 11.0 Hz, 1H), 5.10 (t, J = 4.2 Hz, 1H), 4.99 (dd, J = 9.2, 4.3 Hz, 1H), 4.32-4.17 (m, 1H), 4.15- 4.01 (m, 1H), 3.89 (br dd, J = 10.8, 4.2 Hz, 1H), 2.15 (d, J = 2.0 Hz, 3H) ppm. |
| 69 | 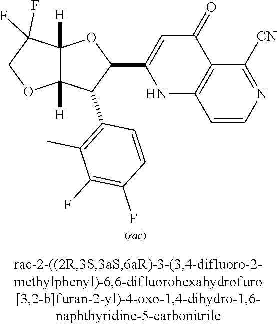 | C-20 | | ESI-MS m/z calc. 445.105, found 445.9 (M + 1)⁺. | ¹H NMR (400 MHz, Methanol-d₄) δ 8.68 (d, J = 5.9 Hz, 1H), 7.85 (d, J = 5.9 Hz, 1H), 7.44 (ddd, J = 8.8, 4.9, 1.8 Hz, 1H), 7.19-7.08 (m, 1H), 6.06 (s, 1H), 5.46 (d, J = 11.0 Hz, 1H), 5.06-4.96 (m, 2H), 4.32- 4.17 (m, 1H), 4.10-3.98 (m, 1H), 3.78 (dd, J = 11.0, 4.1 Hz, 1H), 2.13 (s, 3H) ppm; exchange- able H not observed. |
| 70 | 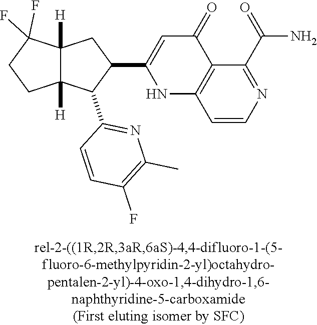 [SMILES: Cc1nc([C@@H]2[C@@H]3CCC(F)(F)[C@@H]3C[C@H]2c2cc(=O)c3c(C(N)=O)nccc3[nH]2)ccc1F] | C-8o | Chiralpak ® IC column, 5 μm particle size, 25 cm × 21.2 mm from Daicel Corporation (Mobile phase: 36% MeOH (supplemented with 20 mM ammonia), 64% CO₂; Flow rate 64 mL/min) | ESI-MS m/z calc. 442.162, found 443.4 (M + 1)⁺. | ¹H NMR (400 MHz, Methanol-d₄) δ 8.49 (d, J = 5.9 Hz, 1H), 7.56 (d, J = 6.0 Hz, 1H), 7.37 (dd, J = 9.3, 8.4 Hz, 1H), 7.18 (dd, J = 8.5, 3.6 Hz, 1H), 6.28 (s, 1H), 3.85- 3.67 (m, 2H), 3.35-3.29 (m, 1H), 3.12-2.96 (m, 1H), 2.58 (dd, J = 13.3, 6.4 Hz, 1H), 2.41 (d, J = 3.0 Hz, 3H), 2.15-1.93 (m, 3H), 1.46-1.36 (m, 2H) ppm; exchangeable H not observed. |
| 71 | 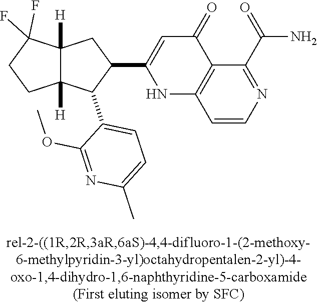 | C-8p | Chiralpak ® IC column, 5 μm particle size, 25 cm × 21.2 mm from Daicel Corporation (Mobile phase: 38% MeOH (supplemented with 20 mM ammonia), 62% CO₂; Flow rate 70 mL/min) | ESI-MS m/z calc. 454.182, found 455.6 (M + 1)⁺. | ¹H NMR (400 MHz, Methanol-d₄) δ 8.49 (d, J = 5.9 Hz, 1H), 7.56 (d, J = 5.9 Hz, 1H), 7.38 (d, J = 7.5 Hz, 1H), 6.67 (d, J = 7.5 Hz, 1H), 6.25 (s, 1H), 3.96 (s, 3H), 3.83 (dd, J = 12.8, 7.9 Hz, 1H), 3.53-3.39 (m, 2H), 3.09-2.91 (m, 1H), 2.60 (dd, J = 13.4, 6.7 Hz, 1H), 2.35 (s, 3H), 2.13- 1.89 (m, 3H), 1.44-1.30 (m, 1H), 1.29-1.15 (m, 1H) ppm; exchangeable H not observed. |
| 72 | 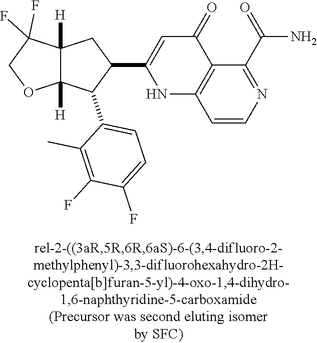 | C-21 | Lux ® Cellulose-2 column, 5 μm particle size, 25 cm × 30 mm from Phenomene × (Mobile phase: 40% EtOH (supplemented with 0.1% DEA), 60% CO₂; Flow rate 100 mL/min) | ESI-MS m/z calc. 461.136, found 462.1 (M + 1)⁺. | ¹H NMR (400 MHz, DMSO-d₆) δ 11.76 (br s, 1H), 8.44 (d, J = 5.6 Hz, 1H), 7.43-7.35 (m, 2H), 7.20 (br s, 1H), 7.18- 7.08 (m, 2H), 6.11 (s, 1H), 4.84 (dd, J = 6.1, 4.6 Hz, 1H), 4.13-4.02 (m, 1H), 3.87-3.75 (m, 2H), 3.66-3.55 (m, 1H), 3.29-3.17 (m, 1H), 2.51 (overlapped by DMSO, m, 1H), 2.32 (d, J = 2.2 Hz, 3H), 2.17-2.05 (m, 1H) ppm. |

*SFC separation performed at the last step except if specified otherwise.

### Intermediate C-1

### rac-(1R,2R)-2-(3,4-Difluoro-2-methoxyphenyl)-4,4-dimethylcyclopentane-1-carboxylic acid

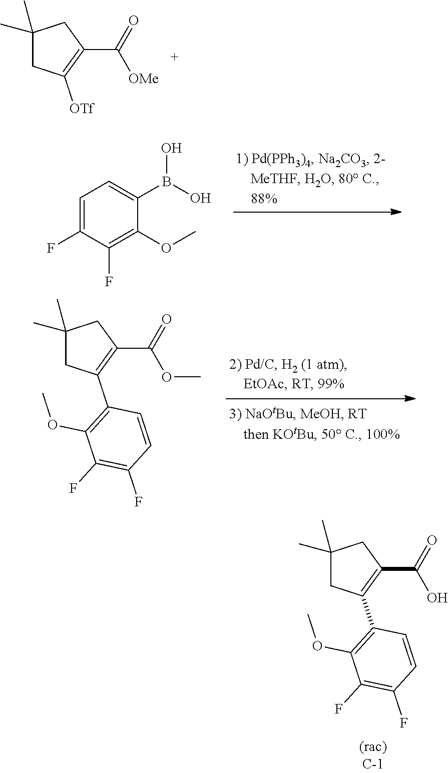

### Step 1

Nitrogen gas was passed through a stirred solution of methyl 4,4-dimethyl-2-(((trifluoromethyl)sulfonyl)oxy)cyclopent-1-ene-1-carboxylate (13.35 g, 42.532 mmol) and (3,4-difluoro-2-methoxyphenyl)boronic acid (9.6 g, 51.081 mmol) in a mixture of 2-MeTHF (200 mL) and a 2 M aqueous Na₂CO₃ solution (53 mL, 106 mmol) for 30 min. Pd(PPh₃)₄(2.4 g, 2.077 mmol) was added and the reaction mixture was stirred at 80° C. for 3 h. The reaction was cooled to ambient temperature, diluted with water (200 mL) and EtOAc (200 mL). The aqueous layer was separated and extracted with EtOAc (100 mL). The combined organic extracts were dried (Na₂SO₄), filtered and concentrated in vacuo. Purification by flash chromatography (SiO₂, 0 to 10% EtOAc in heptane) gave methyl 2-(3,4-difluoro-2-methoxyphenyl)-4,4-dimethylcyclopent-1-ene-1-carboxylate (11.12 g, 88%) as a pale yellow oil. ¹H NMR (400 MHz, Chloroform-d) δ 6.89-6.74 (m, 2H), 3.87 (d, J=1.7 Hz, 3H), 3.58 (s, 3H), 2.63-2.59 (m, 2H), 2.58-2.54 (m, 2H), 1.18 (s, 6H) ppm. ESI-MS m/z calc. 296.122, found 297.2 (M+1)⁺.

### Step 2

Under a nitrogen atmosphere, 5% Pd/C (3.84 g, 1.804 mmol) was added to a solution of methyl 2-(3,4-difluoro-2-methoxyphenyl)-4,4-dimethylcyclopent-1-ene-1-carboxylate (10.9 g, 36.749 mmol) in EtOAc (250 mL). Hydrogen gas was passed through the solution for 30 min and the reaction mixture was stirred under an atmospheric pressure of hydrogen gas for 16 h. Additional 5% Pd/C (1 g, 0.470 mmol) was added and the reaction mixture was stirred under an atmospheric pressure of hydrogen gas for a further 16 h. The mixture was filtered through a pad of Celite®, washing the filtered cake with EtOAc (150 mL). The filtrates were concentrated in vacuo to give methyl dia-(1R′,2R²)-2-(3,4-difluoro-2-methoxyphenyl)-4,4-dimethylcyclopentane-1-carboxylate (10.89 g, 99%) as a 66:34 mixture of cis and trans diastereomers and as a colourless oil. ¹H NMR (400 MHz, Chloroform-d) δ 6.94-6.87 (m, 0.34H), 6.87-6.71 (m, 1.66H), 4.02 (d, J=2.2 Hz, 1.98H), 3.98-3.88 (m, 1.66H), 3.77 (td, J=10.9, 8.2 Hz, 0.34H), 3.59 (s, 1.02H), 3.34 (ddd, J=9.7, 8.4, 6.6 Hz, 0.66H), 3.19 (s, 1.98H), 3.10-3.00 (m, 0.34H), 2.06-1.89 (m, 2.02H), 1.86-1.79 (m, 0.34H), 1.75 (ddd, J=13.3, 8.4, 1.6 Hz, 0.66H), 1.60 (ddd, J=12.2, 6.4, 1.5 Hz, 0.66H), 1.55-1.48 (m, 0.34H), 1.24 (s, 1.98H), 1.15 (s, 1.02H), 1.12 (s, 1.02H), 1.10 (s, 1.98H) ppm. ESI-MS m/z calc. 298.138, found 299.1 (M+1)⁺.

### Step 3

NaO^{t}Bu (19 mg, 0.198 mmol) was added to a solution of a 66:34 mixture of cis and trans diastereomers of methyl dia-(1R′,2R²)-2-(3,4-difluoro-2-methoxyphenyl)-4,4-dimethylcyclopentane-1-carboxylate (40 mg, 0.113 mmol) in MeOH (1 mL) at ambient temperature and the reaction mixture was stirred at ambient temperature for 16 h. KO^{t}Bu (20 mg, 0.178 mmol) was added and the mixture was stirred at 50° C. for 4 h. The reaction was cooled to ambient temperature and partitioned between MTBE (20 mL) and a 0.25 N aqueous HCl solution (20 mL). The aqueous layer was separated and extracted with MTBE (20 mL). The combined organic extracts were dried (Na₂SO₄), filtered and concentrated in vacuo to give rac-(1R,2R)-2-(3,4-difluoro-2-methoxyphenyl)-4,4-dimethylcyclopentane-1-carboxylic acid (C-1, 38 mg, 100%) as a colourless oil. ¹H NMR (400 MHz, Chloroform-d) δ 6.94-6.87 (m, 1H), 6.85-6.76 (m, 1H), 3.91 (d, J=2.0 Hz, 3H), 3.76 (td, J=10.9, 8.2 Hz, 1H), 3.10-3.01 (m, 1H), 1.95 (ddd, J=19.1, 12.8, 8.4 Hz, 2H), 1.88-1.80 (m, 1H), 1.59-1.49 (m, 1H), 1.14 (s, 3H), 1.12 (s, 3H) ppm; exchangeable H not observed. ESI-MS m/z calc. 284.122, found 285.2 (M+1)⁺.

### Intermediate C-2

### rac-(2R,3R)-3-(3,4-Difluoro-2-methoxyphenyl)spiro[4.5]decane-2-carboxylic acid

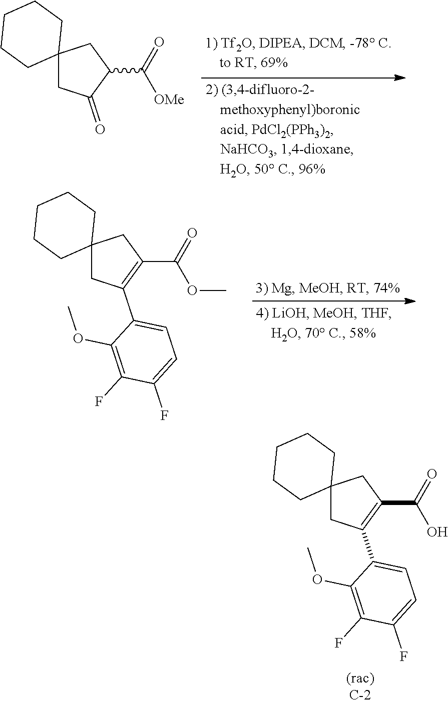

### Step 1

Under a nitrogen atmosphere, Tf₂O (1.5 mL, 8.916 mmol) was added dropwise to a stirred solution of methyl rac-3-oxospiro[4.5]decane-2-carboxylate (1.5 g, 7.134 mmol) and DIPEA (1.8 mL, 10.33 mmol) in DCM (16.5 mL) at −78° C. and the reaction mixture was stirred at −78° C. for 5 min and at ambient temperature for 1 h. The reaction was quenched by addition of a saturated NH₄Cl solution. The aqueous phase was separated and extracted with DCM. The combined organic extracts were dried (MgSO₄), filtered and concentrated in vacuo. Purification by flash chromatography (SiO₂, 0 to 60% EtOAc in hexanes) gave methyl 3-(((trifluoromethyl)sulfonyl)oxy)spiro[4.5]dec-2-ene-2-carboxylate (1.69 g, 69%) as an orange oil. ¹H NMR (400 MHz, Chloroform-d) δ 3.79 (s, 3H), 2.56 (t, J=2.6 Hz, 2H), 2.50 (t, J=2.6 Hz, 2H), 1.56-1.37 (m, 10H) ppm. ESI-MS m/z calc. 342.075, found 343.1 (M+1)⁺.

### Step 2

In a vial, nitrogen gas was passed through a stirred mixture of methyl 3-(((trifluoromethyl)sulfonyl)oxy)spiro[4.5]dec-2-ene-2-carboxylate (1 g, 2.921 mmol), PdCl₂(PPh₃)₂(164 mg, 0.234 mmol) and (3,4-difluoro-2-methoxyphenyl)boronic acid (600 mg, 3.193 mmol) in a mixture of 1,4-dioxane (6.6 mL) and a saturated aqueous NaHCO₃ solution (3.4 mL) for 5 min. The vial was sealed and the reaction mixture was stirred at 50° C. for 1.5 h. The mixture was cooled to ambient temperature and partitioned between EtOAc and water. The organic layer was separated, washed with brine, dried (MgSO₄), filtered and concentrated in vacuo. Purification by flash chromatography (SiO₂, 1 to 60% EtOAc in hexanes) gave methyl 3-(3,4-difluoro-2-methoxyphenyl)spiro[4.5]dec-2-ene-2-carboxylate (945 mg, 96%) as a yellow oil. ¹H NMR (400 MHz, Chloroform-d) δ 6.95-6.72 (m, 2H), 3.86 (d, J=1.8 Hz, 3H), 3.58 (s, 3H), 2.60 (dt, J=17.3, 2.3 Hz, 4H), 1.59-1.40 (m, 10H) ppm. ESI-MS m/z calc. 336.154, found 337.1 (M+1)⁺.

### Step 3

Mg (315 mg, 11.97 mmol) was added to a solution of methyl 3-(3,4-difluoro-2-methoxyphenyl)spiro[4.5]dec-2-ene-2-carboxylate (500 mg, 1.486 mmol) in MeOH (15.5 mL) and the reaction mixture was vigorously stirred at ambient temperature for 2 h. The mixture was partitioned between 1 N aqueous HCl and EtOAc. The aqueous layer was separated and extracted with EtOAc. The combined organic layers were washed with brine, dried (MgSO₄), filtered and concentrated in vacuo. Purification by flash chromatography (SiO₂, 1 to 60% EtOAc in hexanes) gave methyl dia-(2R¹,3R²)-3-(3,4-difluoro-2-methoxyphenyl)spiro[4.5]decane-2-carboxylate (370 mg, 74%) as an orange oil. ESI-MS m/z calc. 338.169, found 339.2 (M+1)⁺.

### Step 4

In a vial, LiOH·H₂O (186 mg, 4.433 mmol) was added to a stirred solution of methyl dia-(2R¹,3R²)-3-(3,4-difluoro-2-methoxyphenyl)spiro[4.5]decane-2-carboxylate (300 mg, 0.887 mmol) in a mixture of THF (5.1 mL), MeOH (2.6 mL) and water (2.6 mL). The vial was capped and placed in a pre-heated heating block set at 70° C. for 5 h. The reaction was cooled to ambient temperature and partitioned between 1 N aqueous HCl and EtOAc. The aqueous layer was separated and extracted with EtOAc. The combined organic extracts were washed with water, brine, dried (MgSO₄), filtered and concentrated in vacuo. Purification by flash chromatography (SiO₂, 0 to 60% of EtOAc in hexanes) gave rac-(2R,3R)-3-(3,4-difluoro-2-methoxyphenyl)spiro[4.5]decane-2-carboxylic acid 2-(3,4-difluoro-2-methoxy-phenyl)spiro[4.5]decane-3-carboxylic acid (C-2, 167 mg, 58%) as a yellow oil. ESI-MS m/z calc. 324.154, found 325.3 (M+1)⁺.

The following Intermediates were made using the method described in Intermediate C-2, except that, in Step 1, different starting materials were used in place of methyl rac-3-oxospiro[4.5]decane-2-carboxylate. In Step 2, in some occurrences, different Suzuki coupling partners were used in place of (3,4-difluoro-2-methoxyphenyl)boronic acid. In the case of Intermediate C-18, Steps 2, 3 and 4 were respectively carried out at 75, 50 and 50° C.:

| | | LC/MS | | |
|---|---|---|---|---|
| Inter. | | (m/z | Found | |
| No. | Intermediate Name | calc.) | M + 1 | NMR (shifts in ppm) |
| C - 3a | dia-(1R¹,2R¹,4S²)-2-(4- | 320.104 | 321.2 | ¹H NMR (400 MHz, Chloroform-d) δ |
| | fluoro-2-methoxy-3- | | | 7.10-6.96 (m, 1H), 6.87-6.76 (m, 1H), |
| | methylphenyl)-4- | | | 3.74-3.71 (m, 3H), 3.76-3.65 (m, 1H), |
| | (trifluoromethyl)cyclopentane- | | | 3.12-2.98 (m, 1H), 2.97-2.84 (m, 1H), |
| | 1-carboxylic acid | | | 2.49-2.24 (m, 2H), 2.20 (d, J = 2.1 Hz, |
| | | | | 3H), 2.19-2.11 (m, 1H), 1.91-1.71 (m, |
| | | | | 1H) ppm; exchangeable H not observed. |
| C - 6 | Mixture of 8 isomers of | 310.138 | 311.1 | |
| | dia-(1R¹,2R¹,3aR²,7aR²)- | | | |
| | 1-(3,4-difluoro-2- | | | |
| | methoxyphenyl)octahydro- | | | |
| | 1H-indene-2-carboxylic | | | |
| | acid and dia- | | | |
| | (1R¹,2R¹,3aS²,7aR²)-1- | | | |
| | (3,4-difluoro-2- | | | |
| | methoxyphenyl)octahydro- | | | |
| | 1H-indene-2-carboxylic | | | |
| | acid | | | |
| C - 7 | dia-(1R¹,2R¹,3R²)-2-(3,4- | 270.107 | 271.2 | |
| | difluoro-2- | | | |
| | methoxyphenyl)-3- | | | |
| | methylcyclopentane-1- | | | |
| | carboxylic acid | | | |
| C - 18 | dia-(1R¹,2R¹,4R²)-4-(tert- | 326.169 | 327.2 | |
| | butyl)-2-(3,4-difluoro-2- | | | |
| | methoxyphenyl)cyclohexane- | | | |
| | 1-carboxylic acid | | | |

The following Intermediate was made using the method described in Intermediate C-2, except that Step 1 was omitted. In Step 2, a different starting material was used in place of methyl rac-3-oxospiro[4.5]decane-2-carboxylate and the reaction was carried out at 75° C. In Step 3, the reaction was carried out at reflux of the solvent:

| | | LC/MS | |
|---|---|---|---|
| Inter. | | (m/z | Found |
| No. | Intermediate Name | calc.) | M + 1 |
| C - 17 | rac-(1R,2R)-2-(3,4- | 306.088 | 307.2 |
| | difluoro-2- | | |
| | methoxyphenyl)-5,5- | | |
| | difluorocyclohexane-1- | | |
| | carboxylic acid | | |

The following Intermediates were made using the method described in Intermediate C-2, except that, in Step 1, a different starting material was used in place of methyl rac-3-oxospiro[4.5]decane-2-carboxylate. The reaction was carried out at 0° C. in the case of C-3f and at −78° C. in the case of C-3g and C-3h. In Step 2, different Suzuki coupling partners were used in place of (3,4-difluoro-2-methoxyphenyl)boronic acid. Step 3 was carried out at 0° C. The conditions used in Step 4 were those of Intermediate C-16 Step 5:

| | | LC/MS | |
|---|---|---|---|
| Inter. | | (m/z | Found |
| No. | Intermediate Name | calc.) | M + 1 |
| C - 3f | rac-(1R,2R,4S)-2-(3- | 340.049 | 341.2 |
| | chloro-4-fluoro-2- | | |
| | methoxyphenyl)-4- | | |
| | (trifluoromethyl)cyclopentane- | | |
| | 1-carboxylic acid | | |
| C - 3g | rac-(1R,2R,4S)-2-(3- | 324.054 | 325.3 |
| | chloro-4-fluoro-2- | | |
| | methylphenyl)-4- | | |
| | (trifluoromethyl)cyclopentane- | | |
| | 1-carboxylic acid | | |
| C - 3h | rac-(1R,2R,4S)-2-(2- | 310.038 | 311.2 |
| | chloro-4-fluorophenyl)-4- | | |
| | (trifluoromethyl)cyclopentane- | | |
| | 1-carboxylic acid | | |

### Intermediate C-3d

### rac-(1R,2R,4S)-2-(2-Cyclopropoxy-3,4-difluorophenyl)-4-(trifluoromethyl)cyclopentane-1-carboxylic acid

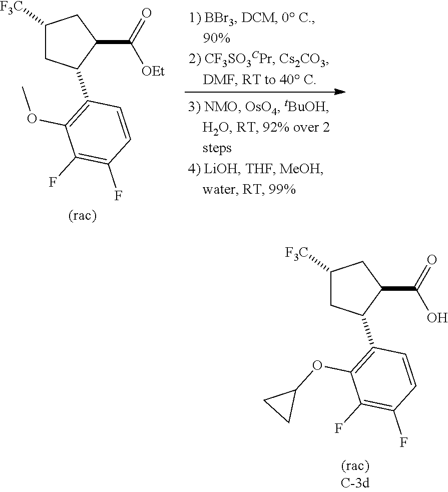 [SMILES: CCOC(=O)[C@@H]1C[C@@H](C(F)(F)F)C[C@H]1c1ccc(F)c(F)c1OC ; O=C(O)[C@@H]1C[C@@H](C(F)(F)F)C[C@H]1c1ccc(F)c(F)c1OC1CC1]

### Step 1

Ethyl rac-(1R,2R,4S)-2-(3,4-difluoro-2-methoxyphenyl)-4-(trifluoromethyl)cyclopentane-1-carboxylate was prepared using the method described in Intermediate C-8a except that Steps 1 to 6 were omitted. In Steps 7 and 8, ethyl rac-2-oxo-4-(trifluoromethyl)cyclopentane-1-carboxylate was used as the starting material in place of rac-(3aR,6aR)-4,4-difluorohexahydropentalen-1(2H)-one and the conditions were those of Intermediate C-2 Steps 1 and 2. The hydrogenation Step 9 was carried out in EtOH at 60 psi and 60° C. and Pd(OH)₂ was used as the catalyst in place of Pd/C. Step 10 was omitted.

BBr₃ (264 mg, 0.1 mL, 1.054 mmol) was added to a stirred solution of ethyl rac-(1R,2R,4S)-2-(3,4-difluoro-2-methoxyphenyl)-4-(trifluoromethyl)cyclopentane-1-carboxylate (210 mg, 0.537 mmol) in DCM (4 mL) at 0° C. and the reaction mixture was stirred at 0° C. for 2.5 h. The reaction was quenched by slowly pouring the mixture over a stirred mixture of a saturated aqueous NaHCO₃ solution (15 mL) and DCM (20 mL). The aqueous layer was separated and extracted with DCM (20 mL). The combined organic layers were dried (Na₂SO₄), filtered and concentrated in vacuo. Purification by flash chromatography (SiO₂, 0 to 30% EtOAc in heptane) gave ethyl rac-(1R,2R,4S)-2-(3,4-difluoro-2-hydroxyphenyl)-4-(trifluoromethyl)cyclopentane-1-carboxylate (180 mg, 90%) as a clear oil. ESI-MS m z calc. 338.094, found 339.1 (M+1)⁺.

### Step 2

Cs₂CO₃ (320 mg, 0.982 mmol) was added to a solution of ethyl rac-(1R,2R,4S)-2-(3,4-difluoro-2-hydroxyphenyl)-4-(trifluoromethyl)cyclopentane-1-carboxylate (175 mg, 0.471 mmol) in DMF (3 mL) and the reaction mixture was stirred at ambient temperature for 30 min. A solution of CF₃SO₃^{c}Pr (312 mg, 1.477 mmol) in DMF (1 mL) was added and the mixture was stirred at 40° C. for 4 h. The reaction was cooled to ambient temperature and partitioned between water (50 mL) and MTBE (30 mL). The aqueous layer was separated and extracted with MTBE (2×30 mL). The combined organic layers were washed with water (4×40 mL) and brine (40 mL), dried (Na₂SO₄), filtered and concentrated in vacuo to give a crude mixture of ethyl rac-(1R,2R,4S)-2-(2-cyclopropoxy-3,4-difluorophenyl)-4-(trifluoromethyl)cyclopentane-1-carboxylate containing 13% of ethyl rac-(1R,2R,4S)-2-(2-(allyloxy)-3,4-difluorophenyl)-4-(trifluoromethyl)cyclopentane-1-carboxylate (200 mg) as a brown oil. ESI-MS m z calc. 378.125, found 379.1 (M+1)⁺.

### Step 3

NMO (21 mg, 0.179 mmol) and a solution of OsO₄ (12.2 mg, 2.5% w/w in ^{t}BuOH, 0.001 mmol) in ^{t}BuOH (1 mL) were successively added to a solution of a crude mixture of ethyl rac-(1R,2R,4S)-2-(2-cyclopropoxy-3,4-difluorophenyl)-4-(trifluoromethyl)cyclopentane-1-carboxylate containing 13% of ethyl rac-(1R,2R,4S)-2-(2-(allyloxy)-3,4-difluorophenyl)-4-(trifluoromethyl)cyclopentane-1-carboxylate (200 mg) in a mixture of ^{t}BuOH (3 mL) and water (3 mL) at ambient temperature and the reaction mixture was stirred at ambient temperature for 16 h. The reaction was quenched by addition of a 10% aqueous Na₂S₂O₃ solution (10 mL) and extracted with MTBE (2×20 mL). The combined organic layers were washed with water (3×10 mL) and brine (10 mL), dried (Na₂SO₄), filtered and concentrated in vacuo. Purification by flash chromatography (SiO₂, 0 to 30% EtOAc in heptane) gave ethyl rac-(1R,2R,4S)-2-(2-cyclopropoxy-3,4-difluorophenyl)-4-(trifluoromethyl)cyclopentane-1-carboxylate (155 mg, 92% over 2 steps) as a clear oil. ¹H NMR (400 MHz, Chloroform-d) δ 6.95-6.80 (m, 2H), 4.29-4.23 (m, 1H), 4.06 (q, J=7.1 Hz, 2H), 3.49 (td, J=11.3, 7.3 Hz, 1H), 3.07-2.96 (m, 1H), 2.95-2.82 (m, 1H), 2.33-2.21 (m, 3H), 1.80 (td, J=12.5, 9.8 Hz, 1H), 1.15 (t, J=7.1 Hz, 3H), 0.87-0.80 (m, 2H, overlapped with grease), 0.71-0.62 (m, 2H) ppm. ESI-MS m/z calc. 378.125, found 379.1 (M+1)⁺. Ethyl rac-(1R,2R,4S)-2-(2-(2,3-dihydroxypropoxy)-3,4-difluorophenyl)-4-(trifluoromethyl)cyclopentane-1-carboxylate was not isolated.

### Step 4

A solution of ethyl rac-(1R,2R,4S)-2-(2-cyclopropoxy-3,4-difluorophenyl)-4-(trifluoromethyl)cyclopentane-1-carboxylate (80 mg, 0.192 mmol) and LiOH·H₂O (20 mg, 0.477 mmol) in a mixture of THF (1.6 mL), MeOH (1 mL) and water (1 mL) was stirred at ambient temperature for 48 h. The reaction was concentrated in vacuo to remove MeOH and THF. The aqueous solution was diluted with 1 N aqueous HCl solution (10 mL) and extracted with MTBE (2×20 mL). The combined organic layers were washed with brine (10 mL), dried (Na₂SO₄), filtered and concentrated in vacuo to give rac-(1R,2R,4S)-2-(2-cyclopropoxy-3,4-difluorophenyl)-4-(trifluoromethyl)cyclopentane-1-carboxylic acid (C-3d, 67 mg, 99%) as a pale yellow oil. ¹H NMR (400 MHz, Chloroform-d) δ 6.94-6.88 (m, 1H), 6.88-6.80 (m, 1H), 4.25 (td, J=6.4, 3.4 Hz, 1H), 3.58-3.49 (m, 1H), 3.10-3.02 (m, 1H), 2.95-2.83 (m, 1H), 2.33-2.23 (m, 3H), 1.86-1.79 (m, 1H), 0.88-0.75 (m, 2H, overlapped with grease), 0.70-0.60 (m, 2H) ppm; exchangeable H not observed. ESI-MS m/z calc. 350.094, found 349.0 (M-1)-.

### Intermediate C-3e

### rac-(1R,2R,4S)-2-(2-Cyclopropyl-3,4-difluorophenyl)-4-(trifluoromethyl)cyclopentane-1-carboxylic acid

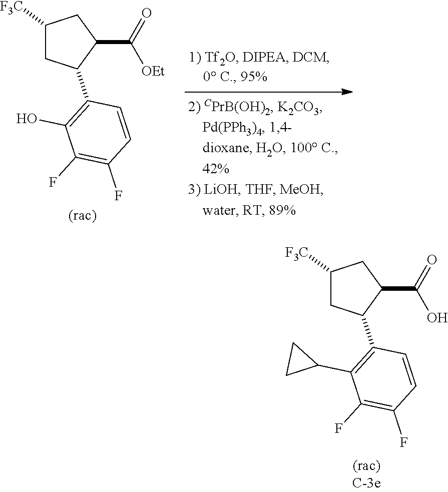 [SMILES: CCOC(=O)[C@@H]1C[C@@H](C(F)(F)F)C[C@H]1c1ccc(F)c(F)c1O ; O=C(O)[C@@H]1C[C@@H](C(F)(F)F)C[C@H]1c1ccc(F)c(F)c1C1CC1]

### Step 1

Tf₂O (461.18 mg, 0.275 mL, 1.635 mmol) was added dropwise to a stirred solution of ethyl rac-(1R,2R,4S)-2-(3,4-difluoro-2-hydroxyphenyl)-4-(trifluoromethyl)cyclopentane-1-carboxylate (500 mg, 1.323 mmol) and DIPEA (333.9 mg, 0.45 mL, 2.584 mmol) in DCM (15 mL) at 0° C. and the reaction mixture was stirred at 0° C. for 0.5 h. The reaction was diluted with DCM (45 mL), washed with water (3×15 mL), dried (Na₂SO₄), filtered and concentrated in vacuo. Purification by flash chromatography gave ethyl rac-(1R,2R,4S)-2-(3,4-difluoro-2-(((trifluoromethyl)sulfonyl)oxy)phenyl)-4-(trifluoromethyl)cyclopentane-1-carboxylate (590 mg, 95%) as a colourless oil. ¹H NMR (400 MHz, Chloroform-d) δ 7.31-7.23 (m, 1H, overlapped with solvent), 7.22-7.17 (m, 1H), 4.07 (q, J=7.1 Hz, 2H), 3.70-3.58 (m, 1H), 3.06-2.90 (m, 2H), 2.52 (dt, J=13.4, 7.9 Hz, 1H), 2.37-2.28 (m, 2H), 1.86-1.74 (m, 1H), 1.15 (t, J=7.1 Hz, 3H) ppm. ESI-MS m/z calc. 470.043, found 471.0 (M+1)⁺.

### Step 2

To a mixture of ethyl rac-(1R,2R,4S)-2-(3,4-difluoro-2-(((trifluoromethyl)sulfonyl)oxy)phenyl)-4-(trifluoromethyl)cyclopentane-1-carboxylate (585 mg, 1.243 mmol), ^{c}PrB(OH)₂ (225 mg, 2.619 mmol) and Pd(PPh₃)₄(50 mg, 0.043 mmol) in 1,4-dioxane (12 mL) was added powdered K₂CO₃ (440 mg, 3.184 mmol) and the reaction mixture was stirred at 100° C. for 2 h. The mixture was cooled to ambient temperature. Water (3 mL) and additional ^{c}PrB(OH)₂ (110 mg, 1.281 mmol) were added and nitrogen gas was passed through the mixture for 5 min. Additional Pd(PPh₃)₄(100 mg, 0.087 mmol) was added and nitrogen gas was passed through the mixture for an additional 3 min. The reaction mixture was heated at 100° C. for 3 h, cooled to ambient temperature, filtered through a pad of Celite®, washing the filtered cake with EtOAc (2×30 mL). The combined filtrates were concentrated in vacuo. Purification by flash chromatography (SiO₂, 0 to 100% EtOAc in heptane) gave ethyl rac-(1R,2R,4S)-2-(2-cyclopropyl-3,4-difluorophenyl)-4-(trifluoromethyl)cyclopentane-1-carboxylate (192 mg, 42%) as a light yellow oil. ¹H NMR (400 MHz, Chloroform-d) δ 7.08-6.95 (m, 2H), 4.16-4.08 (m, 1H), 4.04 (q, J=7.1 Hz, 2H), 3.09-2.88 (m, 2H), 2.41 (dt, J=13.3, 7.7 Hz, 1H), 2.35-2.23 (m, 2H), 1.79-1.64 (m, 2H), 1.13 (t, J=7.2 Hz, 3H), 1.09-1.02 (m, 2H), 0.83-0.75 (m, 1H), 0.71-0.57 (m, 1H) ppm. ESI-MS m/z calc. 362.131, found 363.0 (M+1)⁺. Ethyl rac-(1R,2R,4S)-2-(3,4-difluoro-2-hydroxyphenyl)-4-(trifluoromethyl)cyclopentane-1-carboxylate (136 mg, 32%) was also obtained as a light yellow oil. ¹H NMR (400 MHz, Chloroform-d) δ 6.94-6.88 (m, 1H), 6.76-6.66 (m, 1H), 6.50-6.47 (m, 1H), 4.16-4.08 (m, 2H), 3.67-3.56 (m, 1H), 3.10-2.99 (m, 1H), 2.97-2.82 (m, 1H), 2.40-2.28 (m, 2H), 2.25-2.15 (m, 1H), 2.11-2.00 (m, 1H), 1.19 (t, J=7.1 Hz, 3H). ESI-MS m/z calc. 338.094, found 339.0 (M+1)⁺.

### Step 3

A mixture of ethyl rac-(1R,2R,4S)-2-(2-cyclopropyl-3,4-difluorophenyl)-4-(trifluoromethyl)cyclopentane-1-carboxylate (185 mg, 0.511 mmol) and LiOH·H₂O (60 mg, 1.430 mmol) in a mixture of water (3 mL), THF (3 mL) and MeOH (3 mL) was stirred at ambient temperature for 2 h. The reaction was acidified to pH 0-1 by addition of an aqueous HCl solution (1.65 mL, 1 M solution, 1.650 mmol). The aqueous layer was extracted with EtOAc (3×25 mL). The combined organic layers were washed with brine (10 mL), dried (Na₂SO₄), filtered and concentrated in vacuo to give rac-(1R,2R,4S)-2-(2-cyclopropyl-3,4-difluorophenyl)-4-(trifluoromethyl)cyclopentane-1-carboxylic acid (C-3e, 174 mg, 89%) as a white solid. ¹H NMR (400 MHz, Chloroform-d) δ 7.08-6.95 (m, 2H), 4.17-4.05 (m, 1H), 3.13-3.01 (m, 1H), 3.00-2.87 (m, 1H), 2.49-2.23 (m, 3H), 1.81-1.61 (m, 2H), 1.09-0.98 (m, 2H), 0.79-0.71 (m, 1H), 0.70-0.62 (m, 1H) ppm; exchangeable H not observed. ESI-MS m/z calc. 334.099, found 333.1 (M-1)-.

### Intermediate C-4

### rac-(6R,7R)-7-(3,4-Difluoro-2-methoxyphenyl)-2,2-difluorospiro[3.4]octane-6-carboxylic acid

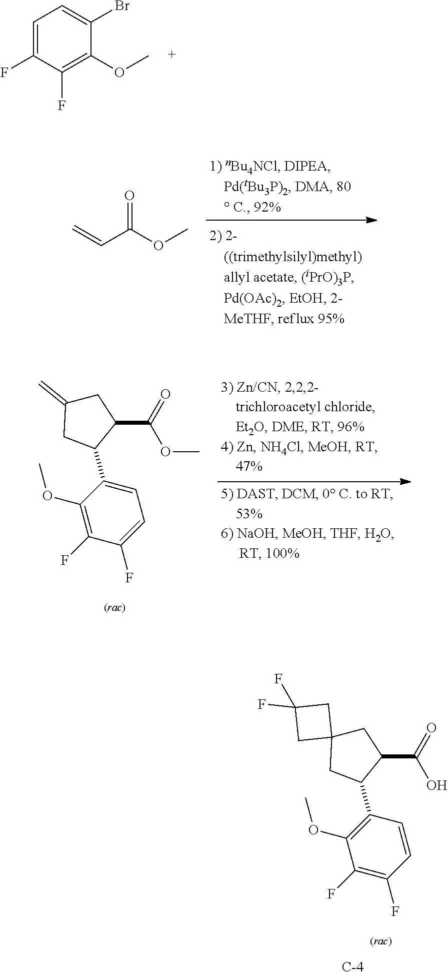

### Step 1

A stirred mixture of ⁿBu₄NCl (650 mg, 2.339 mmol) and Pd(^{t}Bu₃P)₂ (240 mg, 0.470 mmol) in DMA (25 mL) was degassed by passing argon gas through the solution for 15 min. A solution of 1-bromo-3,4-difluoro-2-methoxybenzene (4.6 g, 20.626 mmol) in DMA (7.5 mL), methyl acrylate (3.824 g, 4 mL, 44.419 mmol) and DIPEA (4.452 g, 6 mL, 34.447 mmol) were successively added and the reaction mixture was heated to 80° C. and stirred for 2 h under an argon atmosphere. The reaction was cooled to ambient temperature, diluted with EtOAc (100 mL), washed with water (2×100 mL) and brine (100 mL), dried (MgSO₄), filtered and concentrated in vacuo to give methyl (E)-3-(3,4-difluoro-2-methoxyphenyl)acrylate (4.35 g, 92%) as a pale yellow solid. ¹H NMR (400 MHz, Chloroform-d) δ 7.85 (d, J=16.5 Hz, 1H), 7.26-7.23 (m, 1H), 6.89 (td, J=9.2, 7.3 Hz, 1H), 6.46 (d, J=15.6 Hz, 1H), 4.03 (d, J=2.3 Hz, 3H), 3.81 (s, 3H) ppm.

### Step 2

2-((Trimethylsilyl)methyl)allyl acetate (6 mL, 28.240 mmol) and (ⁱPrO)₃P (1.70 mL, 6.890 mmol) were successively added to a stirred suspension of methyl (E)-3-(3,4-difluoro-2-methoxyphenyl)acrylate (5 g, 21.910 mmol) and Pd(OAc)₂ (368 mg, 1.639 mmol) in 2-MeTHF (60 mL). The reaction mixture was degassed (vacuum nitrogen cycles x 3) and heated at reflux for 1 h. The mixture was cooled to ambient temperature, diluted with EtOAc (200 mL), washed with brine (3×100 mL), dried (MgSO₄), filtered and concentrated in vacuo. Purification by flash chromatography (SiO₂, 0 to 30% EtOAc in hexanes) gave methyl rac-(1R,2R)-2-(3,4-difluoro-2-methoxyphenyl)-4-methylenecyclopentane-1-carboxylate (5.86 g, 95%) as a colourless oil. ¹H NMR (500 MHz, Chloroform-d) δ 6.90 (ddd, J=8.2, 5.8, 2.2 Hz, 1H), 6.82 (td, J=9.2, 7.4 Hz, 1H), 4.93 (dt, J=7.6, 2.3 Hz, 2H), 3.96 (d, J=2.1 Hz, 3H), 3.69 (td, J=10.4, 8.1 Hz, 1H), 3.60 (d, J=0.8 Hz, 3H), 3.04 (td, J=10.2, 8.0 Hz, 1H), 2.81 (dddd, J=17.8, 9.6, 6.2, 4.8 Hz, 2H), 2.74-2.65 (m, 1H), 2.38 (ddq, J=15.9, 10.7, 2.6 Hz, 1H) ppm.

### Step 3

A solution of trichloroacetyl chloride (645 μL, 5.778 mmol) in DME (2 mL) was added dropwise to a stirred suspension of Zn/Cu couple (1.66 g, 12.87 mmol) and methyl rac-(1R,2R)-2-(3,4-difluoro-2-methoxyphenyl)-4-methylenecyclopentane-1-carboxylate (500 mg, 1.771 mmol) in Et₂O (6 mL) at ambient temperature and the reaction mixture was stirred at ambient temperature for 6 h. The mixture was poured into a 0° C. saturated NaHCO₃ solution and filtered. The filtrates were collected and extracted with EtOAc. The organic extracts were washed with brine, dried (MgSO₄), filtered and concentrated in vacuo. Purification by flash chromatography (40 g SiO₂, 0 to 80% EtOAc in hexanes) gave methyl dia-(4R′,6R²,7R²)-1,1-dichloro-7-(3,4-difluoro-2-methoxyphenyl)-2-oxospiro[3.4]octane-6-carboxylate (667 mg, 96%), which was used without further purification in the next step.

### Step 4

Zinc (300 mg, 4.587 mmol) was added portionwise to a stirred solution of methyl dia-(4R′,6R²,7R²)-1,1-dichloro-7-(3,4-difluoro-2-methoxyphenyl)-2-oxospiro[3.4]octane-6-carboxylate (667 mg) in a 1:1 mixture of a saturated NH₄Cl solution (2 mL) and MeOH (2 mL) at ambient temperature and the reaction mixture was stirred at ambient temperature for 2 h. The mixture was filtered and the filtered cake was washed with MeOH. The filtrates were collected and concentrated in vacuo. Purification by flash chromatography (40 g SiO₂, 5 to 70% heptane in EtOAc) gave methyl rac-(6R,7R)-7-(3,4-difluoro-2-methoxyphenyl)-2-oxospiro[3.4]octane-6-carboxylate (269 mg, 47%). ¹H NMR (500 MHz, Chloroform-d) δ 6.78 (ddd, J=8.1, 5.7, 2.1 Hz, 1H), 6.71 (td, J=9.1, 7.2 Hz, 1H), 3.85 (d, J=2.4 Hz, 3H), 3.62 (ddd, J=10.8, 9.5, 7.7 Hz, 1H), 3.52 (s, 3H), 3.08-2.88 (m, 5H), 2.31-2.14 (m, 3H), 1.96 (dd, J=12.7, 10.9 Hz, 1H) ppm.

### Step 5

DAST (275 μL, 2.081 mmol) was added dropwise to a stirred solution of methyl rac-(6R,7R)-7-(3,4-difluoro-2-methoxyphenyl)-2-oxospiro[3.4]octane-6-carboxylate (269 mg, 0.829 mmol) in DCM (4 mL) at 0° C. The reaction mixture was slowly warmed to ambient temperature over 2 h, keeping the ice bath present, and stirred at ambient temperature for 36 h. Additional DAST (275 μL, 2.081 mmol) was added and the mixture was stirred at ambient temperature for a further 48 h. The reaction was slowly poured into an ice-cold saturated aqueous NaHCO₃ solution and the mixture was stirred for 30 min. The aqueous layer was separated and extracted with DCM (3 x). The combined organic extracts were washed with brine, dried (MgSO₄), filtered and concentrated in vacuo. Purification by flash chromatography (24 g SiO₂, 0 to 50% EtOAc in heptane) gave methyl rac-(6R,7R)-7-(3,4-difluoro-2-methoxyphenyl)-2,2-difluorospiro[3.4]octane-6-carboxylate (152 mg, 53%). ¹H NMR (500 MHz, Chloroform-d) δ 6.94-6.76 (m, 2H), 3.97 (d, J=2.4 Hz, 3H), 3.71-3.59 (m, 4H), 3.00 (td, J=9.4, 7.1 Hz, 1H), 2.70-2.52 (m, 4H), 2.27 (ddd, J=12.8, 8.6, 7.0 Hz, 2H), 2.19 (ddd, J=13.3, 7.2, 1.2 Hz, 1H), 1.93 (dd, J=12.8, 10.8 Hz, 1H) ppm.

### Step 6

NaOH (880 μL, 1 M aqueous solution, 0.880 mmol) was added to a solution methyl rac-(6R,7R)-7-(3,4-difluoro-2-methoxyphenyl)-2,2-difluorospiro[3.4]octane-6-carboxylate (152 mg, 0.439 mmol) in a mixture of MeOH (0.5 mL), water (0.5 mL) and THF (2 mL) and the solution was stirred at ambient temperature for 4 h. 1 M aqueous HCl (0.5 mL) was added until pH 5-6 was reached. The volatile organic solvents were removed in vacuo and the mixture was further diluted with water and DCM. The organic layer was separated and 1 M aqueous HCl was added until pH<3 was reached. The acidic aqueous layer was further extracted with DCM. The combined organic extracts were dried (MgSO₄), filtered and concentrated in vacuo to give rac-(6R,7R)-7-(3,4-Difluoro-2-methoxyphenyl)-2,2-difluorospiro[3.4]octane-6-carboxylic acid (C-4, 196 mg, 100%). ¹H NMR (500 MHz, DMSO-d₆) δ 12.17 (s, 1H), 7.24-6.97 (m, 2H), 3.88 (d, J=1.6 Hz, 3H), 3.64-3.51 (m, 2H), 2.94 (td, J=9.5, 7.3 Hz, 1H), 2.56 (t, J=26.3 Hz, 2H), 2.25 (dd, J=13.1, 9.3 Hz, 1H), 2.16 (dd, J=12.7, 7.8 Hz, 1H), 2.08 (dd, J=13.1, 7.2 Hz, 1H), 1.84 (dd, J=12.6, 11.0 Hz, 1H), 1.81-1.73 (m, 1H) ppm.

### Intermediate C-5

### rac-(3R,5R,6R)-6-(3,4-Difluoro-2-methoxyphenyl)-1,1-difluorospiro[2.4]heptane-5-carboxylic acid and rac-(3S,5R,6R)-6-(3,4-difluoro-2-methoxyphenyl)-1,1-difluorospiro[2.4]heptane-5-carboxylic acid

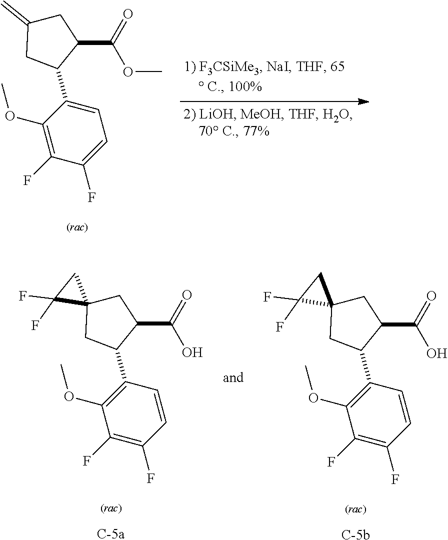

### Step 1

In a 25 mL pressure tube, anhydrous NaI (100 mg, 0.667 mmol) and F₃CSiMe₃ (1.24 mL, 8.389 mmol) were successively added to a stirred and degassed solution of methyl rac-(1R,2R)-2-(3,4-difluoro-2-methoxyphenyl)-4-methylenecyclopentane-1-carboxylate (1 g, product of Intermediate C-4 Step 2, 3.365 mmol) in THF (10 mL), under a nitrogen atmosphere. The reaction vessel was sealed and heated at 65° C. for 2.5 h. The mixture was concentrated in vacuo and the residue was partitioned between EtOAc (60 mL) and water (30 mL). The organic phase was separated, washed with a saturated Na₂SO₃ solution (30 mL), a saturated NaHCO₃ solution (30 mL) and water (30 mL), dried (Na₂SO₄), filtered and concentrated in vacuo. Purification by flash chromatography (24 g SiO₂, 0 to 20% EtOAc in hexanes) gave methyl dia-(3S′,5R²,6R²)-6-(3,4-difluoro-2-methoxyphenyl)-1,1-difluorospiro[2.4]heptane-5-carboxylate (1.118 g, 100%) as a colourless oil. ESI-MS m/z calc. 332.104, found 333.0 (M+1)⁺.

### Step 2

LiOH·H₂O (1.01 g, 24.068 mmol) was added to a stirred solution of a 1:1 mixture of diastereomers of methyl dia-(3S′,5R²,6R²)-6-(3,4-difluoro-2-methoxyphenyl)-1,1-difluorospiro[2.4]heptane-5-carboxylate (1.975 g, 5.926 mmol) in a mixture of THF (44 mL), MeOH (22 mL) and water (22 mL). The flask was placed in a pre-heated oil bath set at 70° C. for 17 h. The reaction was cooled to ambient temperature and partitioned between a 5% aqueous citric acid solution (300 mL) and EtOAc (200 mL). The aqueous layer was separated and extracted with EtOAc (150 mL). The combined organic extracts were washed with water (200 mL), brine (150 mL), dried (Na₂SO₄), filtered and concentrated in vacuo. Purification by reverse phase chromatography (C₁₈ column, 5 to 100% MeOH in water with 0.05% TFA) gave:

First Eluting Isomers: rac-(3R,5R,6R)-6-(3,4-difluoro-2-methoxyphenyl)-1,1-difluorospiro[2.4]heptane-5-carboxylic acid (C-5a, 800 mg, 42%) as a colourless oil. ¹H NMR (400 MHz, Chloroform-d) δ 6.96-6.89 (m, 1H), 6.88-6.78 (m, 1H), 3.97 (d, J=2.4 Hz, 3H), 3.70 (td, J=10.7, 7.9 Hz, 1H), 3.30-3.19 (m, 1H), 2.44-2.29 (m, 2H), 2.13-2.06 (m, 1H), 1.85 (ddd, J=13.1, 11.1, 6.6 Hz, 1H), 1.37-1.22 (m, 2H) ppm; exchangeable H not observed. ESI-MS m/z calc. 318.088, found 317.1 (M-1)-.

Second Eluting Isomers: rac-(3S,5R,6R)-6-(3,4-difluoro-2-methoxyphenyl)-1,1-difluorospiro[2.4]heptane-5-carboxylic acid (C-5b, 670 mg, 35%) as a colorless oil. ¹H NMR (400 MHz, Chloroform-d) δ 6.95-6.88 (m, 1H), 6.88-6.79 (m, 1H), 3.97 (d, J=2.4 Hz, 3H), 3.87 (q, J=9.5 Hz, 1H), 3.11 (td, J=10.1, 7.9 Hz, 1H), 2.38 (dd, J=13.2, 7.8 Hz, 1H), 2.16-2.06 (m, 3H), 1.38-1.29 (m, 2H) ppm; exchangeable H not observed. ESI-MS m/z calc. 318.088, found 317.0 (M-1)-.

### Intermediate C-8a

### rac-(1R,2R,3aR,6aS)-1-(3,4-Difluoro-2-methylphenyl)-4,4-difluorooctahydropentalene-2-carboxylic acid

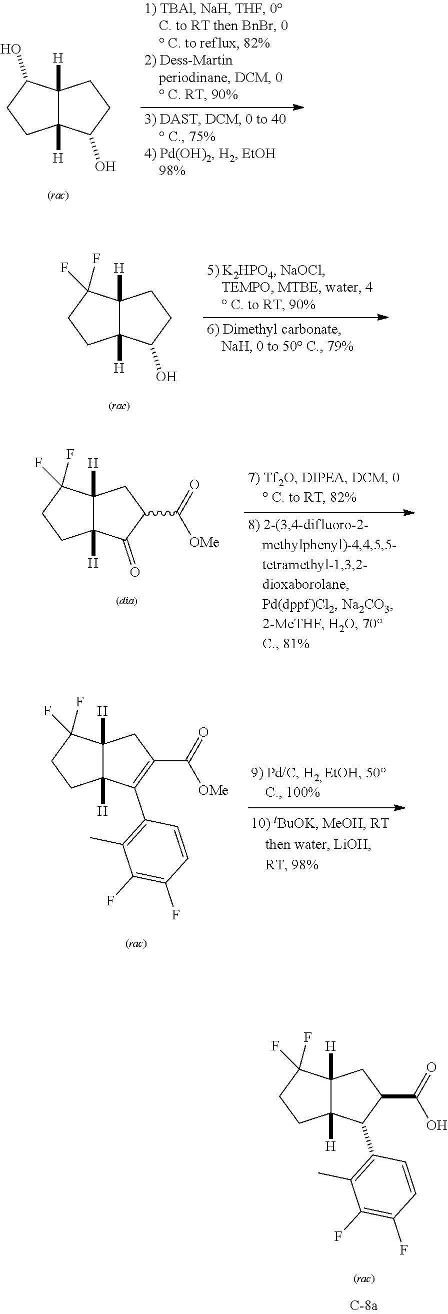 [SMILES: O[C@H]1CC[C@@H]2[C@H]1CC[C@@H]2O ; O[C@H]1CC[C@@H]2[C@H]1CCC2(F)F ; COC(=O)C1C[C@@H]2[C@@H](CCC2(F)F)C1=O ; COC(=O)C1=C(c2ccc(F)c(F)c2C)[C@@H]2CCC(F)(F)[C@@H]2C1 ; Cc1c([C@@H]2[C@@H]3CCC(F)(F)[C@@H]3C[C@H]2C(=O)O)ccc(F)c1F]

### Step 1

NaH (5.3 g, 60% dispersion in mineral oil, 132.51 mmol) was added portionwise to a stirred mixture of rac-(1S,3aR,4S,6aR)-octahydropentalene-1,4-diol (18.95 g, 113.28 mmol) and TBAI (4.45 g, 12.048 mmol) in THF (900 mL) at 0° C. and the reaction mixture was stirred at ambient temperature for 45 min. The mixture was cooled to 0° C. BnBr (22.32 g, 15.5 mL, 130.5 mmol) was added dropwise over 15 min and the reaction was stirred under reflux for 3.5 h. The mixture was cooled to ambient temperature, quenched by addition of a saturated aqueous NH₄Cl solution (900 mL) and diluted with EtOAc (250 mL). The aqueous layer was separated and extracted with EtOAc (500 mL). The combined organic layers were washed with brine (500 mL), dried (Na₂SO₄), filtered and concentrated in vacuo. Purification by flash chromatography (SiO₂, 0 to 30% EtOAc in heptane) gave rac-(1S,3aR,4S,6aR)-4-(benzyloxy)octahydropentalen-1-ol (23.43 g, 82%) as a light orange oil. ¹H NMR (400 MHz, Chloroform-d) δ 7.39-7.27 (m, 5H), 4.64 (d, J=11.7 Hz, 1H), 4.45 (d, J=11.7 Hz, 1H), 3.95-3.88 (m, 1H), 3.80-3.76 (m, 1H), 3.10 (d, J=10.3 Hz, 1H), 2.63-2.51 (m, 2H), 2.14-2.05 (m, 1H), 1.97-1.83 (m, 3H), 1.79-1.57 (m, 4H, overlapped with water) ppm. ESI-MS m/z calc. 232.146, found 233.2 (M+1)⁺.

### Step 2

Dess-Martin periodinane (30.88 g, 72.806 mmol) was added to a stirred solution of rac-(1S,3aR,4S,6aR)-4-(benzyloxy)octahydropentalen-1-ol (15.13 g, 61.675 mmol) in DCM (350 mL) at 0° C. and the reaction mixture was stirred at ambient temperature for 3 h. The mixture was diluted with DCM (200 mL), washed with a 1:1 mixture of saturated aqueous NaHCO₃ solution and a 10% aqueous Na₂S₂O₃ solution (3×300 mL), dried (Na₂SO₄), filtered and concentrated in vacuo. The residue was suspended in heptane (300 mL) and filtered. The solid was washed with heptane (500 mL). The filtrates were concentrated in vacuo to give a yellow oil. Purification by flash chromatography (SiO₂, 0 to 20% EtOAc in heptane) gave rac-(3aR,4S,6aR)-4-(benzyloxy)hexahydropentalen-1(2H)-one (12.74 g, 90%) as a yellow oil. ¹H NMR (400 MHz, Chloroform-d) δ 7.39-7.23 (m, 5H), 4.54 (d, J=11.9 Hz, 1H), 4.42 (d, J=11.9 Hz, 1H), 4.00 (q, J=5.2 Hz, 1H), 2.88-2.79 (m, 1H), 2.65-2.56 (m, 1H), 2.42-2.19 (m, 2H), 2.15-2.03 (m, 1H), 2.02-1.65 (m, 5H) ppm. ESI-MS m/z calc. 230.131, found 231.2 (M+1)⁺.

### Step 3

DAST (54.9 g, 45 mL, 340.590 mmol) was added dropwise to a solution of rac-(3aR,4S,6aR)-4-(benzyloxy)hexahydropentalen-1(2H)-one (12.74 g, 55.260 mmol) in DCM (200 mL) at 0° C. and the reaction mixture was stirred at 40° C. for 3 days. An additional amount of DAST (9.150 g, 7.5 mL, 56.770 mmol) was added at ambient temperature and the reaction mixture was stirred at 40° C. for 22 h. The reaction was quenched by poured the mixture into a stirred mixture of a saturated aqueous NaHCO₃ solution (250 mL) and DCM (250 mL). The aqueous layer was separated and extracted with DCM (2×200 mL). The combined organic layers were dried (Na₂SO₄), filtered and concentrated in vacuo. Purification by flash chromatography (SiO₂, 0 to 10% EtOAc in heptane) gave rac-(3aR,4S,6aR)-4-(benzyloxy)-1,1-difluorooctahydropentalene (10.89 g, 75%) as a yellow oil. ¹H NMR (400 MHz, Chloroform-d) δ 7.38-7.26 (m, 5H), 4.54-4.43 (m, 2H), 3.96-3.88 (m, 1H), 2.83-2.74 (m, 1H), 2.66-2.52 (m, 1H), 2.09-1.92 (m, 3H), 1.85-1.58 (m, 5H) ppm.

### Step 4

Pd(OH)₂ (14.14 g, 20% on carbon, 64% wet, 7.250 mmol) was added to a solution of rac-(3aR,4S,6aR)-4-(benzyloxy)-1,1-difluorooctahydropentalene (45.571 g, 176.330 mmol) in EtOH (320 mL) at ambient temperature. The reaction mixture was stirred under a 50 psi pressure of hydrogen at ambient temperature overnight. The mixture was filtered over Celite®, washing the filtered cake with EtOAc (2×150 mL). The filtrates were concentrated in vacuo to give rac-(1S,3aR,6aR)-4,4-difluorooctahydropentalen-1-ol (28.71 g, 98%) as an orange oil. ¹H NMR (400 MHz, chloroform-d) δ 4.25 (q, J=5.6 Hz, 1H), 2.73-2.55 (m, 2H), 2.15-1.94 (m, 2H), 1.91-1.63 (m, 7H, overlapped with water) ppm.

### Step 5

NaOCl (60 mL, 10% w/v solution in water, 80.600 mmol) was added over 40 min to a biphasic solution of rac-(1S,3aR,6aR)-4,4-difluorooctahydropentalen-1-ol, K₂HPO₄ (21 g, 120.570 mmol) and TEMPO (630 mg, 4.030 mmol) in a mixture of MTBE (120 mL) and water (18 mL) at 4° C., keeping the internal temperature below 10° C. The reaction mixture was stirred at ambient temperature for 3 h and quenched by addition of a 10% aqueous Na₂S₂O₃ solution (200 mL). The aqueous layer was separated and extracted with MTBE (2×200 mL). The combined organic layers were dried (Na₂SO₄), filtered and concentrated in vacuo. The residue was subjected another 3 times to the same reaction conditions using NaOCl (60 mL then, 2×30 mL), K₂HPO₄ (21 g then 2×11 g) and TEMPO (630 mg then, 2×300 mg) in a mixture of MTBE (120 mL on all occasions) and water (18 mL on all occasions). Purification by flash chromatography (SiO₂, 0 to 10% MTBE in pentane) gave rac-(3aR,6aR)-4,4-difluorohexahydropentalen-1(2H)-one (14.46 g, 90%) as an orange oil. ¹H NMR (400 MHz, Chloroform-d) δ 3.09-2.96 (m, 1H), 2.85-2.78 (m, 1H), 2.40-2.30 (m, 2H), 2.22-2.07 (m, 4H, overlapped with EtOAc), 2.04-1.93 (m, 2H) ppm.

### Step 6

NaH (2.21 g, 60% dispersion in mineral oil, 55.255 mmol) was added portionwise to a stirred solution of rac-(3aR,6aR)-4,4-difluorohexahydropentalen-1(2H)-one (4.58 g, 22.020 mmol) in dimethyl carbonate (81.24 g, 76 mL, 901.930 mmol) at 0° C. and the reaction mixture was heated to 50° C. for 4 h. The mixture was cooled to ambient temperature and the reaction was quenched by pouring the solution into a stirred saturated aqueous NH₄Cl solution (150 mL). The mixture was extracted with EtOAc (3×100 mL). The combined organic layers were washed with water (100 mL) and brine (100 mL), dried (Na₂SO₄), filtered and concentrated in vacuo. Purification by flash chromatography (SiO₂, 0 to 10% EtOAc in heptane) gave methyl dia-(2R¹,3aR²,6aR²)-4,4-difluoro-1-oxooctahydropentalene-2-carboxylate (4.66 g, 79%) as a yellow oil. ¹H NMR (400 MHz, chloroform-d) δ 3.79-3.73 (m, 3H), 3.48-3.31 (m, 1H), 3.13-2.33 (m, 3H), 2.17-1.68 (m, 5H) ppm.

### Step 7

A solution of Tf₂O (10.062 g, 6 mL, 35.663 mmol) in DCM (30 mL) was added dropwise to a stirred mixture of methyl dia-(2R¹,3aR²,6aR²)-4,4-difluoro-1-oxooctahydropentalene-2-carboxylate and DIPEA (6.678 g, 9 mL, 51.670 mmol) in DCM (50 mL) at 0° C. and the reaction mixture was stirred at ambient temperature overnight. The reaction was diluted with DCM (50 mL) and quenched by addition of a saturated aqueous NaHCO₃ solution (100 mL). The aqueous phase was separated and extracted with DCM (2×50 mL). The combined organic layers were dried (Na₂SO₄), filtered and concentrated in vacuo. Purification by flash chromatography (SiO₂, 0 to 15% EtOAc in heptane) gave methyl rac-(3aR,6aR)-6,6-difluoro-3-(((trifluoromethyl)sulfonyl)oxy)-1,3a,4,5,6,6a-hexahydropentalene-2-carboxylate (5.27 g, 82%) as a yellow oil. ¹H NMR (400 MHz, chloroform-d) δ 3.81 (s, 3H), 3.67-3.57 (m, 1H), 3.01-2.88 (m, 1H), 2.88-2.80 (m, 2H), 2.20-2.08 (m, 1H), 2.08-1.91 (m, 3H) ppm. ESI-MS m/z calc. 350.025, found 351.1 (M+1)⁺.

### Step 8

Na₂CO₃ (7 mL, 2 M aqueous solution, 14 mmol) was added to a solution of methyl rac-(3aR,6aR)-6,6-difluoro-3-(((trifluoromethyl)sulfonyl)oxy)-1,3a,4,5,6,6a-hexahydropentalene-2-carboxylate (2.020 g, 5.414 mmol) and 2-(3,4-difluoro-2-methylphenyl)-4,4,5,5-tetramethyl-1,3,2-dioxaborolane (1.770 g, 6.964 mmol) in 2-MeTHF (27 mL) and nitrogen gas was passed through the solution for 10 min. Pd(dppf)Cl₂ (56.3 mg, 0.068 mmol) was added and the reaction mixture was heated at 70° C. for 2 h. The reaction was cooled to ambient temperature and diluted with water (20 mL). The organic phase was separated, washed with water (20 mL) and brine (20 mL), dried (Na₂SO₄), filtered and concentrated in vacuo to a volume of 50 mL. Activated charcoal (200 mg, 16.652 mmol) was added to the solution and the mixture was stirred at ambient temperature for 17 h. The mixture was filtered through a pad of Celite® and the mother liquors were concentrated in vacuo. Purification by flash chromatography (15 g SiO₂, 0 to 10% MTBE in heptane) gave methyl rac-(3aR,6aR)-3-(3,4-difluoro-2-methylphenyl)-6,6-difluoro-1,3a,4,5,6,6a-hexahydropentalene-2-carboxylate (1.464 g, 81%) as a white solid. ¹H NMR (400 MHz, Chloroform-d) δ 7.04-6.95 (m, 1H), 6.70 (br s, 1H), 3.65 (br s, 1H), 3.59 (s, 3H), 3.08-2.95 (m, 3H), 2.19-1.76 (m, 7H) ppm. ESI-MS m/z calc. 328.109, found 329.1 (M+1)⁺.

### Step 9

Pd/C (306 mg, 10% on carbon, 50% wet, 0.144 mmol) was added to a solution of methyl rac-(3aR,6aR)-3-(3,4-difluoro-2-methylphenyl)-6,6-difluoro-1,3a,4,5,6,6a-hexahydropentalene-2-carboxylate (469 mg, 1.427 mmol) in EtOH (12 mL) and the reaction mixture was stirred at 50° C. under 60 psi of hydrogen at ambient temperature for 18 h. The reaction was placed under a nitrogen atmosphere. Additional Pd/C (366 mg, 10% on carbon, 50% wet, 0.149 mmol) was added and the reaction mixture was stirred at 50° C. under 60 psi of hydrogen at ambient temperature for 18 h. The reaction was placed under a nitrogen atmosphere, diluted with EtOAC (10 mL) and filtered through a pad of Celite®. The filtrates were concentrated in vacuo to give methyl rac-(1R,2S,3aR,6aS)-1-(3,4-difluoro-2-methylphenyl)-4,4-difluorooctahydropentalene-2-carboxylate (471 mg, 100%) as a light yellow solid. ¹H NMR (400 MHz, Chloroform-d) δ 6.94-6.88 (m, 1H), 6.88-6.81 (m, 1H), 3.94 (t, J=9.2 Hz, 1H), 3.40 (s, 3H), 3.37-3.28 (m, 1H), 3.20-3.10 (m, 1H), 2.91-2.76 (m, 1H), 2.39-2.23 (m, 2H), 2.21 (d, J=2.6 Hz, 3H), 1.93-1.75 (m, 2H), 1.61-1.50 (m, 1H), 1.15-1.04 (m, 1H) ppm. ESI-MS m/z calc. 330.124, found 311.2 (M−19)⁺.

### Step 10

KO^{t}Bu (19.3 g, 21.397 mmol) was added to a solution of methyl rac-(1R,2S,3aR,6aS)-1-(3,4-difluoro-2-methylphenyl)-4,4-difluorooctahydropentalene-2-carboxylate (32.9 g, 97.908 mmol) in MeOH (500 mL) and the reaction mixture was stirred at ambient temperature for 3 days. Water (125 mL) and LiOH·H₂O (8.5 g, 202.56 mmol) were successively added and the reaction was stirred at ambient temperature for 18 h. The reaction was concentrated in vacuo. The residue was diluted with water (150 mL) and the solution was acidified to pH 1-2 by addition of a 1 M aqueous HCl solution and extracted with MTBE (2×150 mL). The combined organic layers were washed with brine (100 mL), dried (Na₂SO₄), filtered and concentrated in vacuo to give rac-(1R,2R,3aR,6aS)-1-(3,4-difluoro-2-methylphenyl)-4,4-difluorooctahydropentalene-2-carboxylic acid (C-8a, 30.3 g, 98%) as a white solid. H NMR (400 MHz, Chloroform-d) δ 12.56-10.21 (br, s, 1H), 6.98-6.89 (m, 2H), 3.54 (dd, J=12.1, 8.2 Hz, 1H), 3.18-3.06 (m, 2H), 2.96-2.82 (m, 1H), 2.46 (dd, J=13.3, 6.7 Hz, 1H), 2.27 (d, J=2.4 Hz, 3H), 2.03-1.80 (m, 3H), 1.31-1.18 (m, 1H), 1.15-1.04 (m, 1H) ppm. ESI-MS m/z calc. 316.109, found 317.1 (M+1)⁺.

The following Intermediate was made using the method described in Intermediate C-8a, except that in Step 8, Pd(PPh₃)₄ and a different Suzuki coupling partner were respectively used in place of Pd(dppf)Cl₂ and 2-(3,4-difluoro-2-methylphenyl)-4,4,5,5-tetramethyl-1,3,2-dioxaborolane and the reaction was carried out at 80° C. In the hydrogenation Step 9, MeOH was used as the solvent in place of EtOH and the reaction was carried out at ambient temperature:

| | | LC/MS | | |
|---|---|---|---|---|
| Intermediate | | (m/z | Found | |
| Number | Intermediate Name | calc.) | M + 1 | NMR (shifts in ppm) |
| C - 8b | rac-(1R,2R,3aR,6aS)-1- | 332.104 | 333.2 | ¹H NMR (400 MHz, DMSO-d₆) δ 12.31 |
| | (3,4-difluoro-2- | | | (br s, 1H), 7.16-7.03 (m, 2H), 3.92 (d, |
| | methoxyphenyl)-4,4- | | | J = 2.0 Hz, 3H), 3.55 (dd, J = 12.6, 7.9 |
| | difluorooctahydropentalene- | | | Hz, 1H), 3.12-2.97 (m, 2H), 2.96- |
| | 2-carboxylic acid | | | 2.82 (m, 1H), 2.20 (dd, J = 13.2, 6.8 |
| | | | | Hz, 1H), 2.01-1.82 (m, 3H), 1.30- |
| | | | | 1.18 (m, 1H), 1.08-0.95 (m, 1H) ppm. |

The following Intermediate was made using the method described in Intermediate C-8a, except that in Step 8, Pd(PPh₃)₄ and a different Suzuki coupling partner were respectively used in place of Pd(dppf)Cl₂ and 2-(3,4-difluoro-2-methylphenyl)-4,4,5,5-tetramethyl-1,3,2-dioxaborolane and the reaction was carried out at 80° C. In the hydrogenation Step 9, EtOAc was used as the solvent in place of EtOH and the reaction was carried out at ambient temperature and at atmospheric pressure:

| | | LC/MS | | |
|---|---|---|---|---|
| Intermediate | | (m/z | Found | |
| Number | Intermediate Name | calc.) | M + 1 | NMR (shifts in ppm) |
| C - 8c | rac-(1R,2R,3aR,6aS)-4,4- | 298.118 | 299.1 | ¹H NMR (400 MHz, Chloroform-d) δ |
| | difluoro-1-(4-fluoro-3- | | | 7.02-6.87 (m, 3H), 3.49 (dd, J = 12.2, |
| | methylphenyl)octahydropentalene- | | | 8.3 Hz, 1H), 3.10-2.97 (m, 2H), 2.94- |
| | 2-carboxylic | | | 2.78 (m, 1H), 2.43 (dd, J = 13.4, 6.8 |
| | acid | | | Hz, 1H), 2.24 (d, J = 1.7 Hz, 3H), 2.10- |
| | | | | 1.78 (m, 3H), 1.39-1.25 (m, 1H), 1.22- |
| | | | | 1.06 (m, 1H) ppm; exchangeable H not |
| | | | | observed. |

The following Intermediates were made using the method described in Intermediate C-8a, except that, the conditions used in Step 8 to 10 were those of Intermediate C-2 Steps 2 to 4. In Step 8, different Suzuki coupling partners were used as the starting material. In Step 10, in the case of Intermediate C-8d, the reaction was carried out in absence of THF:

| | | LC/MS | | |
|---|---|---|---|---|
| Intermediate | | (m/z | Found | |
| Number | Intermediate Name | calc.) | M + 1 | NMR (shifts in ppm) |
| C - 8d | rac-(1R,2R,3aR,6aS)-4,4- | 312.134 | 313.3 | ¹H NMR (400 MHz, Chloroform-d) δ |
| | difluoro-1-(4-fluoro-2,3- | | | 7.04 (dd, J = 8.7, 5.6 Hz, 1H), 6.85 (t, J = |
| | dimethylphenyl)octahydropentalene- | | | 8.9 Hz, 1H), 3.64 (dd, J = 12.2, 8.1 |
| | 2-carboxylic | | | Hz, 1H), 3.24-3.06 (m, 2H), 2.98- |
| | acid | | | 2.81 (m, 1H), 2.47 (dd, J = 13.4, 6.6 |
| | | | | Hz, 1H), 2.26 (s, 3H), 2.20 (d, J = 2.0 |
| | | | | Hz, 3H), 2.10-1.81 (m, 3H), 1.27- |
| | | | | 1.20 (m, 1H), 1.20-1.08 (m, 1H) ppm; |
| | | | | exchangeable H not observed. |
| C - 8j | dia-(1R¹,2R¹,3aR²,6aS²)- | 316.109 | 317.3 | |
| | 1-(2,4-difluoro-3- | | | |
| | methylphenyl)-4,4- | | | |
| | difluorooctahydropentalene- | | | |
| | 2-carboxylic acid | | | |

The following Intermediates were made using the method described in Intermediate C-8a, except that in Step 8, Pd(dppf)Cl₂·DCM and different Suzuki coupling partners were respectively used in place of Pd(dppf)Cl_{z} and 2-(3,4-difluoro-2-methylphenyl)-4,4,5,5-tetramethyl-1,3,2-dioxaborolane. In the hydrogenation Step 9, Pd(OH)_{z} was used as the catalyst in place of Pd/C and the reaction was carried out at ambient temperature in MeOH as the solvent. In the case of Intermediate C-8e, the reaction was carried out at a pressure of 100 psi. In the case of Intermediate C-8g, the reaction was carried out in a mixture of MeOH and EtOAc at a pressure of 150 psi:

| | | LC/MS | | |
|---|---|---|---|---|
| Intermediate | | (m/z | Found | |
| Number | Intermediate Name | calc.) | M + 1 | NMR (shifts in ppm) |
| C - 8e | rac-(1R,2R,3aR,6aS)-4,4- | 328.129 | 329.2 | ¹H NMR (400 MHz, Chloroform-d) δ |
| | difluoro-1-(4-fluoro-2- | | | 6.98 (dd, J = 8.6, 6.3 Hz, 1H), 6.76 (t, J = |
| | methoxy-3- | | | 8.7 Hz, 1H), 3.73 (s, 3H), 3.64 (dd, J = |
| | methylphenyl)octahydropentalene- | | | 12.5, 8.2 Hz, 1H), 3.28-3.11 (m, |
| | 2-carboxylic | | | 1H), 3.11-2.97 (m, 1H), 2.96-2.76 (m, |
| | acid | | | 1H), 2.44 (dd, J = 13.4, 6.6 Hz, 1H), |
| | | | | 2.20 (s, 3H), 2.06-1.74 (m, 3H), 1.37- |
| | | | | 1.17 (m, 1H), 1.17-0.99 (m, 1H) ppm; |
| | | | | exchangeable H not observed. |
| C - 8g | rac-(1R,2R,3aR,6aS)-4,4- | 324.134 | 325.1 | ¹H NMR (400 MHz, Chloroform-d) δ |
| | difluoro-1-(7-fluoro-2,3- | | | 6.98 (dd, J = 8.5, 4.7 Hz, 1H), 6.78 (t, J = |
| | dihydro-1H-inden-4- | | | 8.5 Hz, 1H), 3.49 (dd, J = 12.3, 8.2 |
| | yl)octahydropentalene-2- | | | Hz, 1H), 3.19-3.03 (m, 2H), 3.02- |
| | carboxylic acid | | | 2.73 (m, 5H), 2.43 (dd, J = 13.5, 6.7 |
| | | | | Hz, 1H), 2.20-2.06 (m, 2H), 2.02- |
| | | | | 1.79 (m, 3H), 1.30-1.20 (m, 1H), 1.21- |
| | | | | 1.08 (m, 1H) ppm; exchangeable H not |
| | | | | observed. |

The following Intermediate was made using the method described in Intermediate C-8a, except that, in Step 8, Pd(dppf)Cl₂·DCM and a different Suzuki coupling partner were respectively used in place of Pd(dppf)Cl₂ and 2-(3,4-difluoro-2-methylphenyl)-4,4,5,5-tetramethyl-1,3,2-dioxaborolane. The conditions used in Steps 9 and 10 were those of Intermediate C-2 Steps 3 and 4 and the reactions were respectively carried out at 65° C. and at ambient temperature.

| Intermediate | |
|---|---|
| Number | Intermediate Name |
| C - 8f | rac-(1R,2R,3aR,6aS)-1- |
| | (2-chloro-4-fluoro-3- |
| | methylphenyl)-4,4- |
| | difluorooctahydropentalene- |
| | 2-carboxylic acid |

The following Intermediate was made using the method described in Intermediate C-8a, except that, the conditions of Steps 8 and 9 were those of Intermediate C-2 Steps 2 and 3. In Step 8, a different Suzuki coupling partner was used as the starting material and the reaction was carried out at 80° C. In Step 9, the reaction was carried out at 0° C.:

| | | LC/MS | |
|---|---|---|---|
| Intermediate | | (m/z | Found |
| Number | Intermediate Name | calc.) | M + 1 |
| C - 8h | dia-(1R¹,2R¹,3aR²,6aS²)- | 332.079 | 333.3 |
| | 1-(3-chloro-4-fluoro-2- | | |
| | methylphenyl)-4,4- | | |
| | difluorooctahydropentalene- | | |
| | 2-carboxylic acid | | |

The following Intermediate was made using the method described in Intermediate C-8a, except that, in Step 8, Pd(dppf)Cl₂·DCM and a different Suzuki coupling partner were respectively used in place of Pd(dppf)Cl₂ and 2-(3,4-difluoro-2-methylphenyl)-4,4,5,5-tetramethyl-1,3,2-dioxaborolane. The conditions used in Steps 9 and 10 were those of Intermediate C-2 Steps 3 and 4 and the reactions were respectively carried out at 65 and 50° C.:

| | | LC/MS | |
|---|---|---|---|
| Intermediate | | (m/z | Found |
| Number | Intermediate Name | calc.) | M + 1 |
| C - 8i | dia-(1R¹,2R¹,3aR²,6aS²)- | 332.079 | 333.3 |
| | 1-(3-chloro-4- | | |
| | fluorophenyl)-4,4- | | |
| | difluorooctahydropentalene- | | |
| | 2-carboxylic acid | | |

The following Intermediate was made using the method described in Intermediate C-8a, except that in Step 8, Pd(PPh₃)₄ and a different Suzuki coupling partner were respectively used in place of Pd(dppf)Cl₂ and 2-(3,4-difluoro-2-methylphenyl)-4,4,5,5-tetramethyl-1,3,2-dioxaborolane and the reaction was carried out at 80° C. in a mixture of toluene, MeOH and water as the solvents. In the hydrogenation Step 9, Pd(OH)₂ was used as the catalyst in place of Pd/C and the reaction was carried out at 70° C. in EtOAc at a pressure of 120 psi:

| | | LC/MS | | |
|---|---|---|---|---|
| Intermediate | | (m/z | Found | |
| Number | Intermediate Name | calc.) | M + 1 | NMR (shifts in ppm) |
| C - 8k | rac-(1R,2R,3aR,6aS)-1- | 446.190 | 447.2 | ¹H NMR (400 MHz, Chloroform-d) δ |
| | (2-(((tert- | | | 7.13-6.94 (m, 2H), 4.86-4.77 (m, 1H), |
| | butyldimethylsilyl)oxy)methyl)- | | | 4.75-4.65 (m, 1H), 3.79 (dd, J = 12.3, |
| | 3,4-difluorophenyl)-4,4- | | | 8.3 Hz, 1H), 3.24 (quin, J = 8.7 Hz, |
| | difluorooctahydropentalene- | | | 1H), 3.12 (td, J = 12.0, 6.6 Hz, 1H), |
| | 2-carboxylic acid | | | 2.95-2.79 (m, 1H), 2.45 (dd, J = 13.3, |
| | | | | 6.6 Hz, 1H), 2.04-1.83 (m, 3H), 1.35- |
| | | | | 1.27 (m, 1H), 1.22-1.11 (m, 1H), 0.90- |
| | | | | 0.84 (m, 9H), 0.10 (d, J = 9.7 Hz, 6H) |
| | | | | ppm; exchangeable H not observed. |

The following Intermediate was made using the method described in Intermediate C-8a, except that in Step 1, (1S,3aR,4S,6aR)-octahydropentalene-1,4-diol was used as the starting material in place of rac-(1S,3aR,4S,6aR)-octahydropentalene-1,4-diol. In Step 8, Pd(dppf)Cl₂·DCM and a different Suzuki coupling partner were respectively used in place of Pd(dppf)Cl₂ and 2-(3,4-difluoro-2-methylphenyl)-4,4,5,5-tetramethyl-1,3,2-dioxaborolane. In the hydrogenation Step 9, Pd(OH)₂ was used as the catalyst in place of Pd/C and the reaction was carried out at 50° C. under a pressure of 60 psi of hydrogen:

| | | LC/MS | | |
|---|---|---|---|---|
| Inter. | | (m/z | Found | |
| No. | Intermediate Name | calc.) | M + 1 | NMR (shifts in ppm) |
| C - 8l | (1R,2R,3aR,6aS)-1-(3,4- | 316.109 | 317.0 | ¹H NMR (400 MHz, DMSO-d₆) δ 12.54 |
| | difluoro-2- | | | (br s, 1H), 7.14-7.23 (m, 1H), 7.02- |
| | methylphenyl)-4,4- | | | 7.10 (m, 1H), 3.44-3.52 (m, 1H), 3.02- |
| | difluorooctahydropentalene- | | | 3.16 (m, 2H), 2.82-2.94 (m, 1H), 2.17- |
| | 2-carboxylic acid | | | 2.28 (m, 4H), 1.79-2.01 (m, 3H), 1.13- |
| | | | | 1.27 (m, 1H), 0.92-1.04 (m, 1H) ppm. |

The following Intermediates were made using the method described in Intermediate-a, except that, the conditions used in Steps 8 and 9 were those of Intermediate C-2 Steps 2 and 3. In Step 8, different Suzuki coupling partners were used as the starting material and the reaction was carried out at 70° C. In Step 10, the reaction was carried out at 65° C.:

| | | LC/MS | |
|---|---|---|---|
| Inter. | | (m/z | Found |
| No. | Intermediate Name | calc.) | M + 1 |
| C - 8m | rac-(1R,2R,3aR,6aS)-4,4- | 349.110 | 350.1 |
| | difluoro-1-(2-methyl-6- | | |
| | (trifluoromethyl)pyridin- | | |
| | 3-yl)octahydropentalene- | | |
| | 2-carboxylic acid | | |
| C - 8n | rac-(1R,2R,3aR,6aS)-4,4- | 365.105 | 366.0 |
| | difluoro-1-(2-methoxy-6- | | |
| | (trifluoromethyl)pyridin- | | |
| | 3-yl)octahydropentalene- | | |
| | 2-carboxylic acid | | |
| C - 8p | rac-(1R,2R,3aR,6aS)-4,4- | 311.133 | 312.5 |
| | difluoro-1-(2-methoxy-6- | | |
| | methylpyridin-3- | | |
| | yl)octahydropentalene-2- | | |
| | carboxylic acid | | |

### Intermediate C-8o

### rac-(1R,2R,3aR,6aS)-4,4-Difluoro-1-(5-fluoro-6-methylpyridin-2-yl)octahydropentalene-2-carboxylic acid

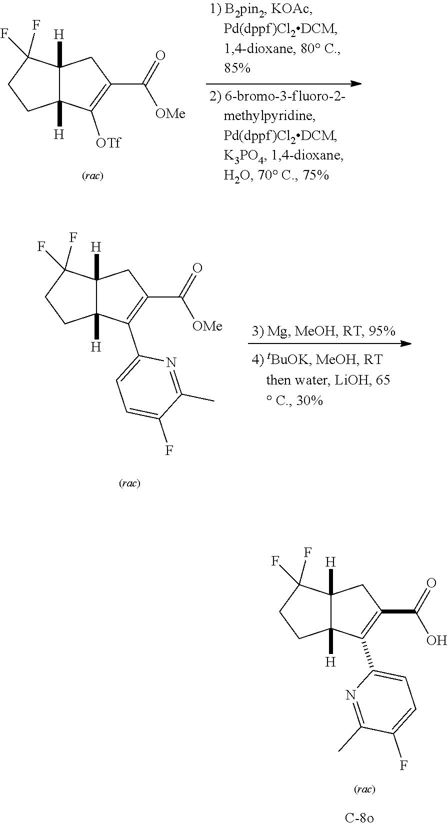

### Step 1

KOAc (1.4 g, 14.26 mmol) was added to a solution of methyl rac-(3aR,6aR)-6,6-difluoro-3-(((trifluoromethyl)sulfonyl)oxy)-1,3a,4,5,6,6a-hexahydropentalene-2-carboxylate (1.5 g, 4.283 mmol) and B₂pin₂ (1.7 g, 6.694 mmol) in 1,4-dioxane (22 mL) and nitrogen gas was passed through the solution for 10 min. Pd(dppf)Cl₂·DCM (354 mg, 0.433 mmol) was added and the reaction mixture was stirred at 80° C. for 17 h. The reaction was cooled to ambient temperature and filtered over a pad of Celite®, washing the filtered cake with EtOAc (100 mL). The filtrates were collected and washed with water (2×50 mL) and brine (50 mL). The organic layer was dried (MgSO₄), filtered and concentrated in vacuo to give methyl rac-(3aR,6aR)-6,6-difluoro-3-(4,4,5,5-tetramethyl-1,3,2-dioxaborolan-2-yl)-1,3a,4,5,6,6a-hexahydropentalene-2-carboxylate (1.2 g, 85%) as a dark-amber oil. ESI-MS m/z calc. 328.166, found 329.2 (M+1)⁺.

### Step 2

K₃PO₄ (2.28 g, 10.74 mmol) was added to a solution of methyl rac-(3aR,6aR)-6,6-difluoro-3-(4,4,5,5-tetramethyl-1,3,2-dioxaborolan-2-yl)-1,3a,4,5,6,6a-hexahydropentalene-2-carboxylate (1.2 g, 3.657 mmol) and 6-bromo-3-fluoro-2-methylpyridine (700 mg, 3.684 mmol) in a mixture of 1,4-dioxane (15 mL) and water (5 mL) and nitrogen gas was passed through the solution for 5 min. Pd(dppf)Cl₂·DCM (213 mg, 0.261 mmol) was added and the reaction mixture was heated at 70° C. for 5 h. The reaction mixture was cooled to ambient temperature, partitioned between EtOAc (20 mL) and water (10 mL). The organic layer was separated, washed with brine (1×10 mL), dried (MgSO₄), filtered and concentrated in vacuo. Purification by flash chromatography (SiO₂, 0 to 25% EtOAc in hexanes) gave methyl rac-(3aR,6aR)-6,6-difluoro-3-(5-fluoro-6-methylpyridin-2-yl)-1,3a,4,5,6,6a-hexahydropentalene-2-carboxylate (945 mg, 75%) as a clear oil. ESI-MS m/z calc. 311.113, found 312.5 (M+1)⁺.

### Step 3

Mg powder (670 mg, 27.57 mmol) was added in one portion to a solution of methyl rac-(3aR,6aR)-6,6-difluoro-3-(5-fluoro-6-methylpyridin-2-yl)-1,3a,4,5,6,6a-hexahydropentalene-2-carboxylate (945 mg, 2.732 mmol) in MeOH (55 mL) and the reaction mixture was stirred at ambient temperature for 3 h. The reaction was diluted with a 1 N aqueous HCl solution, stirred vigorously until it became homogeneous (15 min) and the aqueous solution was extracted with EtOAc. The combined organic layers were washed with a saturated aqueous NaHCO₃ solution and brine, dried (MgSO₄), filtered and concentrated in vacuo to give methyl dia-(1R,2S,3aR¹,6aS²)-4,4-difluoro-1-(5-fluoro-6-methylpyridin-2-yl)octahydropentalene-2-carboxylate (856 mg, 95%). ESI-MS m/z calc. 313.129, found 314.5 (M+1)⁺.

### Step 4

^{t}BuOK (457 mg, 4.073 mmol) was added to a solution of methyl dia-(1R,2S,3aR¹,6aS¹)-4,4-difluoro-1-(5-fluoro-6-methylpyridin-2-yl)octahydropentalene-2-carboxylate (856 mg, 2.595 mmol) in MeOH (25 mL) and the reaction mixture was stirred at ambient temperature overnight. Water (10 mL) and LiOH monohydrate (266 mg, 6.339 mmol) were successively added and the reaction mixture was heated at 65° C. for 4 h. The reaction was cooled to ambient temperature, partitioned between a 1 N aqueous HCl solution (150 mL) and EtOAc (50 mL). The aqueous layer was separated and extracted with EtOAc (2×50 mL). The combined organic layers were washed with water (2×40 mL) and brine (40 mL), dried (MgSO₄), filtered and concentrated in vacuo. Purification by flash chromatography (SiO₂, 0 to 50% of EtOAc in heptane) gave:

First eluting isomer: rac-(1S,2S,3aR,6aS)-4,4-difluoro-1-(5-fluoro-6-methylpyridin-2-yl)octahydropentalene-2-carboxylic acid (225 mg, 29%) as an off-white solid. ESI-MS m/z calc. 299.113, found 300.4 (M+1)⁺.

Second eluting isomer: rac-(1R,2R,3aR,6aS)-4,4-difluoro-1-(5-fluoro-6-methylpyridin-2-yl)octahydropentalene-2-carboxylic acid (C-8o, 233 mg, 30%) as an off-white solid. ESI-MS m/z calc. 299.113, found 300.4 (M+1)⁺.

### Intermediate C-9a

### dia-(1R¹,2R¹,4S²)-2-(3,4-Difluoro-2-methylphenyl)-4-methyl-4-(trifluoromethyl)cyclopentane-1-carboxylic acid

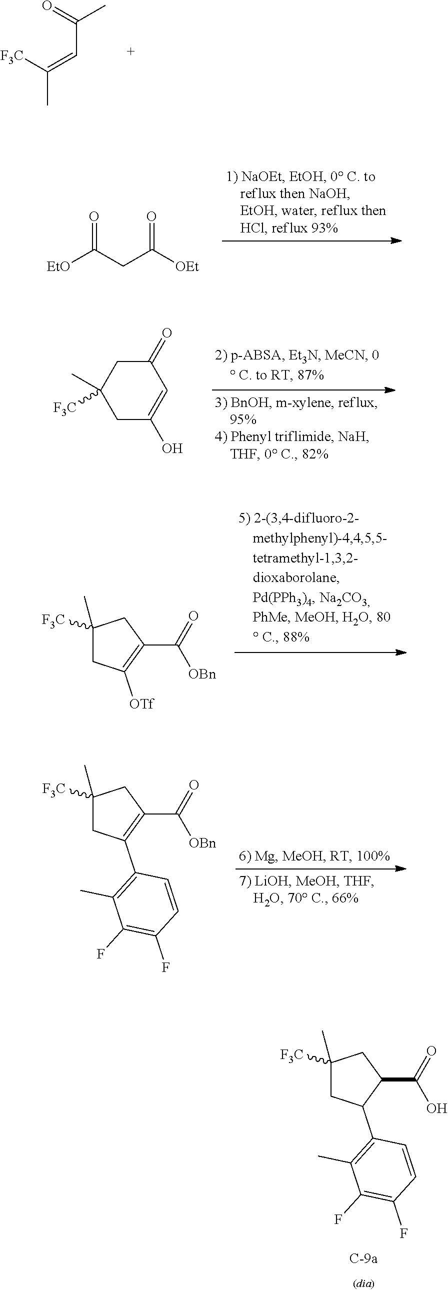

### Step 1

Diethyl malonate (56 mL, 368.900 mmol) was added dropwise to a solution of NaOEt (76.5 mL, 21% w/v in EtOH, 204.900 mmol) in EtOH (210 mL) at 0° C. and the reaction mixture was stirred at ambient temperature for 2 h. The mixture was cooled to 0° C., (Z)-5,5,5-trifluoro-4-methylpent-3-en-2-one (27.8 g, 182.800 mmol) was added dropwise and the mixture was stirred at ambient temperature overnight then, at reflux for 18 h. The mixture was cooled to ambient temperature and concentrated in vacuo. The residue was taken in a mixture of EtOH (200 mL) and water (150 mL). NaOH (66.12 g, 1.653 mol) was added, and the mixture was heated to reflux for 1.5 h. The reaction was concentrated to half of its volume. 12 M HCl (154 mL, 1.848 mol) was added dropwise and the reaction mixture was heated to reflux for 30 min. The reaction was cooled to ambient temperature. The solid was filtered, washed with water (3 x) and heptane (2 x), dried in an oven at 40° C. under vacuum overnight to give rac-3-hydroxy-5-methyl-5-(trifluoromethyl)cyclohex-2-en-1-one (33 g, 93%). ¹H NMR (300 MHz, DMSO-d₆) δ 11.45 (s, 1H), 5.29 (d, J=0.9 Hz, 1H), 2.62 (d, J=16.8 Hz, 2H), 2.25 (d, J=16.7 Hz, 2H), 1.21 (s, 3H) ppm. ESI-MS m z calc. 194.055, found 195.3 (M+1)⁺.

### Step 2

Et₃N (13 mL, 93.270 mmol) was added dropwise to a suspension of rac-3-hydroxy-5-methyl-5-(trifluoromethyl)cyclohex-2-en-1-one (16.4 g, 84.470 mmol) and p-ABSA (20.3 g, 84.500 mmol) in MeCN (200 mL) at 0° C. and the reaction mixture was stirred at ambient temperature overnight. The reaction was concentrated in vacuo. The residue was suspended in MTBE and filtered through a pad of Celite®. The filtrates were concentrated in vacuo. Purification by flash chromatography (330 g SiO₂, 0 to 50% of EtOAc in heptane) gave rac-2-diazo-5-methyl-5-(trifluoromethyl)cyclohexane-1,3-dione (16.2 g, 87%) as a clear yellow oil which solidified on standing. ¹H NMR (300 MHz, Chloroform-d) δ 2.84 (dd, J=17.1, 1.1 Hz, 2H), 2.71-2.57 (m, 2H), 1.35 (s, 3H) ppm. ESI-MS m/z calc. 220.046, found 221.1 (M+1)⁺.

### Step 3

A stirred mixture of rac-2-diazo-5-methyl-5-(trifluoromethyl)cyclohexane-1,3-dione (16.2 g, 73.590 mmol) and BnOH (15 mL, 145 mmol) in m-xylene (170 mL) was heated at 145° C. for 2.5 h. The reaction mixture was cooled to ambient temperature and concentrated in vacuo. Purification by flash chromatography (330 g SiO₂, 0 to 40% of EtOAc in heptane) gave benzyl dia-(1R′,4R²)-4-methyl-2-oxo-4-(trifluoromethyl)cyclopentane-1-carboxylate (21 g, 95%) as a clear oil, which solidified on standing. ¹H NMR (300 MHz, Chloroform-d) δ 7.40 (d, J=3.6 Hz, 5H), 5.32-5.16 (m, 2H), 3.66-3.44 (m, 1H), 3.08-2.87 (m, 1H), 2.84-2.61 (m, 1H), 2.46-2.14 (m, 2H), 1.48-1.28 (m, 3H) ppm. ESI-MS m/z calc. 300.097, found 301.2 (M+1)⁺.

### Step 4

NaH (6 g, 60% dispersion in mineral oil, 150 mmol) was added to a solution of benzyl dia-(1R′,4R²)-4-methyl-2-oxo-4-(trifluoromethyl)cyclopentane-1-carboxylate (30.8 g, 102.600 mmol) in dry THF (300 mL) at 0° C. and the reaction mixture was stirred at 0° C. for 15 min. Phenyl triflimide (50 g, 140 mmol) was added and the reaction was stirred overnight. The mixture was concentrated in vacuo. The residue was suspended in MTBE (400 mL) and the solids were filtered off. The filtrates were collected, washed with brine (2 x), dried (Na₂SO₄), filtered and concentrated in vacuo. Purification by flash chromatography (800 g SiO₂, 0 to 40% EtOAc in heptane) gave benzyl rac-4-methyl-4-(trifluoromethyl)-2-(((trifluoromethyl)sulfonyl)oxy)cyclopent-1-ene-1-carboxylate (43 g, 82%). ¹H NMR (300 MHz, Chloroform-d) δ 7.48-7.30 (m, 5H), 5.27 (s, 2H), 3.29-3.01 (m, 2H), 2.56 (dt, J=17.2, 2.6 Hz, 2H), 1.39 (s, 3H) ppm.

### Step 5

In a sealed tube, nitrogen gas was passed through a stirred mixture of benzyl rac-4-methyl-4-(trifluoromethyl)-2-(((trifluoromethyl)sulfonyl)oxy)cyclopent-1-ene-1-carboxylate (4 g, 9.252 mmol), 2-(3,4-difluoro-2-methylphenyl)-4,4,5,5-tetramethyl-1,3,2-dioxaborolane (3 g, 11.807 mmol) and Na₂CO₃ (2.5 g, 23.588 mmol) in a mixture of toluene (44 mL), MeOH (4 mL) and water (4 mL) for 10 min and Pd(PPh₃)₄(1 g, 0.865 mmol) was added to the reaction mixture. The tube was sealed and the mixture was stirred at 80° C. overnight. The reaction was cooled to ambient temperature and concentrated in vacuo. Purification by flash chromatography (SiO₂, 0 to 10% EtOAc in heptane) gave benzyl rac-2-(3,4-difluoro-2-methylphenyl)-4-methyl-4-(trifluoromethyl)cyclopent-1-ene-1-carboxylate (3.47 g, 88%) as a clear oil. ¹H NMR (400 MHz, Chloroform-d) δ 7.33-7.27 (m, 3H), 7.05-6.99 (m, 2H), 6.96-6.87 (m, 1H), 6.74-6.68 (m, 1H), 4.99 (s, 2H), 3.27 (dt, J=17.2, 2.3 Hz, 1H), 3.15 (dt, J=18.7, 2.4 Hz, 1H), 2.72 (dd, J=17.1, 2.0 Hz, 1H), 2.50 (dd, J=18.6, 2.2 Hz, 1H), 2.05 (d, J=2.0 Hz, 3H, overlapped with EtOAc), 1.41 (s, 3H) ppm. ESI-MS m/z calc. 410.131, found 411.2 (M+1)⁺.

### Step 6

Mg (5 g, 205.720 mmol) was added portionwise to a solution of benzyl rac-2-(3,4-difluoro-2-methylphenyl)-4-methyl-4-(trifluoromethyl)cyclopent-1-ene-1-carboxylate (3.47 g, 8.143 mmol) in MeOH (130 mL) and the reaction mixture was stirred at ambient temperature for 17 h. Additional Mg (1 g, 41.144 mmol) was added and the mixture was stirred at ambient temperature for 5 h. The reaction was quenched by slow addition of 6 M aqueous HCl (100 mL), stirred at ambient temperature for 1 h and extracted with MTBE (2×200 mL). The combined organic extracts were dried (Na₂SO₄), filtered and concentrated in vacuo. Purification by flash chromatography (SiO₂, 0 to 20% EtOAc in heptane) gave methyl rac-2-(3,4-difluoro-2-methylphenyl)-4-methyl-4-(trifluoromethyl)cyclopentane-1-carboxylate (3.08 g, 100%) as a yellow oil. ESI-MS m/z calc. 336.115, found 337.2 (M+1)⁺.

### Step 7

A solution of methyl rac-2-(3,4-difluoro-2-methylphenyl)-4-methyl-4-(trifluoromethyl)cyclopentane-1-carboxylate (3.08 g, 8.151 mmol) and LiOH·H₂O (3.95 g, 65.857 mmol) in a mixture of THF (50 mL), MeOH (25 mL) and water (25 mL) was stirred at 70° C. overnight. The mixture was cooled to ambient temperature, diluted with 1 N aqueous HCl (200 mL) and extracted with EtOAc (2×250 mL). The combined organic extracts were washed with brine (100 mL), dried (Na₂SO₄), filtered and concentrated in vacuo. Purification by reverse phase chromatography (Cis column, 5 to 100% MeCN in water with 0.1% formic acid) gave dia-(1R¹,2R¹,4S²)-2-(3,4-difluoro-2-methylphenyl)-4-methyl-4-(trifluoromethyl)cyclopentane-1-carboxylic acid (C-9a, 2 g, 66%) as a tan solid. ¹H NMR (400 MHz, Chloroform-d) δ 7.05-6.92 (m, 2H), 3.71-3.57 (m, 1H), 3.19-3.09 (m, 1H), 2.64-2.52 (m, 1H), 2.37 (t, J=12.6 Hz, 0.5H), 2.28 (dd, J=5.7, 2.6 Hz, 3H), 2.11-2.04 (m, 1H), 1.98 (d, J=10.0 Hz, 1H), 1.86 (dd, J=14.2, 10.8 Hz, 0.5H), 1.37 (d, J=15.2 Hz, 3H) ppm; exchangeable H not observed. ESI-MS m/z calc. 322.099, found 323.2 (M+1)⁺.

The following Intermediates were made using the method described in Intermediate C-9, except that, the conditions used in Step 5 were those of Intermediate C-2 Step 2 and different Suzuki coupling partners were used in place of 2-(3,4-difluoro-2-methylphenyl)-4,4,5,5-tetramethyl-1,3,2-dioxaborolane. In the case of Intermediate C-9f, Step 6 was carried out at 0° C. In the case of Intermediate C-9j, the conditions used in Step 7 were those of Intermediate C-8a Step 10 and the hydrolysis was carried out at 50° C.:

| | | LC/MS | |
|---|---|---|---|
| Inter. | | (m/z | Found |
| No. | Intermediate Name | calc.) | M + 1 |
| C - 9b | rac-(1R,2R,4S)-2-(4- | 334.119 | 335.2 |
| | fluoro-2-methoxy-3- | | |
| | methylphenyl)-4-methyl-4- | | |
| | (trifluoromethyl)cyclopentane- | | |
| | 1-carboxylic acid | | |
| C - 9d | dia-(1R¹,2R¹,4S²)-2-(4- | 318.124 | 319.3 |
| | fluoro-2,3- | | |
| | dimethylphenyl)-4- | | |
| | methyl-4- | | |
| | (trifluoromethyl)cyclopentane- | | |
| | 1-carboxylic acid | | |
| C - 9e | rac-(1R,2R,4S)-2-(3- | 354.065 | No |
| | chloro-4-fluoro-2- | | ionisation |
| | methoxyphenyl)-4- | | |
| | methyl-4- | | |
| | (trifluoromethyl)cyclopentane- | | |
| | 1-carboxylic acid | | |
| C - 9f | dia-(1R¹,2R¹,4S²)-2-(3- | 338.070 | 339.0 |
| | chloro-4-fluoro-2- | | |
| | methylphenyl)-4-methyl-4- | | |
| | (trifluoromethyl)cyclopentane- | | |
| | 1-carboxylic acid | | |
| C - 9g | rac-(1R,2R,4S)-2-(3- | 324.054 | 325.3 |
| | chloro-4-fluorophenyl)-4- | | |
| | methyl-4- | | |
| | (trifluoromethyl)cyclopentane- | | |
| | 1-carboxylic acid | | |
| C - 9h | rac-(1R,2R,4R)-2-(2- | 336.115 | 337.3 |
| | ethyl-3,4- | | |
| | difluorophenyl)-4- | | |
| | methyl-4- | | |
| | (trifluoromethyl)cyclopentane- | | |
| | 1-carboxylic acid | | |
| C - 9i | rac-(1R,2R,4S)-2-(2- | 336.115 | 337.3 |
| | ethyl-3,4- | | |
| | difluorophenyl)-4- | | |
| | methyl-4- | | |
| | (trifluoromethyl)cyclopentane- | | |
| | 1-carboxylic acid | | |
| C - 9j | rac-(1R,2R,4S)-2-(2- | 348.115 | 349.3 |
| | cyclopropyl-3,4- | | |
| | difluorophenyl)-4- | | |
| | methyl-4- | | |
| | (trifluoromethyl)cyclopentane- | | |
| | 1-carboxylic acid | | |

The following Intermediate was made using the method described in Intermediate C-9, except that, in Step 5, a different Suzuki coupling partner was used in place of 2-(3,4-difluoro-2-methylphenyl)-4,4,5,5-tetramethyl-1,3,2-dioxaborolane:

| | | LC/MS | | |
|---|---|---|---|---|
| Inter. | | (m/z | Found | |
| No. | Intermediate Name | calc.) | M + 1 | NMR (shifts in ppm) |
| C - 9c | dia-(1R¹,2R¹,4S²)-2-(3,4- | 338.094 | 339.1 | ¹H NMR (400 MHz, Methanol-d₄) δ |
| | difluoro-2- | | | 7.12-7.01 (m, 1H), 6.98-6.90 (m, 1H), |
| | methoxyphenyl)-4- | | | 3.96 (t, J = 2.0 Hz, 3H), 3.80-3.71 (m, |
| | methyl-4- | | | 0.5H), 3.70-3.60 (m, 0.5H), 3.28-3.18 |
| | (trifluoromethyl)cyclopentane- | | | (m, 0.5H), 3.18-3.09 (m, 0.5H), 2.55- |
| | 1-carboxylic acid | | | 2.44 (m, 1H), 2.35-2.27 (m, 0.5H), |
| | | | | 2.14-2.06 (m, 0.5H), 2.04-1.84 (m, |
| | | | | 1.5H), 1.61-1.52 (m, 0.5H), 1.36 (d, J = |
| | | | | 8.3 Hz, 3H) ppm; exchangeable H not |
| | | | | observed. |

The following Intermediates were made using the method described in Intermediate C-9, except that, Steps i to 4 were omitted. In step 5, different Suzuki coupling partners were used in place of 2-(3,4-difluoro-2-methylphenyl)-4,4,5,5-tetramethyl-1,3,2-dioxaborolane and/or benzyl rac-4-methyl-4-(trifluoromethyl)-2-(((trifluoromethyl) sulfonyl)oxy)cyclopent-1-ene-1-carboxylate:

| | | LC/MS | | |
|---|---|---|---|---|
| Inter. | | (m/z | Found | |
| No. | Intermediate Name | calc.) | M + 1 | NMR (shifts in ppm) |
| C - 11a | rac-(1R,2R)-2-(3,4- | 298.138 | 299.2 | ¹H NMR (400 MHz, Chloroform-d) δ |
| | difluoro-2- | | | 6.87-6.78 (m, 2H), 3.95 (s, 3H), 3.46- |
| | methoxyphenyl)-4,4- | | | 3.35 (m, 1H), 2.62 (td, J = 12.0, 3.5 Hz, |
| | dimethylcyclohexane-1- | | | 1H), 2.02-1.93 (m, 1H), 1.82 (qd, J = |
| | carboxylic acid | | | 13.2, 3.8 Hz, 1H), 1.62-1.45 (m, 2H), |
| | | | | 1.39-1.19 (m, 2H), 1.08 (s, 3H), 0.96 |
| | | | | (s, 3H) ppm; exchangeable H not |
| | | | | observed. |
| C - 11b | dia-(1R¹,2R²)-2-(3,4- | 282.143 | 283.1 | |
| | difluoro-2- | | | |
| | methylphenyl)-4,4- | | | |
| | dimethylcyclohexane-1- | | | |
| | carboxylic acid | | | |

### Intermediate C-12c and C-10c

### Mixture of dia-(1R¹,2R²,3S²,5R¹)-5-(3-chloro-4-fluorophenyl)-2,3-dimethyl-3-(trifluoromethyl)cyclopentane-1-carboxylic acid and rac-(1R,2R,3S,4R)-2-(3-chloro-4-fluorophenyl)-3,4-dimethyl-4-(trifluoromethyl)cyclopentane-1-carboxylic acid

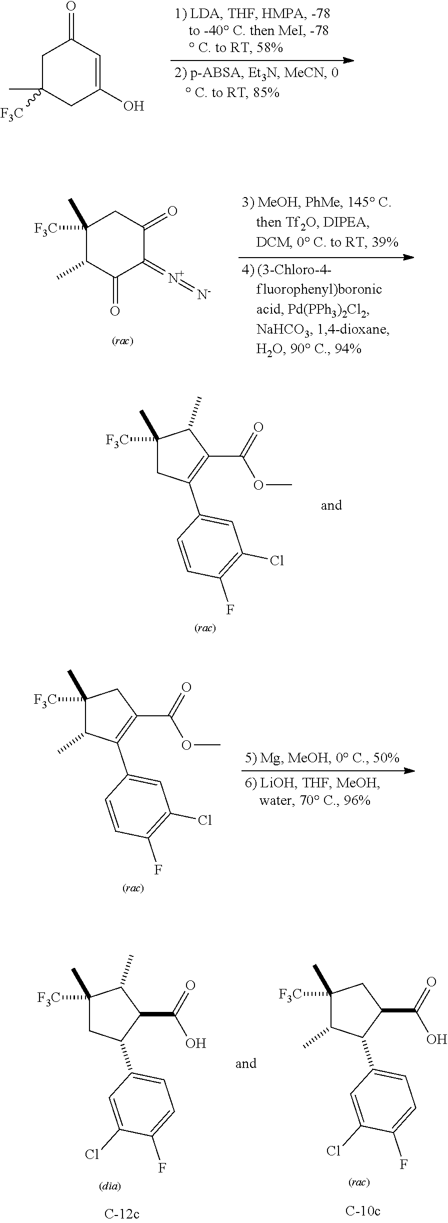 [SMILES: CC1(C(F)(F)F)CC(=O)C=C(O)C1 ; C[C@H]1C(=O)C(=[N+]=[N-])C(=O)C[C@@]1(C)C(F)(F)F ; COC(=O)C1=C(c2ccc(F)c(Cl)c2)C[C@](C)(C(F)(F)F)[C@@H]1C ; COC(=O)C1=C(c2ccc(F)c(Cl)c2)[C@H](C)[C@](C)(C(F)(F)F)C1 ; C[C@@H]1[C@@H](C(=O)O)[C@H](c2ccc(F)c(Cl)c2)C[C@]1(C)C(F)(F)F ; C[C@H]1[C@@H](c2ccc(F)c(Cl)c2)[C@H](C(=O)O)C[C@@]1(C)C(F)(F)F]

### Step 1

LDA (42 mL, 2 M solution in hexanes, 84 mmol) was slowly added to a stirred solution of rac-3-hydroxy-5-methyl-5-(trifluoromethyl)cyclohex-2-en-1-one (6 g, 30.900 mmol) and HMPA (11 mL, 63.220 mmol) in THF (60 mL) at −78° C., keeping the internal temperature below −60° C., and the reaction mixture was stirred at −78° C. for 1 h. The reaction was warmed to 0° C., re-cooled to −40° C. and stirred for 20 min. The mixture was cooled to −78° C. and Mel (3.6 mL, 57.830 mmol) was added rapidly. The reaction mixture was slowly warmed to ambient temperature and stirred for 16 h. The reaction was quenched by addition of a saturated NH₄Cl solution, diluted with a 3 N aqueous HCl solution (30 mL) and extracted with EtOAc (3×30 mL). The combined organic layers were washed with a 1 N aqueous HCl solution (20 mL) and brine (20 mL), dried (Na₂SO₄), filtered and concentrated in vacuo. Purification by flash chromatography (SiO₂,0 to 20% of EtOAc in hexanes) gave dia-(4R′,5R²)-3-hydroxy-4,5-dimethyl-5-(trifluoromethyl)cyclohex-2-en-1-one (4.5 g, 58%). H NMR (400 MHz, DMSO-d₆) δ 11.58-11.28 (m, 1H), 5.20 (s, 1H), 2.86-2.69 (m, 1H), 2.46-2.25 (m, 2H), 1.25 (s, 3H), 1.18-1.04 (m, 3H) ppm. ESI-MS m/z calc. 208.071, found 209.4 (M+1)⁺.

### Step 2

Et₃N (4.25 mL, 30.49 mmol) was added dropwise to a suspension of dia-(4R′,5R²)-3-hydroxy-4,5-dimethyl-5-(trifluoromethyl)cyclohex-2-en-1-one (5 g, 24.020 mmol) and p-ABSA (7.7 g, 32.050 mmol) in MeCN (60 mL) at 0° C. and the reaction mixture was stirred at 0° C. for 15 min and at ambient temperature for 1 h. The insoluble material was removed by filtration. The filtrates were collected and concentrated in vacuo. The residue was suspended in DCM and the precipitate was removed by filtration. The filtrates were collected and concentrated in vacuo. Purification by flash chromatography (SiO₂, 0% to 20% of EtOAc in hexanes) gave:

First Eluting Isomers: rac-(4R,5S)-2-diazo-4,5-dimethyl-5-(trifluoromethyl)cyclohexane-1,3-dione as a pale yellow solid (2.58 g, 46%). ¹H NMR (400 MHz, DMSO-d₆) δ 3.06-2.88 (m, 2H), 2.70 (d, J=17.1 Hz, 1H), 1.20 (dd, J=7.1, 1.4 Hz, 3H), 1.17 (s, 3H) ppm. ESI-MS m/z calc. 234.061, found 235.5 (M+1)⁺.

Second Eluting Isomers: rac-(4R,5R)-2-diazo-4,5-dimethyl-5-(trifluoromethyl)cyclohexane-1,3-dione (2.33 g, 39%) as a pale yellow oil. ¹H NMR (400 MHz, DMSO-d₆) δ 2.98-2.87 (m, 2H), 2.79 (d, J=17.4 Hz, 1H), 1.33 (s, 3H), 1.30-1.20 (m, 3H) ppm. ESI-MS m/z calc. 234.061, found 235.2 (M+1)⁺.

### Step 3

In a high-pressure vessel, a mixture of rac-(4R,5R)-2-diazo-4,5-dimethyl-5-(trifluoromethyl)cyclohexane-1,3-dione (2.3 g, 9.822 mmol) and MeOH (1.5 mL, 37.030 mmol) in toluene (40 mL) was heated at 145° C. for 5 h. The reaction was cooled to ambient temperature and concentrated in vacuo. Tf₂O (2 mL, 11.890 mmol) was added dropwise over 5 min to a solution of the residue and DIPEA (4.4 mL, 25.260 mmol) in DCM (23 mL) at 0° C. and the reaction was warmed and stirred at ambient temperature overnight. The mixture was quenched by addition of a saturated aqueous NH₄Cl solution (10 mL). The aqueous layer was separated and extracted with DCM (30 mL). The combined organic layers were washed with brine (30 mL), dried (MgSO₄), filtered and concentrated in vacuo. Purification by flash chromatography (SiO₂, 0 to 20% EtOAc in hexanes) gave a mixture of methyl rac-(4S,5R)-4,5-dimethyl-4-(trifluoromethyl)-2-(((trifluoromethyl)sulfonyl)oxy)cyclopent-1-ene-1-carboxylate and methyl rac-(3R,4R)-3,4-dimethyl-4-(trifluoromethyl)-2-(((trifluoromethyl)sulfonyl)oxy)cyclopent-1-ene-1-carboxylate (1.58 g, 39%). ESI-MS m/z calc. 370.031, found 371.2 (M+1)⁺.

### Step 4

A stirred suspension of a mixture of methyl rac-(4S,5R)-4,5-dimethyl-4-(trifluoromethyl)-2-(((trifluoromethyl)sulfonyl)oxy)cyclopent-1-ene-1-carboxylate and methyl rac-(3R,4R)-3,4-dimethyl-4-(trifluoromethyl)-2-(((trifluoromethyl)sulfonyl)oxy)cyclopent-1-ene-1-carboxylate (1.58 g, 3.841 mmol), (3-chloro-4-fluorophenyl)boronic acid (685 mg, 3.930 mmol) and Pd(PPh₃)₂Cl₂ (30 mg, 0.043 mmol) in a mixture of 1,4-dioxane (12 mL) and a saturated aqueous NaHCO₃ solution (7 mL) was degassed by passing nitrogen gas through the mixture for 5 min and the reaction vessel was sealed and heated at 90° C. for 2 h. The reaction was cooled to ambient temperature and partitioned between EtOAc and water. The organic phase was separated, dried (MgSO₄), filtered and concentrated in vacuo. Purification by flash chromatography (SiO₂, 0 to 10% EtOAc in hexanes) gave a mixture of methyl rac-(4S,5R)-2-(3-chloro-4-fluorophenyl)-4,5-dimethyl-4-(trifluoromethyl)cyclopent-1-ene-1-carboxylate and methyl rac-(3S,4R)-2-(3-chloro-4-fluorophenyl)-3,4-dimethyl-4-(trifluoromethyl)cyclopent-1-ene-1-carboxylate (1.3 g, 94%). ESI-MS m/z calc. 350.070, found 351.4 (M+1)⁺.

### Step 5

Mg powder (100 mg, 3.799 mmol) was added to a stirred solution of a mixture of methyl rac-(4S,5R)-2-(3-chloro-4-fluorophenyl)-4,5-dimethyl-4-(trifluoromethyl)cyclopent-1-ene-1-carboxylate and methyl rac-(3S,4R)-2-(3-chloro-4-fluorophenyl)-3,4-dimethyl-4-(trifluoromethyl)cyclopent-1-ene-1-carboxylate (1.3 g, 3.706 mmol) in MeOH (20 mL) at 0° C. and the reaction mixture was stirred vigorously for 40 min. The reaction was partitioned between 1 N aqueous HCl and EtOAc. The aqueous layer was separated and extracted with EtOAc (3 x). The combined organic layers were dried (MgSO₄), filtered and concentrated in vacuo. Purification by flash chromatography (SiO₂, 0 to 10% EtOAc in hexanes) gave:

First Eluting Isomers: mixture of methyl dia-(1S′,2R²,3S²,5R′)-5-(3-chloro-4-fluorophenyl)-2,3-dimethyl-3-(trifluoromethyl)cyclopentane-1-carboxylate and rac-(1S,2R,3S,4R)-2-(3-chloro-4-fluorophenyl)-3,4-dimethyl-4-(trifluoromethyl)cyclopentane-1-carboxylic acid (655 mg, 50%). ESI-MS m/z calc. 352.085, found 353.2 (M+1)⁺.

Second Eluting Isomers: rac-(1R,2S,3S,4R)-2-(3-chloro-4-fluorophenyl)-3,4-dimethyl-4-(trifluoromethyl)cyclopentane-1-carboxylic acid was not collected.

### Step 6

LiOH·H₂O (800 mg, 19.07 mmol) was added to a solution of a mixture of methyl dia-(1S′,2R²,3S²,5R′)-5-(3-chloro-4-fluorophenyl)-2,3-dimethyl-3-(trifluoromethyl)cyclopentane-1-carboxylate and rac-(1S,2R,3S,4R)-2-(3-chloro-4-fluorophenyl)-3,4-dimethyl-4-(trifluoromethyl)cyclopentane-1-carboxylic acid (650 mg, 1.843 mmol) in THF (5 mL), MeOH (2.5 mL) and water (2.5 mL) and the reaction mixture was stirred and heated at 70° C. for 30 h. The reaction was cooled to ambient temperature and partitioned between 1 N HCl and EtOAc. The aqueous layer was separated and extracted with EtOAc (2 x). The combined organic layers were dried (MgSO₄), filtered and concentrated in vacuo to give a mixture of dia-(1R¹,2R²,3S,²5R¹)-5-(3-chloro-4-fluorophenyl)-2,3-dimethyl-3-(trifluoromethyl)cyclopentane-1-carboxylic acid (C-12c) and rac-(1R,2R,3S,4R)-2-(3-chloro-4-fluorophenyl)-3,4-dimethyl-4-(trifluoromethyl)cyclopentane-1-carboxylic acid (C-10c) (600 mg, 96%). ESI-MS m/z calc. 338.070, found 339.2 (M+1)⁺.

The following Intermediate was made using the method described in Intermediate C-10c, except that, in Step 4, a different Suzuki coupling partner was used in place of (3-chloro-4-fluorophenyl)boronic acid:

| | | LC/MS | |
|---|---|---|---|
| Inter. | | (m/z | Found |
| No. | Intermediate Name | calc.) | M − 1 |
| C - 10a | rac-(1R,2R,3S,4R)-2-(3- | 352.085 | 353.4 |
| | chloro-4-fluoro-2- | | |
| | methylphenyl)-3,4- | | |
| | dimethyl-4- | | |
| | (trifluoromethyl)cyclopentane- | | |
| | 1-carboxylic acid | | |

The following Intermediate was made using the method described in Intermediate C-10c, except that, the conditions used in Steps 4 to 6 were those of Intermediate C-12a Steps 2 to 4. In Step 4, a different Suzuki coupling partner was used as the starting material. The reduction Step 5 was carried out in absence of EtOAc at 60° C. and at a hydrogen pressure of 50 psi:

| | | LC/MS | | |
|---|---|---|---|---|
| Inter. | | (m/z | Found | |
| No. | Intermediate Name | calc.) | M − 1 | NMR (shifts in ppm) |
| C - 10b | rac-(1R,2R,3S,4R)-2-(3,4- | 352.110 | 353.1 | ¹H NMR (400 MHz, Chloroform-d) δ |
| | difluoro-2- | | | 6.93-6.80 (m, 2H), 4.05 (dd, J = 12.3, |
| | methoxyphenyl)-3,4- | | | 7.2 Hz, 1H), 4.00 (d, J = 2.4 Hz, 3H), |
| | dimethyl-4- | | | 3.39 (dt, J = 12.3, 9.4 Hz, 1H), 2.59 |
| | (trifluoromethyl)cyclopentane- | | | (dd, J = 14.1, 9.9 Hz, 1H), 2.40 (quin, J = |
| | 1-carboxylic acid | | | 7.4 Hz, 1H), 1.87 (dd, J = 14.2, 8.8 |
| | | | | Hz, 1H), 1.48 (s, 3H), 0.71-0.59 (m, |
| | | | | 3H) ppm; exchangeable H not observed. |

The following Intermediate was made using the method described in Intermediate C-10c, except that the conditions used in Steps 4 and 5 were those of Intermediate C-12a Steps 2 and 3. In Step 4, a different Suzuki coupling partner was used as the starting material. Step 6 was carried out at 0° C. to ambient temperature in THF as the solvent, using KO^{t}Bu as the base:

| | | LC/MS | | |
|---|---|---|---|---|
| Inter. | | (m/z | Found | |
| No. | Intermediate Name | calc.) | M − 1 | NMR (shifts in ppm) |
| C - 10d | rac-(1R,2R,3S,4R)-2-(3,4- | 336.115 | 337.1 | ¹H NMR (400 MHz, Chloroform-d) δ |
| | difluoro-2- | | | 7.09-6.93 (m, 2H), 3.30 (t, J = 11.5 |
| | methylphenyl)-3,4- | | | Hz, 1H), 2.99 (q, J = 9.8 Hz, 1H), 2.63 |
| | dimethyl-4- | | | (dd, J = 14.3, 9.4 Hz, 1H), 2.40-2.30 |
| | (trifluoromethyl)cyclopentane- | | | (m, 1H), 2.29 (d, J = 2.2 Hz, 3H), 1.87 |
| | 1-carboxylic acid | | | (dd, J = 14.5, 9.4 Hz, 1H), 1.27 (s, 3H, |
| | | | | overlapped with EtOAc), 0.85 (d, J = |
| | | | | 6.6 Hz, 3H) ppm; exchangeable H not |
| | | | | observed. |

The following Intermediates were made using the method described in Intermediate C-10c, except that Step 1 was omitted. In Step 2, rac-5-(trifluoromethyl)cyclohexane-1,3-dione was used as the starting material in place of dia-(4R′,5R²)-3-hydroxy-4,5-dimethyl-5-(trifluoromethyl)cyclohex-2-en-1-one. In Step 3, BnOH what used in place of MeOH and the triflation was carried out in DCM in place of toluene. In Step 4, different Suzuki coupling partners were used in place of (3-chloro-4-fluorophenyl)boronic acid and the conditions used were those of Intermediate C-1 Step 1. Step 5 was carried out at ambient temperature:

| | | LC/MS | | |
|---|---|---|---|---|
| Inter. | | (m/z | Found | |
| No. | Intermediate Name | calc.) | M − 1 | NMR (shifts in ppm) |
| C - 3b | dia-(1R¹,2R¹,4S²)-2-(3,4- | 324.079 | 323.0 | ¹H NMR (400 MHz, Chloroform-d) δ |
| | difluoro-2- | | | 7.03-6.70 (m, 2H), 4.06-3.86 (m, 3H), |
| | methoxyphenyl)-4- | | | 3.80-3.56 (m, 1H), 3.16-3.02 (m, 1H), |
| | (trifluoromethyl)cyclopentane- | | | 2.99-2.84 (m, 1H), 2.48-2.07 (m, 3H), |
| | 1-carboxylic acid | | | 2.01-1.81 (m, 1H) ppm; exchangeable |
| | | | | H not observed. |
| C - 3c | dia-(1R¹,2R¹,4S²)-2-(3,4- | 308.084 | 307.0 | ¹H NMR (400 MHz, Chloroform-d) δ |
| | difluoro-2-methyphenyl)-4- | | | 7.04-6.83 (m, 2H), 3.74-3.52 (m, 1H), |
| | (trifluoromethyl)cyclopentane- | | | 3.14-2.99 (m, 1H), 2.99-2.85 (m, 1H), |
| | 1-carboxylic acid | | | 2.53-2.31 (m, 2H), 2.31-2.23 (m, 3H), |
| | | | | 2.20-2.08 (m, 1H), 1.86-1.69 (m, 1H) |
| | | | | ppm; exchangeable H not observed. |

### Intermediate C-12a

### dia-(1R′,2R²,3S²,5R′)-5-(3,4-Difluoro-2-methoxyphenyl)-2,3-dimethyl-3-(trifluoromethyl)cyclopentane-1-carboxylic acid

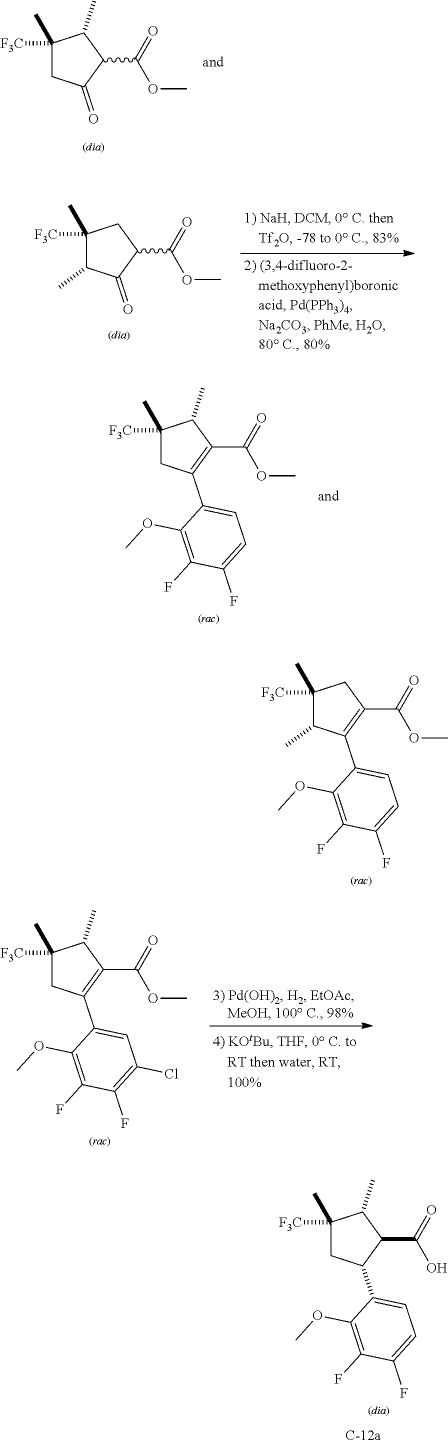

### Step 1

A solution of a mixture of methyl dia-(1R¹,2R²,3S²)-2,3-dimethyl-5-oxo-3-(trifluoromethyl)cyclopentane-1-carboxylate and methyl dia-(1R¹,3R²,4R²)-3,4-dimethyl-2-oxo-4-(trifluoromethyl)cyclopentane-1-carboxylate (10.6 g, products of Intermediate C-10c Step 3 before triflation, 43.298 mmol) in DCM (50 mL) was added to a stirred suspension of NaH (3.7 g, 60% dispersion in mineral oil, 92.509 mmol) in DCM (150 mL) at 0° C. and the reaction mixture was stirred at 0° C. for 30 min. Tf₂O (17.608 g, 10.5 mL, 62.409 mmol) was added dropwise at −78° C. and the temperature of the reaction was maintained at −78° C. for 2 h. The reaction was placed in an ice-water bath and quenched by addition of brine. The mixture was diluted with water (100 mL), brine (50 mL) and DCM (100 mL). The aqueous layer was separated and extracted with DCM (2×80 mL). The combined organic layers were dried (Na₂SO₄), filtered and concentrated in vacuo. Purification by flash chromatography (SiO₂, 0% to 10% of EtOAc in heptane) gave:

First Eluting Isomers: mixture of methyl rac-(4S,5R)-4,5-dimethyl-4-(trifluoromethyl)-2-(((trifluoromethyl)sulfonyl)oxy)cyclopent-1-ene-1-carboxylate and methyl rac-(3R,4R)-3,4-dimethyl-4-(trifluoromethyl)-2-(((trifluoromethyl)sulfonyl)oxy)cyclopent-1-ene-1-carboxylate (9.4 g, 59%) as a light yellow oil. ESI-MS m/z calc. 370.031, found 371.0 (M+1)⁺.

Second Eluting Isomers: methyl rac-(3S,4R)-3,4-dimethyl-4-(trifluoromethyl)-2-(((trifluoromethyl)sulfonyl)oxy)cyclopent-1-ene-1-carboxylate (4.16 g, 24%) as a yellow oil. ¹H NMR (400 MHz, Chloroform-d) δ 3.83 (s, 3H), 3.41-3.31 (m, 1H), 3.09 (dd, J=16.9, 2.4 Hz, 1H), 2.49 (dd, J=16.6, 2.7 Hz, 1H), 1.26 (s, 3H), 1.21 (d, J=7.3 Hz, 3H) ppm. ESI-MS m/z calc. 370.031, found 371.1 (M+1)⁺.

### Step 2

Na₂CO₃ (8 mL, 2 M aqueous solution, 16 mmol) was added to a stirred solution of a mixture of methyl rac-(4S,5R)-4,5-dimethyl-4-(trifluoromethyl)-2-(((trifluoromethyl)sulfonyl)oxy)cyclopent-1-ene-1-carboxylate, methyl rac-(3R,4R)-3,4-dimethyl-4-(trifluoromethyl)-2-(((trifluoromethyl)sulfonyl)oxy)cyclopent-1-ene-1-carboxylate (1 g, 2.698 mmol) and (3,4-difluoro-2-methoxyphenyl)boronic acid (1 g, 5.321 mmol) in toluene (13 mL) and the mixture was degassed by passing nitrogen gas through the solution for 5 min. Pd(PPh₃)₄(420 mg, 0.364 mmol) was added and the reaction mixture was stirred at 80° C. for 4 h. The reaction was cooled to ambient temperature and filtered over a pad of Celite®, washing the filtered cake with EtOAc (30 mL). The filtrates were collected and partitioned between water (50 mL) and EtOAc (30 mL). The aqueous layer was separated and extracted with EtOAc (2×30 mL). The combined organic layers were washed with water (30 mL) and brine (30 mL), dried (Na₂SO₄), filtered and concentrated in vacuo. Purification by flash chromatography (SiO₂, 0 to 8% EtOAc in heptane) gave:

First Eluting Isomers: methyl rac-(4S,5R)-2-(3,4-difluoro-2-methoxyphenyl)-4,5-dimethyl-4-(trifluoromethyl)cyclopent-1-ene-1-carboxylate (621 mg, 63%) as a colourless oil. ¹H NMR (400 MHz, Chloroform-d) δ 6.95-6.77 (m, 2H), 3.91 (d, J=2.2 Hz, 3H), 3.62 (s, 3H), 3.29 (dd, J=17.6, 2.0 Hz, 1H), 3.05 (qd, J=7.1, 2.0 Hz, 1H), 2.44 (d, J=17.6 Hz, 1H), 1.42 (s, 3H), 1.35-1.27 (m, 3H) ppm. ESI-MS m/z calc. 364.110, found 365.1 (M+1)⁺.

Second Eluting Isomers: methyl rac-(3S,4R)-2-(3,4-difluoro-2-methoxyphenyl)-3,4-dimethyl-4-(trifluoromethyl)cyclopent-1-ene-1-carboxylate (171 mg, 17%) as a colourless oil. ¹H NMR (400 MHz, chloroform-d) δ 6.89 (td, J=9.2, 7.1 Hz, 1H), 6.75 (ddd, J=8.5, 5.9, 2.2 Hz, 1H), 3.93 (d, J=2.2 Hz, 3H), 3.62 (s, 3H), 3.24 (dd, J=16.8, 2.3 Hz, 1H), 2.93 (qd, J=7.4, 2.0 Hz, 1H), 2.58 (d, J=16.6 Hz, 1H), 1.45 (s, 3H), 1.05 (dq, J=7.3, 2.3 Hz, 3H) ppm. ESI-MS m/z calc. 364.110, found 365.1 (M+1)⁺.

### Step 3

Under a nitrogen atmosphere, Pd(OH)₂ (2.4 g, 20% on carbon, 64% wet, 1.709 mmol) was added to a solution of methyl rac-(4S,5R)-2-(3,4-difluoro-2-methoxyphenyl)-4,5-dimethyl-4-(trifluoromethyl)cyclopent-1-ene-1-carboxylate (620 mg, 1.700 mmol) in a mixture of EtOAc (75 mL) and MeOH (75 mL) and the reaction mixture was stirred at 100° C. under 200 psi of hydrogen for 72 h. The reaction mixture was filtered through a pad of Celite®, washing the filtered cake with EtOAc (100 mL). The filtrates were concentrated in vacuo to give methyl dia-(1S′,2R²,3S²,5R′)-5-(3-chloro-4-fluorophenyl)-2,3-dimethyl-3-(trifluoromethyl)cyclopentane-1-carboxylate (609 mg, 98%) as a colourless oil. ESI-MS m/z calc. 366.125, found 367.2 (M+1)⁺.

### Step 4

KO^{t}Bu (675 mg, 6.02 mmol) was added to a solution of methyl dia-(1S′,2R²,3S²,5R′)-5-(3-chloro-4-fluorophenyl)-2,3-dimethyl-3-(trifluoromethyl)cyclopentane-1-carboxylate (609 mg, 1.663 mmol) in THF (6 mL) at 0° C. and the reaction mixture was warmed and stirred at ambient temperature overnight. A few drops of water were added and reaction mixture was stirred for 20 h. The reaction was quenched by addition of 1 N aqueous HCl (20 mL) and extracted with EtOAc (3×20 mL). The combined organic layers were dried (Na₂SO₄), filtered and concentrated in vacuo to give dia-(1R′,2R²,3S²,5R′)-5-(3,4-difluoro-2-methoxyphenyl)-2,3-dimethyl-3-(trifluoromethyl)cyclopentane-1-carboxylic (C-12a, 597 mg, 100%) as a yellow oil. ESI-MS m/z calc. 352.110, found 353.1 (M+1)⁺.

### Intermediate C-13a

### rac-(1R,2R,4R)-2-(3,4-Difluoro-2-methoxyphenyl)-4-methyl-4-(trifluoromethyl)cyclohexane-1-carboxylic acid

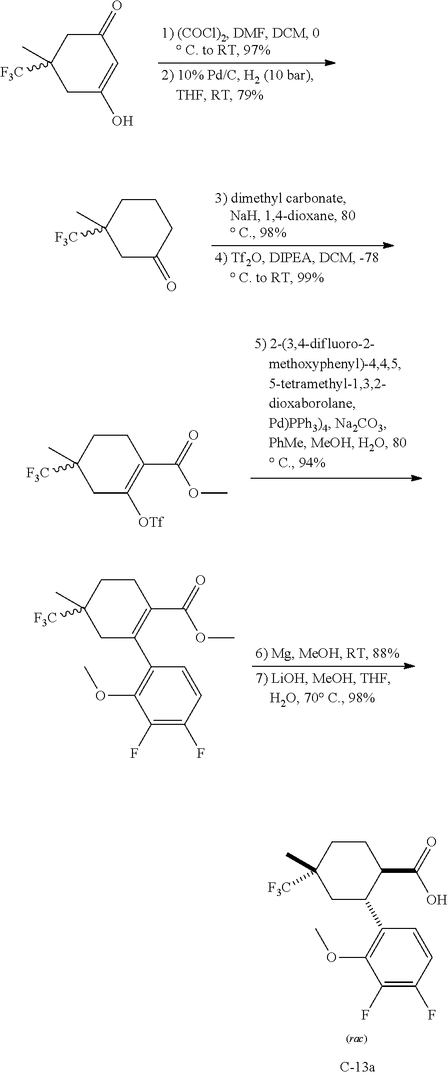 [SMILES: CC1(C(F)(F)F)CC(=O)C=C(O)C1 ; CC1(C(F)(F)F)CCCC(=O)C1 ; COC(=O)C1=C(OS(=O)(=O)C(F)(F)F)CC(C)(C(F)(F)F)CC1 ; COC(=O)C1=C(c2ccc(F)c(F)c2OC)CC(C)(C(F)(F)F)CC1 ; COc1c([C@@H]2C[C@](C)(C(F)(F)F)CC[C@H]2C(=O)O)ccc(F)c1F]

### Step 1

Oxalyl chloride (8.526 g, 5.8 mL, 65.830 mmol) was added dropwise over 30 min to a stirred mixture of rac-3-hydroxy-5-methyl-5-(trifluoromethyl)cyclohex-2-en-1-one (12.02 g, product of Intermediate C-9 Step 1, 58.815 mmol) and DMF (5.381 g, 5.7 mL, 73.615 mmol) in DCM (156 mL) at 0° C. The reaction mixture was warmed to ambient temperature and stirred until gas evolution subsided. The reaction was poured over a mixture of Et₂O (300 mL) and water (120 mL). The aqueous phase was separated and extracted with Et₂O (3×100 mL). The combined organic extracts were dried (MgSO₄), filtered and concentrated in vacuo. Purification by flash chromatography (SiO₂, 10 to 40% EtOAc in hexanes) gave rac-3-chloro-5-methyl-5-(trifluoromethyl)cyclohex-2-en-1-one (12.48 g, 97%) as a pale yellow oil. ¹H NMR (400 MHz, DMSO-d₆) δ 6.38 (dd, J=2.0, 1.3 Hz, 1H), 3.10 (dd, J=18.7, 2.0 Hz, 1H), 2.82 (dt, J=18.6, 1.2 Hz, 1H), 2.71 (d, J=16.2 Hz, 1H), 2.45 (d, J=1.0 Hz, 1H), 1.24 (s, 3H) ppm. ESI-MS m/z calc. 212.022, found 254.0 (M+MeCN)⁺.

### Step 2

Under an argon atmosphere, a solution of rac-3-chloro-5-methyl-5-(trifluoromethyl)cyclohex-2-en-1-one (10.84 g, 47.929 mmol) in THF (217 mL) was added to a Parr bottle containing Pd/C (1.08 g, 10% w/w on carbon, 1.015 mmol) and the mixture was thoroughly degassed (vacuum argon cycles x 3). The flask was saturated with 10 bar of hydrogen gas and the mixture was stirred at ambient temperature overnight. The solution was filtered through a pad of Celite® and the mother liquors were concentrated in vacuo. Purification by flash chromatography (SiO2, 0 to 20% EtOAc in hexanes) gave rac-3-methyl-3-(trifluoromethyl)cyclohexan-1-one (7.45 g, 79%) as a colourless oil. ¹H NMR (300 MHz, DMSO-d₆) δ 2.59 (d, J=13.9 Hz, 1H), 2.45-2.31 (m, 1H), 2.30-2.13 (m, 2H), 1.97-1.64 (m, 4H), 1.08 (s, 3H) ppm.

### Step 3

Under an argon atmosphere, rac-3-methyl-3-(trifluoromethyl)cyclohexan-1-one (16.76 g, 80.931 mmol) was added dropwise over 30 min to a suspension of dimethyl carbonate (30.479 g, 28.8 mL, 334.980 mmol) and NaH (6.48 g, 60% dispersion in mineral oil, 162.020 mmol) in 1,4-dioxane (168 mL) at 80° C. and the reaction mixture was stirred at 80° C. for 2 h. The mixture was cooled to ambient temperature, quenched by addition of 1 N aqueous HCl (20 mL) and concentrated in vacuo to remove the organic solvent. The aqueous residue was extracted with EtOAc (3×15 mL). The combined organic extracts were washed with brine, dried (MgSO₄), filtered and concentrated in vacuo. Purification by flash chromatography (300 g SiO₂, 0 to 10% EtOAc in hexanes then, SiO₂, 10% Et₂O in hexanes) gave methyl rac-2-hydroxy-4-methyl-4-(trifluoromethyl)cyclohex-1-ene-1-carboxylate (19.45 g, 98%) as an off-white semi-solid. ¹H NMR (400 MHz, Chloroform-d) δ 12.13 (s, 1H), 3.78 (s, 3H), 2.57 (d, J=18.5 Hz, 1H), 2.51-2.38 (m, 1H), 2.28-2.09 (m, 2H), 1.75-1.60 (m, 2H), 1.15 (s, 3H) ppm. ESI-MS m/z calc. 238.082, found 239.1 (M+1)⁺.

### Step 4

Tf₂O (587 mg, 0.35 mL, 2.080 mmol) was added dropwise to a stirred solution of methyl rac-2-hydroxy-4-methyl-4-(trifluoromethyl)cyclohex-1-ene-1-carboxylate (400 mg, 1.610 mmol) and DIPEA (415 mg, 0.56 mL, 3.220 mmol) in DCM (5 mL) at −78° C. and the reaction mixture was stirred for 1.5 h while slowly warming the reaction up to ambient temperature. The reaction was quenched by addition of ice-cold water (15 mL) and the aqueous phase was separated and extracted with DCM (2×5 mL). The combined organic extracts were washed with ice-cold water (2×5 mL), brine (5 mL), dried (Na₂SO₄), filtered and concentrated in vacuo to give methyl rac-4-methyl-4-(trifluoromethyl)-2-(((trifluoromethyl)sulfonyl)oxy)cyclohex-1-ene-1-carboxylate (740 mg, 99%), which was used without further purification in the next step. ¹H NMR (300 MHz, Chloroform-d) δ 3.82 (s, 3H), 2.81-2.61 (m, 2H), 2.48 (dddt, J=15.6, 11.2, 6.3, 2.7 Hz, 1H), 2.28-2.11 (m, 1H), 1.81-1.69 (m, 2H), 1.20 (s, 3H) ppm.

### Step 5 to 7

Methyl rac-4-methyl-4-(trifluoromethyl)-2-(((trifluoromethyl)sulfonyl)oxy)cyclohex-1-ene-1-carboxylate (3.140 g, 8.480 mmol) and 2-(3,4-difluoro-2-methoxyphenyl)-4,4,5,5-tetramethyl-1,3,2-dioxaborolane (3.0 g, 11.110 mmol) were treated via the method described in Intermediate C-9a Steps 5 to 7 to give, after purification by reverse phase chromatography (C₁₈ column, 2 to 70% MeCN in water with 0.1% formic acid), rac-(1R,2R,4R)-2-(3,4-difluoro-2-methoxyphenyl)-4-methyl-4-(trifluoromethyl)cyclohexane-1-carboxylic acid (C-13a, 665.5 mg, 98%) as a tan solid. ¹HNMR (400 MHz, Chloroform-d) δ 6.85-6.79 (m, 2H), 3.95 (d, J=2.0 Hz, 3H), 3.43-3.32 (m, 1H), 2.69 (td, J=11.8, 3.7 Hz, 1H), 2.11-2.06 (m, 1H), 1.85-1.78 (m, 1H), 1.78-1.69 (m, 2H), 1.68-1.62 (m, 2H), 1.32 (s, 3H) ppm; exchangeable H not observed. ESI-MS m/z calc. 352.110, found 353.1 (M+1)⁺.

The following Intermediate was made using the method described in Intermediate C-13a, except that, 2-(3,4-difluoro-2-methylphenyl)-4,4,5,5-tetramethyl-1,3,2-dioxaborolane was used as the Suzuki coupling partner in place of 2-(3,4-difluoro-2-methoxyphenyl)-4,4,5,5-tetramethyl-1,3,2-dioxaborolane:

| | | LC/MS | |
|---|---|---|---|
| Inter. | | (m/z | Found |
| No. | Intermediate Name | calc.) | M + 1 |
| C - 13b | dia-(1R¹,2R¹,4R²)-2-(3,4- | 336.115 | 337.1 |
| | difluoro-2- | | |
| | methylphenyl)-4-methyl-4- | | |
| | (trifluoromethyl)cyclohexane- | | |
| | 1-carboxylic acid | | |

The following Intermediate was made using the method described in Intermediate C-13a, except that Steps 1 and 2 were omitted. In Step 3, spiro[4.5]decan-7-one was used as the starting material in place of 3-methyl-3-(trifluoromethyl)cyclohexan-1-one. In Step 4, NaH was used as the base in place of DIPEA. The conditions used for the Suzuki coupling Step 5 were those of Intermediate C-2 Step 2:

| | | LC/MS | |
|---|---|---|---|
| Inter. | | (m/z | Found |
| No. | Intermediate Name | calc.) | M + 1 |
| C - 15 | rac-(7R,8R)-7-(3,4- | 324.154 | 325.1 |
| | difluoro-2- | | |
| | methoxyphenyl)spiro[4.5] | | |
| | decane-8-carboxylic acid | | |

### Intermediate C-14

### dia-(1R¹,2R¹,4R²)-2-(4-Fluoro-3-methylphenyl)-2,4-dimethyl-4-(trifluoromethyl)cyclopentane-1-carboxylic acid

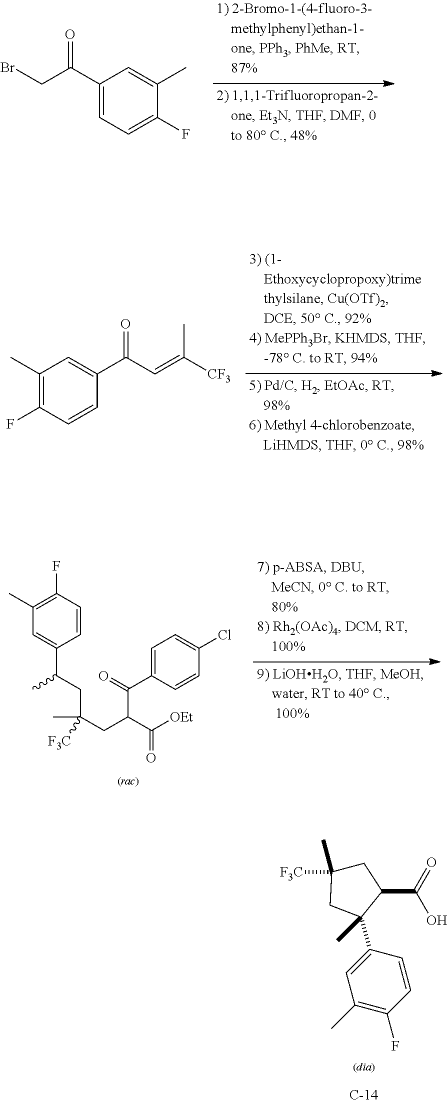

### Step 1

A solution of 2-bromo-1-(4-fluoro-3-methylphenyl)ethan-1-one (4.9 g, 21.185 mmol) in toluene (30 mL) was slowly added to a solution of PPh₃ (5.6 g, 4.947 mL, 21.351 mmol) in toluene (30 mL) and the reaction mixture was stirred at ambient temperature for 23 h. The mixture was concentrated in vacuo. Purification by trituration from Et₂O (50 mL) gave (2-(4-fluoro-3-methylphenyl)-2-oxoethyl)triphenylphosphonium bromide (9.11 g, 87%) as a white solid. ¹H NMR (400 MHz, DMSO-d₆) δ 8.08 (dd, J=7.3, 1.7 Hz, 1H), 8.03-7.96 (m, 1H), 7.92-7.80 (m, 9H), 7.79-7.71 (m, 6H), 7.39 (t, J 9.0 Hz, 1H), 6.20 (d, J=13.2 Hz, 2H), 2.33-2.28 (m, 3H) ppm. ESI-MS m/z calc. 492.065, found 413.2 (M−79)⁺.

### Step 2

A solution of 1,1,1-trifluoropropan-2-one (500.8 mg, 0.4 mL, 4.469 mmol) in DMF (1 mL) was added to a solution of (2-(4-fluoro-3-methylphenyl)-2-oxoethyl)triphenylphosphonium bromide (1.5 g, 3.041 mmol) and Et₃N (312.18 mg, 0.43 mL, 3.085 mmol) in THF (12 mL) at 0° C. and the reaction mixture was stirred at 0° C. for 15 min and at 80° C. for 3 h. The reaction was cooled to ambient temperature, diluted with MTBE (15 mL) and poured into a 0.2 N HCl aqueous solution (50 mL). The aqueous phase was separated and extracted with MTBE (1×75 mL then 2×40 mL). The combined organic layers were dried (Na₂SO₄), filtered and concentrated in vacuo. Purification by flash chromatography (SiO₂, 0 to 15% EtOAc in heptane) gave (E)-4,4,4-trifluoro-1-(4-fluoro-3-methylphenyl)-3-methylbut-2-en-1-one (368 mg, 48%) as a pale yellow oil. ¹H NMR (400 MHz, Chloroform-d) δ 7.84 (dd, J=7.3, 1.7 Hz, 1H), 7.82-7.77 (m, 1H), 7.21 (dt, J=2.9, 1.5 Hz, 1H), 7.13 (t, J=8.8 Hz, 1H), 2.37 (d, J=2.0 Hz, 3H), 2.17 (d, J=1.5 Hz, 3H) ppm.

### Step 3

(1-Ethoxycyclopropoxy)trimethylsilane (2.861 g, 3.3 mL, 16.414 mmol) was added to a solution of (E)-4,4,4-trifluoro-1-(4-fluoro-3-methylphenyl)-3-methylbut-2-en-1-one (2.04 g, 8.170 mmol) and Cu(OTf)₂ (310 mg, 0.857 mmol) in DCE (40 mL) and the reaction mixture was stirred at 50° C. for 18 h. The reaction was cooled to ambient temperature, poured into a mixture of a saturated aqueous NaHCO₃ solution (50 mL) and water (50 mL) and extracted with MTBE (2×100 mL) and EtOAc (100 mL). The combined organic layers were washed with a 4% aqueous NH₄OH solution (100 mL) and brine (100 mL), dried (Na₂SO₄), filtered and concentrated in vacuo. Purification by flash chromatography (SiO₂, 0 to 20% EtOAc in heptane) gave ethyl rac-6-(4-fluoro-3-methylphenyl)-4-methyl-6-oxo-4-(trifluoromethyl)hexanoate (2.61 g, 92%) as a colourless oil. ¹H NMR (400 MHz, Chloroform-d) δ 7.84-7.73 (m, 2H), 7.07 (t, J=8.8 Hz, 1H), 4.12 (q, J=7.1 Hz, 2H), 3.10 (s, 2H), 2.52-2.35 (m, 2H), 2.33 (d, J=2.0 Hz, 3H), 2.20-2.12 (m, 2H), 1.32 (s, 3H), 1.24 (t, J=7.2 Hz, 3H) ppm. ESI-MS m/z calc. 348.135, found 349.2 (M+1)⁺.

### Step 4

KHMDS (5.8 mL, 1 M solution in THF, 5.800 mmol) was added dropwise to a solution of MePPh₃Br (2.3 g, 6.439 mmol) in THF (26 mL) at −78° C. and the reaction was stirred for 5 min at −78° C. The mixture was warmed to ambient temperature for 5 min then cooled back to −78° C. A solution of ethyl rac-6-(4-fluoro-3-methylphenyl)-4-methyl-6-oxo-4-(trifluoromethyl)hexanoate (1 g, 2.868 mmol) in THF (6 mL) was added and after 5 min at −78° C., the resulting mixture was warmed and stirred at ambient temperature for 18 h. The mixture was poured into a saturated aqueous NaHCO₃ solution (100 mL) and water (100 mL) and extracted with MTBE (2×100 mL, 1×50 mL). The combined organic layers were dried (Na₂SO₄), filtered and concentrated in vacuo. Purification by flash chromatography (SiO₂, 0 to 15% EtOAc in heptane gave ethyl rac-6-(4-fluoro-3-methylphenyl)-4-methyl-4-(trifluoromethyl)hept-6-enoate (934 mg, 94%) as a pale yellow oil. ¹H NMR (400 MHz, Chloroform-d) δ 7.21-7.12 (m, 2H), 6.99-6.93 (m, 1H), 5.30 (d, J=1.5 Hz, 1H), 5.15 (d, J=0.7 Hz, 1H), 4.09 (q, J 7.1 Hz, 2H), 2.74 (s, 2H), 2.34-2.27 (m, 5H), 1.78-1.70 (m, 2H), 1.24 (t, J=7.2 Hz, 3H), 0.94 (s, 3H) ppm. ESI-MS m/z calc. 346.156, found 347.2 (M+1)⁺.

### Step 5

Under a nitrogen atmosphere, Pd/C (170 mg, 10% on carbon, 50% wet, 0.080 mmol) was added to a solution of ethyl rac-6-(4-fluoro-3-methylphenyl)-4-methyl-4-(trifluoromethyl)hept-6-enoate (0.93 g, 2.682 mmol) in EtOAc (20 mL). Hydrogen gas was passed through the mixture for 5 min and the reaction mixture was stirred at ambient temperature under a hydrogen atmosphere for 2 h. The mixture was filtered through a pad of Celite® and the filtrates were concentrated in vacuo to give ethyl rac-6-(4-fluoro-3-methylphenyl)-4-methyl-4-(trifluoromethyl)heptanoate (917 mg, 98%) as a colourless oil. ¹H NMR (400 MHz, Chloroform-d) δ 7.03-6.94 (m, 2H), 6.94-6.87 (m, 1H), 4.11 (q, J=6.6 Hz, 1H), 4.05 (q, J=6.8 Hz, 1H), 2.92-2.80 (m, 1H), 2.32-2.05 (m, 5H), 1.95-1.86 (m, 1H), 1.83-1.58 (m, 3H), 1.28-1.19 (m, 6H), 1.02 (s, 1.5H), 0.84 (s, 1.5H) ppm. ESI-MS m/z calc. 348.171, found 349.2 (M+1)⁺.

### Step 6

LiHMDS (2.9 mL, 1 M solution in THF, 2.900 mmol) was added to a solution of ethyl rac-6-(4-fluoro-3-methylphenyl)-4-methyl-4-(trifluoromethyl)heptanoate (500 mg, 1.434 mmol) and methyl 4-chlorobenzoate (490 mg, 2.872 mmol) in THF (7 mL) at 0° C. and the reaction mixture was stirred at 0° C. for 45 min. The mixture was poured into a saturated aqueous NH₄Cl solution (100 mL) and extracted with MTBE (3×50 mL). The combined organic layers were dried (Na₂SO₄), filtered and concentrated in vacuo to give ethyl rac-2-(4-chlorobenzoyl)-6-(4-fluoro-3-methylphenyl)-4-methyl-4-(trifluoromethyl)heptanoate (978 mg, 98%) as a pale and cloudy orange oil. ESI-MS m/z calc. 486.159, found 487.2 (M+1)⁺.

### Step 7

p-ABSA (510 mg, 2.123 mmol) and DBU (325.76 mg, 0.32 mL, 2.140 mmol) were successively added to a solution of ethyl rac-2-(4-chlorobenzoyl)-6-(4-fluoro-3-methylphenyl)-4-methyl-4-(trifluoromethyl)heptanoate (975 mg, 1.402 mmol) in MeCN (7 mL) at 0° C. and the mixture was stirred at ambient temperature for 1.5 h. Water (100 mL) was added and the mixture was extracted with MTBE (3×50 mL). The combined organic layers were dried (Na₂SO₄), filtered and concentrated in vacuo. Purification by flash chromatography (SiO₂, 0 to 15% EtOAc in heptane) followed by reverse phase chromatography (C₁₈ column, 3 to 95% MeCN in water with 0.1% formic acid) gave ethyl rac-2-diazo-6-(4-fluoro-3-methylphenyl)-4-methyl-4-(trifluoromethyl)heptanoate (444 mg, 80%) as a yellow oil. ¹H NMR (400 MHz, Chloroform-d) δ 7.04-6.94 (m, 2H), 6.94-6.87 (m, 1H), 4.28-4.15 (m, 2H), 3.00-2.84 (m, 1H), 2.56-2.31 (m, 2H), 2.29-2.20 (m, 3H), 2.00 (dd, J=14.8, 8.7 Hz, 0.5H), 1.88 (d, J=6.1 Hz, 1H), 1.79 (dd, J=14.7, 3.7 Hz, 0.5H), 1.30-1.24 (m, 3H), 1.24-1.20 (m, 3H), 0.96 (s, 1.5H), 0.89 (s, 1.5H) ppm. ESI-MS m/z calc. 374.162, found 375.2 (M+1)⁺.

### Step 8

A solution of ethyl rac-2-diazo-6-(4-fluoro-3-methylphenyl)-4-methyl-4-(trifluoromethyl)heptanoate (420 mg, 1.057 mmol) in nitrogen degassed DCM (10 mL) was slowly added to a solution of Rh₂(OAc)₄ (9 mg, 0.020 mmol) in nitrogen degassed DCM (10 mL) and the reaction mixture was stirred at ambient temperature for 1.5 h. The reaction was concentrated in vacuo. The residue was dissolved in Et₂O (30 mL) and filtered through a 5 m Nylon nanofilter. The filtrates were concentrated in vacuo and dried to give ethyl rac-2-(4-fluoro-3-methylphenyl)-2,4-dimethyl-4-(trifluoromethyl)cyclopentane-1-carboxylate (388 mg, 100%) as a pale blue-green oil. ESI-MS m/z calc. 346.156, found 347.2 (M+1)⁺.

### Step 9

A solution of LiOH·H₂O (92 mg, 2.192 mmol) in water (2 mL) was added to a solution of ethyl rac-2-(4-fluoro-3-methylphenyl)-2,4-dimethyl-4-(trifluoromethyl)cyclopentane-1-carboxylate (385 mg, 1.112 mmol) in a mixture of THF (3 mL) and MeOH (2 mL) and the reaction mixture was stirred at ambient temperature for 18 h. Additional LiOH·H₂O (48 mg, 1.144 mmol) was added and the reaction was stirred at 40° C. for 20 h. The mixture was cooled to ambient temperature. A 0.2 N aqueous HCl solution (50 mL) was added and the mixture was extracted with MTBE (3×50 mL). The combined organic layers were dried (Na₂SO₄), filtered and concentrated in vacuo to give dia-(1R¹,2R¹,4R²)-2-(4-fluoro-3-methylphenyl)-2,4-dimethyl-4-(trifluoromethyl)cyclopentane-1-carboxylic acid (C-14, 375 mg, 100%) as a pale reddish oil. ESI-MS m/z calc. 318.124, found 317.0 (M-1)-.

### Intermediate C-16

### dia-(1R¹,2R¹,5S²)-2-(3,4-Difluoro-2-methoxyphenyl)-5-methyl-5-(trifluoromethyl)cyclohexane-1-carboxylic acid

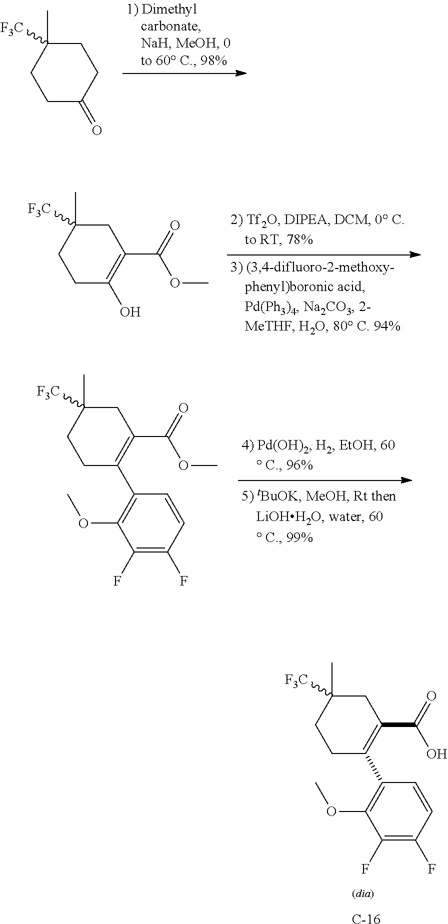

### Step 1

NaH (1.89 g, 60% dispersion in mineral oil, 47.260 mmol) and MeOH (197.75 mg, 0.25 mL, 6.171 mmol) were successively added to a stirred solution of 4-methyl-4-(trifluoromethyl)cyclohexan-1-one (3 g, 15.820 mmol) in dimethyl carbonate (85.52 g, 80 mL, 949.400 mmol) at 0° C. and the reaction mixture was stirred and heated at 60° C. for 4 h. The reaction was cooled to ambient temperature, quenched by addition of a saturated aqueous NH₄Cl solution (10 mL) and stirred at ambient temperature for 30 min. The mixture was extracted with MTBE (3×20 mL). The combined organic layers were washed with water (20 mL) and brine (20 mL), dried (Na₂SO₄), filtered and concentrated in vacuo to give methyl rac-5-methyl-2-oxo-5-(trifluoromethyl)cyclohexane-1-carboxylate (4.9 g, 98%) as a brown oil. ¹H NMR (400 MHz, Chloroform-d) δ 3.70 (s, 3H), 2.41-2.26 (m, 3H), 2.20-2.14 (m, 1H), 1.80-1.70 (m, 1H), 1.67-1.60 (m, 1H), 1.07 (s, 3H) ppm; exchangeable H not observed. ESI-MS m/z calc. 238.082, found 239.1 (M+1)⁺.

### Step 2

A solution of Tf₂O (2.348 g, 1.4 mL, 8.320 mmol) in DCM (10 mL) was added to a stirred solution of methyl rac-5-methyl-2-oxo-5-(trifluoromethyl)cyclohexane-1-carboxylate (1.3 g, 4.090 mmol) and DIPEA (1.558 g, 2.1 mL, 12.060 mmol) in DCM (30 mL) at 0° C. and the reaction mixture was warmed to ambient temperature for 16 h. The reaction was diluted with DCM (100 mL) and a saturated aqueous NaHCO₃ solution (100 mL). The aqueous layer was separated and extracted with DCM (100 mL). The combined organic layers were washed with brine (70 mL), dried (Na₂SO₄), filtered and concentrated in vacuo. Purification by flash chromatography (SiO₂, 0 to 15% EtOAc in heptane) gave methyl rac-5-methyl-5-(trifluoromethyl)-2-(((trifluoromethyl)sulfonyl)oxy)cyclohex-1-ene-1-carboxylate (1.25 g, 78%) as a yellow oil. ¹H NMR (400 MHz, chloroform-d) δ 3.84 (s, 3H), 2.77-2.68 (m, 1H), 2.57-2.47 (m, 3H), 1.98-1.87 (m, 1H), 1.86-1.77 (m, 1H), 1.20 (s, 3H) ppm. ESI-MS m/z calc. 370.031, found 371.1 (M+1)⁺;

### Step 3

Na₂CO₃ (1.7 mL, 2 M aqueous solution, 3.400 mmol) was added to a solution of methyl rac-5-methyl-5-(trifluoromethyl)-2-(((trifluoromethyl)sulfonyl)oxy)cyclohex-1-ene-1-carboxylate (630 mg, 1.633 mmol) and (3,4-difluoro-2-methoxyphenyl)boronic acid (370 mg, 1.870 mmol) in 2-MeTHF (13 mL) and nitrogen gas was passed through the mixture for 5 min. Pd(PPh₃)₄(92 mg, 0.080 mmol) was added and the reaction was stirred at 80° C. for 2.5 h. The mixture was cooled to ambient temperature and partitioned between water (20 mL) and EtOAc (20 mL). The aqueous layer was separated and extracted with EtOAc (20 mL). The combined organic layers were washed with brine (20 mL), dried (Na₂SO₄), filtered and concentrated in vacuo. Purification by flash chromatography (SiO₂, 0 to 10% EtOAc in heptane) gave methyl rac-2-(3,4-difluoro-2-methoxyphenyl)-5-methyl-5-(trifluoromethyl)cyclohex-1-ene-1-carboxylate (620 mg, 94%) as a yellow oil. ¹H NMR (400 MHz, chloroform-d) δ 6.89-6.80 (m, 1H), 6.72-6.62 (m, 1H), 3.91 (d, J=2.0 Hz, 3H), 3.52 (s, 3H), 2.70-2.59 (m, 1H), 2.55-2.24 (m, 3H), 1.92-1.81 (m, 1H), 1.79-1.70 (m, 1H), 1.23 (s, 3H, overlapped with heptane) ppm. ESI-MS m/z calc. 364.110, found 365.1 (M+1)⁺.

### Step 4

In a reactor, a solution of methyl rac-2-(3,4-difluoro-2-methoxyphenyl)-5-methyl-5-(trifluoromethyl)cyclohex-1-ene-1-carboxylate (625 mg, 1.550 mmol) in EtOH (20 mL) was added to Pd(OH)₂ (240 mg, 20% on carbon, 50% water, 0.170 mmol) and the reaction mixture was stirred under a hydrogen pressure of 60 psi at 60° C. for 32 h. The reaction was cooled to ambient temperature, filtered through a pad of Celite®, washing the filtered cake with MTBE (50 mL). The filtrates were concentrated in vacuo to give dia-(1S′,2R′,5S²)-2-(3,4-difluoro-2-methoxyphenyl)-5-methyl-5-(trifluoromethyl)cyclohexane-1-carboxylate (576 mg, 96%) as a mixture of cis diastereomers in a ratio 80:20 and as an amber oil, which was used without further purification in the next step. ESI-MS m/z calc. 366.125, found 367.2 (M+1)⁺. The trans diastereomers are also observed in traces amounts.

### Step 5

KO^{t}Bu (410 mg, 3.654 mmol) was added to a solution of dia-(1S′,2R′,5S²)-2-(3,4-difluoro-2-methoxyphenyl)-5-methyl-5-(trifluoromethyl)cyclohexane-1-carboxylate (570 mg, 1.478 mmol) in MeOH (20 mL) at ambient temperature and the reaction was stirred at ambient temperature for 16 h. Water (5 mL) and LiOH·H₂O (350 mg, 8.341 mmol) were successively added and the reaction was stirred at 60° C. for 3 h. The reaction mixture was cooled to ambient temperature and concentrated in vacuo. The residue was diluted with a 1 M aqueous HCl solution (20 mL) and extracted with MTBE (3×30 mL). The combined organic layers were dried (Na₂SO₄), filtered and concentrated in vacuo to give dia-(1R¹,2R¹,5S²)-2-(3,4-difluoro-2-methoxyphenyl)-5-methyl-5-(trifluoromethyl)cyclohexane-1-carboxylic acid (C-16, 554 mg, 99%) as a mixture of trans diastereomers in a ratio 80:20 and as a pale yellow oil, which was used without further purification in the next step. ESI-MS m/z calc. 352.110, found 353.1 (M+1)⁺. One of the cis diastereomeric pair was also observed in traces amounts.

### Intermediate C-19

### rac-(4S,6R,7R)-7-(3,4-Difluoro-2-methoxyphenyl)-1,1-difluorospiro[3.4]octane-6-carboxylic acid

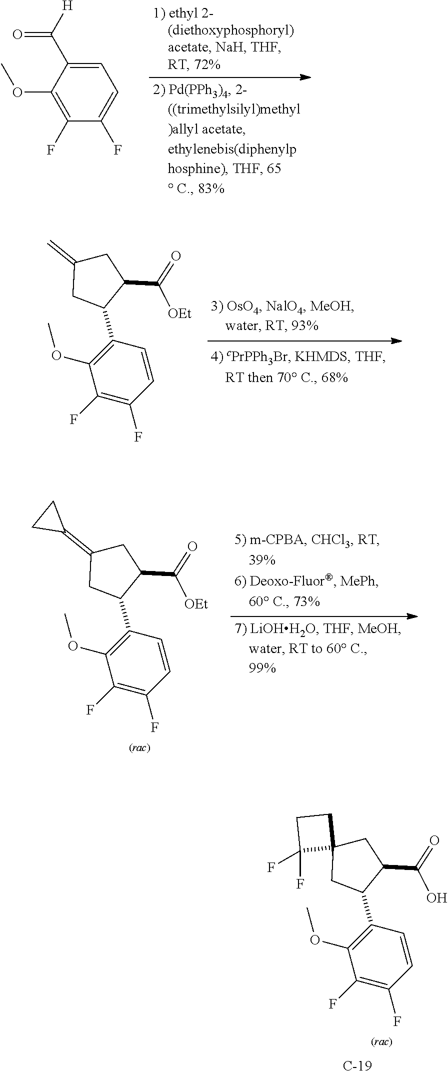

### Step 1

Ethyl 2-(diethoxyphosphoryl)acetate (6.328 g, 5.6 mL, 28.230 mmol) was added dropwise at ambient temperature to a suspension of NaH (957 mg, 60% dispersion in mineral oil, 23.930 mmol) in THF (40 mL) and the reaction mixture was stirred at ambient temperature for 30 min. A solution of 3,4-difluoro-2-methoxybenzaldehyde (3.71 g, 19.270 mmol) in THF (40 mL) was added at once and the mixture was stirred at ambient temperature for 30 min. The reaction was quenched by addition of a saturated aqueous NH₄Cl (200 mL) solution and extracted with EtOAc (2×150 mL). The combined organic layers were washed with brine (75 mL), dried (Na₂SO₄), filtered and concentrated in vacuo. Purification by flash chromatography (SiO₂, 0 to 20% EtOAc in heptane) gave ethyl (E)-3-(3,4-difluoro-2-methoxyphenyl)acrylate (3.48 g, 72%) as a white solid. ¹H NMR (400 MHz, chloroform-d) δ 7.85 (d, J=16.1 Hz, 1H), 7.28-7.23 (m, 1H, overlapped with solvent), 6.94-6.86 (m, 1H), 6.46 (d, J=16.3 Hz, 1H), 4.28 (q, J=7.1 Hz, 2H), 4.04 (d, J=2.3 Hz, 3H), 1.35 (t, J=7.1 Hz, 3H) ppm. ESI-MS m/z calc. 242.076, found 243.1 (M+1)⁺.

### Step 2

Nitrogen gas was passed through a stirred solution of ethyl (E)-3-(3,4-difluoro-2-methoxyphenyl)acrylate (1.25 g, 5.078 mmol), 2-((trimethylsilyl)methyl)allyl acetate (1.140 g, 1.3 mL, 6.119 mmol) and ethylenebis(diphenylphosphine) (205 mg, 0.515 mmol) in THF (15 mL) at ambient temperature for 5 min. Pd(PPh₃)₄(592 mg, 0.512 mmol) was added and the reaction mixture was stirred at 65° C. for 20 h. The mixture was cooled to ambient temperature. Additional amounts of 2-((trimethylsilyl)methyl)allyl acetate (877 mg, 1 mL, 4.710 mmol), ethylenebis(diphenylphosphine) (205 mg, 0.510 mmol) and Pd(PPh₃)₄(600 mg, 0.519 mmol) were added and nitrogen gas was passed through the mixture for 5 min. The reaction was stirred under nitrogen at 65° C. for 2 h. The reaction was cooled to ambient temperature and concentrated in vacuo. Purification by flash chromatography (SiO₂, 0 to 20% EtOAc in heptane) gave ethyl rac-(1R,2R)-2-(3,4-difluoro-2-methoxyphenyl)-4-methylenecyclopentane-1-carboxylate (1.28 g, 83%) as a yellow oil. ¹H NMR (400 MHz, Chloroform-d) δ 6.95-6.89 (m, 1H), 6.87-6.79 (m, 1H), 4.96-4.91 (m, 2H), 4.09-4.03 (m, 2H), 3.96 (d, J=2.2 Hz, 3H), 3.68 (td, J=10.5, 8.1 Hz, 1H), 3.04 (td, J=10.3, 7.9 Hz, 1H), 2.88-2.76 (m, 2H), 2.75-2.65 (m, 1H), 2.44-2.34 (m, 1H), 1.14 (t, J=7.1 Hz, 3H) ppm. ESI-MS m/z calc. 296.122, found 297.1 (M+1)⁺.

### Step 3

A solution of OsO₄ (100 mg, 4 wt % in water, 0.016 mmol) and NaIO₄ (100 mg, 0.468 mmol) were successively added to a solution of ethyl rac-(1R,2R)-2-(3,4-difluoro-2-methoxyphenyl)-4-methylenecyclopentane-1-carboxylat (50 mg, 0.153 mmol) in a mixture of MeOH (5 mL) and water (1 mL) at ambient temperature and the reaction was stirred at ambient temperature for 45 min. The reaction was concentrated in vacuo. Purification by flash chromatography (SiO₂, 0 to 30% EtOAc in heptane) gave ethyl rac-(1R,2R)-2-(3,4-difluoro-2-methoxyphenyl)-4-oxocyclopentane-1-carboxylate (42.5 mg, 93%) as a pale yellow oil. ¹H NMR (400 MHz, Chloroform-d) δ 6.93-6.77 (m, 2H), 4.15-4.06 (m, 2H), 4.01 (d, J=2.7 Hz, 3H), 3.88-3.75 (m, 1H), 3.35-3.22 (m, 1H), 2.81-2.60 (m, 3H), 2.46-2.34 (m, 1H), 1.16 (t, J=7.2 Hz, 3H) ppm. ESI-MS m/z calc. 298.102, found 299.1 (M+1)⁺.

### Step 4

KHMDS (18 mL, 1 M solution in THF, 18 mmol) was added to a suspension of ^{c}PrPPh₃Br (7.2 g, 18.790 mmol) in anhydrous THF (24 mL) at ambient temperature and the reaction mixture was stirred under a nitrogen atmosphere at ambient temperature for 1 h. A solution of ethyl rac-(1R,2R)-2-(3,4-difluoro-2-methoxyphenyl)-4-oxocyclopentane-1-carboxylate (1.5 g, 4.772 mmol) in anhydrous THF (1.5 mL) was added and the reaction mixture was stirred at 70° C. for 20 h. The mixture was cooled to ambient temperature and poured into a stirred biphasic solution of a saturated aqueous NH₄Cl solution (50 mL), EtOAc (60 mL) and MeOH (5 mL). The organic layer was separated, washed with brine (30 mL), dried (Na₂SO₄), filtered and concentrated in vacuo. Purification by flash chromatography (SiO₂, 0 to 20% EtOAc in heptane) gave ethyl rac-(1R,2R)-4-cyclopropylidene-2-(3,4-difluoro-2-methoxyphenyl)cyclopentane-1-carboxylate (1.095 g, 68%) as a pale yellow oil. ¹H NMR (400 MHz, chloroform-d) δ 6.96-6.90 (m, 1H), 6.87-6.80 (m, 1H), 4.11-4.02 (m, 2H), 3.97 (d, J=2.0 Hz, 3H), 3.80-3.68 (m, 1H), 3.08 (td, J=10.4, 8.1 Hz, 1H), 2.95-2.84 (m, 2H), 2.80-2.70 (m, 1H), 2.51-2.37 (m, 1H), 1.15 (t, J=7.2 Hz, 3H), 1.08-1.00 (m, 4H) ppm. ESI-MS m/z calc. 322.138, found 323.2 (M+1)⁺.

### Step 5

m-CPBA (863 mg, 3.851 mmol) was added to a stirred solution of ethyl rac-(1R,2R)-4-cyclopropylidene-2-(3,4-difluoro-2-methoxyphenyl)cyclopentane-1-carboxylate (1.09 g, 3.212 mmol) in CHCl₃ (30 mL) at ambient temperature and the reaction mixture was stirred at ambient temperature for 18 h. The mixture was concentrated in vacuo. Purification by reverse phase chromatography (C₁₈ column, 5 to 80% MeCN in water with 0.1% formic acid) gave ethyl dia-(4S′,6R²,7R²)-7-(3,4-difluoro-2-methoxyphenyl)-1-oxospiro[3.4]octane-6-carboxylate (425 mg, 39%) as a pale yellow oil. ESI-MS m z calc. 338.133, found 339.2 (M+1)⁺.

### Step 6

A solution of Deoxo-Fluor® (3.2 mL, 2.7 M solution in toluene, 8.640 mmol) was added to a stirred solution of ethyl dia-(4S′,6R²,7R²)-7-(3,4-difluoro-2-methoxyphenyl)-1-oxospiro[3.4]octane-6-carboxylate (420 mg, 1.241 mmol) in toluene (7 mL) and the reaction mixture was stirred at 60° C. for 16 h. The mixture was cooled to ambient temperature and slowly poured into a biphasic stirred solution of DCM (50 mL) and a saturated aqueous NaHCO₃ solution (40 mL) at 0° C. The organic layer was separated, washed with a saturated aqueous NaHCO₃ solution (40 mL) and brine (40 mL), dried (Na₂SO₄), filtered and concentrated in vacuo. Purification by reverse phase chromatography (C₁₈ column, 5 to 100% MeCN in water with 0.1% formic acid) gave:

First Eluting Isomers: ethyl rac-(4S,6R,7R)-7-(3,4-difluoro-2-methoxyphenyl)-1,1-difluorospiro[3.4]octane-6-carboxylate (180 mg, 38%) as a yellow oil. ¹H NMR (400 MHz, Chloroform-d) δ 7.05-7.00 (m, 1H), 6.91-6.82 (m, 1H), 4.11-4.04 (m, 2H), 3.96 (d, J=2.1 Hz, 3H), 3.81-3.69 (m, 1H), 3.30 (ddd, J=12.2, 11.0, 6.4 Hz, 1H), 2.69-2.51 (m, 1H), 2.47-2.14 (m, 2H), 2.11-1.94 (m, 1H), 1.19-1.07 (m, 5H), 0.93-0.83 (m, 2H) ppm. ESI-MS m/z calc. 360.135, found 361.1 (M+1)⁺.

Second Eluting Isomers: ethyl rac-(4R,6R,7R)-7-(3,4-difluoro-2-methoxyphenyl)-1,1-difluorospiro[3.4]octane-6-carboxylate (173 mg, 35%) as a yellow oil. ¹H NMR (400 MHz, Chloroform-d) δ 6.98-6.89 (m, 1H), 6.89-6.78 (m, 1H), 4.12-4.05 (m, 2H), 4.01-3.91 (m, 4H), 3.14-3.05 (m, 1H), 2.66-2.27 (m, 3H), 2.22-2.04 (m, 1H), 1.19-1.05 (m, 5H), 0.93-0.84 (m, 2H) ppm. ESI-MS m z calc. 360.135, found 341.1 (M-19)⁺.

### Step 7

A solution of ethyl rac-(4S,6R,7R)-7-(3,4-difluoro-2-methoxyphenyl)-1,1-difluorospiro[3.4]octane-6-carboxylate (175 mg, 0.461 mmol) and LiOH·H₂O (47 mg, 1.120 mmol) in a mixture of THF (3.5 mL), MeOH (1.8 mL) and water (1.8 mL) was stirred at ambient temperature for 16 h. Additional LiOH·H₂O (20 mg, 0.480 mmol) was added and the reaction mixture was stirred at ambient temperature for 1.5 h and at 60° C. for 1.5 h. The reaction was cooled to ambient temperature, diluted with a 1 N aqueous HCl solution (20 mL) and extracted with MTBE (2×30 mL). The combined organic layers were dried (Na₂SO₄), filtered and concentrated in vacuo to give rac-(4S,6R,7R)-7-(3,4-difluoro-2-methoxyphenyl)-1,1-difluorospiro[3.4]octane-6-carboxylic acid (C-19, 185 mg, 99%) as a yellow oil, which was used without further purification in the next step. ¹H NMR (400 MHz, DMSO-d₆) δ 12.33 (br s, 1H), 7.19-7.10 (m, 2H), 3.88-3.86 (m, 3H), 3.71-3.60 (m, 1H), 3.19 (td, J=11.5, 6.6 Hz, 1H), 2.48-2.29 (m, 2H, overlapped with solvent), 2.14-1.98 (m, 1H), 1.96-1.79 (m, 1H), 1.21-1.05 (m, 1H overlapped with MTBE), 1.02-0.96 (m, 2H), 0.90-0.82 (m, 1H) ppm. ESI-MS m/z calc. 332.104, found 333.1 (M+1)⁺.

### Intermediate C-20

### rac-(2R,3R,3aS,6aR)-3-(3,4-Difluoro-2-methylphenyl)-6,6-difluorohexahydrofuro[3,2-b]furan-2-carboxylic acid

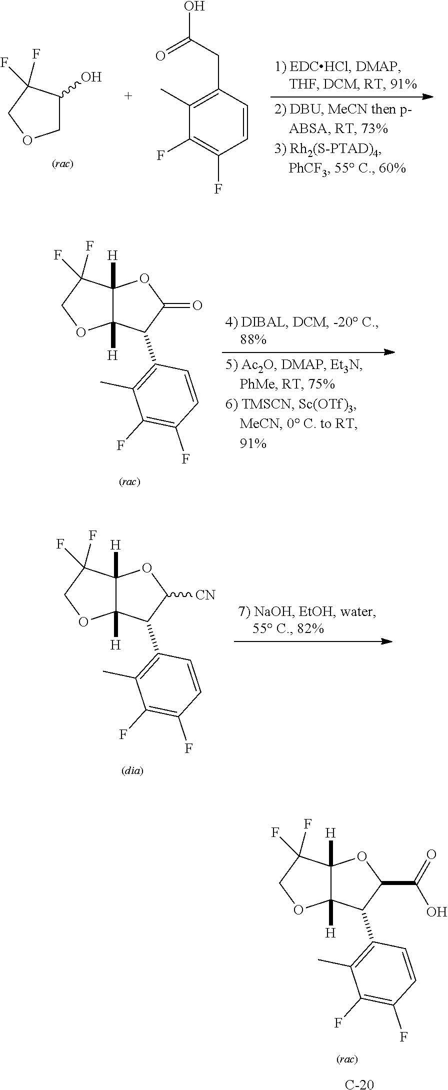

### Step 1

EDC·HCl (3.5 g, 18.26 mmol) and DMAP (740 mg, 6.057 mmol) were successively added to a stirred mixture of rac-4,4-difluorotetrahydrofuran-3-ol (1.5 g, 12.09 mmol) and 2-(3,4-difluoro-2-methylphenyl)acetic acid (2.25 g, 12.09 mmol) in a mixture of THF (7.5 mL) and DCM (7.5 mL) and the suspension was stirred at ambient temperature for 19 h. The reaction mixture was partitioned between water (200 mL) and EtOAc (400 mL). The organic phase was separated and concentrated in vacuo. Purification by flash chromatography (SiO₂, 1 to 50% EtOAc in DCM) gave rac-4,4-difluorotetrahydrofuran-3-yl 2-(3,4-difluoro-2-methylphenyl)acetate (3.2 g, 91%) as a clear oil. ¹H NMR (400 MHz, Chloroform-d) δ 7.07-6.82 (m, 2H), 5.35-5.16 (m, 1H), 4.38-4.17 (m, 1H), 4.07-3.82 (m, 3H), 3.70 (s, 2H), 2.24 (s, 3H) ppm.

### Step 2

DBU (1.1 mL, 7.355 mmol) and p-ABSA (1.5 g, 6.244 mmol) were successively added to a stirred solution of rac-4,4-difluorotetrahydrofuran-3-yl 2-(3,4-difluoro-2-methylphenyl)acetate (1.5 g, 5.133 mmol) in MeCN (15 mL) and the reaction mixture was stirred at ambient temperature for 4 h. The reaction mixture was concentrated in vacuo. Purification by flash chromatography (SiO₂, 1 to 100% EtOAc in heptane) gave rac-4,4-difluorotetrahydrofuran-3-yl 2-diazo-2-(3,4-difluoro-2-methylphenyl)acetate (1.2 g, 73%) as a yellow oil, which crystallized on standing. ¹H NMR (400 MHz, Chloroform-d) δ 7.21-7.02 (m, 2H), 5.43-5.33 (m, 1H), 4.30 (dd, J=11.0, 5.5 Hz, 1H), 4.12-3.80 (m, 3H), 2.27 (s, 3H) ppm.

### Step 3

A solution of rac-4,4-difluorotetrahydrofuran-3-yl 2-diazo-2-(3,4-difluoro-2-methylphenyl)acetate (300 mg, 0.943 mmol) in degassed PhCF₃ (6 mL) was added over 1.5 h to a stirred solution of Rh₂(S-PTAD)₄ (25 mg, 0.046 mmol) in degassed PhCF₃ (24 mL) at 55° C. and the reaction mixture was stirred for 30 min at 55° C. The reaction was cooled to ambient temperature and concentrated in vacuo. Purification by reverse phase preparative HPLC (C₁₈ column, 1 to 99% MeCN in water with 5 mM HCl) gave rac-(3R,3aS,6aR)-3-(3,4-difluoro-2-methylphenyl)-6,6-difluorotetrahydrofuro[3,2-b]furan-2(3H)-one (164.0 mg, 60%) as a white solid. ¹H NMR (400 MHz, Chloroform-d) δ 7.10-6.99 (m, 1H), 6.99-6.91 (m, 1H), 5.02 (t, J=5.0 Hz, 1H), 4.95 (dd, J=8.3, 4.2 Hz, 1H), 4.15 (d, J=5.7 Hz, 1H), 4.12-3.93 (m, 2H), 2.30 (s, 3H) ppm.

### Step 4

Under a nitrogen atmosphere, DIBAL (1.7 mL, 1 M solution in hexane, 1.7 mmol) was added over 10 min to a stirred mixture of rac-(3R,3aS,6aR)-3-(3,4-difluoro-2-methylphenyl)-6,6-difluorotetrahydrofuro[3,2-b]furan-2(3H)-one (283 mg, 0.975 mmol) in DCM (2 mL) at −20° C. and the reaction mixture was stirred at −20° C. for 1 h. The reaction was quenched by addition of acetone (121 μL, 1.648 mmol) and the mixture was warmed to 0° C. Citric Acid (3.0 mL, 1 M solution in water, 3 mmol) was gradually added over 30 min and the reaction mixture was warmed to ambient temperature. The mixture was partitioned between DCM (50 mL) and water (50 mL). The organic phase was separated and concentrated in vacuo. Purification by reverse phase preparative HPLC (C₁₈ column, 1 to 99% MeCN in water with 5 mM HCl) gave dia-(2S′,3R²,3aS²,6aR²)-3-(3,4-difluoro-2-methylphenyl)-6,6-difluorohexahydrofuro[3,2-b]furan-2-ol (250 mg, 88%) as a ˜3:1 mixture of diastereomers and as a clear oil. ¹H NMR (400 MHz, Methanol-d₄) δ 7.22-7.10 (m, 1H), 7.09-6.94 (m, 1H), 5.78 (d, J=6.3 Hz, 1H), 4.93-4.89 (m, 1H), 4.73 (dd, J=9.9, 4.5 Hz, 1H), 4.00-3.75 (m, 2H), 3.47 (t, J=5.9 Hz, 1H), 2.30 (s, 3H) ppm; exchangeable H not observed.

### Step 5

Ac₂O (5.3 mL, 56.17 mmol) was added to a stirred solution of dia-(2S¹,3R²,3aS²,6aR²)-3-(3,4-difluoro-2-methylphenyl)-6,6-difluorohexahydrofuro[3,2-b]furan-2-ol (816 mg, 2.792 mmol), DMAP (170 mg, 1.391 mmol), and Et₃N (7 mL, 50.22 mmol) in toluene (8 mL) and the reaction mixture was stirred at ambient temperature for 3 h. The reaction was diluted with EtOAc (30 mL). The organic layer was separated and washed with a saturated aqueous NaHCO₃ solution (5 mL), a saturated aqueous NH₄Cl solution (5 mL), and brine (5 mL), dried (Na₂SO₄), filtered and concentrated in vacuo. Purification by reverse phase preparative HPLC (C₁₈ column, 1 to 99% MeCN in water with 5 mM HCL) gave rac-(3R,3aS,6aR)-3-(3,4-difluoro-2-methylphenyl)-6,6-difluorohexahydrofuro[3,2-b]furan-2-yl acetate (700 mg, 75%) as a white solid. ¹H NMR (400 MHz, Methanol-d₄) δ 7.27-7.02 (m, 2H), 6.64 (dd, J=4.0, 1.3 Hz, 1H), 5.17-5.03 (m, 1H), 4.83-4.76 (m, 1H), 3.97-3.68 (m, 3H), 2.29 (s, 3H), 2.05 (s, 3H) ppm.

### Step 6

Sc(OTf)₃ (45 mg, 0.091 mmol) was added to a stirred solution of rac-(3R,3aS,6aR)-3-(3,4-difluoro-2-methylphenyl)-6,6-difluorohexahydrofuro[3,2-b]furan-2-yl acetate (100 mg, 0.299 mmol) and TMSCN (180 μL, 1.350 mmol) in MeCN (1 mL) at 0° C. and the reaction mixture was stirred at ambient temperature for 20 h. The reaction was diluted with EtOAc (20 mL) and washed with a saturated aqueous NaHCO₃ solution (5 mL), water (5 mL) and brine (5 mL), dried (Na₂SO₄), filtered and concentrated in vacuo. Purification by reverse phase preparative HPLC (C₁₈ column, 1 to 99% MeCN in water with 5 mM HCl) gave dia-(2R¹,3S²,3aS²,6aR²)-3-(3,4-difluoro-2-methylphenyl)-6,6-difluorohexahydrofuro[3,2-b]furan-2-carbonitrile (82 mg, 91%) as a ˜1:1 mixture of diastereomers and as a light tan oil. ¹H NMR (400 MHz, Methanol-d₄) δ 7.98-6.87 (m, 2H), 5.55-5.16 (m, 1H), 5.13-4.91 (m, 1H), 4.85-4.65 (m, 1H), 4.46-3.81 (m, 3H), 2.41-2.27 (m, 3H) ppm.

### Step 7

NaOH (320 mg, 8.001 mmol) was added to a solution of dia-(2R¹,3S²,3aS²,6aR²)-3-(3,4-difluoro-2-methylphenyl)-6,6-difluorohexahydrofuro[3,2-b]furan-2-carbonitrile (80 mg, 0.266 mmol) in a mixture of EtOH (800 μL) and water (800 μL) and the reaction mixture was stirred at 55° C. for 23 h. The reaction was cooled to ambient temperature and partitioned between EtOAc (10 mL) and HCl (0.5 M aqueous solution, 2 mL). The aqueous layer was separated and extracted with EtOAc (2×10 mL). The combined organic layers were washed with brine (10 mL), dried (Na₂SO₄), filtered and concentrated in vacuo. Purification by reverse phase preparative HPLC (C₁₈ column, 1 to 99% MeCN in water with 5 mM HCl) gave rac-(2R,3R,3aS,6aR)-3-(3,4-difluoro-2-methylphenyl)-6,6-difluorohexahydrofuro[3,2-b]furan-2-carboxylic acid (C-20, 70 mg, 82%) as a white solid. ¹H NMR (400 MHz, DMSO-d₆) δ 7.32-7.07 (m, 2H), 4.88 (t, J=4.9 Hz, 1H), 4.71 (dd, J=10.6, 4.6 Hz, 1H), 4.43 (d, J=9.8 Hz, 1H), 4.01-3.83 (m, 2H), 3.57 (dd, J=9.8, 5.0 Hz, 1H), 2.24 (d, J=2.4 Hz, 3H) ppm.

### Intermediate C-21

### rac-(3aR,5R,6R,6aS)-6-(3,4-Difluoro-2-methylphenyl)-3,3-difluorohexahydro-2H-cyclopenta[b]furan-5-carboxylic acid

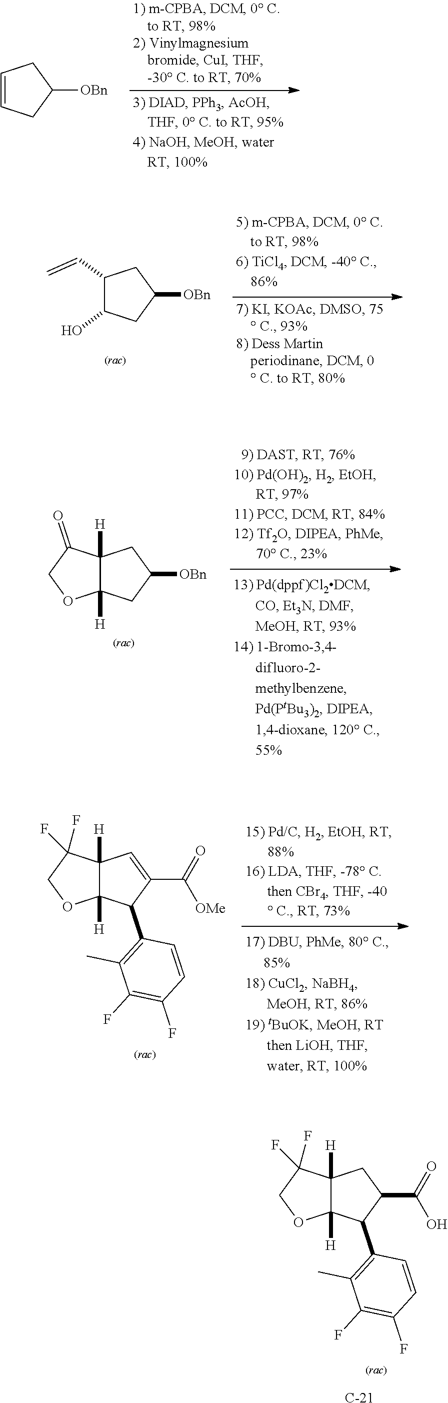

### Step 1

m-CPBA (66.1 g, 77% w/w aqueous solution, 294.94 mmol) was added in one portion to a stirred solution of ((cyclopent-3-en-1-yloxy)methyl)benzene (49.2 g, 268.25 mmol) in DCM (500 mL) at 0° C. and the reaction mixture was stirred at 0° C. for 2 h then slowly warmed to ambient temperature. The reaction was quenched by addition of a 10% aqueous Na₂S₂O₃ solution and a saturated NaHCO₃ solution (1:1, 400 mL). The aqueous layer was separated and extracted with DCM (2×200 mL). The combined organic layers were washed with a saturated aqueous NaHCO₃ solution (2×200 mL), dried (Na₂SO₄), filtered and concentrated in vacuo to give 3-(benzyloxy)-6-oxabicyclo[3.1.0]hexane (52.9 g, 98%) as a 1:1 mixture of cis and trans diastereomers and as a yellow oil. ¹H NMR (400 MHz, Chloroform-d) δ 7.41-7.29 (m, 5H), 4.50 (s, 1H), 4.46 (s, 1H), 4.08 (t, J=7.3 Hz, 0.5H), 3.89 (quin, J=7.0 Hz, 0.5H), 3.56 (s, 1H), 3.54 (s, 1H), 2.55 (d, J=7.2 Hz, 0.5H), 2.52 (d, J=7.2 Hz, 0.5H), 2.27 (s, 0.5H), 2.23 (s, 0.5H), 2.01 (d, J=7.4 Hz, 0.5H), 1.97 (d, J=7.3 Hz, 0.5H), 1.75 (d, J=6.9 Hz, 0.5H), 1.72 (d, J=6.9 Hz, 0.5H) ppm.

### Step 2

A solution of vinylmagnesium bromide (230 mL, 1 M solution in THF, 230 mmol) was added to a stirred suspension of CuI (4 g, 21.003 mmol) in THF (200 mL) at −30° C. and the reaction mixture was stirred at −30° C. for 30 min. A solution of a 1:1 mixture of cis and trans diastereomers of 3-(benzyloxy)-6-oxabicyclo[3.1.0]hexane (19.85 g, 102.88 mmol) in THF (80 mL) was added to the reaction mixture over a period of 1 h. The reaction was gradually warmed to ambient temperature while maintaining the cooling bath in place and stirred at ambient temperature for 20 h. The mixture was cooled in an ice/water bath, quenched by slow addition of a saturated aqueous NH₄Cl solution (200 mL) and water (200 mL) and diluted with MTBE (200 mL) and a 28% aqueous NH₄OH solution (15-20 mL). The aqueous layer was separated and extracted with MTBE (2×200 mL). The combined organic layers were washed with a 2:1 mixture of water and brine (300 mL), brine (150 mL), dried (Na₂SO₄), filtered and concentrated in vacuo. Purification by flash chromatography (SiO₂, 0 to 25% EtOAc in heptane) gave rac-(1R,2S,4R)-4-(benzyloxy)-2-vinylcyclopentan-1-ol (15.8 g, 70%) as a yellow oil. ¹H NMR (400 MHz, Chloroform-d) δ 7.40-7.28 (m, 5H), 5.75 (ddd, J=17.4, 10.0, 7.8 Hz, 1H), 5.16-5.00 (m, 2H), 4.50 (s, 2H), 4.10-4.02 (m, 1H), 3.93-3.86 (m, 1H), 2.81-2.69 (m, 1H), 2.25-2.13 (m, 2H), 1.95-1.86 (m, 1H), 1.63 (ddd, J=14.2, 9.3, 5.9 Hz, 1H) ppm; exchangeable H not observed. ESI-MS m/z calc. 218.131, found 219.2 (M+1)⁺.

### Step 3

DIAD (21.630 g, 21 mL, 106.97 mmol) was added to a stirred solution of rac-(1R,2S,4R)-4-(benzyloxy)-2-vinylcyclopentan-1-ol (16.02 g, 62.6 mmol), acetic acid (7.3920 g, 7 mL, 123.09 mmol) and PPh₃ (26 g, 99.129 mmol) in THF (200 mL) at 0° C. and the reaction mixture was gradually warmed to ambient temperature while maintaining in the ice bath in place. The reaction was stirred at ambient temperature for 21 h and concentrated in vacuo. The residue was partitioned between MTBE (100 mL) and heptane (100 mL) and the suspension was stirred at ambient temperature for 10 min. The solid was filtered and washed with a 1:1 mixture of MTBE in heptane (200 mL). The filtrates were concentrated in vacuo to about half the volume. The solid was filtered and washed with a 1:2 mixture of MTBE in heptane (300 mL). The filtrates were concentrated in vacuo. Purification by flash chromatography (SiO₂, 0 to 20% EtOAc in heptane) gave rac-(1S,2S,4R)-4-(benzyloxy)-2-vinylcyclopentyl acetate (16.48 g, 95%) as a colourless oil. ¹H NMR (400 MHz, Chloroform-d) δ 7.40-7.28 (m, 5H), 5.80 (ddd, J=17.4, 10.2, 7.3 Hz, 1H), 5.33 (td, J=5.4, 2.9 Hz, 1H), 5.14-5.02 (m, 2H), 4.52-4.41 (m, 2H), 4.22-4.15 (m, 1H), 3.03-2.90 (m, 1H), 2.23-2.01 (m, 3H), 2.01 (s, 3H), 1.97-1.86 (m, 1H) ppm. ESI-MS m/z calc. 260.141, found 261.2 (M+1)⁺.

### Step 4

NaOH (90 mL, 2 M aqueous solution, 180 mmol) was added to a solution of rac-(1S,2S,4R)-4-(benzyloxy)-2-vinylcyclopentyl acetate (16.48 g, 59.633 mmol) in MeOH (320 mL) and the reaction mixture was stirred at ambient temperature for 3 h. The reaction was concentrated in vacuo to half of the volume. The solution was acidified with 1 M aqueous HCl (200 mL) and extracted with EtOAc (3×200 mL). The combined organic layers were washed with water (200 mL) and brine (200 mL), dried (Na₂SO₄), filtered and concentrated in vacuo to give rac-(1S,2S,4R)-4-(benzyloxy)-2-vinylcyclopentan-1-ol (14.5 g, 100%) as a colourless oil. ¹H NMR (400 MHz, Chloroform-d) δ 7.39-7.27 (m, 5H), 5.93 (ddd, J=17.4, 10.5, 6.8 Hz, 1H), 5.27-5.16 (m, 2H), 4.52-4.41 (m, 2H), 4.30 (td, J=4.9, 2.0 Hz, 1H), 4.28-4.20 (m, 1H), 2.94-2.82 (m, 1H), 2.19-2.11 (m, 2H), 2.08-1.90 (m, 3H) ppm. ESI-MS m/z calc. 218.131, found 219.2 (M+1)⁺.

### Step 5

m-CPBA (17.9 g, 72.610 mmol) was added portionwise to a stirred solution of rac-(1S,2S,4R)-4-(benzyloxy)-2-vinylcyclopentan-1-ol (14.22 g, 65.077 mmol) in DCM (220 mL) at 0° C. and the reaction mixture was stirred at 0° C. for 30 min. The mixture was gradually warmed to ambient temperature and stirred at ambient temperature overnight. The reaction was quenched by addition of a 10% aqueous Na₂S₂O₃ solution and a saturated aqueous NaHCO₃ solution (1:1, 200 mL). The aqueous was separated and extracted with DCM (2×100 mL). The combined organic layers were washed with a saturated aqueous NaHCO₃ solution (75 mL), dried (Na₂SO₄), filtered and concentrated in vacuo to give dia-(1S′,2R′,4R′)-4-(benzyloxy)-2-((S)-oxiran-2-yl)cyclopentan-1-ol (14.96 g, 98%) as a 30:70 mixture of diastereomers and as a clear oil. ¹H NMR (400 MHz, Chloroform-d) δ 7.40-7.29 (m, 5H), 4.58-4.40 (m, 3H), 4.36-4.19 (m, 1H), 3.24-3.11 (m, 1H), 2.93-2.77 (m, 1H), 2.75-2.53 (m, 1H), 2.34-2.23 (m, 1H), 2.19-1.84 (m, 5H) ppm. ESI-MS m/z calc. 234.126, found 235.2 (M+1)⁺.

### Step 6

TiCl₄ (17.950 g, 10.4 mL, 94.634 mmol) was added dropwise to a solution of a 30:70 mixture of diastereomers of dia-(1S,2R′,4R)-4-(benzyloxy)-2-((S)-oxiran-2-yl)cyclopentan-1-ol (21.9 g, 93.380 mmol) in DCM (450 mL) at −40° C. and the reaction mixture was stirred at −40° C. for 3 h. The cooling bath was removed and the reaction was quenched by addition of a saturated aqueous NaHCO₃ solution (400 mL). The biphasic mixture was filtered through a pad of Celite®, washing the filtered cake with DCM (150 mL). The aqueous layer was separated and extracted with DCM (2×100 mL). The combined organic layers were dried (Na₂SO₄), filtered over a pad of Celite® and concentrated in vacuo to give dia-(3S,3aR¹,5R¹,6aS¹)-5-(benzyloxy)-3-chlorohexahydro-2H-cyclopenta[b]furan (23.74 g, 86%) as a 28:72 mixture of diastereomers and as a pale pink oil. ¹H NMR (400 MHz, Chloroform-d) δ 7.41-7.27 (m, 5H), 4.62-4.42 (m, 3H), 4.31-4.21 (m, 1.3H), 3.94 (td, J=7.7, 3.4 Hz, 0.7H), 3.78-3.56 (m, 2H), 2.42-2.28 (m, 1H), 2.23-2.09 (m, 1H), 2.09-2.01 (m, 2H), 1.88-1.83 (m, 1H) ppm. ESI-MS m/z calc. 252.092, found 253.1 (M+1)⁺.

### Step 7

KI (8.3 g, 49.999 mmol) and KOAc (16.3 g, 166.09 mmol) were successively added to a solution of a 28:72 mixture of diastereomers of dia-(3S,3aR¹,5R¹,6aS¹)-5-(benzyloxy)-3-chlorohexahydro-2H-cyclopenta[b]furan (24.7 g, 83.071 mmol) in DMSO (250 mL) and the reaction mixture was stirred at 75° C. for 16 h. The reaction was cooled to ambient temperature and partitioned between MTBE (250 mL) and water (1000 mL). The aqueous layer was separated and extracted with MTBE (3×100 mL) and EtOAc (4×100 mL). The combined organic layers were washed with water (2×100 mL). The latter aqueous phase was extracted with MTBE (2×50 mL). The combined organic layers were washed with brine (50 mL), dried (Na₂SO₄), filtered and concentrated in vacuo to give dia-(3S,3aS¹,5R¹,6aS¹)-5-(benzyloxy)hexahydro-2H-cyclopenta[b]furan-3-ol (22.16 g, 93%) as a pale orange oil. ESI-MS m/z calc. 234.126, found 235.2 (M+1)⁺.

### Step 8

Dess Martin periodinane (43 g, 101.38 mmol) was added to a solution of dia-(3S,3aS¹,5R¹,6aS¹)-5-(benzyloxy)hexahydro-2H-cyclopenta[b]furan-3-ol (21.1 g, 73.398 mmol) in DCM (350 mL) at 0° C. and the reaction mixture was stirred at ambient temperature for 16 h. The reaction was quenched by addition of a saturated aqueous NaHCO₃ solution (400 mL) and the mixture was vigorously stirred at ambient temperature for 10 min. A 10% aqueous Na₂S₂O₃ solution (400 mL) was added and the mixture was vigorously stirred at ambient temperature for 45 min. The aqueous layer was separated and extracted with DCM (2×200 mL). The combined organic layers were dried (Na₂SO₄), filtered and concentrated in vacuo to give rac-(3aR,5R,6aS)-5-(benzyloxy)tetrahydro-2H-cyclopenta[b]furan-3(3aH)-one (20.58 g, 80%) as an orange oil. ¹H NMR (400 MHz, Chloroform-d) δ 7.38-7.27 (m, 5H), 4.89 (td, J=6.1, 2.0 Hz, 1H), 4.51-4.45 (m, 2H), 4.16 (quin, J=6.1 Hz, 1H), 4.05 (d, J=16.4 Hz, 1H), 3.93 (d, J=17.6 Hz, 1H), 2.93 (dt, J=10.4, 6.1 Hz, 1H), 2.28 (ddd, J=14.2, 5.7, 1.8 Hz, 1H), 2.18-1.97 (m, 3H) ppm. ESI-MS m/z calc. 232.110, found 233.1 (M+1)⁺.

### Step 9

A mixture of DAST (152.50 g, 125 mL, 946.09 mmol) and rac-(3aR,5R,6aS)-5-(benzyloxy)tetrahydro-2H-cyclopenta[b]furan-3(3aH)-one (20.58 g, 58.477 mmol) was stirred at ambient temperature for 16 h. The reaction mixture was diluted with DCM (200 mL) and added dropwise to NaOH (1 μL, 3 M aqueous solution) at 0° C. The aqueous layer was separated and extracted with DCM (2×200 mL). The combined organic layers were dried (Na₂SO₄), filtered through a pad of Celite® and concentrated in vacuo. Purification by flash chromatography (SiO₂, 20 to 100% DCM in heptane, then SiO₂, 0 to 20% EtOAc in heptane) gave rac-(3aR,5R,6aS)-5-(benzyloxy)-3,3-difluorohexahydro-2H-cyclopenta[b]furan (11.9 g, 76%) as a pale yellow oil. ¹H NMR (400 MHz, Chloroform-d) δ 7.38-7.26 (m, 5H), 4.76 (td, J=6.5, 2.2 Hz, 1H), 4.54-4.45 (m, 2H), 4.17 (quin, J=6.0 Hz, 1H), 3.96 (q, J=10.5 Hz, 1H), 3.80 (dt, J=16.4, 10.4 Hz, 1H), 3.04-2.90 (m, 1H), 2.24-2.10 (m, 2H), 1.95-1.83 (m, 2H) ppm.

### Step 10

Under a nitrogen atmosphere, Pd(OH)₂ (3.7 g, 20% on carbon, 50% wet, 2.635 mmol) was added to a stirred solution of rac-(3aR,5R,6aS)-5-(benzyloxy)-3,3-difluorohexahydro-2H-cyclopenta[b]furan (11.9 g, 44.460 mmol) in EtOH (220 mL) at ambient temperature and the reaction mixture was stirred under a hydrogen atmosphere for 16 h. The mixture was diluted with EtOAc (200 mL) and filtered through a pad of Celite®, washing the filtered cake with EtOAc (80 mL). The filtrates were concentrated in vacuo. The residue was diluted with DCM (50 mL) and filtered through a Nylon 5 m nanofilter. The mixture was concentrated in vacuo to give rac-(3aR,5R,6aS)-3,3-difluorohexahydro-2H-cyclopenta[b]furan-5-ol (7.49 g, 97%) as pale yellow oil. ¹H NMR (400 MHz, Chloroform-d) δ 4.77 (td, J=6.6, 2.2 Hz, 1H), 4.49 (quin, J=5.9 Hz, 1H), 3.96 (q, J=10.5 Hz, 1H), 3.80 (dt, J=16.7, 10.4 Hz, 1H), 3.07-2.93 (m, 1H), 2.24-2.09 (m, 2H), 1.86-1.70 (m, 2H) ppm; exchangeable H not observed.

### Step 11

PCC (4.7 g, 21.804 mmol) was added to a solution of rac-(3aR,5R,6aS)-3,3-difluorohexahydro-2H-cyclopenta[b]furan-5-ol (3 g, 18.093 mmol) in DCM (90 mL) and the reaction mixture was stirred at ambient temperature for 20 h. The mixture was concentrated in vacuo (30° C., >140 torr) and the residue was diluted with Et₂O (150 mL). The formed suspension was vigorously stirred for 5 min and the mixture was filtered through a pad of Celite®. The filtrates were concentrated in vacuo (30° C., >140 torr). The residue was diluted with Et₂O (50 mL) and the mixture was stirred for 5 min then filtered through a Nylon 5 μm nanofilter. The filtrates were concentrated in vacuo (30° C., >140 torr). The residue was taken in diethylether (50 mL) and the mixture was stirred for 10 min at 0° C. then filtered through a Nylon 0.45 μm nanofilter. The filtrates were concentrated in vacuo (30° C., >140 torr) to give rac-(3aR,6aS)-3,3-difluorohexahydro-5H-cyclopenta[b]furan-5-one (2.8 g, 84%) as a dark yellow oil. ¹H NMR (400 MHz, Chloroform-d) δ 4.85 (td, J=5.3, 1.7 Hz, 1H), 4.16 (dt, J=13.7, 10.8 Hz, 1H), 4.00 (td, J=14.2, 11.0 Hz, 1H), 3.17-3.05 (m, 1H), 2.57-2.48 (m, 4H) ppm.

### Step 12

Tf₂O (5.3664 g, 3.2 mL, 19.020 mmol) was added to a solution of rac-(3aR,6aS)-3,3-difluorohexahydro-5H-cyclopenta[b]furan-5-one (2.8 g, 15.197 mmol) and DIPEA (2.7454 g, 3.7 mL, 21.242 mmol) in toluene (80 mL) at 70° C. and the reaction mixture was stirred at 70° C. for 90 min. The reaction was cooled to ambient temperature and diluted with water (150 mL). The mixture was vigorously stirred for 5 min and the suspension was filtered. The filtrates were diluted with water (150 mL) and MTBE (100 mL) and the aqueous layer was separated and extracted with MTBE (4×80 mL). The combined organic layers were dried (Na₂SO₄), filtered and concentrated in vacuo to give rac-(3aR,6aS)-3,3-difluoro-3,3a,4,6a-tetrahydro-2H-cyclopenta[b]furan-5-yl trifluoromethanesulfonate (2.59 g, 23%) as a black oil. ¹H NMR (400 MHz, Chloroform-d) δ 5.68-5.64 (m, 1H), 5.43-5.37 (m, 1H), 4.05-3.97 (m, 1H), 3.74-3.63 (m, 1H), 3.20-3.08 (m, 1H), 2.92-2.77 (m, 2H) ppm.

### Step 13

Pd(dppf)Cl₂·DCM (490 mg, 0.6 mmol) was added to a stirred solution of rac-(3aR,6aS)-3,3-difluoro-3,3a,4,6a-tetrahydro-2H-cyclopenta[b]furan-5-yl trifluoromethanesulfonate (2.58 g, 3.508 mmol) and Et₃N (1.815 g, 2.5 mL, 17.937 mmol) in a mixture of DMF (20 mL) and MeOH (30 mL) and CO was passed through the solution for 5 min. The reaction mixture was stirred at ambient temperature under a CO atmosphere for 48 h and concentrated in vacuo. The residue was partitioned between water (120 mL) and MTBE (80 mL). The aqueous layer was separated and extracted with MTBE (2×40 mL). The combined organic layers were washed with water (3×50 mL), dried (Na₂SO₄), filtered and concentrated in vacuo. Purification by flash chromatography (SiO₂, 0 to 40% EtOAc in heptane) gave methyl rac-(3aR,6aS)-3,3-difluoro-3,3a,4,6a-tetrahydro-2H-cyclopenta[b]furan-5-carboxylate (758 mg, 93%, 88% chemical purity) as a pale yellow oil. ¹H NMR (400 MHz, Chloroform-d) δ 6.61-6.54 (m, 1H), 5.52-5.45 (m, 1H), 4.06-3.97 (m, 1H), 3.81 (s, 3H), 3.61 (ddd, J=24.0, 10.8, 5.9 Hz, 1H), 3.21-3.08 (m, 1H), 3.00-2.92 (m, 1H), 2.83-2.73 (m, 1H) ppm. ESI-MS m/z calc. 204.060, found 205.1 (M+1)⁺.

### Step 14

Pd(P^{t}Bu₃)₂(300 mg, 0.587 mmol) was added to a stirred solution of methyl rac-(3aR,6aS)-3,3-difluoro-3,3a,4,6a-tetrahydro-2H-cyclopenta[b]furan-5-carboxylate (757 mg, 3.263 mmol), 1-bromo-3,4-difluoro-2-methylbenzene (1.4 g, 6.763 mmol) and DIPEA (1.261 g, 1.7 mL, 9.760 mmol) in nitrogen degassed 1,4-dioxane (16 mL) and the reaction mixture was stirred at 110° C. for 5 h. Additional amounts of 1-bromo-3,4-difluoro-2-methylbenzene (1.4 g, 6.763 mmol), DIPEA (1.261 g, 1.7 mL, 9.760 mmol) and Pd(P^{t}Bu₃)₂(300 mg, 0.587 mmol) were added and the reaction was stirred at 110° C. for 16 h. The mixture was cooled to ambient temperature, diluted with water (200 mL) and extracted with MTBE (4×50 mL). The combined organic layers were dried (Na₂SO₄), filtered and concentrated in vacuo. Purification by reverse phase chromatography (C₁₈ column, 3 to 90% MeCN in water with 0.1% formic acid) gave methyl rac-(3aR,6R,6aS)-6-(3,4-difluoro-2-methylphenyl)-3,3-difluoro-3,3a,6,6a-tetrahydro-2H-cyclopenta[b]furan-5-carboxylate (590 mg, 55%) as a pale orange oil, ¹HNMR (400 MHz, Chloroform-d) δ 6.92-6.84 (m, 2H), 6.46 (ddd, J=8.6, 4.5, 1.3 Hz, 1H), 4.51 (d, J=5.6 Hz, 1H), 4.47-4.43 (m, 1H), 4.06-3.95 (m, 1H), 3.90 (s, 1H), 3.78-3.70 (m, 1H), 3.69 (s, 3H), 2.41 (d, J=2.4 Hz, 3H) ppm. ESI-MS m/z calc. 330.088, found 331.1 (M+1)⁺.

### Step 15

Under a nitrogen atmosphere, Pd/C (400 mg, 0.188 mmol) was added to a solution of methyl rac-(3aR,6R,6aS)-6-(3,4-difluoro-2-methylphenyl)-3,3-difluoro-3,3a,6,6a-tetrahydro-2H-cyclopenta[b]furan-5-carboxylate (659 mg, 1.993 mmol) in EtOH (10 mL) and the reaction mixture was stirred under a pressure of 80 psi of hydrogen gas for 20 h. The reaction was diluted with EtOAc (20 mL) and filtered through a pad of Celite®, washing the filtered cake with EtOAc. The filtrates were concentrated in vacuo. The hydrogenation conditions were repeated to give methyl rac-(3aR,5S,6S,6aS)-6-(3,4-difluoro-2-methylphenyl)-3,3-difluorohexahydro-2H-cyclopenta[b]furan-5-carboxylate (610 mg, 88%) as a colourless oil. ¹H NMR (400 MHz, Chloroform-d) δ 6.98 (q, J=8.6 Hz, 1H), 6.79 (ddd, J=8.7, 4.7, 1.7 Hz, 1H), 4.59 (dd, J=7.5, 5.0 Hz, 1H), 4.15 (ddd, J=18.5, 10.6, 8.1 Hz, 1H), 4.02-3.91 (m, 1H), 3.77 (dd, J=10.1, 5.0 Hz, 1H), 3.63 (s, 3H), 3.10-2.98 (m, 2H), 2.39-2.26 (m, 5H) ppm. ESI-MS m/z calc. 332.104, found 333.2 (M+1)⁺.

### Step 16

LDA (1.1 mL, 2 M solution in a mixture of THF, heptane and ethylbenzene, 2.2 mmol) was added dropwise to a stirred solution of methyl rac-(3aR,5S,6S,6aS)-6-(3,4-difluoro-2-methylphenyl)-3,3-difluorohexahydro-2H-cyclopenta[b]furan-5-carboxylate (605 mg, 1.821 mmol) in THF (9 mL) at −78° C. and the reaction mixture was stirred at −78° C. for 45 min and at −40° C. for 3 min. A solution of CBr₄ (800 mg, 2.412 mmol) in THF (3 mL) was added dropwise and the reaction was warmed to ambient temperature over 75 min. The reaction was stirred at ambient temperature for 30 min. A saturated aqueous NH₄Cl solution (35 mL) was added and the mixture was extracted with MTBE (1×50 mL then 2×30 mL). The combined organic layers were dried (Na₂SO₄), filtered and concentrated in vacuo. Purification by flash chromatography (SiO₂, 0 to 25% EtOAc in heptane) gave methyl dia-(3aR,5R,6S¹,6aR¹)-5-bromo-6-(3,4-difluoro-2-methylphenyl)-3,3-difluorohexahydro-2H-cyclopenta[b]furan-5-carboxylate (563 mg, 73%) as a 89:11 mixture of diastereomers and as a pale orange oil, ¹H NMR (400 MHz, Chloroform-d) δ 7.15 (ddd, J=8.9, 4.8, 1.5 Hz, 1H), 7.03 (q, J=8.8 Hz, 1H), 4.97 (dd, J=7.3, 5.1 Hz, 1H), 4.15-4.05 (m, 1H), 4.05-3.91 (m, 2H), 3.76 (s, 3H), 3.44-3.29 (m, 1H), 3.22 (dd, J=15.0, 8.2 Hz, 1H), 2.71 (dd, J=15.0, 9.4 Hz, 1H), 2.26 (d, J=2.7 Hz, 3H) ppm. ESI-MS m/z calc. 410.014, found 411.0 (M+1)⁺.

### Step 17

DBU (203.60 mg, 0.2 mL, 1.337 mmol) was added to a solution of a 89:11 mixture of diastereomers of methyl dia-(3aR¹,5R,6S¹,6aR¹)-5-bromo-6-(3,4-difluoro-2-methylphenyl)-3,3-difluorohexahydro-2H-cyclopenta[b]furan-5-carboxylate (560 mg, 1.321 mmol) in toluene (8 mL) and the reaction mixture was stirred at 80° C. for 2 h. The reaction was cooled to ambient temperature, quenched by addition of a 0.2 N aqueous HCl solution (7 mL) and diluted with water (20 mL) and MTBE (10 mL). The aqueous phase was separated and extracted with MTBE (3×20 mL). The combined organic layers were dried (Na₂SO₄), filtered and concentrated in vacuo. Purification by flash chromatography (SiO₂, 0 to 30% EtOAc in heptane) gave methyl rac-(3aR,6aR)-6-(3,4-difluoro-2-methylphenyl)-3,3-difluoro-3,3a,4,6a-tetrahydro-2H-cyclopenta[b]furan-5-carboxylate (370 mg, 85%) as an opaque colourless oil. ¹H NMR (400 MHz, Chloroform-d) δ 7.04-6.96 (m, 1H), 6.75 (ddd, J=8.6, 4.9, 1.7 Hz, 1H), 5.41 (dd, J=7.8, 1.7 Hz, 1H), 4.08-3.98 (m, 1H), 3.80-3.68 (m, 1H), 3.62 (s, 3H), 3.27-3.16 (m, 1H), 3.16-3.08 (m, 1H), 3.03-2.93 (m, 1H), 2.13 (d, J=2.7 Hz, 3H) ppm. ESI-MS m/z calc. 330.088, found 331.1 (M+1)⁺.

### Step 18

NaBH₄ (330 mg, 8.723 mmol) was added portionwise over 4.5 h to a solution of methyl rac-(3aR,6aR)-6-(3,4-difluoro-2-methylphenyl)-3,3-difluoro-3,3a,4,6a-tetrahydro-2H-cyclopenta[b]furan-5-carboxylate (144 mg, 0.436 mmol) and CuCl₂ (295 mg, 2.194 mmol) in MeOH (3.6 mL) at ambient temperature. Two additional amounts of CuCl₂ (295 mg, 2.194 mmol and 101 mg, 0.751 mmol) were added, followed by portionwise additions of NaBH₄ (330 mg, 8.723 mmol and 115 mg, 3.040 mmol) over respectively 3 and 4 h and the mixture was stirred at ambient temperature for respectively 16 h and 4 h. The mixture was poured over a stirred mixture of a saturated aqueous NaHCO₃ solution (60 mL), water (40 mL) and MTBE (30 mL) and the thin black suspension was filtered. The aqueous layer was separated and extracted with MTBE (2×20 mL). The combined organic layers were dried (Na₂SO₄), filtered and concentrated in vacuo. Purification by flash chromatography (SiO₂, 0 to 20% EtOAc in heptane) gave methyl rac-(3aR,5S,6R,6aS)-6-(3,4-difluoro-2-methylphenyl)-3,3-difluorohexahydro-2H-cyclopenta[b]furan-5-carboxylate (125 mg, 86%) as a colorless oil, which contained 25 mol % of epimerized product methyl rac-(3aR,5R,6R,6aS)-6-(3,4-difluoro-2-methylphenyl)-3,3-difluorohexahydro-2H-cyclopenta[b]furan-5-carboxylate. ¹H NMR (400 MHz, Chloroform-d) δ 7.39 (ddd, J=8.7, 5.1, 1.8 Hz, 1H), 6.94-6.85 (m, 1H), 4.84 (t, J=4.2 Hz, 1H), 4.31-4.19 (m, 1H), 4.04-3.91 (m, 1H), 3.56 (dd, J=9.4, 3.5 Hz, 1H), 3.36-3.30 (m, 1H), 3.29 (s, 3H), 2.96-2.84 (m, 1H), 2.74-2.65 (m, 1H), 2.29 (d, J=2.4 Hz, 3H), 2.21 (ddd, J=14.8, 10.9, 8.6 Hz, 1H) ppm. ESI-MS m/z calc. 332.104, found 333.1 (M+1)⁺.

### Step 19

^{t}BuOK (124 mg, 1.105 mmol) was added to a solution of methyl rac-(3aR,5S,6R,6aS)-6-(3,4-difluoro-2-methylphenyl)-3,3-difluorohexahydro-2H-cyclopenta[b]furan-5-carboxylate containing 25 mol % of methyl rac-(3aR,5R,6R,6aS)-6-(3,4-difluoro-2-methylphenyl)-3,3-difluorohexahydro-2H-cyclopenta[b]furan-5-carboxylate (230 mg, 0.692 mmol) in MeOH (3.2 mL) at ambient temperature and the reaction mixture was stirred at ambient temperature for 23 h. THF (8 mL) and a solution of LiOH·H₂O (60 mg, 1.430 mmol) in water (2.6 mL) were added and the reaction mixture was stirred at ambient temperature for 2 h. Most of the organic solvents were removed in vacuo and the mixture was cooled to 0° C. A 0.2 N aqueous HCl solution (15 mL) was added and the mixture was extracted with MTBE (3×20 mL). The combined organic layers were dried (Na₂SO₄), filtered and concentrated in vacuo to give methyl rac-(3aR,5R,6R,6aS)-6-(3,4-difluoro-2-methylphenyl)-3,3-difluorohexahydro-2H-cyclopenta[b]furan-5-carboxylate (C-21, 221 mg, 100%) as a white solid. ¹H NMR (400 MHz, Chloroform-d) δ 7.06-6.92 (m, 2H), 4.67 (dd, J=6.1, 4.6 Hz, 1H), 4.00 (td, J=11.2, 5.9 Hz, 1H), 3.76-3.63 (m, 1H), 3.50-3.41 (m, 1H), 3.32 (td, J=11.9, 6.8 Hz, 1H), 3.15-3.02 (m, 1H), 2.56 (ddd, J=13.7, 6.8, 1.2 Hz, 1H), 2.29 (d, J=2.4 Hz, 3H), 2.11-1.99 (m, 1H) ppm; exchangeable H not observed. ESI-MS m/z calc. 318.088, found 319.1 (M+1)⁺.

### Intermediate Int-4

### 2-(2-Cyclopropyl-3,4-difluorophenyl)-4,4,5,5-tetramethyl-1,3,2-dioxaborolane

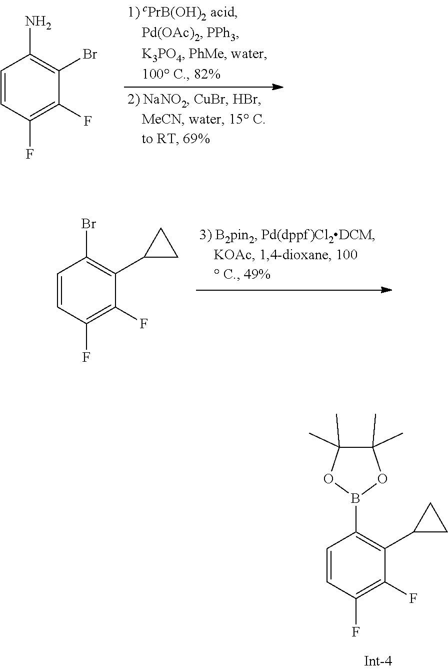 [SMILES: Nc1ccc(F)c(F)c1Br ; Fc1ccc(Br)c(C2CC2)c1F ; CC1(C)OB(c2ccc(F)c(F)c2C2CC2)OC1(C)C]

### Step 1

Argon gas was passed through a mixture of 2-bromo-3,4-difluoroaniline (44.67 g, 214.760 mmol), ^{c}PrB(OH)₂ (24 g, 279.400 mmol), K₃PO₄ (136.7 g, 644 mmol) and PPh₃ (5.67 g, 21.620 mmol) in a mixture of toluene (900 mL) and water (120 mL) for 10 min. Pd(OAc)₂ (2.43 g, 10.824 mmol) was added and the reaction was heated at 100° C. for 16 h. The mixture was cooled to ambient temperature and partitioned between water (750 mL) and EtOAc (1 L). The organic layer was filtered through a pad of Celite®. The filtrates were collected and washed with a 2 N HCl solution (1×500 mL, 1×200 mL). The combined aqueous phases were neutralised to pH 7 by addition of a 2 N NaOH solution and extracted with EtOAc (1 L). The organic phase was washed with brine (300 mL), dried (MgSO₄), filtered and concentrated in vacuo to give 2-cyclopropyl-3,4-difluoroaniline (32.3 g, 82%) as a dark brown oil. ¹H NMR (400 MHz, chloroform-d) δ 6.87-6.77 (m, 1H), 6.34-6.28 (m, 1H), 3.92 (br s, 2H), 1.52-1.42 (m, 1H), 1.04-0.97 (m, 2H), 0.70-0.65 (m, 2H) ppm.

### Step 2

A solution of NaNO₂ (13.3 g, 192.770 mmol) in water (270 mL) was added dropwise to a stirred solution of 2-cyclopropyl-3,4-difluoroaniline (32.3 g, 175.660 mmol) and HBr (73.755 g, 150 mL, 33 wt % in AcOH, 300.810 mmol) in MeCN (570 mL), keeping the internal temperature below 15° C. and the reaction mixture was stirred at ambient temperature for 1 h. CuBr (30.2 g, 210.530 mmol) was added and the reaction was stirred for 1 h. The reaction was quenched by addition of water (750 mL) and extracted with EtOAc (2×1 L). The combined organic phases were washed with a saturated NaHCO₃ solution (1.5 L) and brine (500 mL), dried (MgSO₄), filtered and concentrated in vacuo. Purification by flash chromatography (SiO₂, 100% heptane) gave 1-bromo-2-cyclopropyl-3,4-difluorobenzene (33 g, 69%) as a colourless oil. ¹H NMR (400 MHz, chloroform-d) δ 7.29-7.24 (m, 1H), 6.92-6.85 (m, 1H), 1.86-1.76 (m, 1H), 1.10-1.03 (m, 2H), 0.92-0.84 (m, 2H) ppm.

### Step 3

Argon gas was passed through a stirred mixture of 1-bromo-2-cyclopropyl-3,4-difluorobenzene (10 g, 40.763 mmol), KOAc (16 g, 163.030 mmol) and B₂pin₂ (15.53 g, 61.156 mmol) in 1,4-dioxane (175 mL) for 10 min. Pd(dppf)Cl₂·DCM (1 g, 1.225 mmol) was added and the reaction mixture was heated at 100° C. for 3 h. The reaction was cooled to ambient temperature, diluted with EtOAc (500 mL), washed with water (2×400 mL) and brine (150 mL), dried (MgSO₄), filtered and concentrated in vacuo. Purification by flash chromatography (SiO₂, 0 to 4% EtOAc in heptane) gave 2-(2-cyclopropyl-3,4-difluorophenyl)-4,4,5,5-tetramethyl-1,3,2-dioxaborolane (5.72 g, 49%) as a cream solid. ¹H NMR (400 MHz, chloroform-d) δ 7.42-7.35 (m, 1H), 6.98-6.89 (m, 1H), 2.28-2.19 (m, 1H), 1.36-1.34 (m, 12H), 0.98-0.90 (m, 2H), 0.90-0.82 (m, 2H) ppm.

The following Intermediates were made using the method described in Intermediate Int-4, except that Steps 1 and 2 were omitted. In Step 3, different bromides were used as the starting materials in place of 1-bromo-2-cyclopropyl-3,4-difluorobenzene. In the case of Int-2, the starting material bromide was prepared via a TBS protection step of (6-bromo-2,3-difluorophenyl)methanol, using imidazole and TBSCl in DCM at 0° C. to ambient temperature, conditions well-known in the art:

| | | LC/MS | | |
|---|---|---|---|---|
| Inter. | | (m/z | Found | |
| No. | Intermediate Name | calc.) | M + 1 | NMR (shifts in ppm) |
| Int-1 | 2-(7-fluoro-2,3-dihydro- | 262.154 | No | ¹H NMR (400 MHz, Chloroform-d) δ |
| | 1H-inden-4-yl)-4,4,5,5- | | ionization | 7.61 (dd, J = 8.1, 5.9 Hz, 1H), 6.84 (t, J = |
| | tetramethyl-1,3,2- | | observed in | 8.7 Hz, 1H), 3.17 (t, J = 7.5 Hz, 2H), |
| | dioxaborolane | | ES^{+/-} | 2.94 (t, J = 7.5 Hz, 2H), 2.10 (quin, J = |
| | | | | 7.5 Hz, 2H), 1.35 (s, 12H) ppm. |
| Int-2 | tert-butyl((2,3-difluoro-6- | 384.210 | 385.2 | ¹H NMR (400 MHz, Chloroform-d) δ |
| | (4,4,5,5-tetramethyl- | | | 7.51 (ddd, J = 8.3, 5.5, 1.5 Hz, 1H), |
| | 1,3,2-dioxaborolan-2- | | | 7.11-7.02 (m, 1H), 4.99 (d, J = 1.8 Hz, |
| | yl)benzyl)oxy)dimethylsilane | | | 2H), 1.34 (s, 12H), 0.93-0.87 (m, 9H), |
| | | | | 0.08 (s, 6H) ppm. |
| Int-3 | 2-(3-chloro-4-fluoro-2- | 270.099 | 271.1 | ¹H NMR (400 MHz, DMSO-d₆) δ 7.63 |
| | methylphenyl)-4,4,5,5- | | | (dd, J = 8.4, 6.2 Hz, 1H), 7.24 (t, J = |
| | tetramethyl-1,3,2- | | | 8.7 Hz, 1H), 2.56 (s, 3H), 1.31 (s, 12H) |
| | dioxaborolane | | | ppm. |

### Intermediate Int-5

### 4-Benzyloxy-2-chloro-1,6-naphthyridine

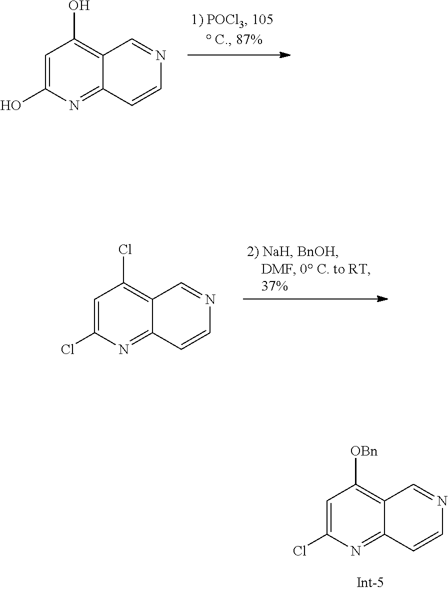 [SMILES: Oc1cc(O)c2cnccc2n1 ; Clc1cc(Cl)c2cnccc2n1 ; Clc1cc(OCc2ccccc2)c2cnccc2n1]

### Step 1

A stirring solution of 1,6-naphthyridine-2,4-diol (7.5 g, 44.4 mmol) in POCl₃ (82 g, 50 mL, 536 mmol) was heated at 105° C. for 18 h. After cooling to ambient temperature, the POCl₃ in excess was removed in vacuo. The resulting solution was slowly poured onto a stirred mixture of Na₂CO₃ (152 g), water (400 mL) and EtOAc (400 mL) and stirred at ambient temperature for 30 min. The mixture was allowed to stand for 18 h and the layers were separated. The aqueous layer was extracted with EtOAc (2×200 mL). The combined organic extracts were washed with a saturated aqueous NaHCO₃ solution (100 mL), water (100 mL) and brine (100 mL), dried (Na₂SO₄), filtered, and concentrated in vacuo to give 2,4-dichloro-1,6-naphthyridine (7.92 g, 87%) as a light brown solid. ¹H NMR (400 MHz, DMSO-d₆) δ 9.60-9.56 (m, 1H), 8.91 (d, J=5.9 Hz, 1H), 8.14 (s, 1H), 7.95 (dd, J=5.9, 0.6 Hz, 1H) ppm. ESI-MS m/z calc. 197.975, found 199.0 (M+1)⁺.

### Step 2

NaH (4.55 g, 60% in mineral oil, 113.8 mmol) was added portionwise to a stirred mixture of 2,4-dichloro-1,6-naphthyridine (20.9 g, 103 mmol) and benzyl alcohol (11.2 g, 10.7 mL, 103 mmol) in a mixture of DMF (200 mL) and 2-MeTHF (200 mL) at 0° C. The reaction mixture was stirred at 0° C. for 1 h, then at ambient temperature for 70 h. The reaction mixture was poured onto a stirred mixture of 0.1 M aqueous HCl (500 mL) and 2-MeTHF (500 mL). The aqueous layer was separated and extracted with 2-MeTHF (2×500 mL). The combined organic extracts were washed with water (100 mL) and brine (100 mL), dried (Na₂SO₄), filtered, and concentrated in vacuo. Purification by flash chromatography (SiO₂, 10 to 50% EtOAc in heptane), followed by trituration from a 4:1 mixture of MTBE and pentane (40 mL) gave 4-benzyloxy-2-chloro-1,6-naphthyridine (Int-5, 10.53 g, 37%) as a white solid. ¹H NMR (400 MHz, Chloroform-d) δ 9.60 (s, 1H), 8.79 (d, J=5.9 Hz, 1H), 7.78-7.74 (m, 1H), 7.54-7.40 (m, 5H), 6.93 (s, 1H), 5.35 (s, 2H) ppm. ESI-MS m/z calc. 270.056, found 271.0 (M+1)⁺.

### E-VIPR Assay Detecting and Measuring Na_{V} Inhibition Properties

Sodium ion channels are voltage-dependent proteins that can be activated by inducing membrane voltage changes by applying electric fields. The electrical stimulation instrument and methods of use, referred to as E-VIPR, are described in International Publication No. WO 2002/008748 A3 and C.-J. Huang et al. Characterization of voltage-gated sodium channel blockers by electrical stimulation and fluorescence detection of membrane potential, 24 Nature Biotech. 439-46 (2006), both of which are incorporated by reference in their entirety. The instrument comprises a microtiter plate handler, an optical system for exciting the coumarin dye while simultaneously recording the coumarin and oxonol emissions, a waveform generator, a current- or voltage-controlled amplifier, and parallel electrode pairs that are inserted into assay plate wells. Under integrated computer control, this instrument passes user-programmed electrical stimulus protocols to cells within the wells of the microtiter plate.

16-20 hours prior to running the assay on E-VIPR, HEK cells expressing a truncated form of human Nav 1.8 with full channel activity were seeded into microtiter 384-well plates, pre-coated with matrigel, at a density of 25,000 cells per well. 2.5-5% KIR2.1 BacMam virus was added to the final cell suspension before seeding into cell plates. HEK cells were grown in Dulbecco's Modified Eagle's Medium (DMEM) supplemented with 10% FBS (Fetal Bovine Serum, qualified; Sigma #F4135), 1% NEAA (Non-Essential Amino Acids, Gibco #11140), 1% HEPES (Gibco #15630), 1% Pen-Strep (Penicillin-Streptomycin; Gibco #15140) and 5 μg/ml Blasticidin (Gibco #R²¹⁰-01). Cells were expanded in 5-layer CellSTACK culture chambers or cell culture flasks with vented caps, with 90-95% humidity and 5% CO₂.

### Reagents and Stock Solutions:

100 mg/mL Pluronic F-127 (Sigma #P2443), in dry DMSO
Compound Plates: Corning 384-well Polypropylene Round Bottom #3656
Cell Plates: 384-well tissue culture treated plates (Greiner #781091-2B)

2.5-5% KIR 2.1 Bacmam virus (produced in-house), prepared as described in Section 3.3 of J. A. Fornwald et al., Gene Expression in Mammalian Cells Using BacMam, a Modified Baculovirus System, 1350 Methods in Molecular Biology 95-116 (2016), the entire contents of which are incorporated by reference. The concentration used can be dependent on viral titer of each batch.

5 mM DiSBAC₆(3), a voltage sensitive oxonol acceptor (CAS number 169211-44-3; 5-[3-(1,3-dihexylhexahydro-4,6-dioxo-2-thioxo-5-pyrimidinyl)-2-propen-1-ylidene]-1,3-dihexyldihydro-2-thioxo-4,6(1H,5H)-pyrimidinedione), in dry DMSO. The preparation of DiSBAC₆(3) is analogous to that of DiSBAC₄(3) as described in Voltage Sensing by Fluorescence Resonance Energy Transfer in Single Cells, Gonzalez, J. E. and Tsien, R. Y. (1995) Biophys. J. 69, 1272-1280.

5 mM CC2-DMPE, a commercially available membrane-bound coumarin phospholipid FRET donor (ThermoFisher Scientific catalog number K1017, CAS number 393782-57-5; tetradecanoic acid, 1,1′-[(1R)-1-[8-(6-chloro-7-hydroxy-2-oxo-2H-1-benzopyran-3-yl)-3-hydroxy-3-oxido-8-oxo-2,4-dioxa-7-aza-3-phosphaoct-1-yl]-1,2-ethanediyl]ester) was prepared in dry DMSO. See also, Improved indicators of cell membrane potential that use fluorescence resonance energy transfer, Gonzalez, J. E. and Tsien, R. Y. (1997) Chem. Biol. 4, 269-277.
Voltage Assay Background Suppression Compound (VABSC-1) is prepared in H₂O (89-363 mM, range used to maintain solubility)
Human Serum (HS, Millipore #S1P1-01KL, or Sigma SLBR5469V and SLBR5470V as a 50%/50% mixture, for 25% assay final concentration)

### Bath 1 Buffer:

Sodium Chloride 160 mM (9.35 g/L), Potassium Chloride, 4.5 mM (0.335 g/L), Glucose 10 mM (1.8 g/L), Magnesium Chloride (Anhydrous) 1 mM (0.095 g/L), Calcium Chloride 2 mM (0.222 g/L), HEPES 10 mM (2.38 g/L) in water.

### Na/TMA C1 Bath 1 Buffer:

Sodium Chloride 96 mM (5.61 g/L), Potassium Chloride 4.5 mM (0.335 g/L),
Tetramethylammonium (TMA)-C1 64 mM (7.01 g/L), Glucose 10 mM (1.8 g/L),
Magnesium Chloride (Anhydrous) 1 mM (0.095 g/L), Calcium Chloride 2 mM (0.222 g/L) HEPES 10 mM (2.38 g/L) in water.
   Hexyl Dye Solution (2× concentration):
Bath 1 Buffer containing 0.5% [3-cyclodextrin (made fresh prior to each use, Sigma #C4767), 8 M CC2-DMPE and 2 M DiSBAC₆(3). The solution was made by adding 10% Pluronic F127 stock equal to combined volumes of CC2-DMPE and DiSBAC₆(3). The order of preparation was first mix Pluronic and CC2-DMPE, then add DiSBAC₆(3), then while vortexing add Bath 1/p-Cyclodextrin.

Compound Loading Buffer (2× concentration): Na/TMA C1 Bath1 Buffer containing HS (omitted in experiments run in the absence of human serum (HS)) 50%, VABSC-1 1 mM, BSA 0.2 mg/ml (in Bath-1), KCl 9 mM, DMSO 0.625%.

### Assay Protocol:

1) To reach the final concentration in each well, 375 nL of each compound was pre-spotted (in neat DMSO) into polypropylene compound plates at 240x desired final concentration from an intermediate stock concentration of either 6 mM, in an 16-point dose response, 3-fold dilution, resulting in a top dose of 25 μM final concentration in the cell plate, or 0.075 mM in an 11-point dose response, 3-fold dilution, resulting in a top dose of 300 nM final concentration in the cell plate. Vehicle control (neat DMSO), and positive control (an established Na_{V}1.8 inhibitor, 25 μM final in assay in DMSO) were added manually to the outermost columns of each plate respectively. The compound plate was backfilled with 45 μL per well of Compound Loading Buffer resulting in a 240-fold dilution of compound following a 1:1 transfer of compound into the cell plate (see Step 6). Final DMSO concentration for all wells in the assay was 0.625% (0.75% DMSO was supplemented to the Compound Loading Buffer for a final DMSO concentration of 0.625%). This assay dilution protocol was adjusted to enable a higher dose range to be tested in the presence of HS or if the final assay volume was altered.

2) Hexyl Dye Solution was prepared.

3) Cell plates were prepared. On the day of the assay, the media was aspirated, and the cells were washed three times with 80 μL of Bath-1 buffer, maintaining 25 μL residual volume in each well.

4) 25 μL per well of Hexyl Dye Solution was dispensed into the cell plates. The cells were incubated for 20 minutes at room temperature or ambient conditions in darkness.

5) 45 μL per well of Compound Loading Buffer was dispensed into compound plates.

6) The cell plates were washed three times with 80 μL per well of Bath-1 Buffer, leaving 25 L of residual volume. Then 25 μL per well from compound plate was transferred to each cell plate. The mixture was incubated for 30 minutes at room temperature/ambient conditions.

7) The cell plate containing compound was read on E-VIPR using the current-controlled amplifier to deliver stimulation wave pulses using a symmetrical biphasic waveform. The user-programmed electrical stimulus protocols were 1.25-4 Amps and 4 millisecond pulse width (dependent on electrode composition) were delivered at 10 Hz for 10 seconds. A pre-stimulus recording was performed for each well for 0.5 seconds to obtain the un-stimulated intensities baseline. The stimulatory waveform was followed by 0.5 seconds of post-stimulation recording to examine the relaxation to the resting state. All E-VIPR responses were measured at 200 Hz acquisition rate.

### Data Analysis:

Data were analyzed and reported as normalized ratios of emission intensities measured in the 460 nm and 580 nm channels. The response as a function of time was reported as the ratios obtained using the following formula:

$$R ( t ) = \frac{( {intensity}_{460 nm} )}{( {intensity}_{580 nm} )}$$

The data were normalized by calculating the initial (R_{I}) and final (R_{f}) ratios. These were the average ratio values during part or all of the pre-stimulation period and during sample points during the stimulation period. The fluorescence ratio (R_{f}/R_{I}) was then calculated and reported as a function of time.

Control responses were obtained by performing assays in the presence of the positive control, and in the absence of pharmacological agents (DMSO vehicle negative control). Responses to the negative (N) and positive (P) controls were calculated as above. The compound antagonist % activity A was then defined as:

$$A = \frac{X - N}{P - N} \times 100$$

where X is the maximum amplitude of the ratio response or number of action potential peaks, at the beginning of the pulse train in the presence of test compound. Using this analysis protocol, dose response curves were plotted and IC₅₀ values were generated for various compounds of the present invention.

Compounds having a measured IC₅₀ value less than 0.01 μM in the E-VIPR Assay described above include: 1, 4, 5, 7, 9, 11, 12, 14, 15, 17, 19-23, 26-37, 39-43, 45-56, 58-65, and 70-72.

Compounds having a measured IC₅₀ value less than 0.1 μM and greater than or equal to 0.01 μM in the E-VIPR Assay described above include: 2, 8, 13, 16, 18, 25, 38, 57, and 66-69.

Compounds having a measured IC₅₀ value less than 1 μM and greater than or equal to 0.1 μM in the E-VIPR Assay described above include: 3, 6, 10, 24, and 44.

Many modifications and variations of the embodiments described herein may be made without departing from the scope, as is apparent to those skilled in the art. The specific embodiments described herein are offered by way of example only.

1. A compound of formula (I): 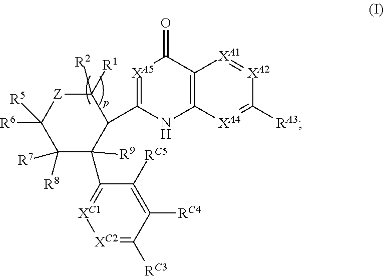
or a pharmaceutically acceptable salt thereof, wherein:
X^{A1} is C—R^{A1}, N, or N⁺—O⁻;
X^{A2} is C—R^{A2}, N, or N⁺—O⁻;
X^{A4} is C—R^{A4}, N, or N⁺—O⁻;
X^{A5} is C—R^{A5} or N;
X^{C1} is C—R^{C1} or N;
X^{C2} is C—R^{C2} or N;
R^{A1}, R^{A2}, R^{A3}, and R^{A4} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R^{A5} is H, OH, halo, CN, C₁-C₆ alkyl, C₁-C₆ haloalkyl, C₁-C₆ alkoxy, C₁-C₆ haloalkoxy, —(CH₂)ₘOₙ(CH₂)ₒOCH₃, —(CH₂)ₘR, —C(O)(CH₂)ₘR, —C(O)OR, —C(O)R, —C(O)NR^{b}R^{c}, —NR^{b}R^{c}, —CR^{d}R^{e}R^{f}, —CR^{b}R^{c}NR^{g}C(O)CR^{h}PRⁱR^{j}, —NR^{b}C(O)CR^{c}R^{g}R^{j}, C₃-C₆ cycloalkyl, 5-6 membered heteroaryl, or 4-10 membered heterocyclyl, wherein said C₃-C₆ cycloalkyl, 5-6 membered heteroaryl, or 4-10 membered heterocyclyl may be unsubstituted or may be substituted with 1-4 substituents independently selected from halo, C₁-C₆ alkyl, C₁-C₆ haloalkyl, C₁-C₆ alkoxy, and C₁-C₆ haloalkoxy;
R¹ and R² are each independently H, halo, OH, C₁-C₆ alkyl, C₁-C₆ haloalkyl, C₃-C₆ cycloalkyl, or C₁-C₆ alkoxy;
R³ and R⁴ are each independently H, halo, OH, C₁-C₆ alkyl, C₁-C₆ haloalkyl, C₃-C₆ cycloalkyl, or C₁-C₆ alkoxy;
p, Z, R⁵, R⁶, R⁷, and R⁸ are defined as follows:
(i) p is 0 or 1;
Z is C(—R³)(—R⁴);
R⁵ and R⁶ are each independently H, halo, OH, C₁-C₆ alkyl, C₁-C₆ haloalkyl, C₃-C₆ cycloalkyl, or C₁-C₆ alkoxy; and
R⁷ and R⁸ are each independently H, halo, OH, C₁-C₆ alkyl, C₁-C₆ haloalkyl, C₃-C₆ cycloalkyl, or C₁-C₆ alkoxy; or
(ii) p is 0 or 1;
Z is C(—R³)(—R⁴);
R⁷ and R⁸ are each independently H, halo, OH, C₁-C₆ alkyl, C₁-C₆ haloalkyl, C₃-C₆ cycloalkyl, or C₁-C₆ alkoxy; and
R⁵ and R⁶, together with the carbon atom to which they are attached, form a C₃-C₆ cycloalkyl optionally substituted with 1-6 R^{y}; or
(iii) p is 0 or 1;
Z is C(—R³)(—R⁴);
R⁵ and R⁷, together with the carbon atoms to which they are attached, form a fused C₃-C₆ cycloalkyl optionally substituted with 1-6 R^{y}; and
R⁶ and R are each independently H, halo, OH, C₁-C₆ alkyl, C₁-C₆ haloalkyl, C₃-C₆ cycloalkyl, or C₁-C₆ alkoxy; or
(iv) p is 0;
Z is C(—R³)(—R⁴) or O;
R⁵ and R⁷, together with the carbon atoms to which they are attached, form a fused ring of formula 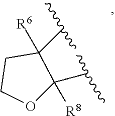
optionally substituted with 1-4 R^{y}; and
R⁶ and R⁸ are each independently H, halo, OH, C₁-C₆ alkyl, C₁-C₆ haloalkyl, C₃-C₆ cycloalkyl, or C₁-C₆ alkoxy;
R⁹ is H or CH₃;
R^{C1} and R^{C2} are each independently H, OH, halo, C₁-C₆ alkyl, C₁-C₆ alkoxy, C₁-C₆ haloalkyl, C₁-C₆ haloalkoxy, —[CH₂]_{q}—C₃-C₆ cycloalkyl, —[CH₂]_{q}—O—C₃-C₆ cycloalkyl, —[CH₂]_{q}—NR^{b}R^{c}, —(CH₂)ₘOR, or —O(CH₂)ₘOR^{b}, wherein said C₁-C₆ alkyl and C₁-C₆ alkoxy of R^{C1} or R^{C2} are each optionally substituted with an independently selected CN or 5-6 membered heteroaryl; or R^{C1} and R^{C2}, together with the carbon atoms to which they are attached, form a ring of formula: 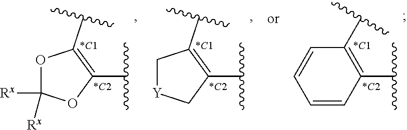
Y is O or CH₂;
R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR^{b}, or —O(CH₂)ₘOCH₃;
each R^{x} is independently H, halo, C₁-C₆ alkyl, or C₁-C₆ haloalkyl;
each R^{y} is independently halo, OH, C₁-C₆ alkyl, or C₁-C₆ haloalkyl;
R^{a} is OH, halo, C₁-C₆ alkoxy, or —NR^{b}R^{c};
R^{b}, R^{c}, R^{g}, R^{h}, and Rⁱ are each independently H or C₁-C₆ alkyl, or R^{b} and R^{c}, together with the nitrogen atom to which they are attached, form a 3-6 membered heterocyclyl;
R^{d}, R^{e}, and R^{f} are each independently H, OH, halo, C₁-C₆ alkyl, or C₁-C₆ alkoxy;
R^{j} is H, C₁-C₆ alkyl, —NR^{b}R^{c}, or —N(CH₃)₃⁺;
m and o are each independently 0, 1, 2, or 3;
q is 0 or 1; and
n is 0 or 1.

2. The compound of claim 1, or a pharmaceutically acceptable salt thereof, wherein R^{C1} and R^{C2} are each independently H, OH, halo, C₁-C₆ alkyl, C₁-C₆ alkoxy, C₁-C₆ haloalkyl, C₁-C₆ haloalkoxy, —[CH₂]_{q}—C₃-C₆ cycloalkyl, —[CH₂]_{q}—O—C₃-C₆ cycloalkyl, —[CH₂]_{q}—NR^{b}R^{c}, —(CH₂)ₘOR^{b}, or —O(CH₂)ₘOCH₃, or R^{C1} and R^{C2}, together with the carbon atoms to which they are attached, form a ring of formula:

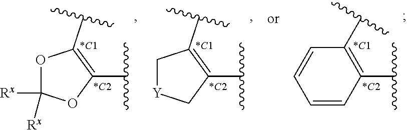

3. The compound of claim 1, or a pharmaceutically acceptable salt thereof, wherein:
p is 0 or 1;
Z is C(—R³)(—R⁴);
R⁵, R⁶, R⁷, and R⁸ are defined as follows:
(i) R⁵ and R⁶ are each independently H, halo, OH, C₁-C₆ alkyl, C₁-C₆ haloalkyl, C₃-C₆ cycloalkyl, or C₁-C₆ alkoxy; and
R⁷ and R⁸ are each independently H, halo, OH, C₁-C₆ alkyl, C₁-C₆ haloalkyl, C₃-C₆ cycloalkyl, or C₁-C₆ alkoxy; or
(ii) R⁷ and R⁸ are each independently H, halo, OH, C₁-C₆ alkyl, C₁-C₆ haloalkyl, C₃-C₆ cycloalkyl, or C₁-C₆ alkoxy; and
R⁵ and R⁶, together with the carbon atom to which they are attached, form a C₃-C₆ cycloalkyl optionally substituted with 1-6 R^{y}; or
(iii) R⁵ and R⁷, together with the carbon atoms to which they are attached, form a fused C₃-C₆ cycloalkyl optionally substituted with 1-6 R^{y}; and
R⁶ and R⁸ are each independently H, halo, OH, C₁-C₆ alkyl, C₁-C₆ haloalkyl, C₃-C₆ cycloalkyl, or C₁-C₆ alkoxy.

4. The compound of claim 1, or a pharmaceutically acceptable salt thereof, wherein the compound is a compound of formula (II): 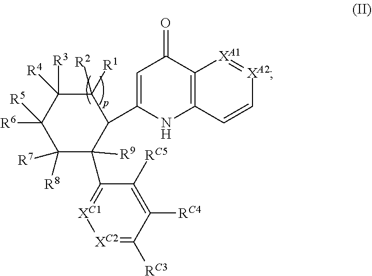
wherein:
X^{A1} is C—R^{A1}, N, or N⁺—O⁻;
X^{A2} is C—R^{A2}, N, or N⁺—O⁻;
p is 0 or 1;
X^{C1} is C—R^{C1} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R¹ and R² are each independently H, OH, C₁-C₆ alkyl, C₁-C₆ haloalkyl, C₃-C₆ cycloalkyl, or C₁-C₆ alkoxy;
R³ and R⁴ are each independently H, halo, OH, C₁-C₆ alkyl, C₁-C₆ haloalkyl, C₃-C₆ cycloalkyl, or C₁-C₆ alkoxy;
R, R⁶, R⁷, and R⁸ are defined as follows:
(i) R⁵ and R⁶ are each independently H, OH, C₁-C₆ alkyl, C₁-C₆ haloalkyl, C₃-C₆ cycloalkyl, or C₁-C₆ alkoxy; and
R⁷ and R⁸ are each independently H, OH, C₁-C₆ alkyl, C₁-C₆ haloalkyl, C₃-C₆ cycloalkyl, or C₁-C₆ alkoxy; or
(ii) R⁷ and R⁸ are each independently H, OH, C₁-C₆ alkyl, C₁-C₆ haloalkyl, C₃-C₆ cycloalkyl, or C₁-C₆ alkoxy; and
R⁵ and R⁶, together with the carbon atom to which they are attached, form a C₃-C₆ cycloalkyl optionally substituted with 1-6 R^{y}; or
(iii) R^{S} and R⁷, together with the carbon atoms to which they are attached, form a fused C₃-C₆ cycloalkyl optionally substituted with 1-6 R^{y}; and
R⁶ and R⁸ are each independently H, OH, C₁-C₆ alkyl, C₁-C₆ haloalkyl, C₃-C₆ cycloalkyl, or C₁-C₆ alkoxy;
R⁹ is H or CH₃;
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
each R^{y} is independently halo, OH, C₁-C₆ alkyl, or C₁-C₆ haloalkyl;
R^{b} and R^{c} are each independently H or C₁-C₆ alkyl, or R and R, together with the nitrogen atom to which they are attached, form a 3-6 membered heterocyclyl; and
m and o are each independently 0, 1, 2, or 3.

5. The compound of claim 1, or a pharmaceutically acceptable salt thereof, wherein the compound is a compound of formula (III): 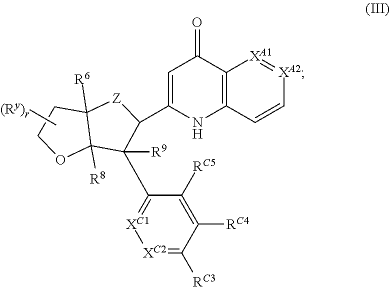
wherein:
X^{A} is C—R^{A1}, N, or N⁺—O⁻;
X^{A2} is C—R^{A2}, N, or N⁺—O⁻;
Z is C(—R³)(—R⁴) or O;
X^{C1} is C—R^{C2} or N;
X^{C2} is C—R^{C2} or N;
R^{A1} and R^{A2} are each independently H, OH, CN, halo, C₁-C₆ alkyl, C₁-C₆ alkoxy, C₁-C₆ haloalkyl, —NHS(O)₂(C₁-C₆ alkyl), —C(O)OR, —C(O)NR^{b}R^{c}, —(CH₂)ₘO(CH₂)ₒCH₃, 5-6 membered heteroaryl, or 5-6 membered heterocyclyl, wherein said 5-6 membered heteroaryl or 5-6 membered heterocyclyl may be unsubstituted or may be substituted with 1-4 substituents independently selected from C₁-C₆ alkyl and —C(O)NR^{b}R^{c};
R³ and R⁴ are each independently H, halo, OH, C₁-C₆ alkyl, C₁-C₆ haloalkyl, C₃-C₆ cycloalkyl, or C₁-C₆ alkoxy;
R⁶ and R⁸ are each independently H, OH, C₁-C₆ alkyl, C₁-C₆ haloalkyl, C₃-C₆ cycloalkyl, or C₁-C₆ alkoxy;
R⁹ is H or CH₃;
R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, —NR^{b}R^{c}, —(CH₂)ₘOR, or —O(CH₂)ₘOCH₃;
each R⁹ is independently halo, OH, C₁-C₆ alkyl, or C₁-C₆ haloalkyl;
R^{b} and R^{c} are each independently H or C₁-C₆ alkyl, or R and R, together with the nitrogen atom to which they are attached, form a 3-6 membered heterocyclyl;
m and o are each independently 0, 1, 2, or 3; and
r is 0, 1, 2, 3, or 4.

6. The compound of claim 1, or a pharmaceutically acceptable salt thereof, wherein:
(i) X^{A1} is C—R^{A1}; and/or
(ii) R^{A1} is CN or —C(O)NR^{b}R^{c}, and R^{b} and R^{c} are each independently H or C₁-C₆ alkyl; and/or
(iii) X^{A2} is N.

7-8. (canceled)

9. The compound of claim 1, or a pharmaceutically acceptable salt thereof, wherein:
X^{A1} is C—R^{A1};
R^{A1} is —C(O)NR^{b}R^{c};
R^{b} and R^{c} are each H; and
X^{A2} is N.

10. The compound of claim 1, or a pharmaceutically acceptable salt thereof, wherein p is 0.

11. The compound of claim 1, or a pharmaceutically acceptable salt thereof, wherein p is 1.

12. The compound of claim 1, or a pharmaceutically acceptable salt thereof, wherein:
(i) R¹ and R² are each H; and/or
(ii) R³ and R⁴ are each independently H or C₁-C₂ haloalkyl, optionally wherein R³ and R⁴ are each independently H or CF₃; or optionally wherein R³ and R⁴ are each H; and/or
(iii) R⁵ and R⁶ are each independently H, OH, C, —C₆ alkyl, C₁-C₆ haloalkyl, C₃-C₆ cycloalkyl, or C₁-C₆ alkoxy or, together with the carbon atom to which they are attached, form a C₃-C₆ cycloalkyl optionally substituted with 1-6 R^{y}, where each R^{y} is independently halo or C₁ haloalkyl; or
R⁵ and R⁶ are each independently H, C₁-C₄ alkyl, or C₁ haloalkyl, optionally wherein R^{S} and R⁶ are each independently H, —CH₃, —C(CH₃)₃ or —CF₃; or
R⁵ and R⁶, together with the carbon atom to which they are attached, form a C₃-C₆ cycloalkyl optionally substituted with 1-3 R^{y}, where each R^{y} is independently halo or C₁ haloalkyl, optionally wherein R⁵ and R⁶, together with the carbon atom to which they are attached, form a C₃-C₆ cycloalkyl optionally substituted with 2 R^{y}, where each R^{y} is independently F; and/or
(iv) R⁷ and R⁸ are each independently H, OH, C₁-C₃ alkyl, C₁-C₃ haloalkyl, or C₁-C₃ alkoxy; optionally wherein R⁷ and R⁸ are each independently H, OH, —CH₃, —CF₃, or —O—CH₃; and/or
(v) R⁹ is H.

13-15. (canceled)

16. The compound of claim 1, or a pharmaceutically acceptable salt thereof, wherein:
R⁵ and R⁷, together with the carbon atoms to which they are attached, form a fused C₃-C₆ cycloalkyl optionally substituted with 1-3 R^{y};
R⁶ and R⁸ are each H; and
each R^{y} is independently halo;
optionally wherein R⁵ and R⁷, together with the carbon atoms to which they are attached, form a fused C₅ or C₆ cycloalkyl optionally substituted with 2 F; and R⁶ and R⁸ are each H, optionally wherein R⁹ is H.

17. The compound of claim 1, or a pharmaceutically acceptable salt thereof, wherein:
(i) Z is O; and/or
(ii) R⁶ and R are each H; and/or
(iii) each R^{y} is independently halo, and r is 2; and/or
(iv) each R^{y} is F, and r is 2; and/or
(v) R⁹ is H.

18-20. (canceled)

22. The compound of claim 1, or a pharmaceutically acceptable salt thereof, wherein:
(i) X^{C1} is C—R^{C1}; and X^{C2} is C—R^{C2}; or
(ii) X^{C1} is C—R^{C1}; and X^{C2} is N; or
(iii) X^{C1} is N; and X^{C2} is C—R^{C2}.

23-24. (canceled)

25. The compound of claim 1, or a pharmaceutically acceptable salt thereof, wherein R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, —O—C₃-C₆ cycloalkyl, or —CH₂OH;
optionally wherein:
(i) R^{C1}, R^{C2}, R^{C3}, R^{C4}, and R^{C5} are each independently H, OH, halo, C₁-C₆ alkyl, C₁-C₆ alkoxy, C₁-C₆ haloalkyl, C₁-C₆ haloalkoxy, C₃-C₆ cycloalkyl, or —CH₂OH; or
(ii) R^{C1}, R^{C2}, R^{C3}, R^{C4}, and R^{C5} are each independently H, OH, F, Cl, —CH₃, —CH₂CH₃, —OCH₃, —CF₃, C₃ cycloalkyl, —O—C₃ cycloalkyl, or —CH₂OH; or
(ii) R^{C1}, R^{C2}, R^{C3}, R^{C4}, and R^{C5} are each independently H, OH, F, Cl, —CH₃, —CH₂CH₃, —OCH₃, C₃ cycloalkyl, or —CH₂OH.

26. The compound of claim 1, or a pharmaceutically acceptable salt thereof, wherein:
R^{C1} is —CH₃, R^{C2} is F, R^{C3} is F, R^{C4} is H, and R^{C5} is H; or
R^{C1} is —OCH₃, R^{C2} is F, R^{C3} is F, R^{C4} is H, and R^{C5} is H; or
R^{C1} is —OCH₃, R^{C2} is Cl, R^{C3} is F, R^{C4} is H, and R^{C5} is H; or
R^{C1} is —CH₃, R^{C2} is Cl, R^{C3} is F, R^{C4} is H, and R^{C5} is H; or
R^{C1} is —CH₂CH₃, R^{C2} is F, R^{C3} is F, R^{C4} is H, and R^{C5} is H; or
X^{C1} is C—R^{C1}, X^{C2} is N, R^{C1} is —OCH₃, R^{C3} is —CH₃, R^{C4} is H, and R^{C5} is H: or
X^{C1} is N, X^{C2} is C—R^{C2}, R^{C2} is —CH₃, R^{C3} is F, R^{C4} is H, and R^{C5} is H.

27-32. (canceled)

33. A compound selected from: 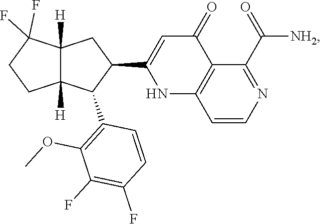 [SMILES: COc1c([C@@H]2[C@@H]3CCC(F)(F)[C@@H]3C[C@H]2c2cc(=O)c3c(C(N)=O)nccc3[nH]2)ccc(F)c1F]
2-((1R,2R,3aR,6aS)-1-(3,4-
difluoro-2-methoxyphenyl)-4,4-
difluorooctahydropentalen-2-yl)-4-
oxo-1,4-dihydro-1,6-naphthyridine-
5-carboxamide 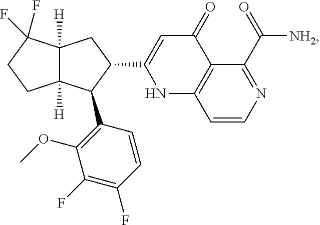 [SMILES: COc1c([C@H]2[C@H]3CCC(F)(F)[C@H]3C[C@@H]2c2cc(=O)c3c(C(N)=O)nccc3[nH]2)ccc(F)c1F]
2-((1S,2S,3aS,6aR)-1-(3,4-
difluoro-2-methoxyphenyl)-4,4-
difluorooctahydropentalen-2-yl)-
4-oxo-1,4-dihydro-1,6-
naphthyridine-5-carboxamide 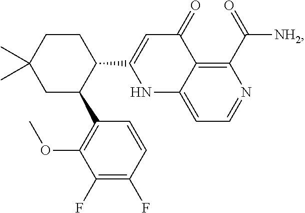 [SMILES: COc1c([C@H]2CC(C)(C)CC[C@@H]2c2cc(=O)c3c(C(N)=O)nccc3[nH]2)ccc(F)c1F]
2-((1S,2S)-2-(3,4-difluoro-2-
methoxyphenyl)-4,4-
dimethylcyclohexyl)-4-oxo-1,4-
dihydro-1,6-naphthyridine-5-
carboxamide 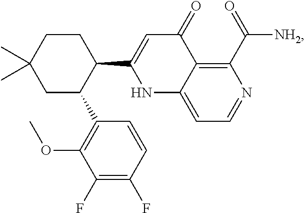 [SMILES: COc1c([C@@H]2CC(C)(C)CC[C@H]2c2cc(=O)c3c(C(N)=O)nccc3[nH]2)ccc(F)c1F]
2-((1R,2R)-2-(3,4-difluoro-2-
methoxyphenyl)-4,4-
dimethylcyclohexyl)-4-oxo-1,4-
dihydro-1,6-naphthyridine-5-
carboxamide 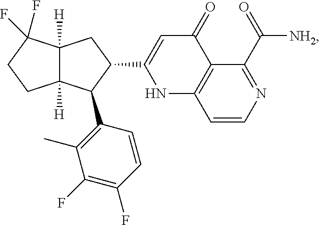 [SMILES: Cc1c([C@H]2[C@H]3CCC(F)(F)[C@H]3C[C@@H]2c2cc(=O)c3c(C(N)=O)nccc3[nH]2)ccc(F)c1F]
2-((1S,2S,3aS,6aR)-1-(3,4-
difluoro-2-methylphenyl)-4,4-
difluorooctahydropentalen-2-yl)-
4-oxo-1,4-dihydro-1,6-
naphthyridine-5-carboxamide 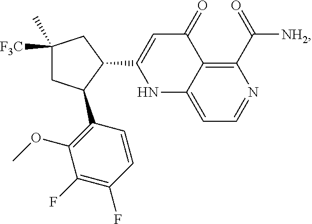 [SMILES: COc1c([C@H]2C[C@@](C)(C(F)(F)F)C[C@@H]2c2cc(=O)c3c(C(N)=O)nccc3[nH]2)ccc(F)c1F]
2-((1S,2S,4R)-2-(3,4-difluoro-2-
methoxyphenyl)-4-methyl-4-
(trifluoromethyl)cyclopentyl)-4-
oxo-1,4-dihydro-1,6-naphthyridine-
5-carboxamide 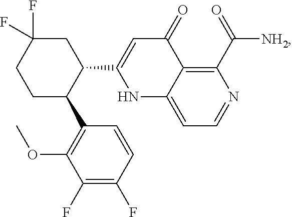 [SMILES: COc1c([C@H]2CCC(F)(F)C[C@@H]2c2cc(=O)c3c(C(N)=O)nccc3[nH]2)ccc(F)c1F]
2-((1S,2S)-2-(3,4-difluoro-2-
methoxyphenyl)-5,5-
difluorocyclohexyl)-4-oxo-1,4-
dihydro-1,6-naphthyridine-5-
carboxamide 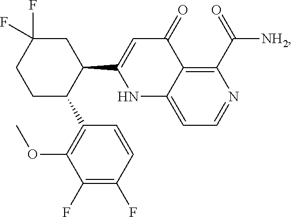 [SMILES: COc1c([C@@H]2CCC(F)(F)C[C@H]2c2cc(=O)c3c(C(N)=O)nccc3[nH]2)ccc(F)c1F]
2-((1R,2R)-2-(3,4-difluoro-2-
methoxyphenyl)-5,5-
difluorocyclohexyl)-4-oxo-1,4-
dihydro-1,6-naphthyridine-5-
carboxamide 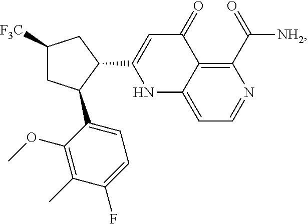 [SMILES: COc1c([C@H]2C[C@@H](C(F)(F)F)C[C@@H]2c2cc(=O)c3c(C(N)=O)nccc3[nH]2)ccc(F)c1C]
2-((1S,2S,4R)-2-(4-fluoro-2-
methoxy-3-methylphenyl)-4-
(trifluoromethyl)cyclopentyl)-4-
oxo-1,4-dihydro-1,6-naphthyridine-
5-carboxamide 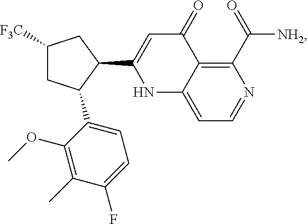 [SMILES: COc1c([C@@H]2C[C@H](C(F)(F)F)C[C@H]2c2cc(=O)c3c(C(N)=O)nccc3[nH]2)ccc(F)c1C]
2-((1R,2R,4S)-2-(4-fluoro-2-
methoxy-3-methylphenyl)-4-
(trifluoromethyl)cyclopentyl)-4-
oxo-1,4-dihydro-1,6-naphthyridine-
5-carboxamide 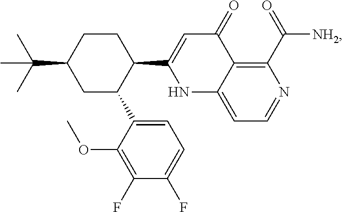 [SMILES: COc1c([C@@H]2C[C@@H](C(C)(C)C)CC[C@H]2c2cc(=O)c3c(C(N)=O)nccc3[nH]2)ccc(F)c1F]
2-((1R,2R,4S)-4-(tert-butyl)-2-
(3,4-difluoro-2-
methoxyphenyl)cyclohexyl)-4-
oxo-1,4-dihydro-1,6-
naphthyridine-5-carboxamide 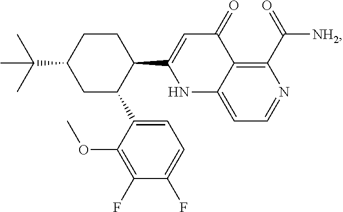 [SMILES: COc1c([C@@H]2C[C@H](C(C)(C)C)CC[C@H]2c2cc(=O)c3c(C(N)=O)nccc3[nH]2)ccc(F)c1F]
2-((1R,2R,4R)-4-(tert-butyl)-2-(3,4-
difluoro-2-
methoxyphenyl)cyclohexyl)-4-oxo-
1,4-dihydro-1,6-naphthyridine-5-
carboxamide 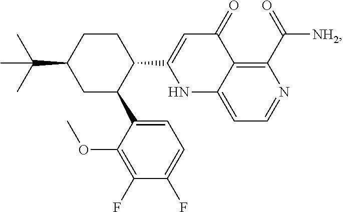 [SMILES: COc1c([C@H]2C[C@@H](C(C)(C)C)CC[C@@H]2c2cc(=O)c3c(C(N)=O)nccc3[nH]2)ccc(F)c1F]
2-((1S,2S,4S)-4-(tert-butyl)-2-(3,4-
difluoro-2-
methoxyphenyl)cyclohexyl)-4-oxo-
1,4-dihydro-1,6-naphthyridine-5-
carboxamide 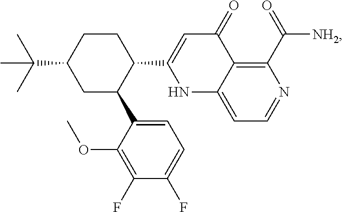 [SMILES: COc1c([C@H]2C[C@H](C(C)(C)C)CC[C@@H]2c2cc(=O)c3c(C(N)=O)nccc3[nH]2)ccc(F)c1F]
2-((1S,2S,4R)-4-(tert-butyl)-2-
(3,4-difluoro-2-
methoxyphenyl)cyclohexyl)-4-
oxo-1,4-dihydro-1,6-
naphthyridine-5-carboxamide 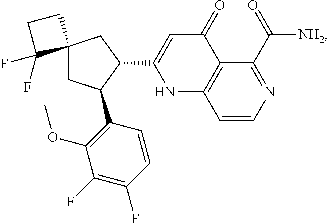 [SMILES: COc1c([C@H]2C[C@]3(CCC3(F)F)C[C@@H]2c2cc(=O)c3c(C(N)=O)nccc3[nH]2)ccc(F)c1F]
2-((4R,6S,7S)-7-(3,4-difluoro-2-
methoxyphenyl)-1,1-
difluorospiro[3.4]octan-6-yl)-4-
oxo-1,4-dihydro-1,6-naphthyridine-
5-carboxamide 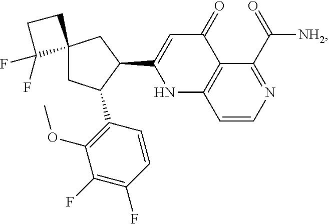 [SMILES: COc1c([C@@H]2C[C@@]3(CCC3(F)F)C[C@H]2c2cc(=O)c3c(C(N)=O)nccc3[nH]2)ccc(F)c1F]
2-((4S,6R,7R)-7-(3,4-difluoro-2-
methoxyphenyl)-1,1-
difluorospiro[3.4]octan-6-yl)-4-
oxo-1,4-dihydro-1,6-naphthyridine-
5-carboxamide 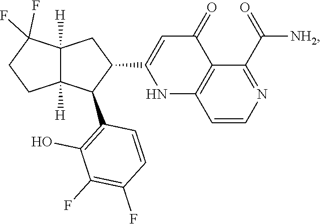 [SMILES: NC(=O)c1nccc2[nH]c([C@H]3C[C@H]4[C@H](CCC4(F)F)[C@@H]3c3ccc(F)c(F)c3O)cc(=O)c12]
2-((1S,2S,3aS,6aR)-1-(3,4-
difluoro-2-hydroxyphenyl)-4,4-
difluorooctahydropentalen-2-yl)-
4-oxo-1,4-dihydro-1,6-
naphthyridine-5-carboxamide 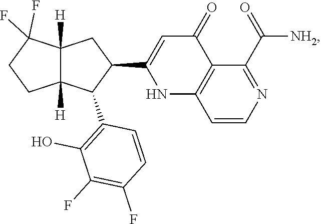 [SMILES: NC(=O)c1nccc2[nH]c([C@@H]3C[C@@H]4[C@@H](CCC4(F)F)[C@H]3c3ccc(F)c(F)c3O)cc(=O)c12]
2-((1R,2R,3aR,6aS)-1-(3,4-
difluoro-2-hydroxyphenyl)-4.4-
difluorooctahydropentalen-2-yl)-4-
oxo-1,4-dihydro-1,6-naphthyridine-
5-carboxamide 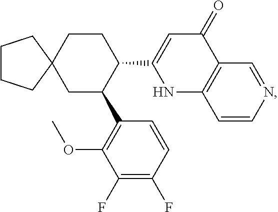 [SMILES: COc1c([C@H]2CC3(CCCC3)CC[C@@H]2c2cc(=O)c3cnccc3[nH]2)ccc(F)c1F]
2-((7S,8S)-7-(3,4-difluoro-2-
methoxyphenyl)spiro[4.5]decan-8-
yl)-1,6-naphthyridin-4(1H)-one 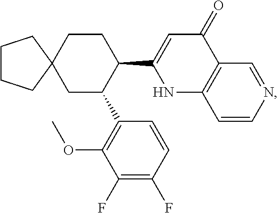 [SMILES: COc1c([C@@H]2CC3(CCCC3)CC[C@H]2c2cc(=O)c3cnccc3[nH]2)ccc(F)c1F]
2-((7R,8R)-7-(3,4-difluoro-2-
methoxyphenyl)spiro[4.5]decan-
8-yl)-1,6-naphthyridin-4(1H)-one 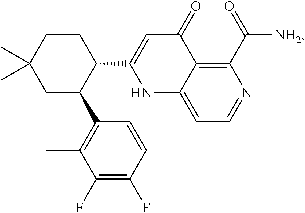 [SMILES: Cc1c([C@H]2CC(C)(C)CC[C@@H]2c2cc(=O)c3c(C(N)=O)nccc3[nH]2)ccc(F)c1F]
2-((1S,2S)-2-(3,4-difluoro-2-
methylphenyl)-4,4-
dimethylcyclohexyl)-4-oxo-1,4-
dihydro-1,6-naphthyridine-5-
carboxamide 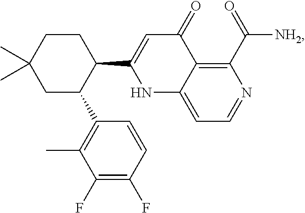 [SMILES: Cc1c([C@@H]2CC(C)(C)CC[C@H]2c2cc(=O)c3c(C(N)=O)nccc3[nH]2)ccc(F)c1F]
2-((1R,2R)-2-(3,4-difluoro-2-
methylphenyl)-4,4-
dimethylcyclohexyl)-4-oxo-1,4-
dihydro-1,6-naphthyridine-5-
carboxamide 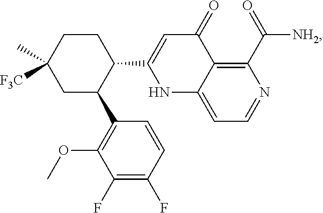 [SMILES: COc1c([C@H]2C[C@@](C)(C(F)(F)F)CC[C@@H]2c2cc(=O)c3c(C(N)=O)nccc3[nH]2)ccc(F)c1F]
2-((1S,2S,4S)-2-(3,4-difluoro-2-
methoxyphenyl)-4-methyl-4-
(trifluoromethyl)cyclohexyl)-4-
oxo-1,4-dihydro-1,6-
naphthyridine-5-carboxamide 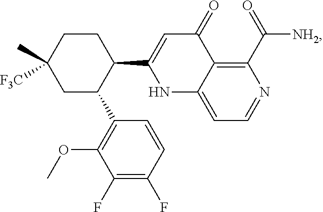 [SMILES: COc1c([C@@H]2C[C@](C)(C(F)(F)F)CC[C@H]2c2cc(=O)c3c(C(N)=O)nccc3[nH]2)ccc(F)c1F]
2-((1R,2R,4R)-2-(3,4-difluoro-2-
methoxyphenyl)-4-methyl-4-
(trifluoromethyl)cyclohexyl)-4-
oxo-1,4-dihydro-1,6-naphthyridine-
5-carboxamide 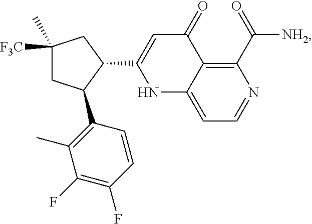 [SMILES: Cc1c([C@H]2C[C@@](C)(C(F)(F)F)C[C@@H]2c2cc(=O)c3c(C(N)=O)nccc3[nH]2)ccc(F)c1F]
2-((1S,2S,4R)-2-(3,4-difluoro-2-
methylphenyl)-4-methyl-4-
(trifluoromethyl)cyclopentyl)-4-
oxo-1,4-dihydro-1,6-naphthyridine-
5-carboxamide 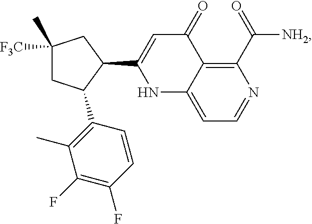 [SMILES: Cc1c([C@@H]2C[C@](C)(C(F)(F)F)C[C@H]2c2cc(=O)c3c(C(N)=O)nccc3[nH]2)ccc(F)c1F]
2-((1R,2R,4S)-2-(3,4-difluoro-2-
methylphenyl)-4-methyl-4-
(trifluoromethyl)cyclopentyl)-4-
oxo-1,4-dihydro-1,6-
naphthyridine-5-carboxamide 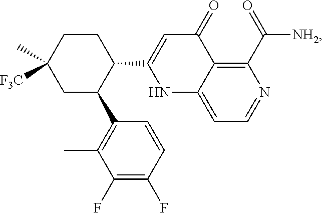 [SMILES: Cc1c([C@H]2C[C@@](C)(C(F)(F)F)CC[C@@H]2c2cc(=O)c3c(C(N)=O)nccc3[nH]2)ccc(F)c1F]
2-((1S,2S,4S)-2-(3,4-difluoro-2-
methylphenyl)-4-methyl-4-
(trifluoromethyl)cyclohexyl)-4-
oxo-1,4-dihydro-1,6-naphthyridine-
5-carboxamide 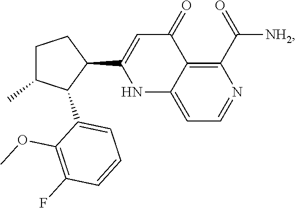 [SMILES: COc1c(F)cccc1[C@@H]1[C@H](C)CC[C@H]1c1cc(=O)c2c(C(N)=O)nccc2[nH]1]
2-((1R,2R,3R)-2-(3-fluoro-2-
methoxyphenyl)-3-
methylcyclopentyl)-4-oxo-1,4-
dihydro-1,6-naphthyridine-5-
carboxamide 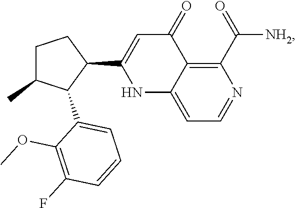 [SMILES: COc1c(F)cccc1[C@@H]1[C@@H](C)CC[C@H]1c1cc(=O)c2c(C(N)=O)nccc2[nH]1]
2-((1R,2R,3S)-2-(3-fluoro-2-
methoxyphenyl)-3-
methylcyclopentyl)-4-oxo-1,4-
dihydro-1,6-naphthyridine-5-
carboxamide 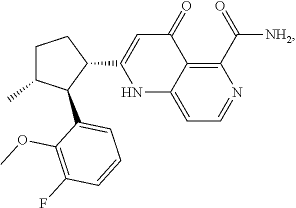 [SMILES: COc1c(F)cccc1[C@H]1[C@H](C)CC[C@@H]1c1cc(=O)c2c(C(N)=O)nccc2[nH]1]
2-((1S,2S,3R)-2-(3-fluoro-2-
methoxyphenyl)-3-
methylcyclopentyl)-4-oxo-1,4-
dihydro-1,6-naphthyridine-5-
carboxamide 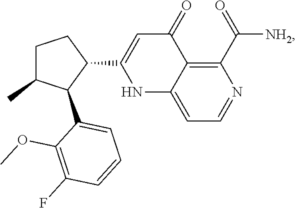 [SMILES: COc1c(F)cccc1[C@@H]1[C@@H](c2cc(=O)c3c(C(N)=O)nccc3[nH]2)CC[C@@H]1C]
2-((1S,2S,3S)-2-(3-fluoro-2-
methoxyphenyl)-3-
methylcyclopentyl)-4-oxo-1,4-
dihydro-1,6-naphthyridine-5-
carboxamide 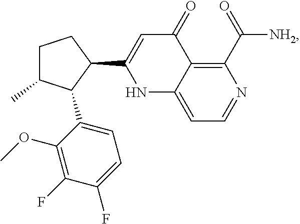 [SMILES: COc1c([C@@H]2[C@H](C)CC[C@H]2c2cc(=O)c3c(C(N)=O)nccc3[nH]2)ccc(F)c1F]
2-((1R,2R,3R)-2-(3,4-difluoro-2-
methoxyphenyl)-3-
methylcyclopentyl)-4-oxo-1,4-
dihydro-1,6-naphthyridine-5-
carboxamide 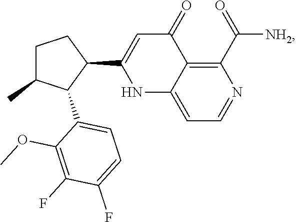 [SMILES: COc1c([C@@H]2[C@@H](C)CC[C@H]2c2cc(=O)c3c(C(N)=O)nccc3[nH]2)ccc(F)c1F]
2-((1R,2R,3S)-2-(3,4-difluoro-2-
methoxyphenyl)-3-
methylcyclopentyl)-4-oxo-1,4-
dihydro-1,6-naphthyridine-5-
carboxamide 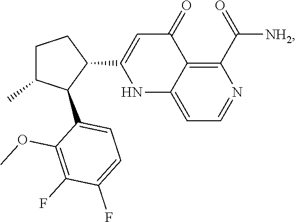 [SMILES: COc1c([C@H]2[C@H](C)CC[C@@H]2c2cc(=O)c3c(C(N)=O)nccc3[nH]2)ccc(F)c1F]
2-((1S,2S,3R)-2-(3,4-difluoro-2-
methoxyphenyl)-3-
methylcyclopentyl)-4-oxo-1,4-
dihydro-1,6-naphthyridine-5-
carboxamide 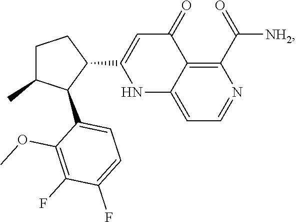 [SMILES: COc1c([C@@H]2[C@@H](c3cc(=O)c4c(C(N)=O)nccc4[nH]3)CC[C@@H]2C)ccc(F)c1F]
2-((1S,2S,3S)-2-(3,4-difluoro-2-
methoxyphenyl)-3-
methylcyclopentyl)-4-oxo-1,4-
dihydro-1,6-naphthyridine-5-
carboxamide 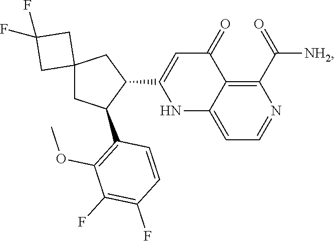 [SMILES: COc1c([C@H]2CC3(C[C@@H]2c2cc(=O)c4c(C(N)=O)nccc4[nH]2)CC(F)(F)C3)ccc(F)c1F]
2-((6S,7S)-7-(3,4-difluoro-2-
methoxyphenyl)-2,2-
difluorospiro[3.4]octan-6-yl)-4-
oxo-1,4-dihydro-1,6-naphthyridine-
5-carboxamide 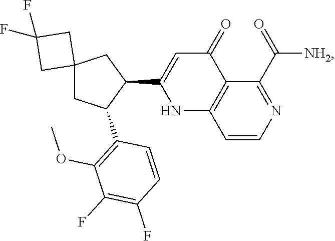 [SMILES: COc1c([C@@H]2CC3(C[C@H]2c2cc(=O)c4c(C(N)=O)nccc4[nH]2)CC(F)(F)C3)ccc(F)c1F]
2-((6R,7R)-7-(3,4-difluoro-2-
methoxyphenyl)-2,2-
difluorospiro[3.4]octan-6-yl)-4-
oxo-1,4-dihydro-1,6-naphthyridine-
5-carboxamide 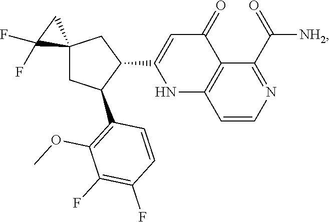 [SMILES: COc1c([C@H]2C[C@@]3(C[C@@H]2c2cc(=O)c4c(C(N)=O)nccc4[nH]2)CC3(F)F)ccc(F)c1F]
2-((3R,5S,6S)-6-(3,4-difluoro-2-
methoxyphenyl)-1,1-
difluorospiro[2.4]heptan-5-yl)-4-
oxo-1,4-dihydro-1,6-
naphthyridine-5-carboxamide 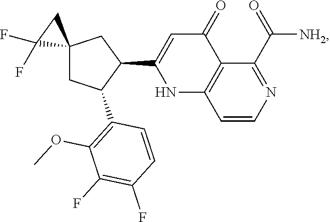 [SMILES: COc1c([C@@H]2C[C@]3(C[C@H]2c2cc(=O)c4c(C(N)=O)nccc4[nH]2)CC3(F)F)ccc(F)c1F]
2-((3S,5R,6R)-6-(3,4-difluoro-2-
methoxyphenyl)-1,1-
difluorospiro[2.4]heptan-5-yl)-4-
oxo-1,4-dihydro-1,6-naphthyridine-
5-carboxamide 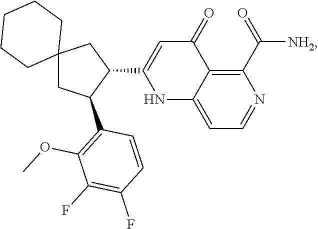 [SMILES: COc1c([C@H]2CC3(CCCCC3)C[C@@H]2c2cc(=O)c3c(C(N)=O)nccc3[nH]2)ccc(F)c1F]
2-((2S,3S)-3-(3,4-difluoro-2-
methoxyphenyl)spiro[4.5]decan-2-
yl)-4-oxo-1,4-dihydro-1,6-
naphthyridine-5-carboxamide 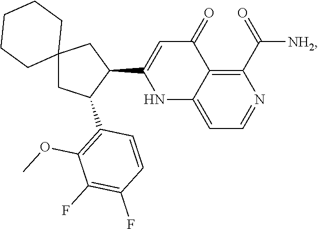 [SMILES: COc1c([C@@H]2CC3(CCCCC3)C[C@H]2c2cc(=O)c3c(C(N)=O)nccc3[nH]2)ccc(F)c1F]
2-((2R,3R)-3-(3,4-difluoro-2-
methoxyphenyl)spiro[4.5]decan-
2-yl)-4-oxo-1,4-dihydro-1,6-
naphthyridine-5-carboxamide 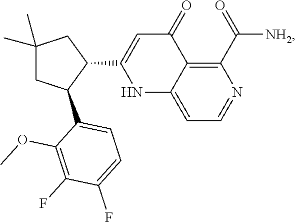 [SMILES: COc1c([C@H]2CC(C)(C)C[C@@H]2c2cc(=O)c3c(C(N)=O)nccc3[nH]2)ccc(F)c1F]
2-((1S,2S)-2-(3,4-difluoro-2-
methoxyphenyl)-4,4-
dimethylcyclopentyl)-4-oxo-1,4-
dihydro-1,6-naphthyridine-5-
carboxamide 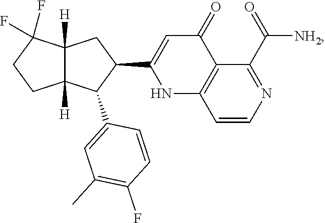 [SMILES: Cc1cc([C@@H]2[C@@H]3CCC(F)(F)[C@@H]3C[C@H]2c2cc(=O)c3c(C(N)=O)nccc3[nH]2)ccc1F]
2-((1R,2R,3aR,6aS)-4,4-difluoro-1-
(4-fluoro-3-
methylphenyl)octahydropentalen-
2-y1)-4-oxo-1,4-dihydro-1,6-
naphthyridine-5-carboxamide 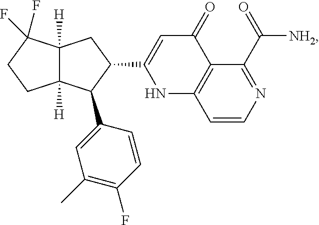 [SMILES: Cc1cc([C@H]2[C@H]3CCC(F)(F)[C@H]3C[C@@H]2c2cc(=O)c3c(C(N)=O)nccc3[nH]2)ccc1F]
2-((1S,2S,3aS,6aR)-4,4-difluoro-
1-(4-fluoro-3-
methylphenyl)octahydropentalen-
2-yl)-4-oxo-1,4-dihydro-1,6-
naphthyridine-5-carboxamide 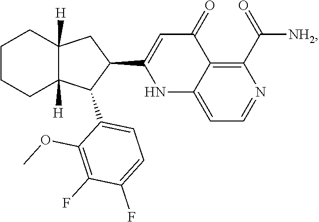 [SMILES: COc1c([C@@H]2[C@@H]3CCCC[C@@H]3C[C@H]2c2cc(=O)c3c(C(N)=O)nccc3[nH]2)ccc(F)c1F]
2-((1R,2R,3aR,7aR)-1-(3,4-
difluoro-2-
methoxyphenyl)octahydro-1H-
inden-2-yl)-4-oxo-1,4-dihydro-1,6-
naphthyridine-5-carboxamide 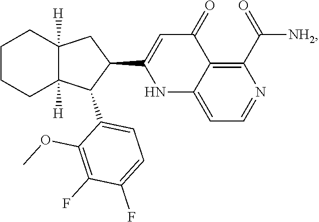 [SMILES: COc1c([C@@H]2[C@H]3CCCC[C@H]3C[C@H]2c2cc(=O)c3c(C(N)=O)nccc3[nH]2)ccc(F)c1F]
2-((1R,2R,3aS,7aS)-1-(3,4-difluoro-
2-methoxyphenyl)octahydro-1H-
inden-2-yl)-4-oxo-1,4-dihydro-1,6-
naphthyridine-5-carboxamide 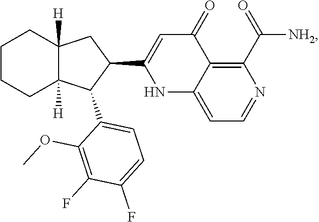 [SMILES: COc1c([C@@H]2[C@H]3CCCC[C@@H]3C[C@H]2c2cc(=O)c3c(C(N)=O)nccc3[nH]2)ccc(F)c1F]
2-((1R,2R,3aR,7aS)-1-(3,4-
difluoro-2-
methoxyphenyl)octahydro-1H-
inden-2-yl)-4-oxo-1,4-dihydro-
1,6-naphthyridine-5-
carboxamide 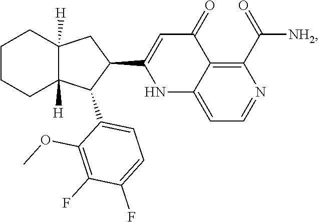 [SMILES: COc1c([C@@H]2[C@@H]3CCCC[C@H]3C[C@H]2c2cc(=O)c3c(C(N)=O)nccc3[nH]2)ccc(F)c1F]
2-((1R,2R,3aS,7aR)-1-(3,4-
difluoro-2-
methoxyphenyl)octahydro-1H-
inden-2-y1)-4-oxo-1,4-dihydro-1,6-
naphthyridine-5-carboxamide 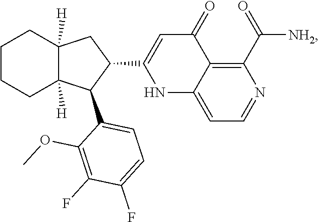 [SMILES: COc1c([C@H]2[C@H]3CCCC[C@H]3C[C@@H]2c2cc(=O)c3c(C(N)=O)nccc3[nH]2)ccc(F)c1F]
2-((15,2S,3aS,7aS)-1-(3,4-difluoro-
2-methoxyphenyl)octahydro-1H-
inden-2-yl)-4-oxo-1,4-dihydro-1,6-
naphthyridine-5-carboxamide 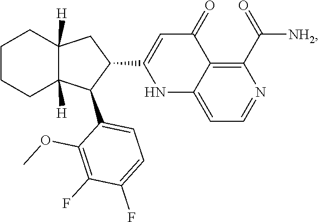 [SMILES: COc1c([C@H]2[C@@H]3CCCC[C@@H]3C[C@@H]2c2cc(=O)c3c(C(N)=O)nccc3[nH]2)ccc(F)c1F]
2-((1S,2S,3aR,7aR)-1-(3,4-
difluoro-2-
methoxyphenyl)octahydro-1H-
inden-2-yl)-4-oxo-1,4-dihydro-
1,6-naphthyridine-5-carboxamide 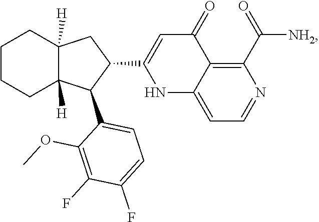 [SMILES: COc1c([C@H]2[C@@H]3CCCC[C@H]3C[C@@H]2c2cc(=O)c3c(C(N)=O)nccc3[nH]2)ccc(F)c1F]
2-((1S,2S,3aS,7aR)-1-(3,4-difluoro-
2-methoxyphenyl)octahydro-1H-
inden-2-yl)-4-oxo-1,4-dihydro-1,6-
naphthyridine-5-carboxamide 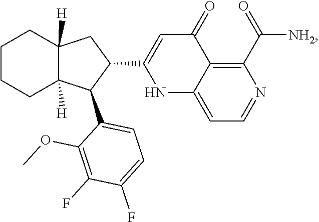 [SMILES: COc1c([C@H]2[C@H]3CCCC[C@@H]3C[C@@H]2c2cc(=O)c3c(C(N)=O)nccc3[nH]2)ccc(F)c1F]
2-((1S,2S,3aR,7aS)-1-(3,4-difluoro-
2-methoxyphenyl)octahydro-1H-
inden-2-yl)-4-oxo-1,4-dihydro-1,6-
naphthyridine-5-carboxamide 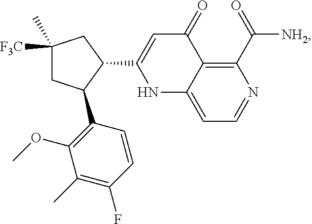 [SMILES: COc1c([C@H]2C[C@@](C)(C(F)(F)F)C[C@@H]2c2cc(=O)c3c(C(N)=O)nccc3[nH]2)ccc(F)c1C]
2-((1S,2S,4R)-2-(4-fluoro-2-
methoxy-3-methylphenyl)-4-
methyl-4-
(trifluoromethyl)cyclopentyl)-4-
oxo-1,4-dihydro-1,6-
naphthyridine-5-carboxamide 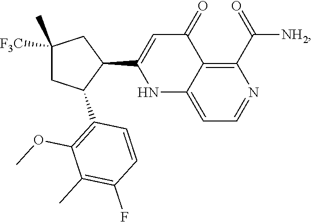 [SMILES: COc1c([C@@H]2C[C@](C)(C(F)(F)F)C[C@H]2c2cc(=O)c3c(C(N)=O)nccc3[nH]2)ccc(F)c1C]
2-((1R,2R,4S)-2-(4-fluoro-2-
methoxy-3-methylphenyl)-4-
methyl-4-
(trifluoromethyl)cyclopentyl)-4-
oxo-1,4-dihydro-1,6-naphthyridine-
5-carboxamide 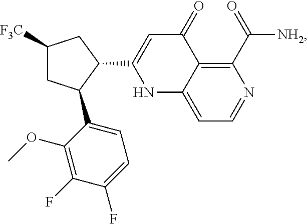 [SMILES: COc1c([C@H]2C[C@@H](C(F)(F)F)C[C@@H]2c2cc(=O)c3c(C(N)=O)nccc3[nH]2)ccc(F)c1F]
2-((1S,2S,4R)-2-(3,4-difluoro-2-
methoxyphenyl)-4-
(trifluoromethyl)cyclopentyl)-4-
oxo-1,4-dihydro-1,6-naphthyridine-
5-carboxamide 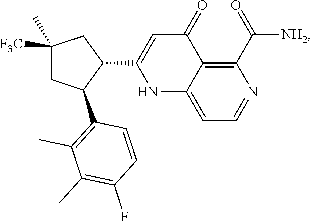 [SMILES: Cc1c(F)ccc([C@H]2C[C@@](C)(C(F)(F)F)C[C@@H]2c2cc(=O)c3c(C(N)=O)nccc3[nH]2)c1C]
2-((1S,2S,4R)-2-(4-fluoro-2,3-
dimethylphenyl)-4-methyl-4-
(trifluoromethyl)cyclopentyl)-4-
oxo-1,4-dihydro-1,6-
naphthyridine-5-carboxamide 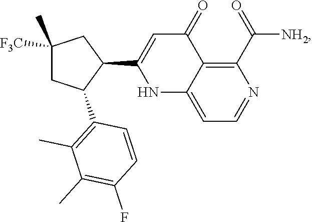 [SMILES: Cc1c(F)ccc([C@@H]2C[C@](C)(C(F)(F)F)C[C@H]2c2cc(=O)c3c(C(N)=O)nccc3[nH]2)c1C]
2-((1R,2R,4S)-2-(4-fluoro-2,3-
dimethylphenyl)-4-methyl-4-
(trifluoromethyl)cyclopentyl)-4-
oxo-1,4-dihydro-1,6-naphthyridine-
5-carboxamide 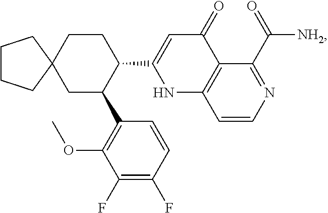 [SMILES: COc1c([C@H]2CC3(CCCC3)CC[C@@H]2c2cc(=O)c3c(C(N)=O)nccc3[nH]2)ccc(F)c1F]
2-((7S,8S)-7-(3,4-difluoro-2-
methoxyphenyl)spiro[4.5]decan-8-
yl)-4-oxo-1,4-dihydro-1,6-
naphthyridine-5-carboxamide 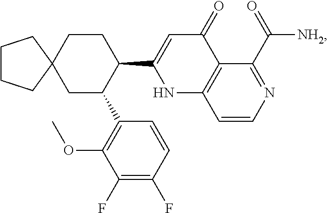 [SMILES: COc1c([C@@H]2CC3(CCCC3)CC[C@H]2c2cc(=O)c3c(C(N)=O)nccc3[nH]2)ccc(F)c1F]
2-((7R,8R)-7-(3,4-difluoro-2-
methoxyphenyl)spiro[4.5]decan-
8-yl)-4-oxo-1,4-dihydro-1,6-
naphthyridine-5-carboxamide 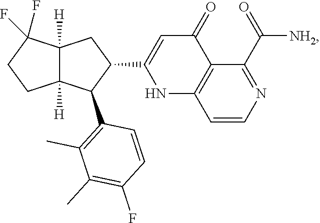 [SMILES: Cc1c(F)ccc([C@H]2[C@H]3CCC(F)(F)[C@H]3C[C@@H]2c2cc(=O)c3c(C(N)=O)nccc3[nH]2)c1C]
2-((1S,2S,3aS,6aR)-4,4-difluoro-1-
(4-fluoro-2,3-
dimethylphenyl)octahydropentalen-
2-y1)-4-oxo-1,4-dihydro-1,6-
naphthyridine-5-carboxamide 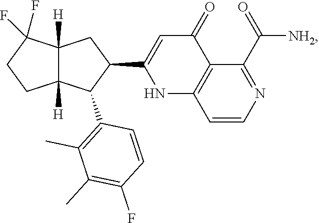 [SMILES: Cc1c(F)ccc([C@@H]2[C@@H]3CCC(F)(F)[C@@H]3C[C@H]2c2cc(=O)c3c(C(N)=O)nccc3[nH]2)c1C]
2-((1R,2R,3aR,6aS)-4,4-difluoro-1-
(4-fluoro-2,3-
dimethylphenyl)octahydropentalen-
2-yl)-4-oxo-1,4-dihydro-1,6-
naphthyridine-5-carboxamide 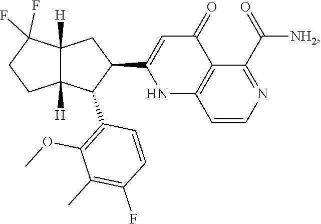 [SMILES: COc1c([C@@H]2[C@@H]3CCC(F)(F)[C@@H]3C[C@H]2c2cc(=O)c3c(C(N)=O)nccc3[nH]2)ccc(F)c1C]
2-((1R,2R,3aR,6aS)-4,4-difluoro-
1-(4-fluoro-2-methoxy-3-
methylphenyl)octahydropentalen-
2-yl)-4-oxo-1,4-dihydro-1,6-
naphthyridine-5-carboxamide 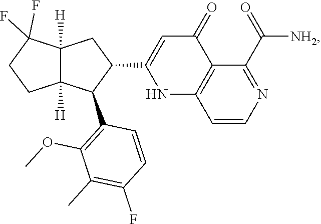 [SMILES: COc1c([C@H]2[C@H]3CCC(F)(F)[C@H]3C[C@@H]2c2cc(=O)c3c(C(N)=O)nccc3[nH]2)ccc(F)c1C]
2-((1S,2S,3aS,6aR)-4,4-difluoro-1-
(4-fluoro-2-methoxy-3-
methylphenyl)octahydropentalen-
2-yl)-4-oxo-1,4-dihydro-1,6-
naphthyridine-5-carboxamide 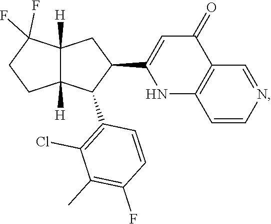 [SMILES: Cc1c(F)ccc([C@@H]2[C@@H]3CCC(F)(F)[C@@H]3C[C@H]2c2cc(=O)c3cnccc3[nH]2)c1Cl]
2-((1R,2R,3aR,6aS)-1-(2-chloro-4-
fluoro-3-methylphenyl)-4,4-
difluorooctahydropentalen-2-yl)-
1,6-naphthyridin-4(1H)-one 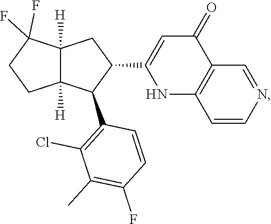 [SMILES: Cc1c(F)ccc([C@H]2[C@H]3CCC(F)(F)[C@H]3C[C@@H]2c2cc(=O)c3cnccc3[nH]2)c1Cl]
2-((1S,2S,3aS,6aR)-1-(2-chloro-
4-fluoro-3-methylphenyl)-4,4-
difluorooctahydropentalen-2-yl)-
1,6-naphthyridin-4(1H)-one 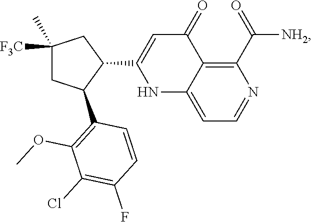 [SMILES: COc1c([C@H]2C[C@@](C)(C(F)(F)F)C[C@@H]2c2cc(=O)c3c(C(N)=O)nccc3[nH]2)ccc(F)c1Cl]
2-((1S,2S,4R)-2-(3-chloro-4-fluoro-
2-methoxyphenyl)-4-methyl-4-
(trifluoromethyl)cyclopentyl)-4-
oxo-1,4-dihydro-1,6-naphthyridine-
5-carboxamide 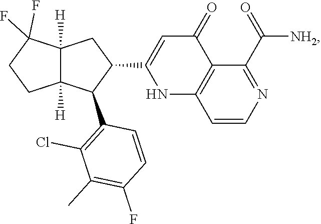 [SMILES: Cc1c(F)ccc([C@H]2[C@H]3CCC(F)(F)[C@H]3C[C@@H]2c2cc(=O)c3c(C(N)=O)nccc3[nH]2)c1Cl]
2-((1S,2S,3aS,6aR)-1-(2-chloro-4-
fluoro-3-methylphenyl)-4,4-
difluorooctahydropentalen-2-yl)-4-
oxo-1,4-dihydro-1,6-naphthyridine-
5-carboxamide 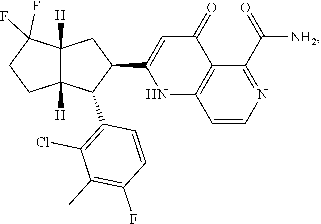 [SMILES: Cc1c(F)ccc([C@@H]2[C@@H]3CCC(F)(F)[C@@H]3C[C@H]2c2cc(=O)c3c(C(N)=O)nccc3[nH]2)c1Cl]
2-((1R,2R,3aR,6aS)-1-(2-chloro-
4-fluoro-3-methylphenyl)-4,4-
difluorooctahydropentalen-2-yl)-
4-oxo-1,4-dihydro-1,6-
naphthyridine-5-carboxamide 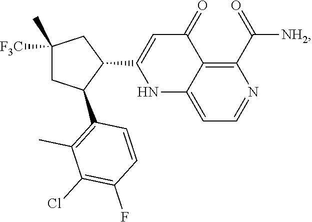 [SMILES: Cc1c([C@H]2C[C@](C)(C(F)(F)F)C[C@@H]2c2cc(=O)c3c(C(N)=O)nccc3[nH]2)ccc(F)c1Cl]
2-((1S,2S,4S)-2-(3-chloro-4-fluoro-
2-methylphenyl)-4-methyl-4-
(trifluoromethyl)cyclopentyl)-4-
oxo-1,4-dihydro-1,6-naphthyridine-
5-carboxamide 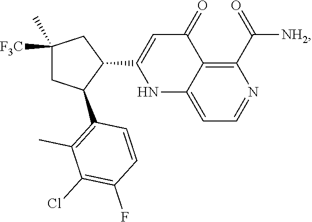 [SMILES: Cc1c([C@H]2C[C@@](C)(C(F)(F)F)C[C@@H]2c2cc(=O)c3c(C(N)=O)nccc3[nH]2)ccc(F)c1Cl]
2-((1S,2S,4R)-2-(3-chloro-4-fluoro-
2-methylphenyl)-4-methyl-4-
(trifluoromethyl)cyclopentyl)-4-
oxo-1,4-dihydro-1,6-naphthyridine-
5-carboxamide 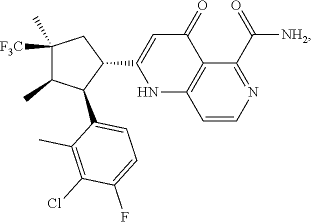 [SMILES: Cc1c([C@@H]2[C@@H](c3cc(=O)c4c(C(N)=O)nccc4[nH]3)C[C@](C)(C(F)(F)F)[C@@H]2C)ccc(F)c1Cl]
2-((1S,2S,3R,4S)-2-(3-chloro-4-
fluoro-2-methylphenyl)-3,4-
dimethyl-4-
(trifluoromethyl)cyclopentyl)-4-
oxo-1,4-dihydro-1,6-
naphthyridine-5-carboxamide 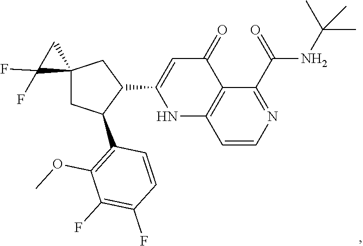
N-(tert-butyl)-2-((3R,5S,6S)-6-(3,4-
difluoro-2-methoxyphenyl)-1,1-
difluorospiro[2.4]heptan-5-yl)-4-
oxo-1,4-dihydro-1,6-naphthyridine-
5-carboxamide 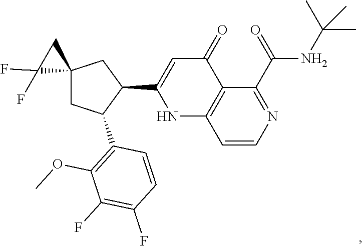 [SMILES: COc1c([C@@H]2C[C@]3(C[C@H]2c2cc(=O)c4c(C(=O)NC(C)(C)C)nccc4[nH]2)CC3(F)F)ccc(F)c1F]
N-(tert-butyl)-2-((3S,5R,6R)-6-
(3,4-difluoro-2-methoxyphenyl)-
1,1-difluorospiro[2.4]heptan-5-y1)-
4-oxo-1,4-dihydro-1,6-
naphthyridine-5-carboxamide 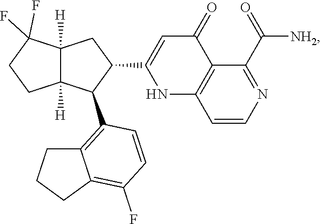 [SMILES: NC(=O)c1nccc2[nH]c([C@H]3C[C@H]4[C@H](CCC4(F)F)[C@@H]3c3ccc(F)c4c3CCC4)cc(=O)c12]
2-((1S,2S,3aS,6aR)-4,4-difluoro-
1-(7-fluoro-2,3-dihydro-1H-
inden-4-yl)octahydropentalen-2-
yl)-4-oxo-1,4-dihydro-1,6-
naphthyridine-5-carboxamide 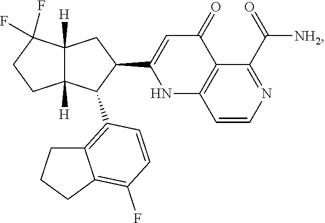 [SMILES: NC(=O)c1nccc2[nH]c([C@@H]3C[C@@H]4[C@@H](CCC4(F)F)[C@H]3c3ccc(F)c4c3CCC4)cc(=O)c12]
2-((1R,2R,3aR,6aS)-4,4-difluoro-1-
(7-fluoro-2,3-dihydro-1H-inden-4-
yl)octahydropentalen-2-y1)-4-oxo-
1,4-dihydro-1,6-naphthyridine-5-
carboxamide 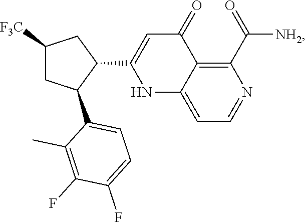 [SMILES: Cc1c([C@H]2C[C@@H](C(F)(F)F)C[C@@H]2c2cc(=O)c3c(C(N)=O)nccc3[nH]2)ccc(F)c1F]
2-((1S,2S,4R)-2-(3,4-difluoro-2-
methylphenyl)-4-
(trifluoromethyl)cyclopentyl)-4-
oxo-1,4-dihydro-1,6-naphthyridine-
5-carboxamide 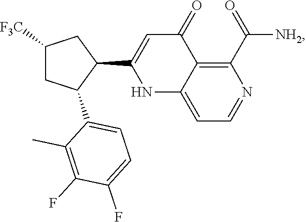 [SMILES: Cc1c([C@@H]2C[C@H](C(F)(F)F)C[C@H]2c2cc(=O)c3c(C(N)=O)nccc3[nH]2)ccc(F)c1F]
2-((1R,2R,4S)-2-(3,4-difluoro-2-
methylphenyl)-4-
(trifluoromethyl)cyclopentyl)-4-
oxo-1,4-dihydro-1,6-
naphthyridine-5-carboxamide 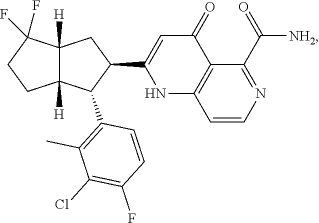 [SMILES: Cc1c([C@@H]2[C@@H]3CCC(F)(F)[C@@H]3C[C@H]2c2cc(=O)c3c(C(N)=O)nccc3[nH]2)ccc(F)c1Cl]
2-((1R,2R,3aR,6aS)-1-(3-chloro-4-
fluoro-2-methylphenyl)-4,4-
difluorooctahydropentalen-2-y1)-4-
oxo-1,4-dihydro-1,6-naphthyridine-
5-carboxamide 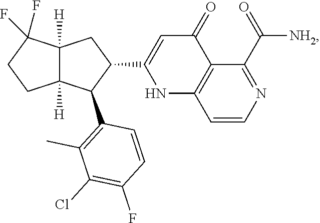 [SMILES: Cc1c([C@H]2[C@H]3CCC(F)(F)[C@H]3C[C@@H]2c2cc(=O)c3c(C(N)=O)nccc3[nH]2)ccc(F)c1Cl]
2-((1S,2S,3aS,6aR)-1-(3-chloro-4-
fluoro-2-methylphenyl)-4,4-
difluorooctahydropentalen-2-yl)-4-
oxo-1,4-dihydro-1,6-naphthyridine-
5-carboxamide 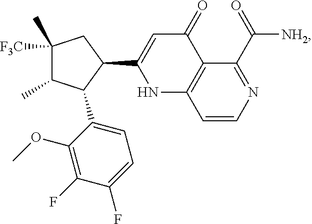 [SMILES: COc1c([C@H]2[C@H](c3cc(=O)c4c(C(N)=O)nccc4[nH]3)C[C@@](C)(C(F)(F)F)[C@H]2C)ccc(F)c1F]
2-((1R,2R,3S,4R)-2-(3,4-difluoro-
2-methoxyphenyl)-3,4-dimethyl-
4-(trifluoromethyl)cyclopentyl)-
4-oxo-1,4-dihydro-1,6-
naphthyridine-5-carboxamide 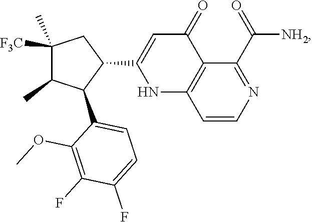 [SMILES: COc1c([C@@H]2[C@@H](c3cc(=O)c4c(C(N)=O)nccc4[nH]3)C[C@](C)(C(F)(F)F)[C@@H]2C)ccc(F)c1F]
2-((1S,2S,3R,4S)-2-(3,4-difluoro-2-
methoxyphenyl)-3,4-dimethyl-4-
(trifluoromethyl)cyclopentyl)-4-
oxo-1,4-dihydro-1,6-naphthyridine-
5-carboxamide 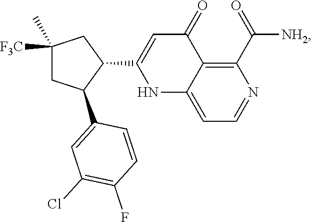 [SMILES: C[C@@]1(C(F)(F)F)C[C@H](c2ccc(F)c(Cl)c2)[C@@H](c2cc(=O)c3c(C(N)=O)nccc3[nH]2)C1]
2-((1S,2S,4R)-2-(3-chloro-4-
fluorophenyl)-4-methyl-4-
(trifluoromethyl)cyclopentyl)-4-
oxo-1,4-dihydro-1,6-naphthyridine-
5-carboxamide 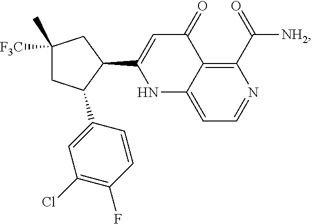 [SMILES: C[C@]1(C(F)(F)F)C[C@@H](c2ccc(F)c(Cl)c2)[C@H](c2cc(=O)c3c(C(N)=O)nccc3[nH]2)C1]
2-((1R,2R,4S)-2-(3-chloro-4-
fluorophenyl)-4-methyl-4-
(trifluoromethyl)cyclopentyl)-4-
oxo-1,4-dihydro-1,6-
naphthyridine-5-carboxamide 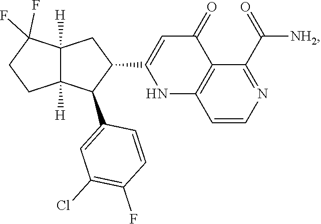 [SMILES: NC(=O)c1nccc2[nH]c([C@H]3C[C@H]4[C@H](CCC4(F)F)[C@@H]3c3ccc(F)c(Cl)c3)cc(=O)c12]
2-((1S,2S,3aS,6aR)-1-(3-chloro-4-
fluorophenyl)-4,4-
difluorooctahydropentalen-2-yl)-4-
oxo-1,4-dihydro-1,6-naphthyridine-
5-carboxamide 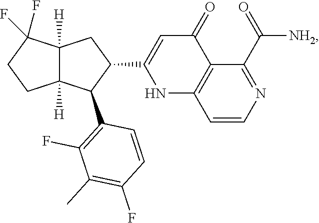 [SMILES: Cc1c(F)ccc([C@H]2[C@H]3CCC(F)(F)[C@H]3C[C@@H]2c2cc(=O)c3c(C(N)=O)nccc3[nH]2)c1F]
2-((1S,2S,3aS,6aR)-1-(2,4-difluoro-
3-methylphenyl)-4,4-
difluorooctahydropentalen-2-yl)-4-
oxo-1,4-dihydro-1,6-naphthyridine-
5-carboxamide 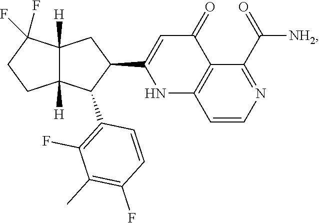 [SMILES: Cc1c(F)ccc([C@@H]2[C@@H]3CCC(F)(F)[C@@H]3C[C@H]2c2cc(=O)c3c(C(N)=O)nccc3[nH]2)c1F]
2-((1R,2R,3aR,6aS)-1-(2,4-
difluoro-3-methylphenyl)-4,4-
difluorooctahydropentalen-2-yl)-
4-oxo-1,4-dihydro-1,6-
naphthyridine-5-carboxamide 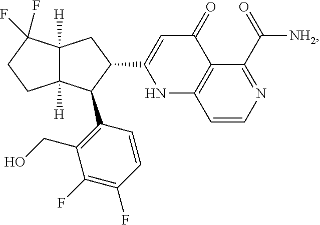 [SMILES: NC(=O)c1nccc2[nH]c([C@H]3C[C@H]4[C@H](CCC4(F)F)[C@@H]3c3ccc(F)c(F)c3CO)cc(=O)c12]
2-((1S,2S,3aS,6aR)-1-(3,4-difluoro-
2-(hydroxymethyl)phenyl)-4,4-
difluorooctahydropentalen-2-yl)-4-
oxo-1,4-dihydro-1,6-naphthyridine-
5-carboxamide 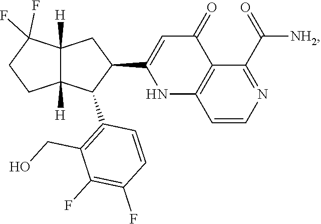 [SMILES: NC(=O)c1nccc2[nH]c([C@@H]3C[C@@H]4[C@@H](CCC4(F)F)[C@H]3c3ccc(F)c(F)c3CO)cc(=O)c12]
2-((1R,2R,3aR,6aS)-1-(3,4-
difluoro-2-
(hydroxymethyl)phenyl)-4,4-
difluorooctahydropentalen-2-yl)-4-
oxo-1,4-dihydro-1,6-naphthyridine-
5-carboxamide 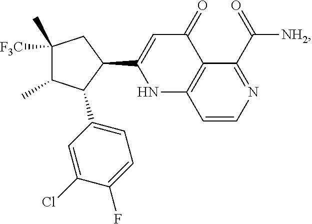 [SMILES: C[C@H]1[C@@H](c2ccc(F)c(Cl)c2)[C@H](c2cc(=O)c3c(C(N)=O)nccc3[nH]2)C[C@@]1(C)C(F)(F)F]
2-((1R,2R,3S,4R)-2-(3-chloro-4-
fluorophenyl)-3,4-dimethyl-4-
(trifluoromethyl)cyclopentyl)-4-
oxo-1,4-dihydro-1,6-
naphthyridine-5-carboxamide 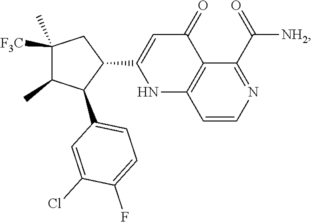 [SMILES: C[C@@H]1[C@H](c2ccc(F)c(Cl)c2)[C@@H](c2cc(=O)c3c(C(N)=O)nccc3[nH]2)C[C@]1(C)C(F)(F)F]
2-((1S,2S,3R,4S)-2-(3-chloro-4-
fluorophenyl)-3,4-dimethyl-4-
(trifluoromethyl)cyclopentyl)-4-
oxo-1,4-dihydro-1,6-naphthyridine-
5-carboxamide 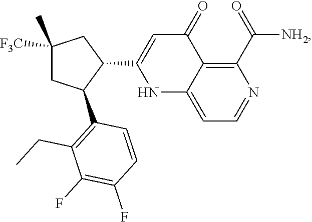 [SMILES: CCc1c([C@H]2C[C@](C)(C(F)(F)F)C[C@@H]2c2cc(=O)c3c(C(N)=O)nccc3[nH]2)ccc(F)c1F]
2-((1S,2S,4S)-2-(2-ethyl-3,4-
difluorophenyl)-4-methyl-4-
(trifluoromethyl)cyclopentyl)-4-
oxo-1,4-dihydro-1,6-naphthyridine-
5-carboxamide 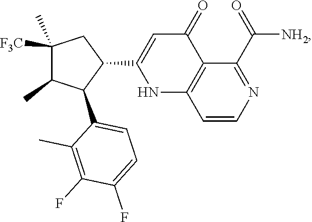 [SMILES: Cc1c([C@@H]2[C@@H](c3cc(=O)c4c(C(N)=O)nccc4[nH]3)C[C@](C)(C(F)(F)F)[C@@H]2C)ccc(F)c1F]
2-((1S,2S,3R,4S)-2-(3,4-difluoro-
2-methylphenyl)-3,4-dimethyl-4-
(trifluoromethyl)cyclopentyl)-4-
oxo-1,4-dihydro-1,6-
naphthyridine-5-carboxamide 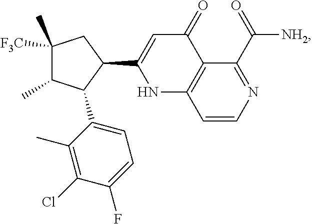 [SMILES: Cc1c([C@H]2[C@H](c3cc(=O)c4c(C(N)=O)nccc4[nH]3)C[C@@](C)(C(F)(F)F)[C@H]2C)ccc(F)c1Cl]
2-((1R.2R,3S,4R)-2-(3,4-difluoro-
2-methylphenyl)-3,4-dimethyl-4-
(trifluoromethyl)cyclopentyl)-4-
oxo-1,4-dihydro-1,6-naphthyridine-
5-carboxamide 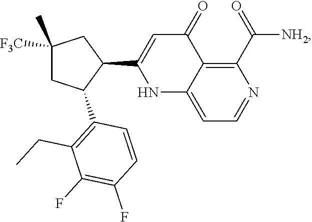 [SMILES: CCc1c([C@@H]2C[C@](C)(C(F)(F)F)C[C@H]2c2cc(=O)c3c(C(N)=O)nccc3[nH]2)ccc(F)c1F]
2-((1R,2R,4S)-2-(2-ethyl-3,4-
difluorophenyl)-4-methyl-4-
(trifluoromethyl)cyclopentyl)-4-
oxo-1,4-dihydro-1,6-naphthyridine-
5-carboxamide 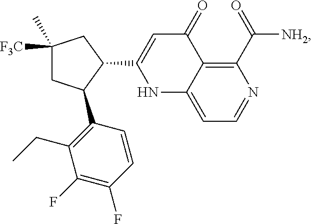 [SMILES: CCc1c([C@H]2C[C@@](C)(C(F)(F)F)C[C@@H]2c2cc(=O)c3c(C(N)=O)nccc3[nH]2)ccc(F)c1F]
2-((1S.2S,4R)-2-(2-ethyl-3,4-
difluorophenyl)-4-methyl-4-
(trifluoromethyl)cyclopentyl)-4-
oxo-1,4-dihydro-1,6-
naphthyridine-5-carboxamide 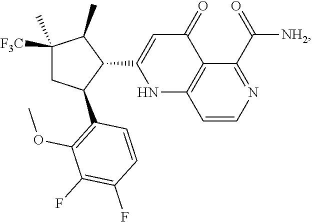 [SMILES: COc1c([C@H]2C[C@@](C)(C(F)(F)F)[C@@H](C)[C@@H]2c2cc(=O)c3c(C(N)=O)nccc3[nH]2)ccc(F)c1F]
2-((1S,2S,3R,5S)-5-(3,4-difluoro-2-
methoxyphenyl)-2,3-dimethyl-3-
(trifluoromethyl)cyclopentyl)-4-
oxo-1,4-dihydro-1,6-naphthyridine-
5-carboxamide 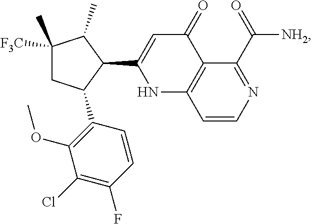 [SMILES: COc1c([C@@H]2C[C@](C)(C(F)(F)F)[C@H](C)[C@H]2c2cc(=O)c3c(C(N)=O)nccc3[nH]2)ccc(F)c1Cl]
2-((1R,2R,3S,5R)-5-(3,4-difluoro-
2-methoxyphenyl)-2,3-dimethyl-3-
(trifluoromethyl)cyclopentyl)-4-
oxo-1,4-dihydro-1,6-naphthyridine-
5-carboxamide 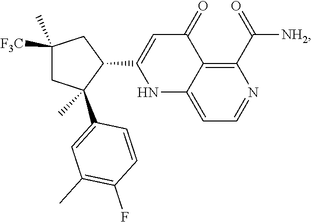 [SMILES: Cc1cc([C@@]2(C)C[C@@](C)(C(F)(F)F)C[C@@H]2c2cc(=O)c3c(C(N)=O)nccc3[nH]2)ccc1F]
2-((1S,2S,4S)-2-(4-fluoro-3-
methylphenyl)-2,4-dimethyl-4-
(trifluoromethyl)cyclopentyl)-4-
oxo-1,4-dihydro-1,6-
naphthyridine-5-carboxamide 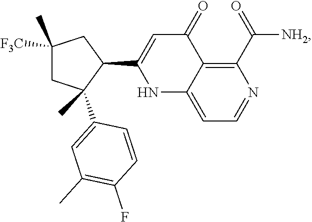 [SMILES: Cc1cc([C@]2(C)C[C@](C)(C(F)(F)F)C[C@H]2c2cc(=O)c3c(C(N)=O)nccc3[nH]2)ccc1F]
2-((1R,2R,4R)-2-(4-fluoro-3-
methylphenyl)-2,4-dimethyl-4-
(trifluoromethyl)cyclopentyl)-4-
oxo-1,4-dihydro-1,6-naphthyridine-
5-carboxamide 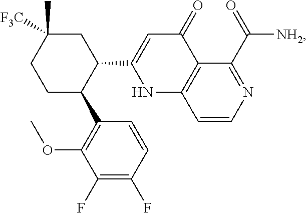 [SMILES: COc1c([C@H]2CC[C@](C)(C(F)(F)F)C[C@@H]2c2cc(=O)c3c(C(N)=O)nccc3[nH]2)ccc(F)c1F]
2-((1S,2S,5S)-2-(3,4-difluoro-2-
methoxyphenyl)-5-methyl-5-
(trifluoromethyl)cyclohexyl)-4-
oxo-1,4-dihydro-1,6-naphthyridine-
5-carboxamide 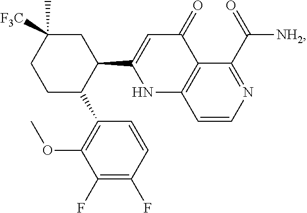
2-((1R,2R,5R)-2-(3,4-difluoro-2-
methoxyphenyl)-5-methyl-5-
(trifluoromethyl)cyclohexyl)-4-
oxo-1,4-dihydro-1,6-
naphthyridine-5-carboxamide 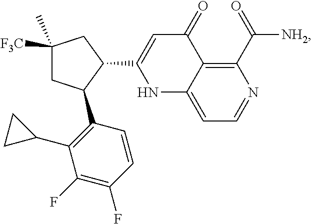 [SMILES: C[C@]1(C(F)(F)F)C[C@H](c2cc(=O)c3c(C(N)=O)nccc3[nH]2)[C@@H](c2ccc(F)c(F)c2C2CC2)C1]
2-((1S,2S,4R)-2-(2-cyclopropyl-
3,4-difluorophenyl)-4-methyl-4-
(trifluoromethyl)cyclopentyl)-4-
oxo-1,4-dihydro-1,6-naphthyridine-
5-carboxamide 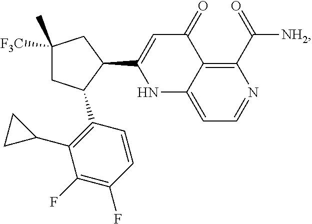 [SMILES: C[C@@]1(C(F)(F)F)C[C@@H](c2cc(=O)c3c(C(N)=O)nccc3[nH]2)[C@H](c2ccc(F)c(F)c2C2CC2)C1]
2-((1R,2R,4S)-2-(2-cyclopropyl-
3,4-difluorophenyl)-4-methyl-4-
(trifluoromethyl)cyclopentyl)-4-
oxo-1,4-dihydro-1,6-naphthyridine-
5-carboxamide 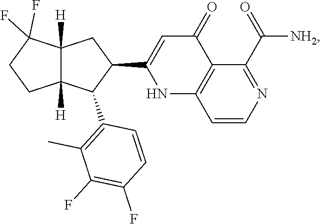 [SMILES: Cc1c([C@@H]2[C@@H]3CCC(F)(F)[C@@H]3C[C@H]2c2cc(=O)c3c(C(N)=O)nccc3[nH]2)ccc(F)c1F]
2-((1R,2R,3aR,6aS)-1-(3,4-
difluoro-2-methylphenyl)-4,4-
difluorooctahydropentalen-2-yl)-
4-oxo-1,4-dihydro-1,6-
naphthyridine-5-carboxamide 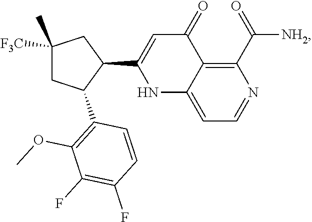 [SMILES: COc1c([C@@H]2C[C@](C)(C(F)(F)F)C[C@H]2c2cc(=O)c3c(C(N)=O)nccc3[nH]2)ccc(F)c1F]
2-((1R,2R,4S)-2-(3,4-difluoro-2-
methoxyphenyl)-4-methyl-4-
(trifluoromethyl)cyclopentyl)-4-
oxo-1,4-dihydro-1,6-naphthyridine-
5-carboxamide 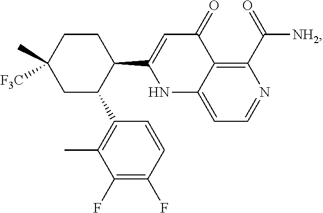 [SMILES: Cc1c([C@@H]2C[C@](C)(C(F)(F)F)CC[C@H]2c2cc(=O)c3c(C(N)=O)nccc3[nH]2)ccc(F)c1F]
2-((1R,2R,4R)-2-(3,4-difluoro-2-
methylphenyl)-4-methyl-4-
(trifluoromethyl)cyclohexyl)-4-
oxo-1,4-dihydro-1,6-naphthyridine-
5-carboxamide 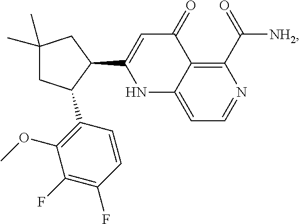 [SMILES: COc1c([C@@H]2CC(C)(C)C[C@H]2c2cc(=O)c3c(C(N)=O)nccc3[nH]2)ccc(F)c1F]
2-((1R,2R)-2-(3,4-difluoro-2-
methoxyphenyl)-4,4-
dimethylcyclopentyl)-4-oxo-1,4-
dihydro-1,6-naphthyridine-5-
carboxamide 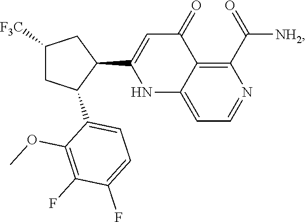 [SMILES: COc1c([C@@H]2C[C@H](C(F)(F)F)C[C@H]2c2cc(=O)c3c(C(N)=O)nccc3[nH]2)ccc(F)c1F]
2-((1R,2R,4S)-2-(3,4-difluoro-2-
methoxyphenyl)-4-
(trifluoromethyl)cyclopentyl)-4-
oxo-1,4-dihydro-1,6-naphthyridine-
5-carboxamide 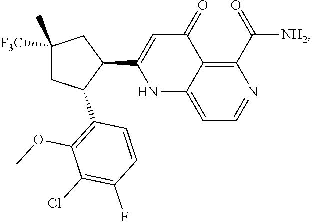 [SMILES: COc1c([C@@H]2C[C@](C)(C(F)(F)F)C[C@H]2c2cc(=O)c3c(C(N)=O)nccc3[nH]2)ccc(F)c1Cl]
2-((1R,2R,4S)-2-(3-chloro-4-
fluoro-2-methoxyphenyl)-4-
methyl-4-
(trifluoromethyl)cyclopentyl)-4-
oxo-1,4-dihydro-1,6-naphthyridine-
5-carboxamide 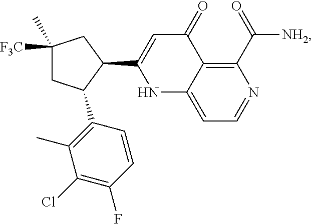 [SMILES: Cc1c([C@@H]2C[C@@](C)(C(F)(F)F)C[C@H]2c2cc(=O)c3c(C(N)=O)nccc3[nH]2)ccc(F)c1Cl]
2-((1R,2R,4R)-2-(3-chloro-4-
fluoro-2-methylphenyl)-4-
methyl-4-
(trifluoromethyl)cyclopentyl)-4-
oxo-1,4-dihydro-1,6-
naphthyridine-5-carboxamide 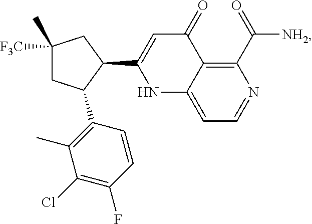 [SMILES: Cc1c([C@@H]2C[C@](C)(C(F)(F)F)C[C@H]2c2cc(=O)c3c(C(N)=O)nccc3[nH]2)ccc(F)c1Cl]
2-((1R,2R,4S)-2-(3-chloro-4-
fluoro-2-methylphenyl)-4-methyl-
4-(trifluoromethyl)cyclopentyl)-4-
oxo-1,4-dihydro-1,6-naphthyridine-
5-carboxamide 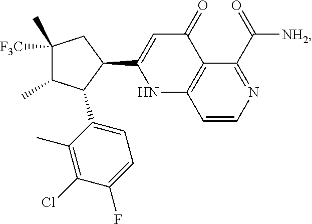 [SMILES: Cc1c([C@H]2[C@H](c3cc(=O)c4c(C(N)=O)nccc4[nH]3)C[C@@](C)(C(F)(F)F)[C@H]2C)ccc(F)c1Cl]
2-((1R,2R,3S,4R)-2-(3-chloro-4-
fluoro-2-methylphenyl)-3,4-
dimethyl-4-
(trifluoromethyl)cyclopentyl)-4-
oxo-1,4-dihydro-1,6-naphthyridine-
5-carboxamide 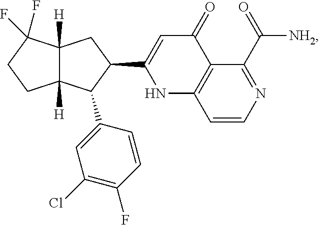 [SMILES: NC(=O)c1nccc2[nH]c([C@@H]3C[C@@H]4[C@@H](CCC4(F)F)[C@H]3c3ccc(F)c(Cl)c3)cc(=O)c12]
and
2-((1R,2R,3aR,6aS)-1-(3-chloro-
4-fluorophenyl)-4,4-
difluorooctahydropentalen-2-yl)-
4-oxo-1,4-dihydro-1,6-
naphthyridine-5-carboxamide 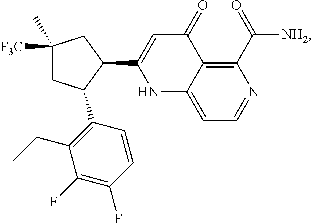 [SMILES: CCc1c([C@@H]2C[C@@](C)(C(F)(F)F)C[C@H]2c2cc(=O)c3c(C(N)=O)nccc3[nH]2)ccc(F)c1F]
2-((1R,2R,4R)-2-(2-ethyl-3,4-
difluorophenyl)-4-methyl-4-
(trifluoromethyl)cyclopentyl)-4-
oxo-1,4-dihydro-1,6-naphthyridine-
5-carboxamide

34. A compound selected from: 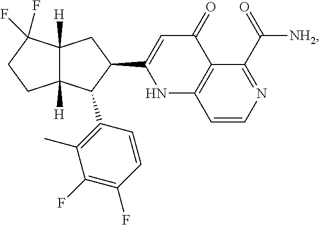 [SMILES: Cc1c([C@@H]2[C@@H]3CCC(F)(F)[C@@H]3C[C@H]2c2cc(=O)c3c(C(N)=O)nccc3[nH]2)ccc(F)c1F]
2-((1R,2R,3aR,6aS)-1-(3,4-
difluoro-2-methylphenyl)-4,4-
difluorooctahydropentalen-2-yl)-4-
oxo-1,4-dihydro-1,6-
naphthyridine-5-carboxamide 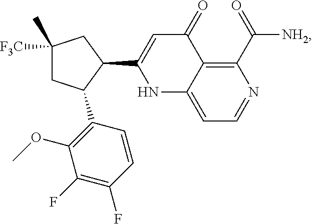 [SMILES: COc1c([C@@H]2C[C@](C)(C(F)(F)F)C[C@H]2c2cc(=O)c3c(C(N)=O)nccc3[nH]2)ccc(F)c1F]
2-((1R,2R,4S)-2-(3,4-difluoro-2-
methoxyphenyl)-4-methyl-4-
(trifluoromethyl)cyclopentyl)-4-
oxo-1,4-dihydro-1,6-
naphthyridine-5-carboxamide 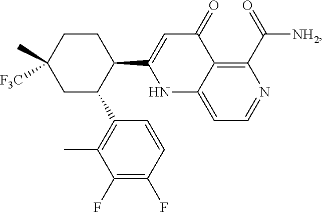 [SMILES: Cc1c([C@@H]2C[C@](C)(C(F)(F)F)CC[C@H]2c2cc(=O)c3c(C(N)=O)nccc3[nH]2)ccc(F)c1F]
2-((1R,2R,4R)-2-(3,4-difluoro-2-
methylphenyl)-4-methyl-4-
(trifluoromethyl)cyclohexyl)-4-
oxo-1,4-dihydro-1,6-
naphthyridine-5-carboxamide 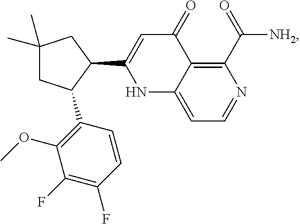 [SMILES: COc1c([C@@H]2CC(C)(C)C[C@H]2c2cc(=O)c3c(C(N)=O)nccc3[nH]2)ccc(F)c1F]
2-((1R,2R)-2-(3,4-difluoro-2-
methoxyphenyl)-4,4-
dimethylcyclopentyl)-4-oxo-1,4-
dihydro-1,6-naphthyridine-5-
carboxamide 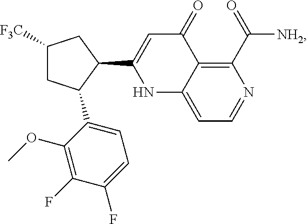 [SMILES: COc1c([C@@H]2C[C@H](C(F)(F)F)C[C@H]2c2cc(=O)c3c(C(N)=O)nccc3[nH]2)ccc(F)c1F]
2-((1R,2R,4S)-2-(3,4-difluoro-2-
methoxyphenyl)-4-
(trifluoromethyl)cyclopentyl)-4-
oxo-1,4-dihydro-1,6-
naphthyridine-5-carboxamide 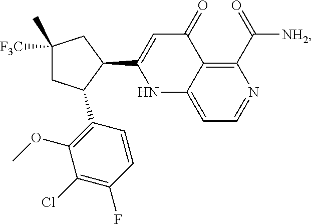 [SMILES: COc1c([C@@H]2C[C@](C)(C(F)(F)F)C[C@H]2c2cc(=O)c3c(C(N)=O)nccc3[nH]2)ccc(F)c1Cl]
2-((1R,2R,4S)-2-(3-chloro-4-
fluoro-2-methoxyphenyl)-4-
methyl-4-
(trifluoromethyl)cyclopenty1)-4-
oxo-1,4-dihydro-1,6-
naphthyridine-5-carboxamide 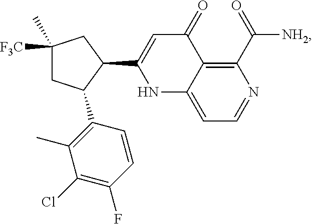 [SMILES: Cc1c([C@@H]2C[C@@](C)(C(F)(F)F)C[C@H]2c2cc(=O)c3c(C(N)=O)nccc3[nH]2)ccc(F)c1Cl]
2-((1R,2R,4R)-2-(3-chloro-4-
fluoro-2-methylphenyl)-4-methyl-
4-(trifluoromethyl)cyclopentyl)-4-
oxo-1,4-dihydro-1,6-
naphthyridine-5-carboxamide 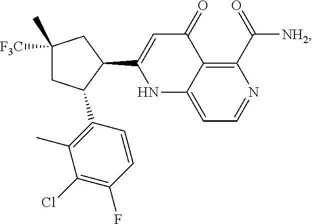 [SMILES: Cc1c([C@@H]2C[C@](C)(C(F)(F)F)C[C@H]2c2cc(=O)c3c(C(N)=O)nccc3[nH]2)ccc(F)c1Cl]
2-((1R,2R.4S)-2-(3-chloro-4-
fluoro-2-methylphenyl)-4-methyl-
4-(trifluoromethyl)cyclopentyl)-4-
oxo-1,4-dihydro-1,6-
naphthyridine-5-carboxamide 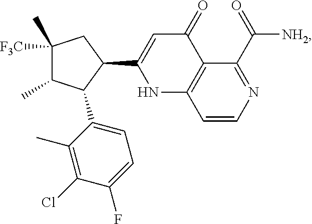 [SMILES: Cc1c([C@H]2[C@H](c3cc(=O)c4c(C(N)=O)nccc4[nH]3)C[C@@](C)(C(F)(F)F)[C@H]2C)ccc(F)c1Cl]
2-((1R,2R,3S,4R)-2-(3-chloro-4-
fluoro-2-methylphenyl)-3,4-
dimethyl-4-
(trifluoromethyl)cyclopentyl)-4-
oxo-1,4-dihydro-1,6-
naphthyridine-5-carboxamide 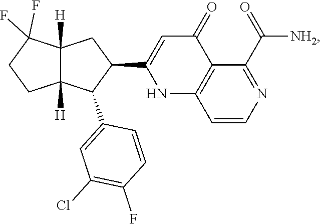 [SMILES: NC(=O)c1nccc2[nH]c([C@@H]3C[C@@H]4[C@@H](CCC4(F)F)[C@H]3c3ccc(F)c(Cl)c3)cc(=O)c12]
and
2-((1R,2R,3aR,6aS)-1-(3-chloro-4-fluorophenyl)-4,4-
difluorooctahydropentalen-2-yl)-4-oxo-1,4-dihydro-
1,6-naphthyridine-5-carboxamide 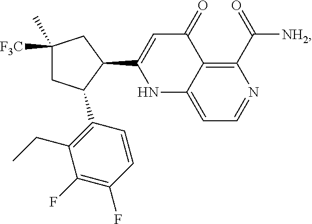 [SMILES: CCc1c([C@@H]2C[C@@](C)(C(F)(F)F)C[C@H]2c2cc(=O)c3c(C(N)=O)nccc3[nH]2)ccc(F)c1F]
2-((1R,2R,4R)-2-(2-ethyl-3,4-difluorophenyl)-4-
methyl-4-(trifluoromethyl)cyclopentyl)-4-oxo-1,4-
dihydro-1,6-naphthyridine-5-carboxamide
or a pharmaceutically acceptable salt thereof.

35. A compound selected from: 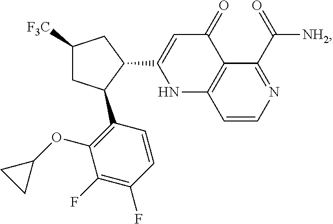 [SMILES: NC(=O)c1nccc2[nH]c([C@H]3C[C@H](C(F)(F)F)C[C@@H]3c3ccc(F)c(F)c3OC3CC3)cc(=O)c12]
2-((1S,2S,4R)-2-(2-cyclopropoxy-
3,4-difluorophenyl)-4-
(trifluoromethyl)cyclopentyl)-4-
oxo-1,4-dihydro-1,6-
naphthyridine-5-carboxamide 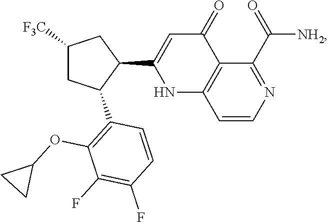 [SMILES: NC(=O)c1nccc2[nH]c([C@@H]3C[C@@H](C(F)(F)F)C[C@H]3c3ccc(F)c(F)c3OC3CC3)cc(=O)c12]
2-((1R,2R,4S)-2-(2-cyclopropoxy-
3,4-difluorophenyl)-4-
(trifluoromethyl)cyclopentyl)-4-
oxo-1,4-dihydro-1,6-
naphthyridine-5-carboxamide 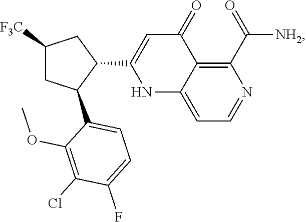 [SMILES: COc1c([C@H]2C[C@@H](C(F)(F)F)C[C@@H]2c2cc(=O)c3c(C(N)=O)nccc3[nH]2)ccc(F)c1Cl]
2-((1S,2S,4R)-2-(3-chloro-4-
fluoro-2-methoxyphenyl)-4-
(trifluoromethyl)cyclopentyl)-4-
oxo-1,4-dihydro-1,6-
naphthyridine-5-carboxamide 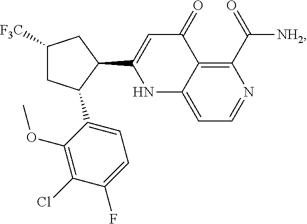 [SMILES: COc1c([C@@H]2C[C@H](C(F)(F)F)C[C@H]2c2cc(=O)c3c(C(N)=O)nccc3[nH]2)ccc(F)c1Cl]
2-((1R,2R,4S)-2-(3-chloro-4-
fluoro-2-methoxyphenyl)-4-
(trifluoromethyl)cyclopentyl)-4-
oxo-1,4-dihydro-1,6-
naphthyridine-5-carboxamide 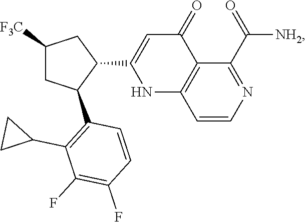 [SMILES: NC(=O)c1nccc2[nH]c([C@H]3C[C@H](C(F)(F)F)C[C@@H]3c3ccc(F)c(F)c3C3CC3)cc(=O)c12]
2-((1S,2S,4R)-2-(2-cyclopropyl-
3,4-difluorophenyl)-4-
(trifluoromethyl)cyclopentyl)-4-
oxo-1,4-dihydro-1,6-
naphthyridine-5-carboxamide 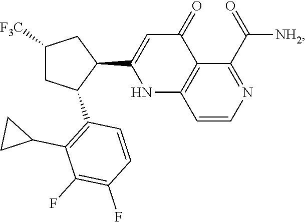 [SMILES: NC(=O)c1nccc2[nH]c([C@@H]3C[C@@H](C(F)(F)F)C[C@H]3c3ccc(F)c(F)c3C3CC3)cc(=O)c12]
2-((1R,2R,4S)-2-(2-cyclopropyl-
3,4-difluorophenyl)-4-
(trifluoromethyl)cyclopentyl)-4-
oxo-1,4-dihydro-1,6-
naphthyridine-5-carboxamide 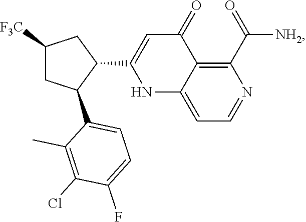 [SMILES: Cc1c([C@H]2C[C@@H](C(F)(F)F)C[C@@H]2c2cc(=O)c3c(C(N)=O)nccc3[nH]2)ccc(F)c1Cl]
2-((1S,2S,4R)-2-(3-chloro-4-
fluoro-2-methylphenyl)-4-
(trifluoromethyl)cyclopentyl)-4-
oxo-1,4-dihydro-1,6-
naphthyridine-5-carboxamide 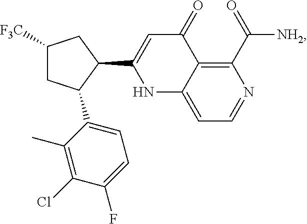 [SMILES: Cc1c([C@@H]2C[C@H](C(F)(F)F)C[C@H]2c2cc(=O)c3c(C(N)=O)nccc3[nH]2)ccc(F)c1Cl]
2-((1R,2R,4S)-2-(3-chloro-4-
fluoro-2-methylphenyl)-4-
(trifluoromethyl)cyclopentyl)-4-
oxo-1,4-dihydro-1,6-
naphthyridine-5-carboxamide 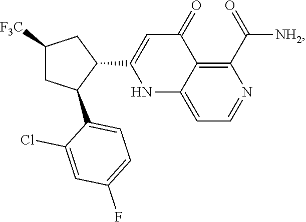 [SMILES: NC(=O)c1nccc2[nH]c([C@H]3C[C@H](C(F)(F)F)C[C@@H]3c3ccc(F)cc3Cl)cc(=O)c12]
2-((1S,2S,4R)-2-(2-chloro-4-
fluorophenyl)-4-
(trifluoromethyl)cyclopentyl)-4-
oxo-1,4-dihydro-1,6-
naphthyridine-5-carboxamide 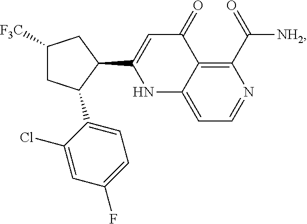 [SMILES: NC(=O)c1nccc2[nH]c([C@@H]3C[C@@H](C(F)(F)F)C[C@H]3c3ccc(F)cc3Cl)cc(=O)c12]
2-((1R,2R,4S)-2-(2-chloro-4-
fluorophenyl)-4-
(trifluoromethyl)cyclopentyl)-4-
oxo-1,4-dihydro-1,6-
naphthyridine-5-carboxamide 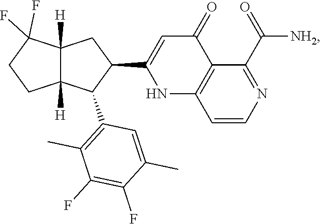 [SMILES: Cc1cc([C@@H]2[C@@H]3CCC(F)(F)[C@@H]3C[C@H]2c2cc(=O)c3c(C(N)=O)nccc3[nH]2)c(C)c(F)c1F]
2-((1R,2R,3aR,6aS)-1-(3,4-
difluoro-2,5-dimethylphenyl)-4,4-
difluorooctahydropentalen-2-yl)-4-
oxo-1,4-dihydro-1,6-
naphthyridine-5-carboxamide 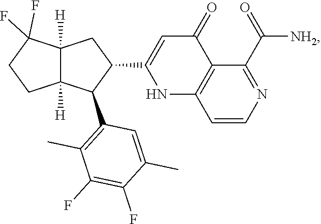 [SMILES: Cc1cc([C@H]2[C@H]3CCC(F)(F)[C@H]3C[C@@H]2c2cc(=O)c3c(C(N)=O)nccc3[nH]2)c(C)c(F)c1F]
2-((1S,2S,3aS,6aR)-1-(3,4-
difluoro-2,5-dimethylphenyl)-4,4-
difluorooctahydropentalen-2-yl)-4-
oxo-1,4-dihydro-1,6-
naphthyridine-5-carboxamide 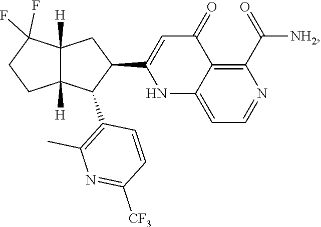 [SMILES: Cc1nc(C(F)(F)F)ccc1[C@@H]1[C@@H]2CCC(F)(F)[C@@H]2C[C@H]1c1cc(=O)c2c(C(N)=O)nccc2[nH]1]
2-((1R,2R,3aR,6aS)-4,4-difluoro-
1-(2-methyl-6-
(trifluoromethyl)pyridin-3-
yl)octahydropentalen-2-y1)-4-oxo-
1,4-dihydro-1,6-naphthyridine-5-
carboxamide 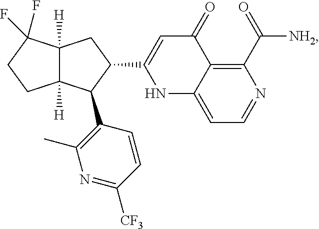 [SMILES: Cc1nc(C(F)(F)F)ccc1[C@H]1[C@H]2CCC(F)(F)[C@H]2C[C@@H]1c1cc(=O)c2c(C(N)=O)nccc2[nH]1]
2-((1S,2S,3aS,6aR)-4,4-difluoro-1-
(2-methyl-6-
(trifluoromethyl)pyridin-3-
yl)octahydropentalen-2-y1)-4-oxo-
1,4-dihydro-1,6-naphthyridine-5-
carboxamide 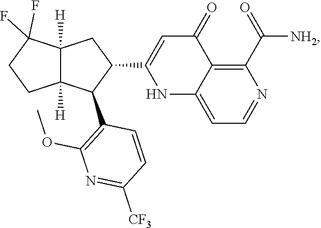 [SMILES: COc1nc(C(F)(F)F)ccc1[C@H]1[C@H]2CCC(F)(F)[C@H]2C[C@@H]1c1cc(=O)c2c(C(N)=O)nccc2[nH]1]
2-((1S,2S,3aS,6aR)-4,4-difluoro-1-
(2-methoxy-6-
(trifluoromethyl)pyridin-3-
yl)octahydropentalen-2-yl)-4-oxo-
1,4-dihydro-1,6-naphthyridine-5-
carboxamide 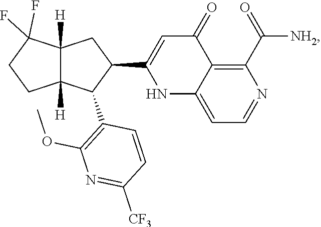 [SMILES: COc1nc(C(F)(F)F)ccc1[C@@H]1[C@@H]2CCC(F)(F)[C@@H]2C[C@H]1c1cc(=O)c2c(C(N)=O)nccc2[nH]1]
2-((1R,2R,3aR,6aS)-4,4-difluoro-
1-(2-methoxy-6-
(trifluoromethyl)pyridin-3-
yl)octahydropentalen-2-yl)-4-oxo-
1,4-dihydro-1,6-naphthyridine-5-
carboxamide 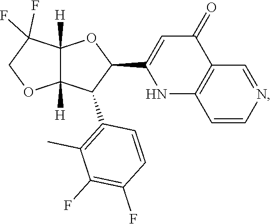 [SMILES: Cc1c([C@@H]2[C@@H]3OCC(F)(F)[C@@H]3O[C@H]2c2cc(=O)c3cnccc3[nH]2)ccc(F)c1F]
2-((2R,3S,3aS,6aR)-3-(3,4-
difluoro-2-methylphenyl)-6,6-
difluorohexahydrofuro 3,2-
b]furan-2-y1)-1,6-naphthyridin-
4(1H)-one 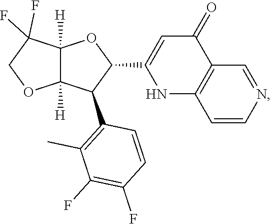 [SMILES: Cc1c([C@H]2[C@H]3OCC(F)(F)[C@H]3O[C@@H]2c2cc(=O)c3cnccc3[nH]2)ccc(F)c1F]
2-((2S,3R,3aR,6aS)-3-(3,4-
difluoro-2-methylphenyl)-6,6-
difluorohexahydrofuro[3,2-
b]furan-2-yl)-1,6-naphthyridin-
4(1H)-one 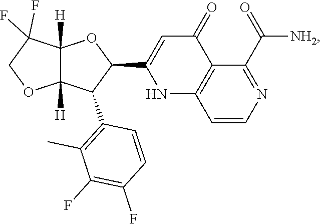 [SMILES: Cc1c([C@@H]2[C@@H]3OCC(F)(F)[C@@H]3O[C@H]2c2cc(=O)c3c(C(N)=O)nccc3[nH]2)ccc(F)c1F]
and
2-((2R,3S,3aS,6aR)-3-(3,4-difluoro-2-methylphenyl)-
6,6-difluorohexahydrofuro[3,2-b]furan-2-y1)-4-oxo-
1,4-dihydro-1,6-naphthyridine-5-carboxamide 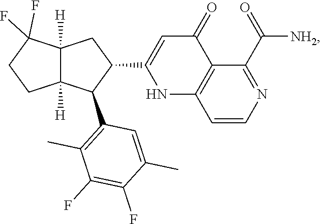 [SMILES: Cc1cc([C@H]2[C@H]3CCC(F)(F)[C@H]3C[C@@H]2c2cc(=O)c3c(C(N)=O)nccc3[nH]2)c(C)c(F)c1F]
2-((2S,3R,3aR,6aS)-3-(3,4-difluoro-2-methylphenyl)-
6,6-difluorohexahydrofuro[3,2-b]furan-2-y1)-4-oxo-
1,4-dihydro-1,6-naphthyridine-5-carboxamide
or a pharmaceutically acceptable salt thereof.

36. A compound selected from: 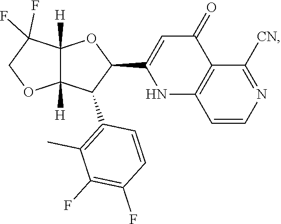 [SMILES: Cc1c([C@@H]2[C@@H]3OCC(F)(F)[C@@H]3O[C@H]2c2cc(=O)c3c(C#N)nccc3[nH]2)ccc(F)c1F]
2-((2R,3S,3aS,6aR)-3-(3,4-
difluoro-2-methylphenyl)-6,6-
difluorohexahydrofuro[3,2-
b]furan-2-yl)-4-oxo-1,4-dihydro-
1,6-naphthyridine-5-carbonitrile 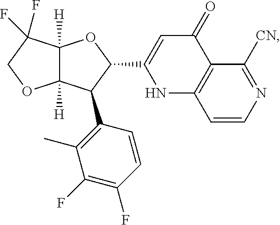 [SMILES: Cc1c([C@H]2[C@H]3OCC(F)(F)[C@H]3O[C@@H]2c2cc(=O)c3c(C#N)nccc3[nH]2)ccc(F)c1F]
2-((2S,3R,3aR,6aS)-3-(3,4-
difluoro-2-methylphenyl)-6,6-
difluorohexahydrofuro|3,2-
b]furan-2-yl)-4-oxo-1,4-dihydro-
1,6-naphthyridine-5-carbonitrile 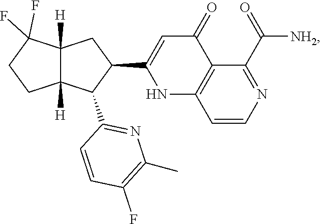 [SMILES: Cc1nc([C@@H]2[C@@H]3CCC(F)(F)[C@@H]3C[C@H]2c2cc(=O)c3c(C(N)=O)nccc3[nH]2)ccc1F]
2-((1R,2R,3aR,6aS)-4,4-difluoro-
1-(5-fluoro-6-methylpyridin-2-
yl)octahydropentalen-2-yl)-4-oxo-
1,4-dihydro-1,6-naphthyridine-5-
carboxamide 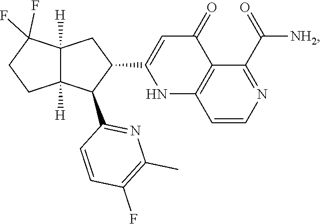 [SMILES: Cc1nc([C@H]2[C@H]3CCC(F)(F)[C@H]3C[C@@H]2c2cc(=O)c3c(C(N)=O)nccc3[nH]2)ccc1F]
2-((1S,2S,3aS,6aR)-4,4-difluoro-1-
(5-fluoro-6-methylpyridin-2-
yl)octahydropentalen-2-yl)-4-oxo-
1,4-dihydro-1,6-naphthyridine-5-
carboxamide 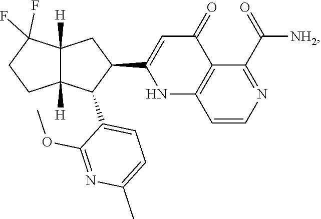 [SMILES: COc1nc(C)ccc1[C@@H]1[C@@H]2CCC(F)(F)[C@@H]2C[C@H]1c1cc(=O)c2c(C(N)=O)nccc2[nH]1]
2-((1R,2R,3aR,6aS)-4,4-difluoro-
1-(2-methoxy-6-methylpyridin-3-
yl)octahydropentalen-2-yl)-4-oxo-
1,4-dihydro-1,6-naphthyridine-5-
carboxamide 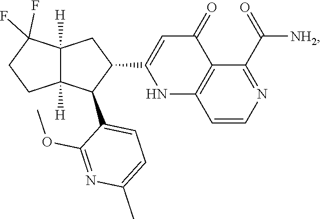 [SMILES: COc1nc(C)ccc1[C@H]1[C@H]2CCC(F)(F)[C@H]2C[C@@H]1c1cc(=O)c2c(C(N)=O)nccc2[nH]1]
2-((1S,2S,3aS,6aR)-4,4-difluoro-1-
(2-methoxy-6-methylpyridin-3-
yl)octahydropentalen-2-y1)-4-oxo-
1,4-dihydro-1,6-naphthyridine-5-
carboxamide 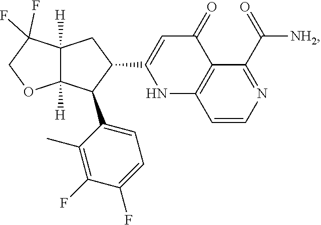 [SMILES: Cc1c([C@H]2[C@H]3OCC(F)(F)[C@H]3C[C@@H]2c2cc(=O)c3c(C(N)=O)nccc3[nH]2)ccc(F)c1F]
2-((3aS,5S,6S,6aR)-6-(3,4-difluoro-2-methylphenyl)-
3,3-difluorohexahydro-2H-cyclopenta[b]furan-5-yl)-
4-oxo-1,4-dihydro-1,6-naphthyridine-5-carboxamide 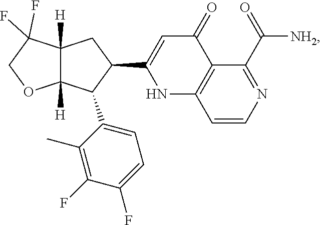 [SMILES: Cc1c([C@@H]2[C@@H]3OCC(F)(F)[C@@H]3C[C@H]2c2cc(=O)c3c(C(N)=O)nccc3[nH]2)ccc(F)c1F]
2-((3aR,5R,6R,6aS)-6-(3,4-difluoro-2-methylphenyl)-
3,3-difluorohexahydro-2H-cyclopenta[b]furan-5-yl)-
4-oxo-1,4-dihydro-1,6-naphthyridine-5-carboxamide
or a pharmaceutically acceptable salt thereof.

37. The compound of claim 1 in non-salt form.

38. A pharmaceutical composition comprising the compound of claim 1, or a pharmaceutically acceptable salt thereof, and one or more pharmaceutically acceptable carriers or vehicles.

39. A method of inhibiting a voltage-gated sodium channel in a subject comprising administering to the subject the compound of claim 1, or a pharmaceutically acceptable salt thereof;
optionally wherein the voltage-gated sodium channel is Na_{V}1.8.

40. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of claim 1, or a pharmaceutically acceptable salt thereof, optionally wherein:
(i) the method comprises treating or lessening the severity in the subject of neuropathic pain, optionally wherein:
(a) the neuropathic pain comprises post-herpetic neuralgia; or
(b) the neuropathic pain comprises small-fiber neuropathy: or
(c) the neuropathic pain comprises idiopathic small-fiber neuropathy; or
(d) the neuropathic pain comprises diabetic neuropathy, optionally wherein the diabetic neuropathy comprises diabetic peripheral neuropathy; or
(ii) the method comprises treating or lessening the severity in the subject of chronic pain, optionally wherein the chronic pain is lumbosacral radiculopathy: or
(iii) the method comprises treating or lessening the severity in the subject of musculoskeletal pain, optionally wherein the musculoskeletal pain comprises osteoarthritis pain; or
(iv) the method comprises treating or lessening the severity in the subject of acute pain, optionally wherein the acute pain comprises acute post-operative pain; or
(v) the method comprises treating or lessening the severity in the subject of postsurgical pain, optionally wherein:
(a) the postsurgical pain comprises bunionectomy pain; or
(b) the postsurgical pain comprises abdominoplasty pain; or
(c) the postsurgical pain comprises herniorrhaphy pain; or
(vi) the method comprises treating or lessening the severity in the subject of visceral pain.

41. (canceled)

42. The method of claim 40, wherein said subject is treated with one or more additional therapeutic agents administered concurrently with, prior to, or subsequent to treatment with the compound, pharmaceutically acceptable salt, or pharmaceutical composition.

43. (canceled)
